(19) United States
(12) Patent Application Publication
(10) Pub. No.: US 2025/0282771 A1
(43) Pub. Date: Sep. 11, 2025

(54) TETRAHYDROISOQUINOLINE HETEROBIFUNCTIONAL BCL-XL DEGRADERS

(71) Applicant: Treeline Biosciences, Inc., Watertown, MA (US)

(72) Inventors: Evan J. Horn, San Diego, CA (US); Joshua D. Hansen, San Diego, CA (US); Matthew D. Alexander, San Diego, CA (US); Fei Huang, San Diego, CA (US); Mark A. Nagy, San Diego, CA (US)

(21) Appl. No.: 18/859,011

(22) PCT Filed: May 4, 2023
(86) PCT No.: PCT/US2023/020987

§ 371 Date: Oct. 22, 2024

Related U.S. Application Data
(60) Provisional application No. 63/339,262, filed on May 6, 2022.
(60) Provisional application No. 63/398,769, filed on Aug. 17, 2022.
(60) Provisional application No. 63/429,814, filed on Dec. 2, 2022.
(60) Provisional application No. 63/454,477, filed on Mar. 24, 2023.

(51) Int. Cl. C07D 417/14; A61K 31/4725; A61K 31/496; A61K 31/519; A61K 31/529; A61K 31/5377; A61K 31/635

(52) U.S. Cl. CPC C07D 417/14; A61K 31/4725; A61K 31/496; A61K 31/519; A61K 31/529; A61K 31/5377; A61K 31/635

(57) ABSTRACT

This disclosure provides compounds of Formula (I) or (II) (e.g. Formula (I-A), (I-B), or (I-C)), or a pharmaceutically acceptable salt thereof, that induce degradation of a BCL-X_{L} protein. These compounds are useful, for example, for treating a cancer in a subject (e.g., a human). This disclosure also provides compositions containing the compounds, or pharmaceutically acceptable salts thereof, provided herein as well as methods of using and making the same.

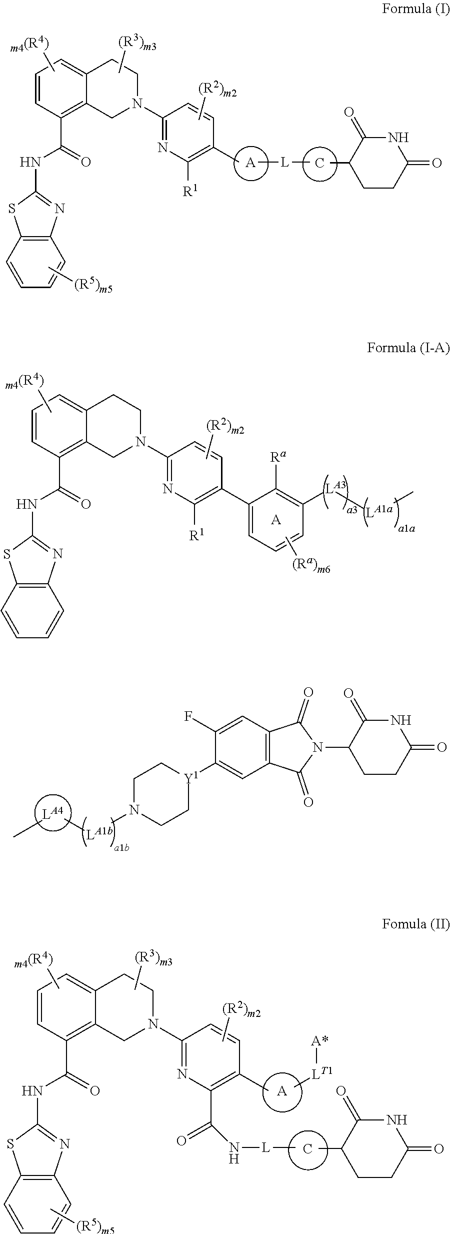

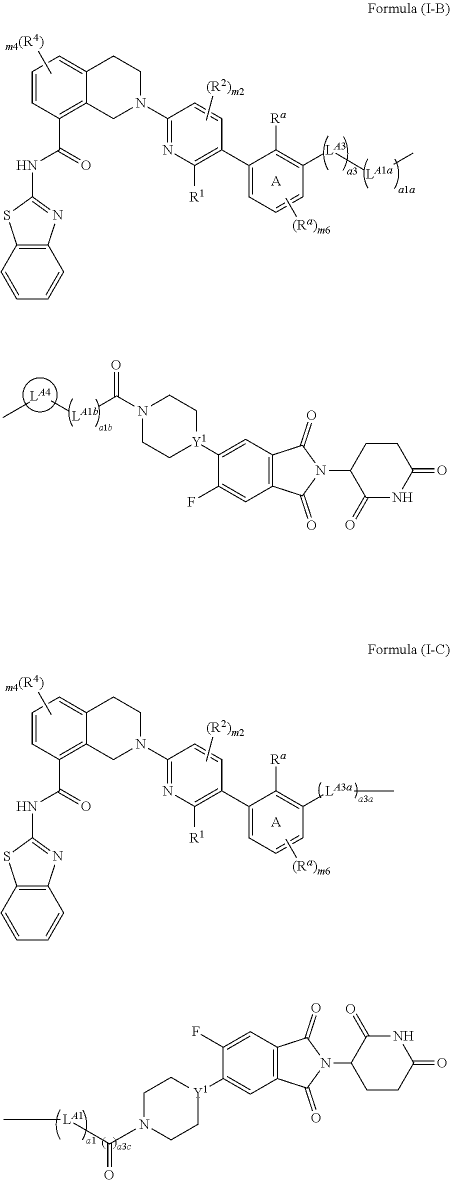

### CROSS-REFERENCE TO RELATED APPLICATIONS

This application claims priority to U.S. Provisional Application Ser. No. 63/454,477, filed Mar. 24, 2023; 63/429,814, filed Dec. 2, 2022; 63/398,769, filed Aug. 17, 2022; and 63/339,262, filed May 6, 2022; each of which is incorporated by reference in its entirety herein.

### TECHNICAL FIELD

This disclosure provides compounds of Formula (I) or (II) (e.g., Formula (I-A), (I-B), or (I-C)), or pharmaceutically acceptable salts thereof, that induce degradation of a BCL-X_{L} protein. These compounds are useful, for example, for treating cancer in a subject (e.g., a human). This disclosure also provides compositions containing the compounds, or pharmaceutically acceptable salts thereof, provided herein as well as methods of using and making the same.

### BACKGROUND

The BCL-2 family of proteins is involved in the regulation of cell apoptosis and includes proteins that are pro-apoptosis, pro-survival, and BH3-only. At a high level, the balance of binding of BH3-only proteins to the pro-apoptosis and pro-survival members of the BCL-2 family can determine whether a cell will undergo apoptosis. The protein BCL-X_{L}, encoded by the BCL2L1 gene, is a pro-survival member of the BCL-2 family. In many cancers, it can be desirable to initiate apoptosis of tumor cells, which may be achieved by decreasing the amount of pro-survival protein (e.g., BCL-X_{L}) available to compete for BH3-only protein binding.

### SUMMARY

This disclosure provides compounds of Formula (I) or (II) (e.g., Formula (I-A), (I-B), or (I-C)), or pharmaceutically acceptable salts thereof, that induce degradation of a BCL-X_{L} protein. These compounds are useful, e.g., for treating a cancer in a subject (e.g., a human). This disclosure also provides compositions containing the compounds, or pharmaceutically acceptable salts thereof, provided herein as well as methods of using and making the same.

Provided herein are compounds of Formula (I) or (II):

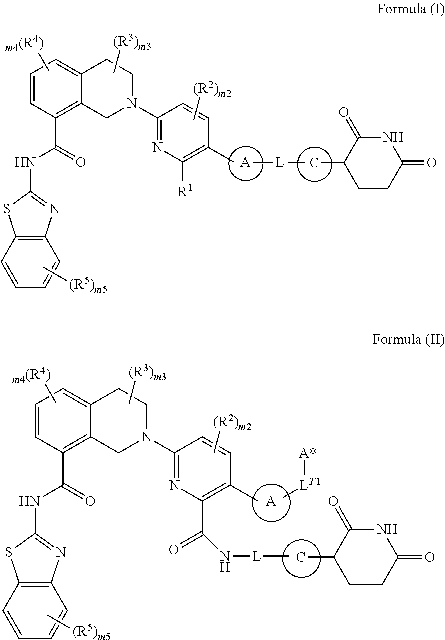

or pharmaceutically acceptable salts thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b};
L^{T1} is a bond or C₁₋₃ alkylene optionally substituted with 1-3 substituents independently selected from the group consisting of: oxo and R^{c}, wherein one CH₂ unit of the C₁₋₃ alkylene is optionally replaced with —O— or —N(R^{d})—;
A* is selected from the group consisting of:
(a) C₃₋₁₅ cycloalkyl or 3-15 membered heterocyclyl, each of which is optionally substituted with 1-6 substituents independently selected from the group consisting of: R^{a} and R^{b}.
(b) C₆₋₁₅ aryl or 5-15 membered heteroaryl, each of which is optionally substituted with 1-6 substituents independently selected from the group consisting of: R^{a} and R^{b}; and
(c) H;
R¹ is selected from the group consisting of:
(a) C(O)OH,
(b) C(O)NR^{d}R^{c}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):

### AA

n1 is an integer from 3 to 15; and
each L^{A} is independently selected from the group consisting of: L^{A1}, L^{A3}, and L^{A4} provided that 1-3 occurrences of L^{A} is L^{A4};

### BB

n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any O—O, N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
Ring C is selected from the group consisting of:

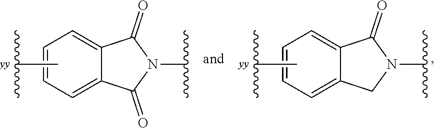

each of which is optionally substituted with 1-3 substituents independently selected from the group consisting of: R^{a} and R^{b}, wherein yy is the point of attachment to L;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{h} is independently selected from the group consisting of: —O—; —N(H)—; —N(C₁₋₃ alkyl)-; —S(O)₀₋₂—; C(═O); and C₁₋₃ alkylene, and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Also provided herein is a pharmaceutical composition comprising a compound of Formula (I) or (II) (e.g., Formula (I-A), (I-B), or (I-C)), or a pharmaceutically acceptable salt thereof, and a pharmaceutically acceptable carrier.

Provided herein is a method for treating cancer in a subject in need thereof, the method comprising administering to the subject a therapeutically effective amount of a compound of Formula (I) or (II) (e.g., Formula (I-A), (I-B), or (I-C)), or a pharmaceutically acceptable salt thereof, or a pharmaceutical composition as provided herein.

Also provided herein is a BCL-X_{L} protein non-covalently bound with a compound of Formula (I) or (II) (e.g., Formula (I-A), (I-B), or (I-C)), or a pharmaceutically acceptable salt thereof.

Also provided herein is a ternary complex comprising a BCL-X_{L} protein, a compound of Formula (I) or (II) (e.g., Formula (I-A), (I-B), or (I-C)), or a pharmaceutically acceptable salt thereof, and a CRBN protein, or a portion thereof.

To facilitate understanding of the disclosure set forth herein, a number of additional terms are provided. Generally, the nomenclature used herein and the laboratory procedures in organic chemistry, medicinal chemistry, and pharmacology described are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The details of one or more embodiments of the invention are set forth in the accompanying drawings and the description below. Other features and advantages of the invention will be apparent from the description and drawings, and from the claims.

### DETAILED DESCRIPTION

This disclosure provides compounds of Formula (I) or (II) (e.g., Formula (I-A), (I-B), or (I-C)), or pharmaceutically acceptable salts thereof, that induce degradation of a BCL-X_{L}(also referred to as Bcl-xL herein) protein. These compounds are useful, e.g., for treating a cancer. This disclosure also provides compositions containing the compounds, or pharmaceutically acceptable salts thereof, provided herein as well as methods of using and making the same.

Without being bound by any particular theory, it is believed that in healthy cells, pro-apoptotic effectors such as BAX and BAK can move between the cytosol and the mitochondrial outer membrane (MOM), on which Voltage Dependent Anion Channel 2 (VDAC2) can act as a receptor. Pro-survival BCL-2 family members (e.g., BCL-2, BCL-X_{L}, and MCL-1) can retrotranslocate BAX back to the cytosol. A BH3-only protein (e.g., BIM) can engage a rear site on a pro-apoptotic effector (e.g., BAX or BAK) and release the C-terminal transmembrane domain (α9) of the effector, enabling binding to the MOM. Binding of BIM to the canonical BH3 binding groove of BAX or BAK releases the N-terminus and α1 of BAX or BAK, and subsequent unfolding of the “latch” domain releases BIM from the BH3 binding groove. If a pro-survival BCL-2 family member then binds to BAX or BAK, apoptotic signaling is generally halted. However, if BAX or BAK are allowed to dimerize, then oligomerize, the MOM can be permeabilized, leading to apoptosis.

An abundance of pro-survival BCL-2 family members is sometimes thought to “prime” cells for death (e.g., via cytotoxic therapies, including BH3 mimetics). It is believed that oncogenic mutations and stresses can cause an upregulation in BH3-only proteins, thus placing a selective pressure on cancer cells for upregulation of the pro-survival BCL-2 family proteins. Thus, with more BH3-only proteins around, the cells are believed to be more sensitive to further manipulations of the BCL-2 family balance. See, e.g., Adams and Cory, Cell Death & Differentiation 25.1 (2018): 27-36.

Compounds that induce degradation of a target protein are sometimes referred to as heterobifunctional compounds, PROTACs, or degraders. Such compounds generally include a moiety that binds to the target protein and a moiety that binds to a ubiquitin E3 ligase (sometimes referred to as an E3 ligase or simply an E3), these two moieties being optionally separated by a linker. To induce degradation, heterobifunctional compounds are believed to induce formation of a ternary complex between the target protein, the compound, and an E3 ligase. Formation of the ternary complex is then followed by ubiquitination of the target protein and degradation of the ubiquitinated target protein by a proteosome. Several E3 ligases have been used as the partner E3 ligase for heterobifunctional degraders. Herein, the cereblon (CRBN) E3 ligase (also referred to herein as a CRBN protein) is used.

A degradation approach for a target protein can have potential advantages compared to, e.g., small molecule inhibition of the target protein. One potential advantage is that the duration of effect of a heterobifunctional compound is generally based on the resynthesis rate of the target protein. Another potential advantage is that many heterobifunctional compounds are believed to be released from the ubiquitinated target protein-E3 ligase complex and made available for formation of further ternary complexes; this is sometimes referred to as “catalytic” turnover of the heterobifunctional compound. Degradation of a target protein can also be advantageous over small molecule inhibition in some cases, as degradation can impair a scaffolding function of a target protein, whereas a small molecule might not. It is also generally believed that for formation of a ternary complex, high affinity to the target protein is not always required.

Heterobifunctional compounds are further described in, for example, International Publication Nos. WO 2017/184995; WO 2019/144117; WO 2020/163823; WO 2021/078301; WO 2021/146536; WO 2021/007307; WO 2021/222114; WO 2021/078301; WO 2022/169780; WO 2023/044046; Chamberlain and Hamann, Nature Chemical Biology 15.10 (2019): 937-944; Li and Song, Journal of Hematology & Oncology 13 (2020): 1-14; Wu, et al. Nature Structural & Molecular Biology 27.7 (2020): 605-614; Dong, et al., Journal of Medicinal Chemistry 64.15 (2021): 10606-10620; Yang, et al., Targeted Oncology 16.1 (2021): 1-12; Lv, et al., Nature Communications 12.1 (2021): 6896.

### Compound Embodiments

Provided herein are compounds of Formula (I) or (II):

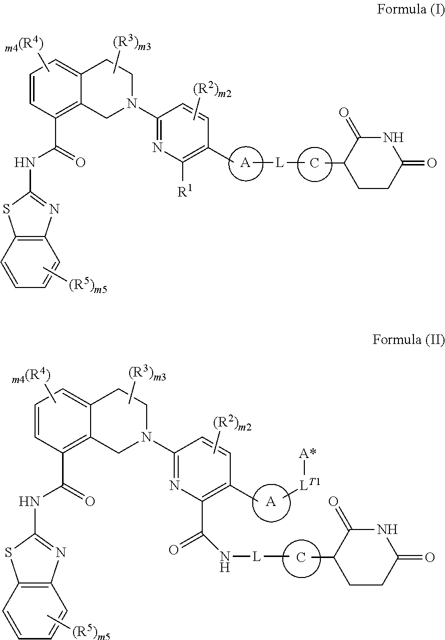

or pharmaceutically acceptable salts thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b};
L^{T1} is a bond or C₁₋₃ alkylene optionally substituted with 1-3 substituents independently selected from the group consisting of: oxo and R^{c}, wherein one CH₂ unit of the C₁₋₃ alkylene is optionally replaced with —O— or —N(R^{d})—;
A* is selected from the group consisting of:
(a) C₃₋₁₅ cycloalkyl or 3-15 membered heterocyclyl, each of which is optionally substituted with 1-6 substituents independently selected from the group consisting of: R^{a} and R^{b};
(b) C₆₋₁₅ aryl or 5-15 membered heteroaryl, each of which is optionally substituted with 1-6 substituents independently selected from the group consisting of: R^{a} and R^{b}; and
(c) H;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{e};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):

### AA

n1 is an integer from 3 to 15; and
each L^{A} is independently selected from the group consisting of: L^{A1}, L^{A3}, and L^{A4} provided that 1-3 occurrences of L^{A} is L^{A4};

### BB

n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any O—O, N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{e};
Ring C is selected from the group consisting of:

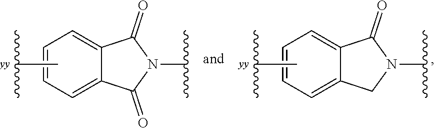

each of which is optionally substituted with 1-3 substituents independently selected from the group consisting of: R^{a} and R^{b}, wherein yy is the point of attachment to L;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene, and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R is independently selected from the group consisting of: H and C₁₋₆ alkyl optionally substituted with 1-3 R^{h};
each R⁹ is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In some embodiments, it is provided that one or both of (i) and (ii) applies:
(i) Ring C is substituted with 1-3 substituents independently selected from the group consisting of: R^{a} and R^{b}; and
(ii) in Formula (I), L does not contain an adamantylene group, and in Formula (II), A* is other than adamantyl.

In some embodiments, it is provided that (i) applies. In some embodiments, it is provided that (ii) applies. In some embodiments, it is provided that both (i) and (ii) apply.

In some embodiments, the compounds are compounds of Formula (I) or pharmaceutically acceptable salts thereof.

In some embodiments, the compounds are compounds of Formula (II) or pharmaceutically acceptable salts thereof.

In some embodiments, Ring A is phenylene or 5-6 membered heteroarylene, each of which is optionally substituted with 1-3 substituents independently selected from the group consisting of: R^{a} and R^{b}.

In some embodiments, Ring A is phenylene optionally substituted with 1-3 substituents independently selected from the group consisting of: R^{a} and R^{b}. In some embodiments, Ring A is a phenylene optionally substituted with 1-3 R^{a}.

In some embodiments, Ring A is

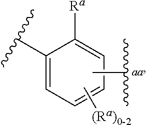

wherein aa represents the point of attachment to L or L^{T1}.

In some embodiments, Ring A is

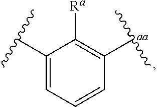

wherein aa represents the point of attachment to L or L^{T1}. For example, Ring A can be

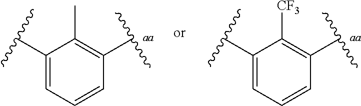

wherein aa represents the point of attachment to L or L^{T1}.

In some embodiments, Ring A is

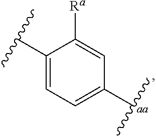

wherein aa represents the point of attachment to L or L^{T1}. For example, Ring A can be

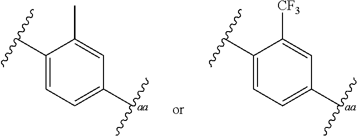

wherein aa represents the point of attachment to L or L^{T}1.

In some embodiments, Ring A is 5-6 membered heteroarylene optionally substituted with 1-3 R^{a}. In some embodiments, Ring A is 5-6 membered heteroarylene optionally substituted with 1-2 R^{a}. In some embodiments, Ring A is 5-membered heteroarylene optionally substituted with 1-2 R^{a}. In some embodiments, Ring A is pyrazolylene optionally substituted with 1-2 R^{a}. For example, Ring A is selected from the group consisting of:

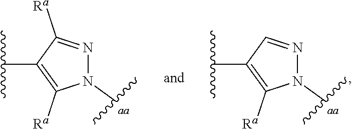

wherein aa represents the point of attachment to L or L^{T1}.

In some embodiments, Ring A is C₃₋₁₀ cycloalkylene optionally substituted with 1-6 R^{a}. In some embodiments, Ring A is C₄₋₆ cycloalkylene optionally substituted with 1-3 R^{a}. In some embodiments, Ring A is cyclohexylene optionally substituted with 1-3 R^{a}. For example, Ring A can be 1,4-cyclohexylene.

In some embodiments, one R^{a} present on Ring A is C₁₋₃ alkyl optionally substituted with 1-3 F. In some embodiments, one R^{a} present on Ring A is methyl or CF₃.

In some embodiments, R¹ is C(O)OH. In some embodiments, R¹ is C(O)NHR^{e}.

In some embodiments, R¹ is C(O)OH; and Ring A is

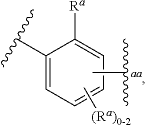

wherein aa represents the point of attachment to L or L^{T1}. In some embodiments, Ring A is

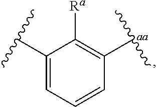

wherein aa represents the point of attachment to L or L^{T}. For example, Ring A can be

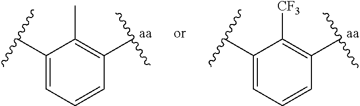

or wherein aa represents the point of attachment to L or L^{T1}. In some embodiments, Ring A is

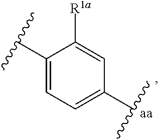

wherein aa represents the point of attachment to L or L^{T1}. For example, Ring A can be

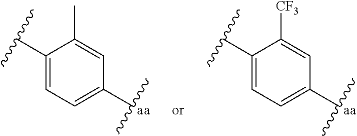

wherein aa represents the point of attachment to L or L^{T1}.

In some embodiments, L^{T1} is C₁₋₃ alkylene. For example, L^{T1} can be —CH₂—.

In some embodiments, A* is H. In some embodiments, A* is C₃₋₁₅ cycloalkyl or 3-15 membered heterocyclyl, each of which is optionally substituted with 1-6 substituents independently selected from the group consisting of: R^{a} and R^{b}. In some embodiments, A* is C₃₋₁₅ cycloalkyl optionally substituted with 1-3 R^{a}. For example, A* can be adamantyl optionally substituted with 1-3 R^{a}.

In some embodiments, m2 is 0. In some embodiments, m3 is 0. In some embodiments, m4 is 0. In some embodiments, m5 is 0.

In some embodiments, m2 is 0; m3 is 0; m4 is 0; and m5 is 0.

In some embodiments, Ring C is selected from the group consisting of:

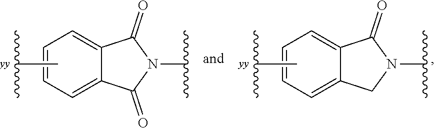

each of which is optionally substituted with 1-3 R^{a}, wherein yy is the point of attachment to L.

In some embodiments, Ring C is selected from the group consisting of:

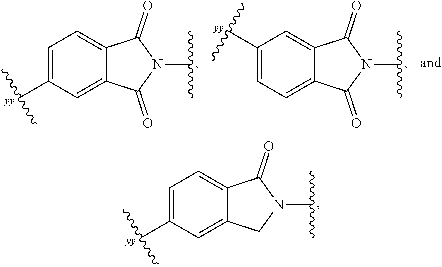 [SMILES: *c1ccc2c(c1)C(=O)N(*)C2=O ; *c1ccc2c(c1)C(=O)N(*)C2=O ; *c1ccc2c(c1)CN(*)C2=O]

each of which is substituted with 1-3 R^{a}, wherein yy is the point of attachment to L.

In some embodiments, Ring C is

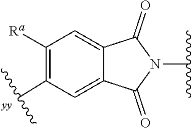

further optionally substituted with 1-2 R^{a} at one or more ring carbon atoms, wherein yy is the point of attachment to L. In some embodiments, Ring C is

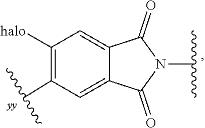

wherein yy is the point of attachment to L. For example, Ring C can be

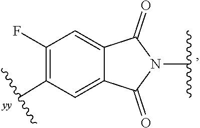

wherein yy is the point of attachment to L.

In some embodiments, L is -(L^{A})ₙ₁-; and L^{A} and n1 are defined according to (AA). In some embodiments, n1 is an integer from 3 to 5. In some embodiments, n1 is an integer from 5 to 9. In some embodiments, n1 is 6, 7, or 8. In some embodiments, n1 is an integer from 9-12.

In some embodiments, L^{A} and n1 are defined according to (AA); and 1-2 occurrences of L^{A} is L^{A4}. In some embodiments, one occurrence of L^{A} is L^{A4}. In some embodiments, two occurrences of L^{A} are L^{A4}. In some embodiments, each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

In some embodiments, L^{A} and n1 are defined according to (AA); and 1-4 occurrences of L^{A} is L^{A3}. In some embodiments, 1-3 (e.g., 1-2 or 2-3) occurrences of L^{A} is L^{A3}. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L^{A} and n1 are defined according to (AA); and 2-7 occurrences of L^{A} are L^{A1}. In some embodiments, 2-5 occurrences of L^{A} are L^{A1}. In some embodiments, 0-2 (e.g., 0-1) occurrences of L^{A1} are —CHR^{L}— or —C(R^{L})₂—; and each remaining occurrence of L^{A1} is —CH₂—. In some embodiments, each occurrence of L^{A1} is —CH₂—. In some embodiments, one occurrence of L^{A1} is —CHR^{L}— or —C(R^{L})₂—; and each remaining occurrence of L^{A1} is —CH₂—. In some embodiments, each R^{L} is independently selected from the group consisting of: —F and —C₁₋₃ alkyl optionally substituted with 1-3 F (e.g., CH₃ or CF₃).

In some embodiments, L^{A} and n1 are defined according to (AA); and 8-13 occurrences of L^{A} are L^{A1}. In some embodiments, 0-2 occurrences of L^{A1} is —CHR^{L}— or —C(R^{L})₂—; and each remaining occurrence of L^{A1} is —CH₂—. In some embodiments, each occurrence of L^{A1} is —CH₂—. In some embodiments, one occurrence of L^{A1} is —CHR^{L}— or —C(R^{L})₂—; and each remaining occurrence of L^{A1} is —CH₂—. In some embodiments, each R^{L} is independently selected from the group consisting of: —F and —C₁₋₃ alkyl optionally substituted with 1-3 F.

In some embodiments, L is -(L^{A})ₙ₁-, and L^{A} and n1 are defined according to (AA), wherein: n1 is an integer from 5 to 9; 2 occurrences of L^{A} are L^{A4}; 2-7 occurrences of L^{A} are L^{A1}; and 1-3 occurrences of L^{A} is L^{A3}. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is -(L^{A})ₙ₁-, and L^{A} and n1 are defined according to (AA), wherein: n1 is an integer from 5 to 13; 1 occurrence of L^{A} is L^{A4}; 2-11 occurrences of L^{A} are L^{A1}; and 1-3 occurrences of L^{A} is L^{A3}. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is selected from the group consisting of: (i) -(L^{A3})₀₋₂-(L^{A1})₀₋₅-L^{A4}-(L^{A1})₀₋₅-L^{A3}-L^{A4}-_{bb}; and (ii) -(L^{A3})₀₋₂-(L^{A1})₀₋₅-L^{A4}-(L^{A1})₀₋₅-L^{A4}-_{bb}; provided that L contains 2-7 L^{A1}; and wherein bb represents the point of attachment to Ring C. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is: -(L^{A3})₀₋₂-(L^{A1})₁₋₁₁-(L^{A3})₀₋₁-L^{A4}-_{bb}, wherein bb represents the point of attachment to Ring C. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is:
-(L^{A3})₀₋₁-(L^{A1})₀₋₅-L^{A4}-(L^{A1})₀₋₅-L^{A4}-_{bb}; or
-(L^{A3})₀₋₁-(L^{A1})₀₋₅-L^{A4}-(L^{A1})₀₋₅-L^{A3}-L^{A4}-_{bb};
provided that L contains 2-7 L^{A1}; and wherein bb represents the point of attachment to Ring C. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is:
-(L^{A3})₀₋₁-(L^{A1})₀₋₅-L^{A4}-(L^{A1})₀₋₅-(4-10 membered heterocyclylene)-_{bb}; or
-(L^{A3})₀₋₁-(L^{A1})₀₋₅-L^{A4}-(L^{A1})₀₋₅-C(═O)-(4-10 membered heterocyclylene)-_{bb}; provided that L contains 2-7 L^{A1}; and wherein bb represents the point of attachment to Ring C. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—.

In some embodiments, L is:
-(L^{A3})₀₋₁-(L^{A1})₀₋₅-L^{A4}-(L^{A1})₀₋₅-(6-membered heterocyclylene)-_{bb}; or
-(L^{A3})₀₋₁-(L^{A1})₀₋₅-L^{A4}-(L^{A1})₀₋₅-C(═O)-(6-membered heterocyclylene)-_{bb};
provided that L contains 2-5 L^{A1}; and wherein bb represents the point of attachment to Ring C. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—.

In some embodiments, L is a divalent group of Formula (L-1) or (L-2):

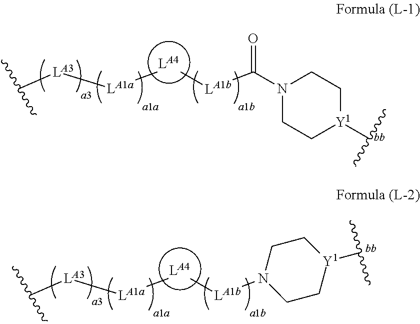

wherein:
Y¹ is N or CH;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

In some embodiments of (L-1) or (L-2), a3 is 1. In some embodiments, L^{A3} is —O—. In some embodiments, a3 is 1 and L^{A3} is —O—.

In some embodiments of (L-1) or (L-2), a1a+a1b is 3 or 4. For example, a1a+a1b can be 3. For example, a1a+a1b can be 4.

In some embodiments of (L-1) or (L-2), a1a+a1b is 2.

In some embodiments of (L-1) or (L-2), each occurrence of L^{A1a} and L^{A1b} is —CH₂—.

In some embodiments of (L-1) or (L-2), one occurrence of L^{A1a} is —CHR^{L}— or —C(R^{L})₂—; each remaining occurrence of L^{A1a} is —CH₂—; and each occurrence of L^{A1b} is —CH₂—.

In some embodiments of (L-1) or (L-2), one occurrence of L^{A1b} is —CHR^{L}— or —C(R^{L})₂—; each remaining occurrence of L^{A1b} is —CH₂—; and each occurrence of L^{A1a} is —CH₂—.

In some embodiments of (L-1) or (L-2), L^{A4} is 4-10 membered heterocyclylene optionally substituted with 1-3 R^{a}. In some embodiments of (L-1) or (L-2), L^{A4} is selected from the group consisting of:

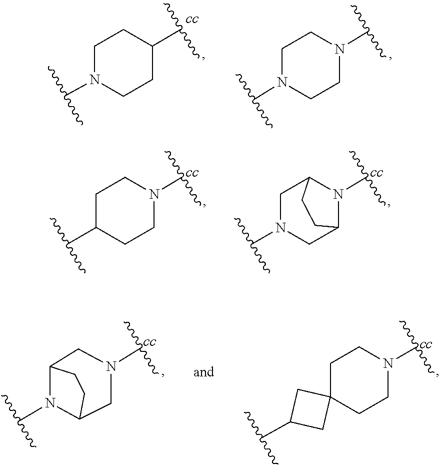 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}. In some embodiments of (L-1) or (L-2), each R^{a} present on L^{A4} is C₁₋₃ alkyl optionally substituted with 1-3 F.

In some embodiments of (L-1) or (L-2), L^{A4} is C₃₋₁₀ cycloalkylene optionally substituted with 1-3 R^{a}. For example, L^{A4} can be 1,4-cyclohexylene optionally substituted with 1-3 R^{a}. For example, L^{A4} can be

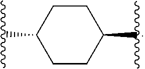 [SMILES: *[C@H]1CC[C@H](*)CC1]

In some embodiments of (L-1) or (L-2), L^{A4} is phenylene or 5-6 membered heteroarylene, each of which is optionally substituted with 1-3 R^{a}. For example, L^{A4} can be 1,4-phenylene optionally substituted with 1-3 R^{a}. For example, L^{A4} can be 1,2-phenylene optionally substituted with 1-3 R^{a}. For example, L^{A4} can be 1,3-phenylene optionally substituted with 1-3 R^{a}.

In some embodiments of (L-1) or (L-2), Y¹ is N. In some embodiments of (L-1) or (L-2), Y¹ is CH.

L is: -(L^{A3})₀₋₁-(L^{A1})₁₋₁₁-(L^{A3})₀₋₁-L^{A4}-_{bb}, wherein bb represents the point of attachment to Ring C. In some embodiments, L is: -(L^{A3})₀₋₁-(L^{A1})₁₋₁₁-(C(═O))₀₋₁-(4-10 membered heterocyclylene)-_{bb}, wherein bb represents the point of attachment to Ring C. In some embodiments, L is: -(L^{A3})₀₋₁-(L^{A1})₁₋₁₁-(C(═O))₀₋₁-(6 membered heterocyclylene)-_{bb}, wherein bb represents the point of attachment to Ring C. In some embodiments, L is: -(L^{A3})₀₋₁-(L^{A1})₁₋₁₁-(C(═O))₀₋₁-(piperazinylene or piperidinylene (e.g., piperazinylene))-_{bb}, wherein bb represents the point of attachment to Ring C.

In some embodiments, L is a divalent group of Formula (L-3):

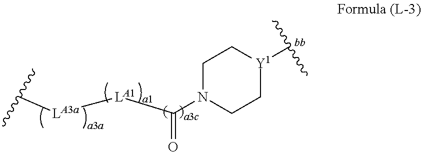

wherein:
Y¹ is N or CH;
a3a is 0 or 1;
L^{A3a} is selected from the group consisting of: —O—, —N(H)—, and —N(C₁₋₃ alkyl)-;
a1 is an integer from 1 to 11;
a3c is 0 or 1; and
bb represents the point of attachment to Ring C.

In some embodiments of (L-3), a3a is 1. In some embodiments, L^{A3a} is —O—. In some embodiments, a3a is 1 and L^{A3a} is —O—.

In some embodiments of (L-3), a3a is 0.

In some embodiments of (L-3), a1 is 1 or 2. In some embodiments of (L-3), a1 is an integer from 3 to 5. In some embodiments of (L-3), a1 is an integer from 5 to 9. In some embodiments of (L-3), a1 is an integer from 9 to 11.

In some embodiments of (L-3), 0-1 occurrence of L^{A1} is —CHR^{L}— or —C(R^{L})₂—; and each remaining occurrence of L^{A1} is —CH₂—.

In some embodiments of (L-3), a3c is 0. In some embodiments of (L-3), a3c is 1.

In some embodiments of (L-3), Y¹ is N. In some embodiments of (L-3), Y¹ is CH.

In some embodiments (e.g., when L is (L-1) or (L-2)), L is selected from the group consisting of:

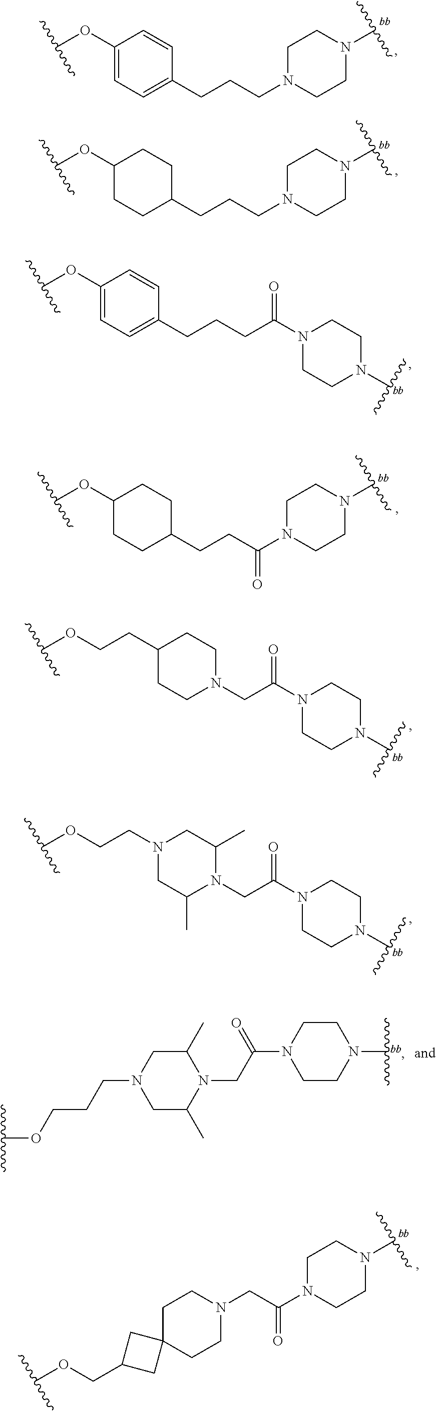

wherein bb represents the point of attachment to Ring C.

In some embodiments (e.g., when L is (L-1) or (L-2)), L is selected from the group consisting of:

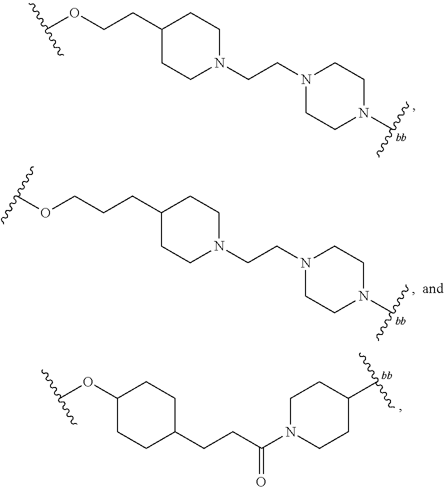

wherein bb represents the point of attachment to Ring C.

In some embodiments (e.g., when L is (L-3)), L is selected from the group consisting of:

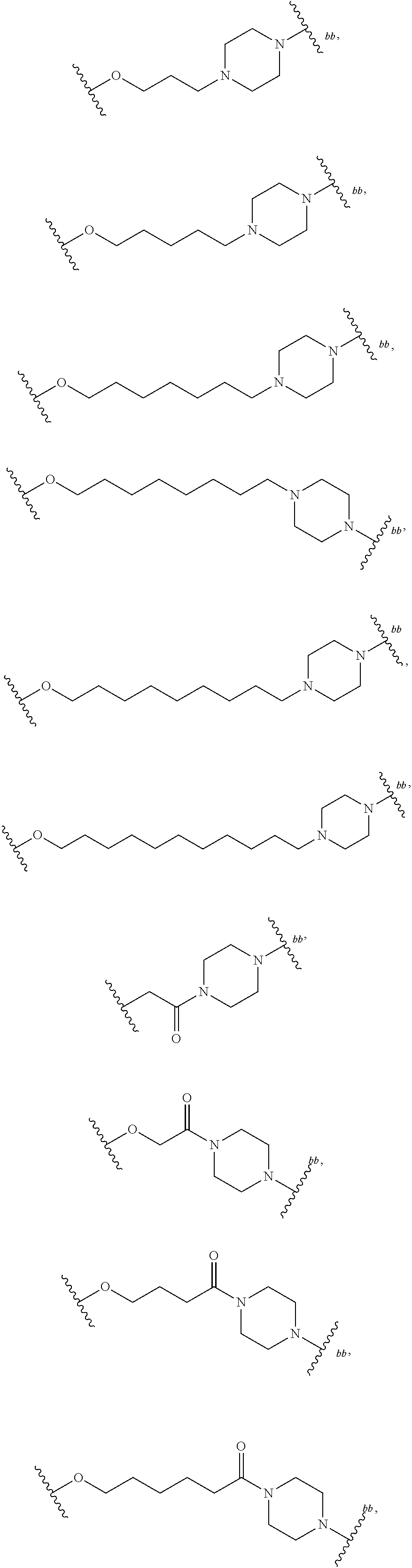

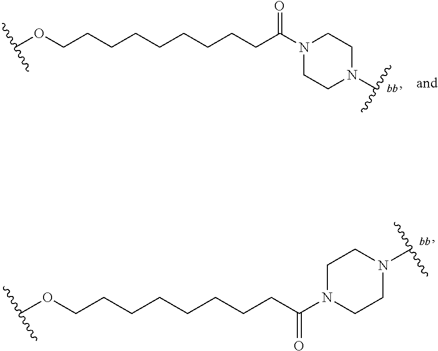

wherein bb represents the point of attachment to Ring C.

In some embodiments, L is (L-2); and the compounds of Formula (I) are compounds of Formula (I-A):

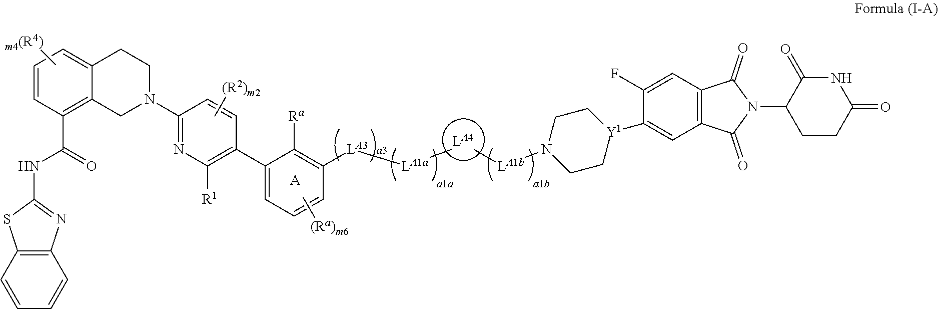

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Y¹ is N or CH.

In some embodiments, L is (L-1); and the compounds of Formula (I) are compounds of Formula (I-B):

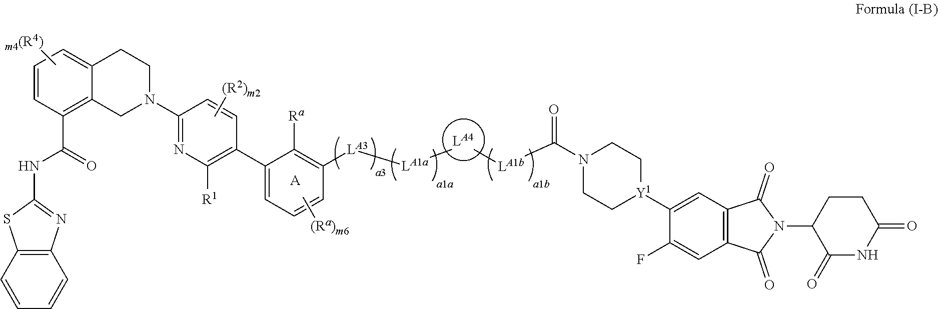

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Y¹ is N or CH.

In some embodiments of Formula (I-A) or (I-B), a3 is 1. In some embodiments, L^{A3} is —O—. In some embodiments, a3 is 1 and L^{A3} is —O—.

In some embodiments of Formula (I-A) or (I-B), a3 is 0.

In some embodiments of Formula (I-A) or (I-B), a1a+a1b is 3.

In some embodiments of Formula (I-A) or (I-B), a1a+a1b is 2.

In some embodiments of Formula (I-A) or (I-B), each occurrence of L^{A1a} and L^{A1b} is —CH₂—.

In some embodiments of Formula (I-A) or (I-B), one occurrence of L^{A1a} is —CHR^{L}— or —C(R^{L})₂—; each remaining occurrence of L^{A1a} is —CH₂—; and each occurrence of L^{A1b} is —CH₂—.

In some embodiments of Formula (I-A) or (I-B), one occurrence of L^{A1b} is —CHR^{L}— or —C(R^{L})₂—; each remaining occurrence of L^{A1b} is —CH₂—; and each occurrence of L^{A1a} is —CH₂—.

In some embodiments of Formula (I-A) or (I-B), L^{A4} is 4-10 membered heterocyclylene optionally substituted with 1-3 R^{a}. In some embodiments of Formula (I-A) or (I-B), L^{A4} is selected from the group consisting of:

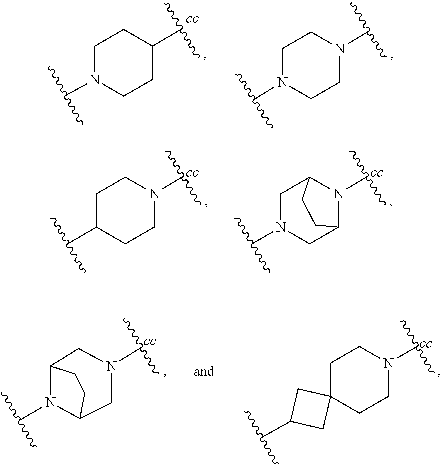 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}. In some embodiments of Formula (I-A) or (I-B), each R^{a} present on L^{A4} is C₁₋₃ alkyl optionally substituted with 1-3 F.

In some embodiments of Formula (I-A) or (I-B), L^{A4} is C₃₋₁₀ cycloalkylene optionally substituted with 1-3 R^{a}. For example, L^{A4} can be 1,4-cyclohexylene optionally substituted with 1-3 R^{a}. For example, L^{A4} can be

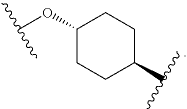 [SMILES: *O[C@H]1CC[C@H](*)CC1]

In some embodiments of Formula (I-A) or (I-B), L^{A4} is phenylene or 5-6 membered heteroarylene, each of which is optionally substituted with 1-3 R^{a}. For example, L^{A4} can be 1,4-phenylene optionally substituted with 1-3 R^{a}. For example, L^{A4} can be 1,3-phenylene optionally substituted with 1-3 R^{a}. For example, L^{A4} can be 1,2-phenylene optionally substituted with 1-3 R^{a}.

In some embodiments, L is (L-3); and the compounds of Formula (I) are compounds of Formula (I-C):

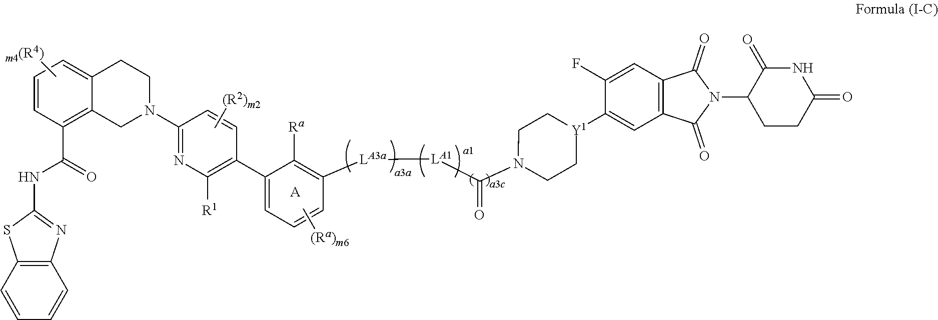

or pharmaceutically acceptable salts thereof, wherein:
m6 is 0 or 1;
a3a is 0 or 1;
L^{A3a} is selected from the group consisting of: —O—, —N(H)—, and —N(C₁₋₃ alkyl)-;
a1 is an integer from 1 to 11;
a3c is 0 or 1; and
Y¹ is N or CH.

In some embodiments of Formula (I-C), a3a is 1. In some embodiments, L^{A3a} is —O—.

In some embodiments, a3a is 1 and L^{A3a} is —O—.

In some embodiments of Formula (I-C), a1 is 1 or 2.

In some embodiments of Formula (I-C), a1 is an integer from 3 to 5. In some embodiments of Formula (I-C), a1 is an integer from 6 to 8. In some embodiments of Formula (I-C), a1 is an integer from 9 to 11.

In some embodiments of Formula (I-C), 0-1 occurrence of L^{A1} is —CHR^{L}— or —C(R^{L})₂—; and each remaining occurrence of L^{A1} is —CH₂—.

In some embodiments of Formula (I-C), a3c is 0.

In some embodiments of Formula (I-C), a3c is 1.

In some embodiments of Formula (I-A), (I-B), or (I-C), m6 is 0.

In some embodiments of Formula (I-A), (I-B), or (I-C), the R^{a} present on Ring A is C₁₋₃ alkyl optionally substituted with 1-3 —F.

In some embodiments of Formula (I-A), (I-B), or (I-C), R¹ is C(O)OH.

In some embodiments of Formula (I-A), (I-B), or (I-C), m2 is 0. In some embodiments of Formula (I-A), (I-B), or (I-C), m4 is 0.

In some embodiments of Formula (I-A), (I-B), or (I-C), Y¹ is N. In some embodiments of Formula (I-A), (I-B), or (I-C), Y¹ is CH.

In some embodiments, the compounds are selected from the group consisting of the compounds in Table C₁, or pharmaceutically acceptable salts thereof.

TABLE C1

| No. | Compound Structure |
|---|---|
| 101 | 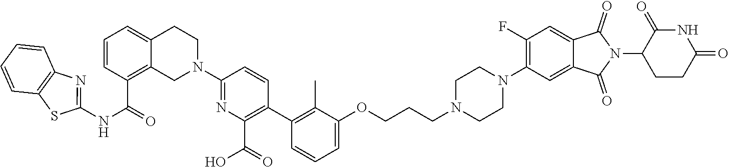 [SMILES: Cc1c(OCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 102 | 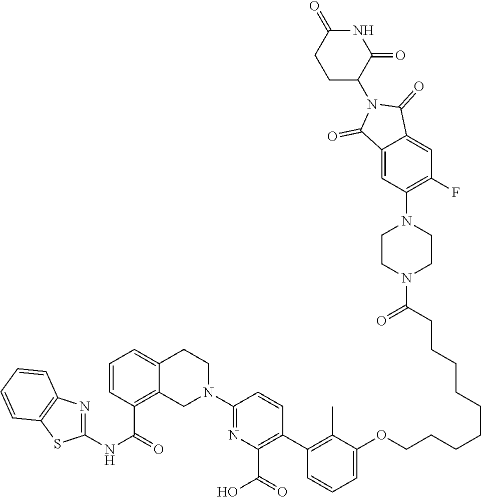 [SMILES: Cc1c(OCCCCCCCCCC(=O)N2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 103 | 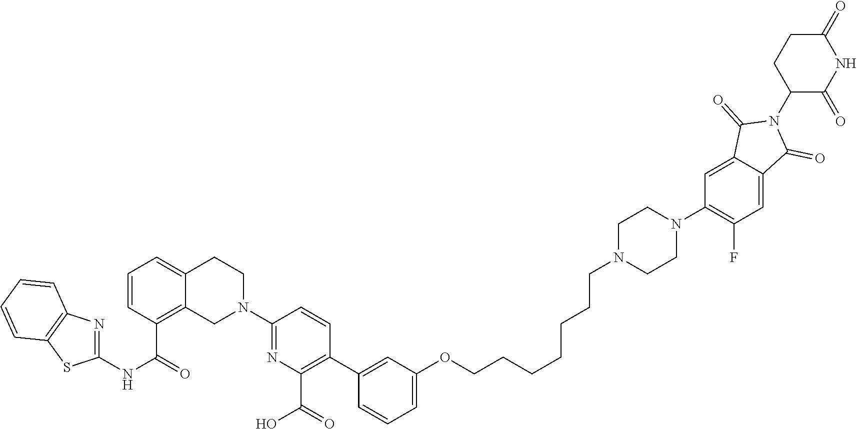 [SMILES: O=C1CCC(N2C(=O)c3cc(F)c(N4CCN(CCCCCCCOc5cccc(-c6ccc(N7CCc8cccc(C(=O)Nc9nc%10ccccc%10s9)c8C7)nc6C(=O)O)c5)CC4)cc3C2=O)C(=O)N1] |
| 104 | 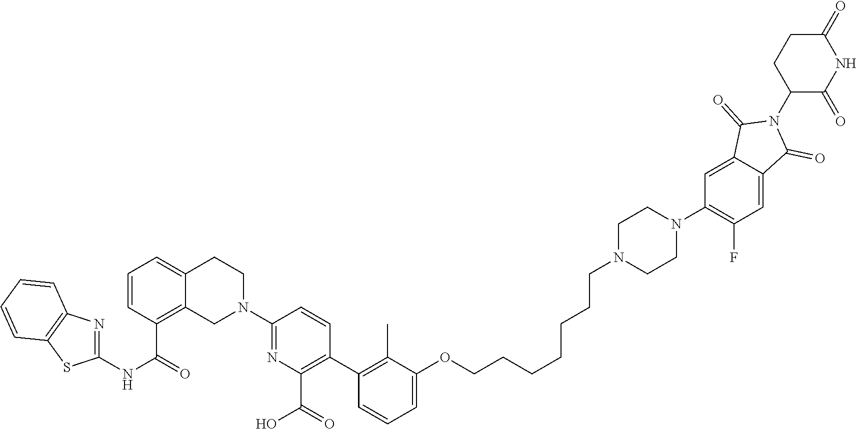 [SMILES: Cc1c(OCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 105 | 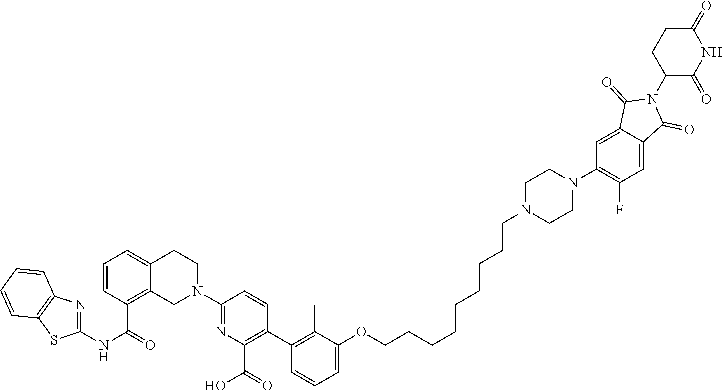 [SMILES: Cc1c(OCCCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 106 | 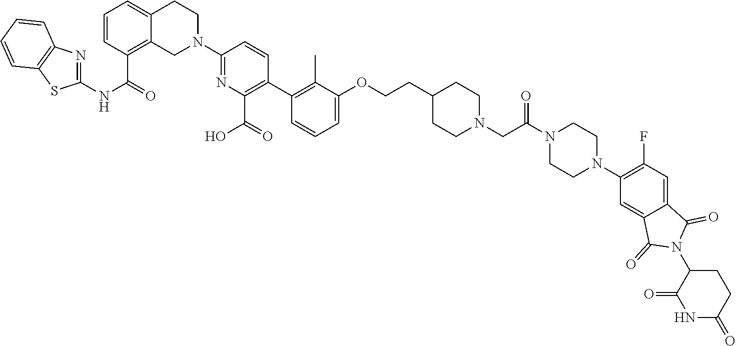 [SMILES: Cc1c(OCCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 107 | 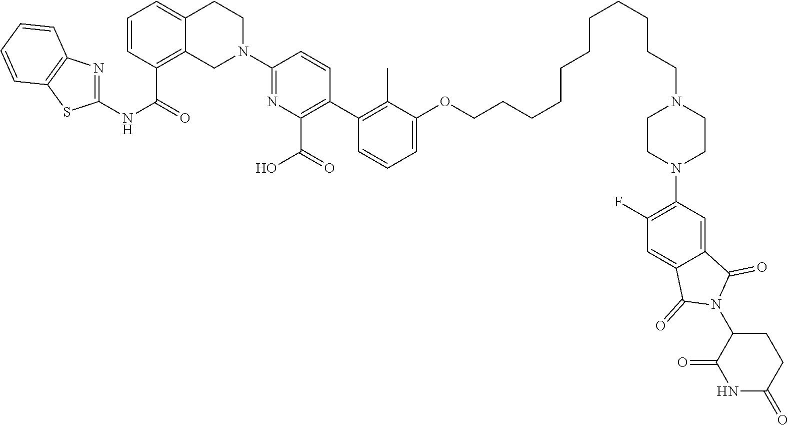 [SMILES: Cc1c(OCCCCCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 108 | 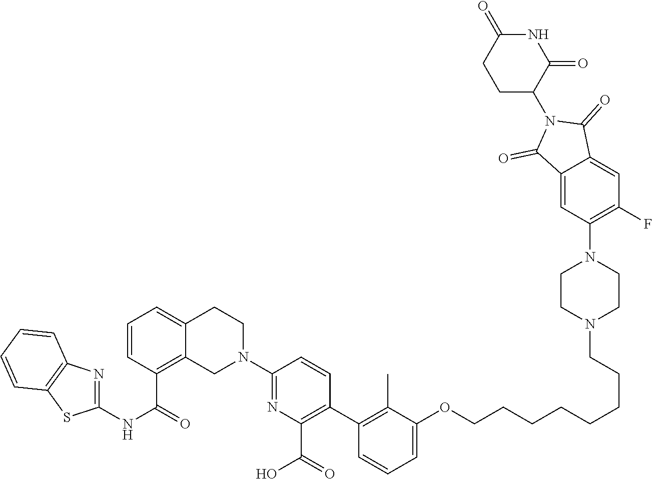 [SMILES: Cc1c(OCCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 109 | 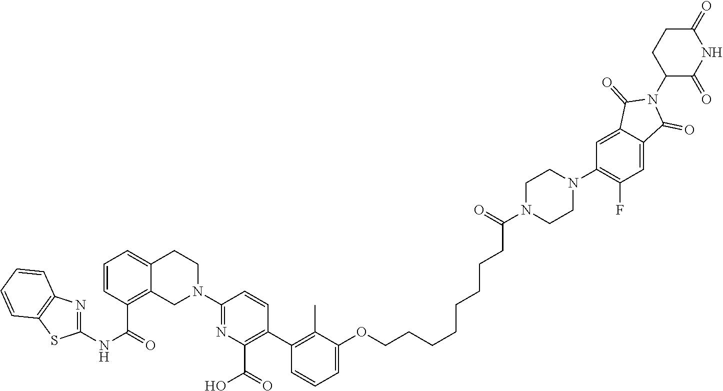 [SMILES: Cc1c(OCCCCCCCCC(=O)N2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 110 | 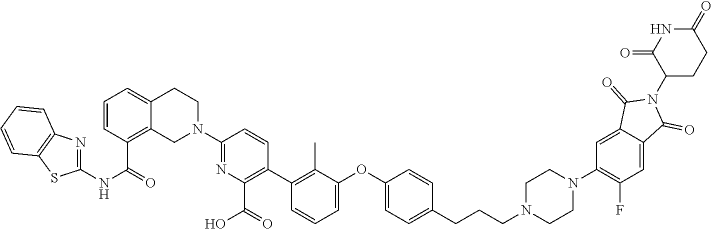 [SMILES: Cc1c(Oc2ccc(CCCN3CCN(c4cc5c(cc4F)C(=O)N(C4CCC(=O)NC4=O)C5=O)CC3)cc2)cccc1-c1ccc(N2CCc3cccc(C(=O)Nc4nc5ccccc5s4)c3C2)nc1C(=O)O] |
| 111 | 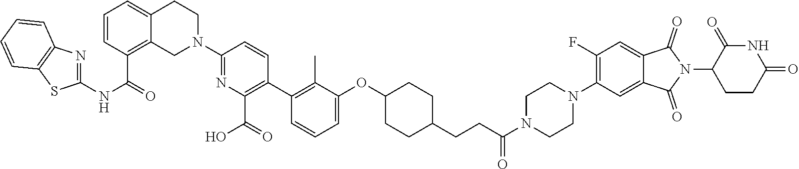 [SMILES: Cc1c(OC2CCC(CCC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 111a | 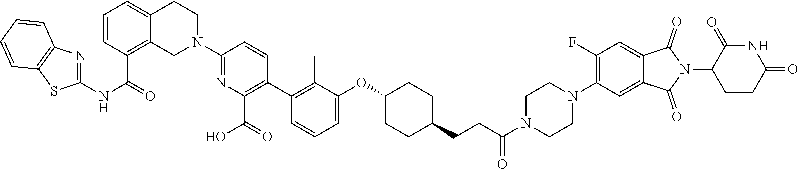 [SMILES: Cc1c(O[C@H]2CC[C@H](CCC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 112 | 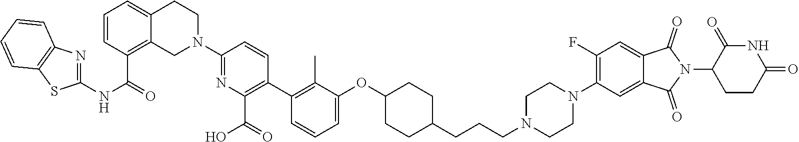 [SMILES: Cc1c(OC2CCC(CCCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 112a | 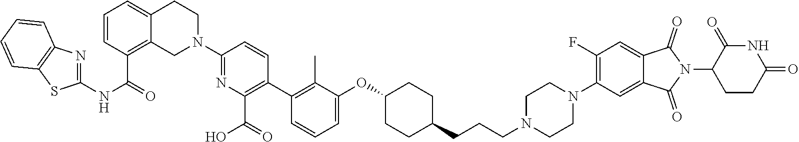 [SMILES: Cc1c(O[C@H]2CC[C@H](CCCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 113 | 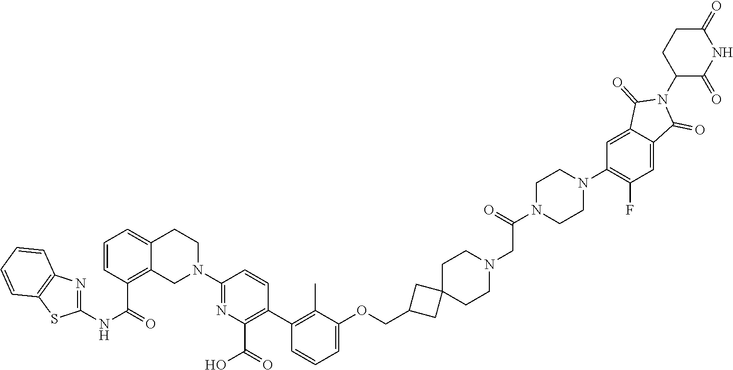 [SMILES: Cc1c(OCC2CC3(CCN(CC(=O)N4CCN(c5cc6c(cc5F)C(=O)N(C5CCC(=O)NC5=O)C6=O)CC4)CC3)C2)cccc1-c1ccc(N2CCc3cccc(C(=O)Nc4nc5ccccc5s4)c3C2)nc1C(=O)O] |
| 114 | 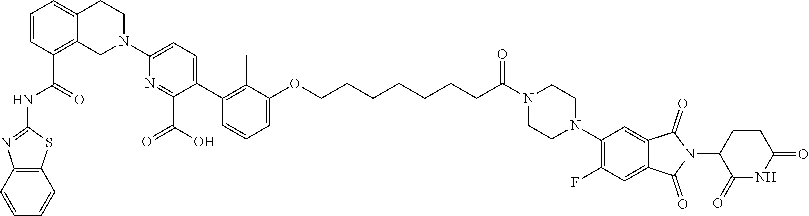 [SMILES: Cc1c(OCCCCCCCC(=O)N2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 115 | 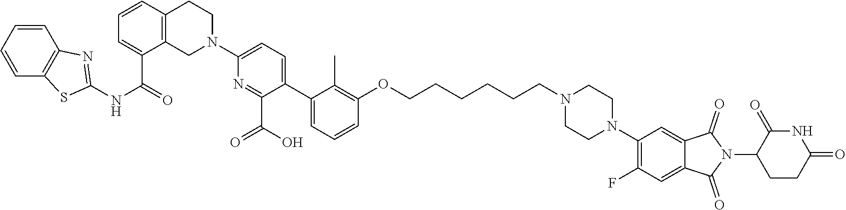 [SMILES: Cc1c(OCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O] |
| 116 | 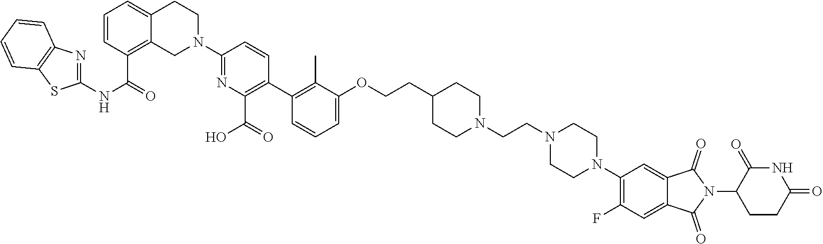 [SMILES: Cc1c(OCCC2CCN(CCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 117 | 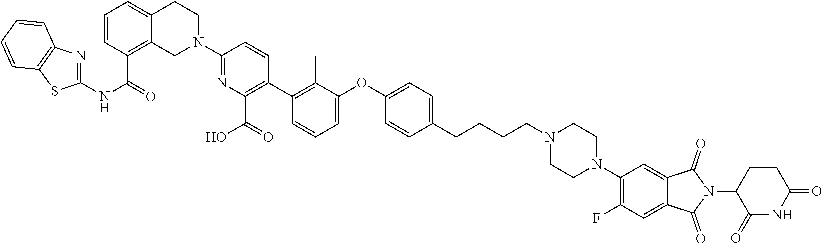 [SMILES: Cc1c(Oc2ccc(CCCCN3CCN(c4cc5c(cc4F)C(=O)N(C4CCC(=O)NC4=O)C5=O)CC3)cc2)cccc1-c1ccc(N2CCc3cccc(C(=O)Nc4nc5ccccc5s4)c3C2)nc1C(=O)O] |
| 118 | 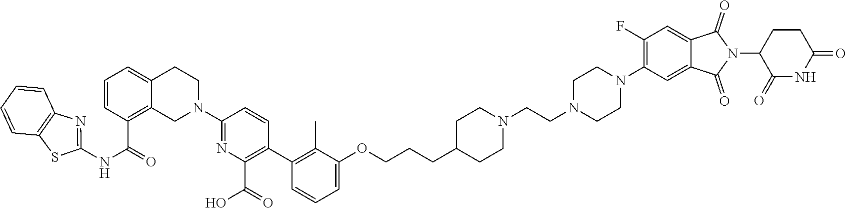 [SMILES: Cc1c(OCCCC2CCN(CCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 119 | 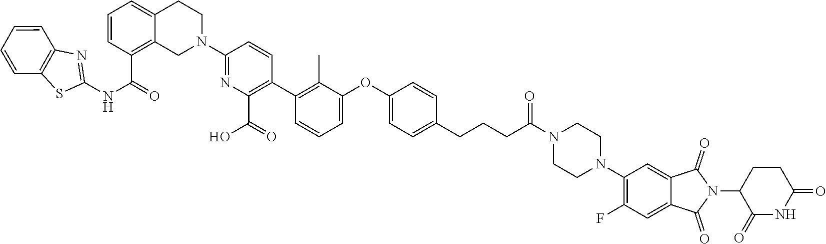 [SMILES: Cc1c(Oc2ccc(CCCC(=O)N3CCN(c4cc5c(cc4F)C(=O)N(C4CCC(=O)NC4=O)C5=O)CC3)cc2)cccc1-c1ccc(N2CCc3cccc(C(=O)Nc4nc5ccccc5s4)c3C2)nc1C(=O)O] |
| 120 | 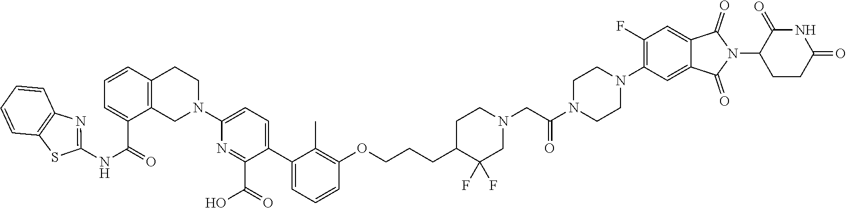 [SMILES: Cc1c(OCCCC2CCN(CC(=O)N3CCN(c4cc5c(cc4F)C(=O)N(C4CCC(=O)NC4=O)C5=O)CC3)CC2(F)F)cccc1-c1ccc(N2CCc3cccc(C(=O)Nc4nc5ccccc5s4)c3C2)nc1C(=O)O] |
| 120a | 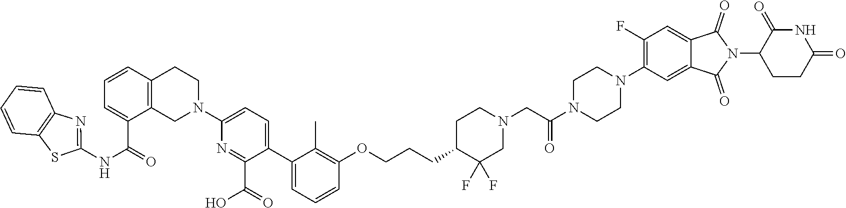 [SMILES: Cc1c(OCCC[C@@H]2CCN(CC(=O)N3CCN(c4cc5c(cc4F)C(=O)N(C4CCC(=O)NC4=O)C5=O)CC3)CC2(F)F)cccc1-c1ccc(N2CCc3cccc(C(=O)Nc4nc5ccccc5s4)c3C2)nc1C(=O)O] |
| 120b | 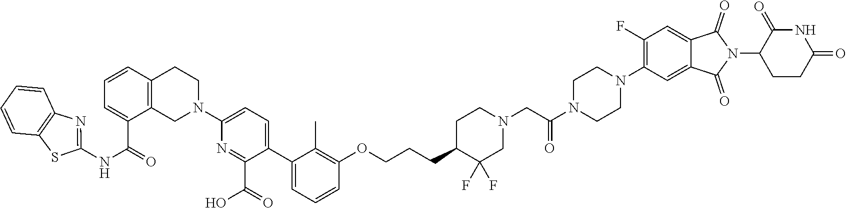 [SMILES: Cc1c(OCCC[C@H]2CCN(CC(=O)N3CCN(c4cc5c(cc4F)C(=O)N(C4CCC(=O)NC4=O)C5=O)CC3)CC2(F)F)cccc1-c1ccc(N2CCc3cccc(C(=O)Nc4nc5ccccc5s4)c3C2)nc1C(=O)O] |
| 121 | 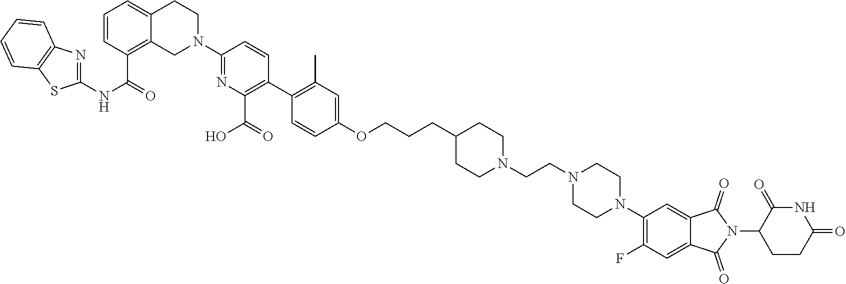 [SMILES: Cc1cc(OCCCC2CCN(CCN3CCN(c4cc5c(cc4F)C(=O)N(C4CCC(=O)NC4=O)C5=O)CC3)CC2)ccc1-c1ccc(N2CCc3cccc(C(=O)Nc4nc5ccccc5s4)c3C2)nc1C(=O)O] |
| 122 | 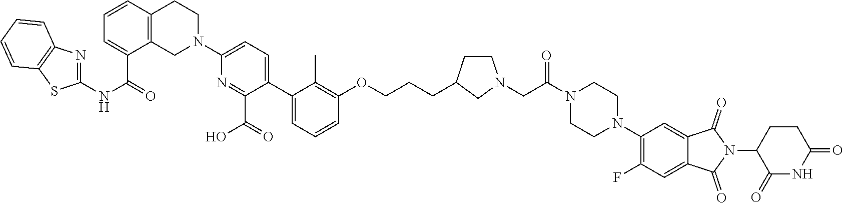 [SMILES: Cc1c(OCCCC2CCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O] |
| 122a | 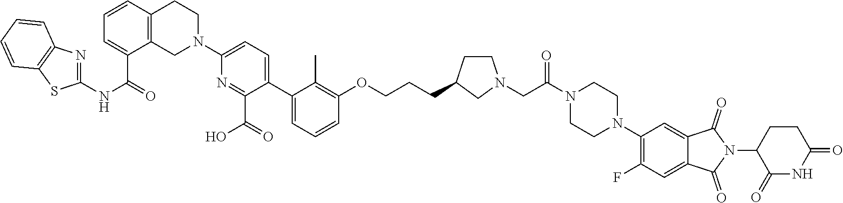 [SMILES: Cc1c(OCCC[C@H]2CCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O] |
| 122b | 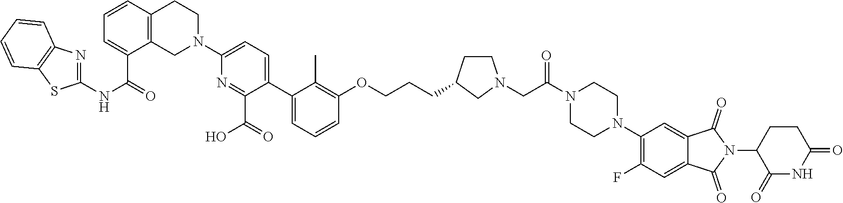 [SMILES: Cc1c(OCCC[C@@H]2CCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O] |
| 123 | 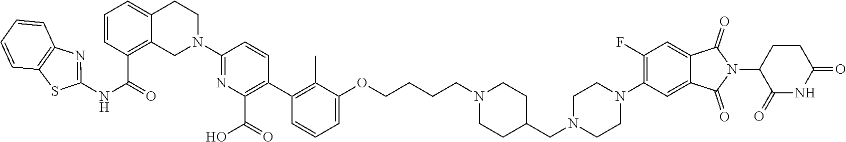 [SMILES: Cc1c(OCCCCN2CCC(CN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 124 | 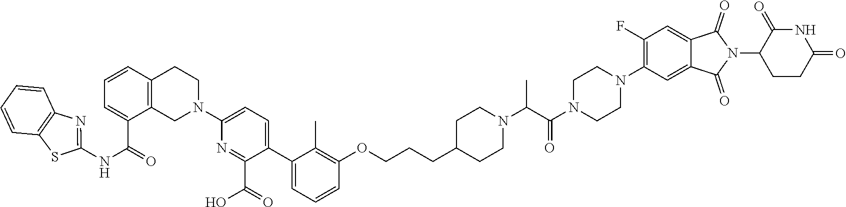 [SMILES: Cc1c(OCCCC2CCN(C(C)C(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 124a | 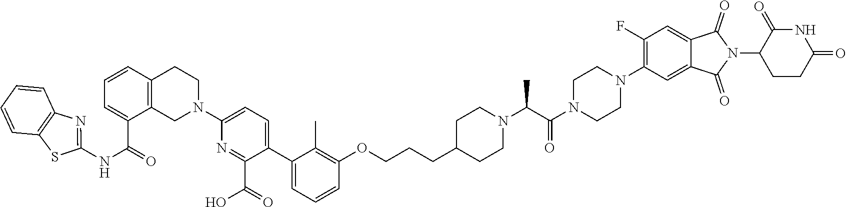 [SMILES: Cc1c(OCCCC2CCN([C@@H](C)C(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 124b | 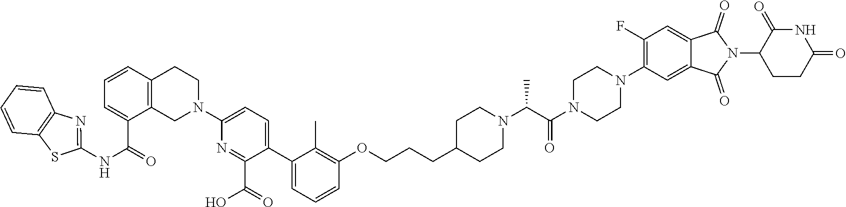 [SMILES: Cc1c(OCCCC2CCN([C@H](C)C(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 125 | 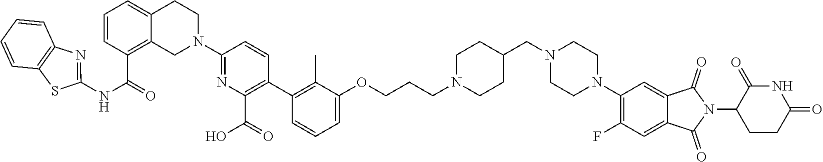 [SMILES: Cc1c(OCCCN2CCC(CN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 126 | 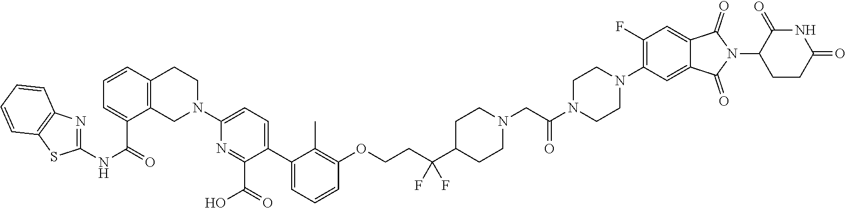 [SMILES: Cc1c(OCCC(F)(F)C2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 127 | 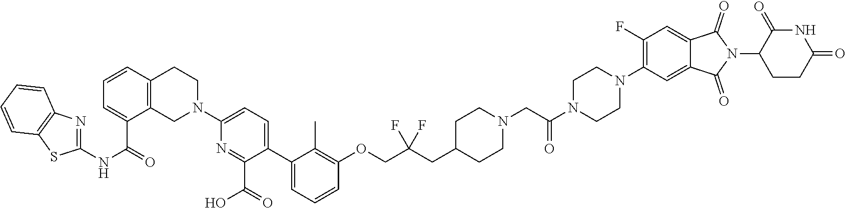 [SMILES: Cc1c(OCC(F)(F)CC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 128 | 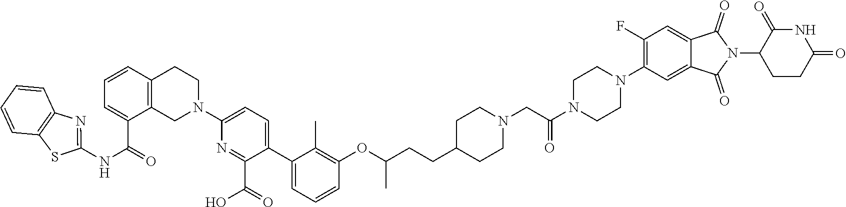 [SMILES: Cc1c(OC(C)CCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 128a | 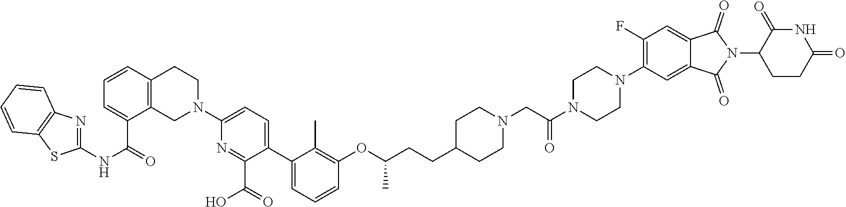 [SMILES: Cc1c(O[C@@H](C)CCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 128b | 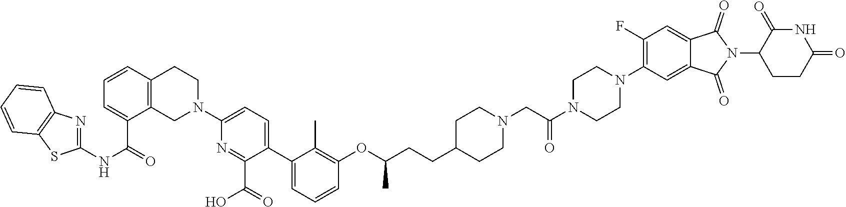 [SMILES: Cc1c(O[C@H](C)CCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 129 | 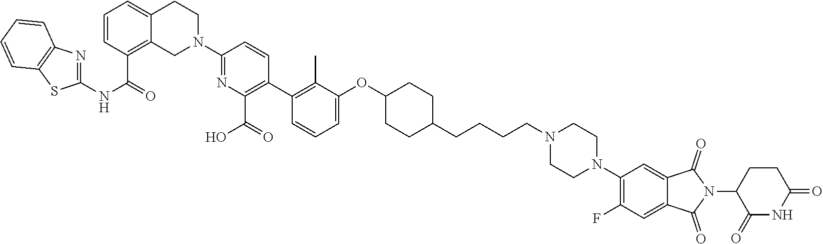 [SMILES: Cc1c(OC2CCC(CCCCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 129a | 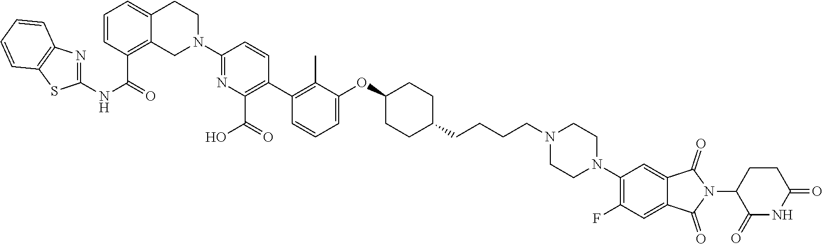 [SMILES: Cc1c(O[C@H]2CC[C@H](CCCCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 130 | 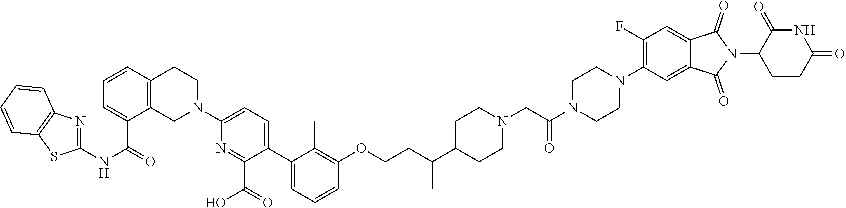 [SMILES: Cc1c(OCCC(C)C2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 130a | 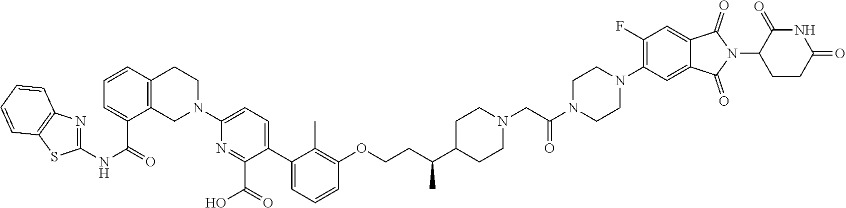 [SMILES: Cc1c(OCC[C@H](C)C2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 130b | 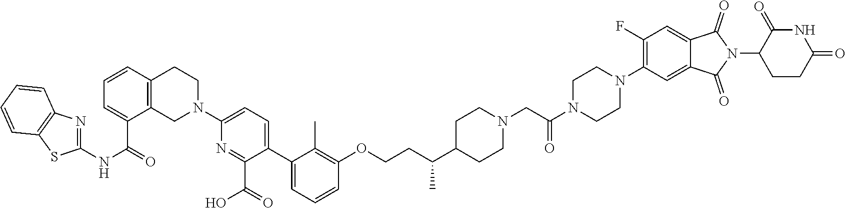 [SMILES: Cc1c(OCC[C@@H](C)C2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 132 | 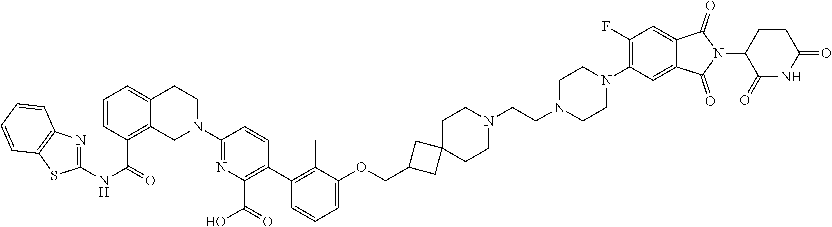 [SMILES: Cc1c(OCC2CC3(CCN(CCN4CCN(c5cc6c(cc5F)C(=O)N(C5CCC(=O)NC5=O)C6=O)CC4)CC3)C2)cccc1-c1ccc(N2CCc3cccc(C(=O)Nc4nc5ccccc5s4)c3C2)nc1C(=O)O] |
| 133 | 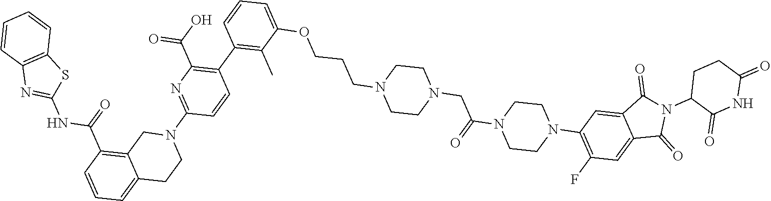 [SMILES: Cc1c(OCCCN2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 135 | 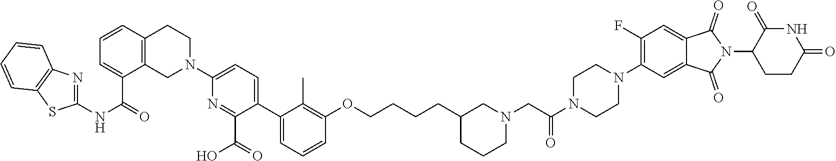 [SMILES: Cc1c(OCCCCC2CCCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O] |
| 135a | 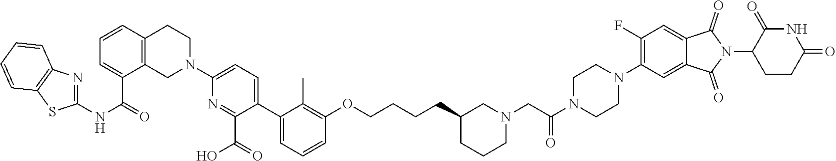 [SMILES: Cc1c(OCCCC[C@@H]2CCCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O] |
| 135b | 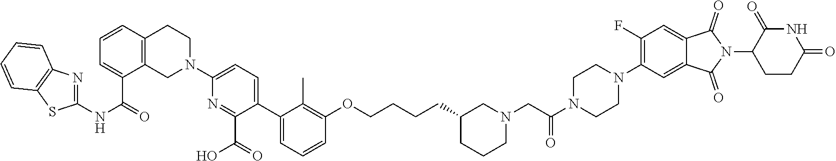 [SMILES: Cc1c(OCCCC[C@H]2CCCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O] |
| 137 | 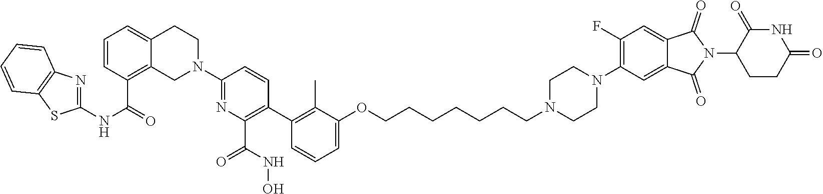 [SMILES: Cc1c(OCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)NO] |
| 138 | 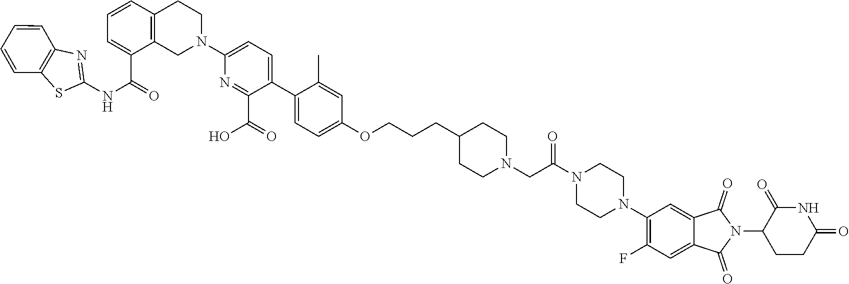 [SMILES: Cc1cc(OCCCC2CCN(CC(=O)N3CCN(c4cc5c(cc4F)C(=O)N(C4CCC(=O)NC4=O)C5=O)CC3)CC2)ccc1-c1ccc(N2CCc3cccc(C(=O)Nc4nc5ccccc5s4)c3C2)nc1C(=O)O] |
| 139 | 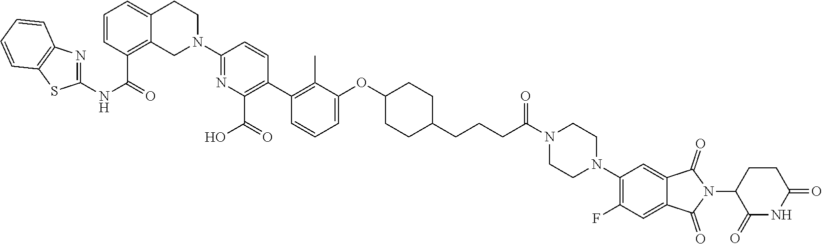 [SMILES: Cc1c(OC2CCC(CCCC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 139a | 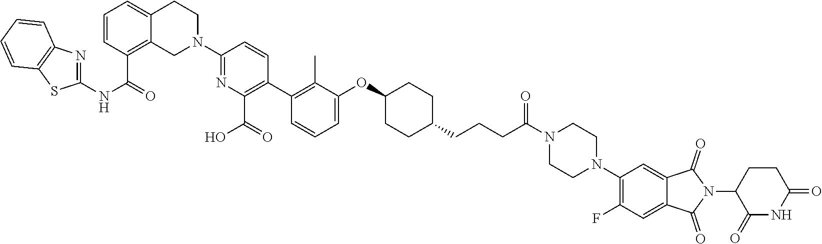 [SMILES: Cc1c(O[C@H]2CC[C@H](CCCC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 140 | 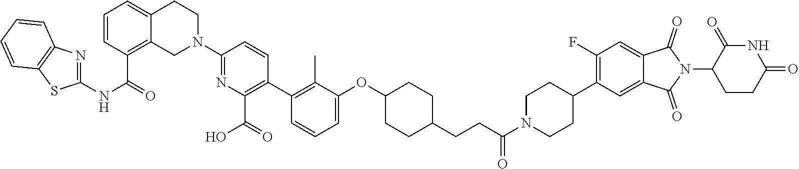 [SMILES: Cc1c(OC2CCC(CCC(=O)N3CCC(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 140a | 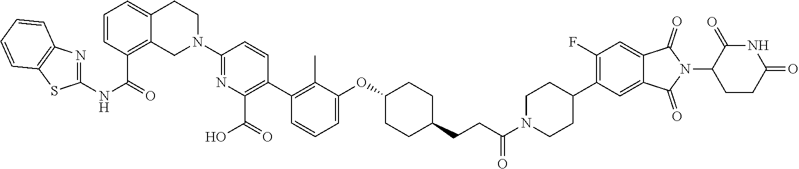 [SMILES: Cc1c(O[C@H]2CC[C@H](CCC(=O)N3CCC(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 141 | 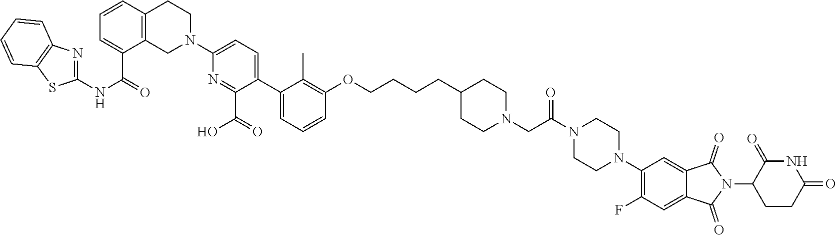 [SMILES: Cc1c(OCCCCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 142 | 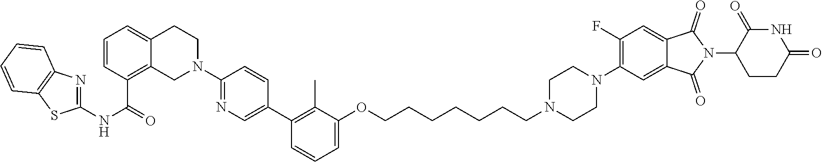 [SMILES: Cc1c(OCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1] |
| 143 | 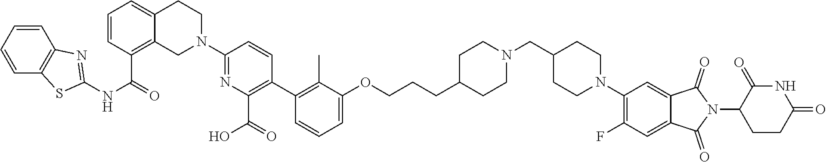 [SMILES: Cc1c(OCCCC2CCN(CC3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 144 | 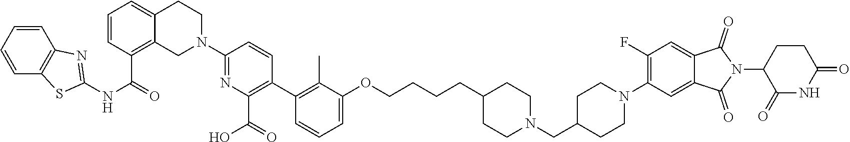 [SMILES: Cc1c(OCCCCC2CCN(CC3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |
| 145 | 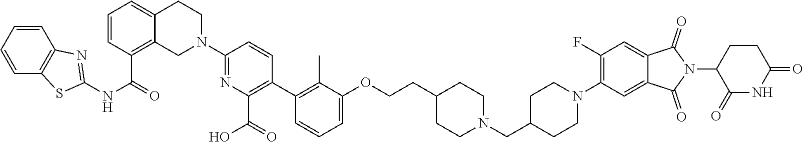 [SMILES: Cc1c(OCCC2CCN(CC3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O] |

Exemplary compounds of Formula (I) or (II) also include those depicted in Table C1 of U.S. Provisional Application Ser. No. 63/454,477, filed Mar. 24, 2023; Table C1 of U.S. Provisional Application Ser. No. 63/429,814, filed Dec. 2, 2022; Table C1 of U.S. Provisional Application Ser. No. 63/398,769, filed Aug. 17, 2022; and Table C1 of U.S. Provisional Application Ser. No. 63/339,262, filed May 6, 2022; or pharmaceutically acceptable salts thereof, wherein each Table C1 is incorporated herein by reference in its entirety.

In some embodiments, the compounds of Formula (I) or (II) (e.g., Formula (I-A), (T-B), or (I-C)), or pharmaceutically acceptable salts thereof, reduce cell viability in a cell line expressing a BCL-X_{L} protein with an EC₅₀ of less than 1 μM (e.g., less than 750 nM, less than 500 nM, or less than 200 nM). In some embodiments, the compounds reduce cell viability in a cell line expressing the BCL-X_{L} protein with an EC₅₀ of less than 200 nM (e.g., less than 150 nM, less than 200 nM, less than 100 nM, less than 10 nM, less than 1 nM). For example, the compounds can reduce cell viability in a cell line expressing the BCL-X_{L} protein with an EC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) or (II) (e.g., Formula (I-A), (I-B), or (I-C)), or pharmaceutically acceptable salts thereof, induce degradation of a BCL-X_{L} protein in a cell line expressing the BCL-X_{L} protein with a DC₅₀ of less than 1 μM (e.g., less than 750 nM, less than 500 nM, or less than 200 nM). In some embodiments, the compounds of Formula (I) or (II) (e.g., Formula (I-A), (I-B), or (I-C)), or a pharmaceutically acceptable salt thereof, induce degradation of a BCL-X_{L} protein in a cell line expressing the BCL-X_{L} protein with a DC₅₀ of less than 200 nM (e.g., less than 150 nM, less than 200 nM, less than 100 nM, less than 10 nM, less than 1 nM). For example, the compounds can induce degradation of a BCL-X_{L} protein in a cell line expressing the BCL-X_{L} protein with a DC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) or (II) (e.g., Formula (I-A), (T-B), or (I-C)), or pharmaceutically acceptable salts thereof, induce degradation of a BCL-X_{L} protein in a cell line expressing the BCL-X_{L} protein with a Yₘᵢₙ of less than 70% (e.g., less than 50%, less than 30%, less than 20%, or less than 10%). In some embodiments, the compounds of Formula (I) or (II) (e.g., Formula (I-A), (I-B), or (I-C)), or a pharmaceutically acceptable salt thereof, induce degradation of a BCL-X_{L} protein in a cell line expressing the BCL-X_{L} protein with a Yₘᵢₙ of less than 50% (e.g., less than 40%, less than 30%, less than 20%, less than 10%, or less than 5%). In some embodiments, the compounds of Formula (I) or (II) (e.g., Formula (I-A), (I-B), or (I-C)), or a pharmaceutically acceptable salt thereof, induce degradation of a BCL-X_{L} protein in a cell line expressing the BCL-X_{L} protein with a Yₘᵢₙ of less than 30% (e.g., less than 25%, less than 20%, less than 15%, less than 10%, or less than 5%). For example, the compounds can induce degradation of a BCL-X_{L} protein in a cell line expressing the BCL-X_{L} protein with a Yₘᵢₙ of about 1% to about 70% (e.g., about 5% to about 50% or about 10% to about 30%).

Also provided herein is a BCL-X_{L} protein non-covalently bound with a compound of Formula (I) or (II) (e.g., Formula (I-A), (I-B), or (I-C)), or a pharmaceutically acceptable salt thereof.

Also provided herein is a ternary complex comprising a BCL-X_{L} protein, a compound of Formula (I) or (II) (e.g., Formula (I-A), (I-B), or (I-C)), or a pharmaceutically acceptable salt thereof, and a CRBN protein, or a portion thereof.

### Chemical Definitions

The term “halo” refers to fluoro (F), chloro (C₁), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo (e.g., —CF₃, —CHF₂, or —CH₂F).

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

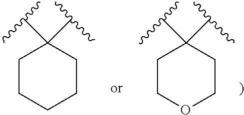 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms))

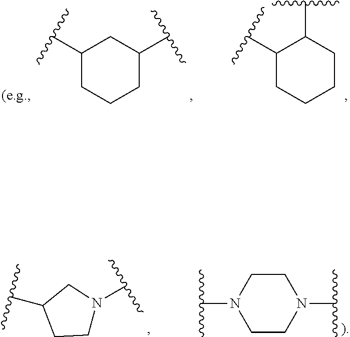 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to mono-, bi-, tri-, or polycyclic (e.g., fused, bridged, or spirocyclic, bi-, tri-, or polycyclic) saturated or partially unsaturated hydrocarbon groups having, e.g., 3 to 20 ring carbons, preferably 3 to 15 ring carbons, and more preferably 3 to 12 ring carbons or 3-10 ring carbons or 3-6 ring carbons, wherein the cycloalkyl group may be optionally substituted. The term “saturated” as used in this context means only single bonds present between constituent carbon atoms. Examples of saturated cycloalkyl groups include, without limitation, cyclopropyl, cyclobutyl, cyclopentyl, cyclohexyl, cycloheptyl, and cyclooctyl. Partially unsaturated cycloalkyl may have any degree of unsaturation provided that one or more double bonds is present in the cycloalkyl, none of the rings in the ring system are aromatic, and the partially unsaturated cycloalkyl group is not fully saturated overall. Examples of partially unsaturated cycloalkyl include, without limitation, cyclopentenyl, cyclohexenyl, cycloheptenyl, and cyclooctenyl. Non-limiting examples of fused/bridged cycloalkyl includes: bicyclo[1.1.0]butyl, bicyclo[2.1.0]pentyl, bicyclo[1.1.1]pentyl, bicyclo[3.1.0]hexyl, bicyclo[2.1.1]hexyl, bicyclo[3.2.0]heptyl, bicyclo[4.1.0]heptyl, bicyclo[2.2.1]heptyl, bicyclo[3.1.1]heptyl, bicyclo[4.2.0]octyl, bicyclo[3.2.1]octyl, bicyclo[2.2.2]octyl, and the like. Non-limiting examples of spirocyclic cycloalkyls include spiro[2.2]pentyl, spiro[2.5]octyl, spiro[3.5]nonyl, spiro[3.5]nonyl, spiro[3.5]nonyl, spiro[4.4]nonyl, spiro[2.6]nonyl, spiro[4.5]decyl, spiro[3.6]decyl, spiro[5.5]undecyl, and the like.

The term “heteroaryl”, as used herein, means a mono-, bi-, tri- or polycyclic group having 5 to 20 ring atoms, alternatively 5, 6, 9, 10, or 15 ring atoms; wherein at least one ring in the system contains one or more heteroatoms independently selected from the group consisting of N, O, S (inclusive of oxidized forms such as:

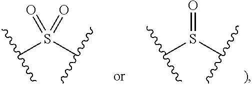

and P (inclusive of oxidized forms such as

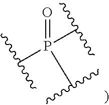 [SMILES: *P(*)(*)=O]

and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

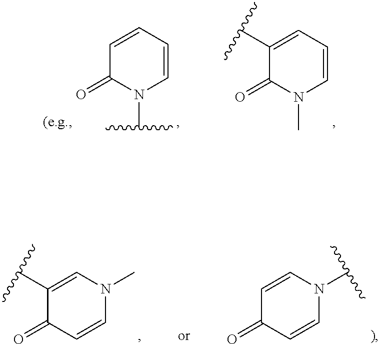

pyrimidone

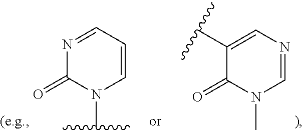

pyridazinone

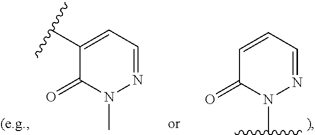

pyrazinone

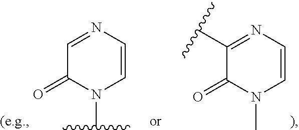

and imidazolone

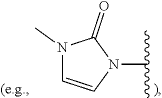 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic (e.g., fused, bridged, or spirocyclic, bi-, tri-, or polycyclic) saturated or partially unsaturated ring system with 3-15 ring atoms (e.g., 5-8 membered monocyclic, 8-12 membered bicyclic, or 11-15 membered tricyclic ring system) having 1-3 heteroatoms if monocyclic, 1-6 heteroatoms if bicyclic, or 1-9 heteroatoms if tricyclic or polycyclic, said heteroatoms selected from O, N, S (inclusive of oxidized forms such as:

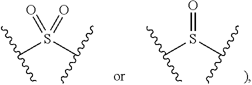

and P (inclusive of oxidized forms such as:

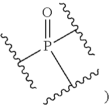 [SMILES: *P(*)(*)=O]

(e.g., carbon atoms and 1-3, 1-6, or 1-9 heteroatoms of N, O, S, or P if monocyclic, bicyclic, or tricyclic, respectively), wherein 0, 1, 2 or 3 atoms of each ring may be substituted by a substituent. The term “saturated” as used in this context means only single bonds present between constituent ring atoms and other available valences occupied by hydrogen and/or other substituents as defined herein. Examples of saturated heterocyclyl groups include piperazinyl, pyrrolidinyl, dioxanyl, morpholinyl, tetrahydrofuranyl, and the like. Partially unsaturated heterocyclyl groups may have any degree of unsaturation provided that one or more double bonds is present in the heterocyclyl, none of the rings in the ring system are aromatic, and the partially unsaturated heterocyclyl group is not fully saturated overall. Examples of partially unsaturated heterocyclyl groups include, without limitation, tetrahydropyridyl, dihydropyrazinyl, dihydropyridyl, dihydropyrrolyl, dihydrofuranyl, dihydrothiophenyl. Non-limiting examples of fused/bridged heteorocyclyl includes: 2-azabicyclo[1.1.0]butyl, 2-azabicyclo[2.1.0]pentyl, 2-azabicyclo[1.1.1]pentyl, 3-azabicyclo[3.1.0]hexyl, 5-azabicyclo[2.1.1]hexyl, 3-azabicyclo[3.2.0]heptyl, octahydrocyclopenta[c]pyrrole, 3-azabicyclo[4.1.0]heptyl, 7-azabicyclo[2.2.1]heptyl, 6-azabicyclo[3.1.1]heptyl, 7-azabicyclo[4.2.0]octyl, 2-azabicyclo[2.2.2]octyl, 3-azabicyclo[3.2.1]octyl, 2-oxabicyclo[1.1.0]butyl, 2-oxabicyclo[2.1.0]pentyl, 2-oxabicyclo[1.1.1]pentyl, 3-oxabicyclo[3.1.0]hexyl, 5-oxabicyclo[2.1.1]hexyl, 3-oxabicyclo[3.2.0]heptyl, 3-oxabicyclo[4.1.0]heptyl, 7-oxabicyclo[2.2.1]heptyl, 6-oxabicyclo[3.1.1]heptyl, 7-oxabicyclo[4.2.0]octyl, 2-oxabicyclo[2.2.2]octyl, 3-oxabicyclo[3.2.1]octyl, and the like. Non-limiting examples of spirocyclic heterocyclyls include 2-azaspiro[2.2]pentyl, 4-azaspiro[2.5]octyl, 1-azaspiro[3.5]nonyl, 2-azaspiro[3.5]nonyl, 7-azaspiro[3.5]nonyl, 2-azaspiro[4.4]nonyl, 6-azaspiro[2.6]nonyl, 1,7-diazaspiro[4.5]decyl, 7-azaspiro[4.5]decyl 2,5-diazaspiro[3.6]decyl, 3-azaspiro[5.5]undecyl, 2-oxaspiro[2.2]pentyl, 4-oxaspiro[2.5]octyl, 1-oxaspiro[3.5]nonyl, 2-oxaspiro[3.5]nonyl, 7-oxaspiro[3.5]nonyl, 2-oxaspiro[4.4]nonyl, 6-oxaspiro[2.6]nonyl, 1,7-dioxaspiro[4.5]decyl, 2,5-dioxaspiro[3.6]decyl, 1-oxaspiro[5.5]undecyl, 3-oxaspiro[5.5]undecyl, 3-oxa-9-azaspiro[5.5]undecyl and the like.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

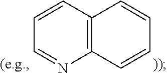

(ii) a single ring atom (spiro-fused ring systems)

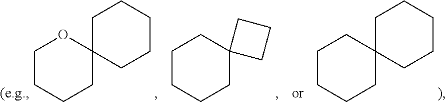

or (iii) a contiguous array of ring atoms (bridged ring systems having all bridge lengths>0)

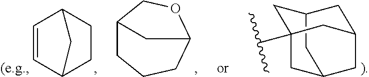

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

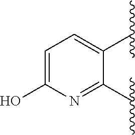 [SMILES: *c1ccc(O)nc1*]

encompasses the tautomeric form containing the moiety:

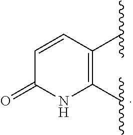 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

### Methods of Treatment

### Indications

Provided herein are methods for inducing degradation of a BCL-X_{L} protein. For example, provided herein are compounds capable of inducing degradation of a BCL-X_{L} protein useful for treating or preventing cancers. See, e.g., Guo, et al. Aging (Albany NY) 13.15 (2021): 19750; Park, et al. Proceedings of the National Academy of Sciences 112.40 (2015): 12492-12497; Zhang, et al. Molecular Cancer 14.1 (2015): 1-9; Beroukhim, et al. Nature 463.7283 (2010): 899-905. Additional methods of evaluating the binding of a compound to BCL-X_{L} protein or the inhibition of a BCL-X_{L} protein are described in, for example, U.S. Patent Publication Nos. 2007/027135; 2010/305122, and 2013/096120.

It will be understood that the effect of protein degradation typically increases over time, though the appearance of degradation (e.g., as expressed by the percentage degradation compared to a control, or the parameters Yₘᵢₙ, DC₅₀, and/or Dₘₐₓ) is affected by the resynthesis rate of the protein. It is common in the art to examine degradation after a specified period of time, such as 6 hours, 12 hours, 18 hours, 1 day, 2 days, 3 days, or more. For example, degradation can be expressed as the percent degradation after 24 hours.

Exemplary assays for validating the degradation-inducing mechanism of a compound as provided herein are known in the art and are described, for example, in International Publication No. WO 2019/144117 and Wu, et al. Nature Structural & Molecular Biology 27.7 (2020): 605-614.

Degradation assays can be used to quantify both on- and off-target degradation-inducing effects of compounds, such as those provided herein. Exemplary assays include, quantitative immunoblotting, other immunoassays (e.g., MesoScale Discovery (MSD) immunoassays), homogenous time resolved florescence (HTRF), and HiBiT. In some embodiments, cells can be contacted with a compound of Formula (I) or (II) (e.g., Formula (I-A), (I-B), or (I-C)), or a pharmaceutically acceptable salt thereof, incubated, and then the lysate can be prepared for gel electrophoresis (e.g., SDS-PAGE), followed by immunoblotting and quantification compared to a control (e.g., a DMSO-treated control). As another example, a cell line can be engineered to express a HiBiT-tagged BCL-X_{L} protein, and the amount of fluorescence observed when the complementary LgBiT peptide is added can be compared between cells treated with a compound of Formula (I) or (II) (e.g., Formula (I-A), (I-B), or (I-C)), or a pharmaceutically acceptable salt thereof, and a control (e.g., a DMSO-treated control).

See, for instance, Example B1 and Example B2. See also, e.g., International Publication Nos. WO 2020/163823 and WO 2019/144117. In some embodiments, off-target degradation inducing effects can be assessed for the proteins Eukaryotic peptide chain release factor GTP-binding subunit ERF3A (GSPT1), Ikaros (IKZF1), Helios (IKZF2), Aiolos (IKZF3), and/or casein kinase I isoform alpha (CK1α).

See also the assays described in International Publication Nos. WO 2023/044046; WO 2022/169780; WO 2021/222114; WO 2021/146536; WO 2021/078301; WO 2021/007307; WO 2020/163823; WO 2019/144117; WO 2017/184995, and Khan, et al. Nature Medicine 25.12 (2019): 1938-1947; Balachander, et al. Clinical Cancer Research 26.24 (2020): 6535-6549.

Binding affinity of a compound of Formula (I) or (II) (e.g., Formula (I-A), (I-B), or (I-C)), or a pharmaceutically acceptable salt thereof, as provided herein to BCL-X_{L} can be determined by, for example, a binding IC₅₀ or Kᵢ value (e.g., using a competition assay), or by a K_{D} value (e.g., using a biophysical assay). A compound with a lower binding IC₅₀ value, as determined under substantially similar conditions, is a more potent binder relative to a compound with a higher binding IC₅₀ value. A compound with a lower binding Kᵢ value, as determined under substantially similar conditions, is a more potent binder relative to a compound with a higher binding Kᵢ value. Similarly, a compound with a lower K_{D} value, as determined under substantially similar conditions, is a more potent binder relative to a compound with a higher K_{D} value. For example, a binding IC₅₀ value can be determined in a fluorescence polarization assay using a fluorescently labeled BH3-only peptide (e.g., BAD or BAX) as the competitor. As another example, a binding Kᵢ value can be determined using a time resolved-fluorescence resonance energy transfer (TR-FRET) assay using a fluorescently labeled BH3-only peptide (e.g., BAK) and a fluorescently labeled antibody that binds to BCL-X_{L}, where the fluorophores are a FRET pair, and using a compound of Formula (I) or (II) (e.g., Formula (I-A), (I-B), or (I-C)), or a pharmaceutically acceptable salt thereof, as provided herein as a competitor for the BH3-only peptide. See, e.g., U.S. Patent Publication Nos. 2007/0027135, 2010/305122, and 2013/096120.

The ability of a compound of Formula (I) or (II) (e.g., Formula (I-A), (I-B), or (I-C)), or a pharmaceutically acceptable salt thereof, as provided herein to inhibit BCL-X_{L} can be determined using an IC₅₀ value. A compound with a lower IC₅₀ value, as determined under substantially similar conditions, is a more potent inhibitor relative to a compound with a higher IC₅₀ value. One way that inhibition of BCL-X_{L} can be measured is measuring the interruption of the formation of a complex of BCL-X_{L} with a BH3-only peptide (e.g., BIM). For example, an electrochemiluminescence-based sandwich ELISA assay (e.g., a Meso Scale Discovery (MSD)-ELISA assay) can be used. See, for example, Phillips, D. C., et al. Blood Cancer Journal 5.11 (2015): e368-e368 and Xiao, Yu, et al. Molecular Cancer Therapeutics 14.8 (2015): 1837-1847. In such assays, cells expressing BCL-X_{L} can be incubated with a compound of Formula (I) or (II) (e.g., Formula (I-A), (I-B), or (I-C)) for a period of time, lysed, and then evaluated in the assay. A tagged anti-BCL-X_{L} antibody (e.g., a biotin-tagged anti-BCL-X_{L} antibody) can be immobilized on an assay plate (e.g., a streptavidin assay plate), and then the lysate can be applied to pull down BCL-X_{L}. An anti-BIM antibody (e.g., a rabbit anti-BIM antibody) can be introduced, followed by addition of a detection antibody (e.g., a sulfo-tagged goat anti-rabbit antibody) and measurement of the detection antibody. As another example, interruption of the formation of a complex of BCL-X_{L} with a BH3-only peptide (e.g., BIM) can be measured using a mammalian two-hybrid assay. In such assays, a plasmid encoding the ‘bait’ and ‘prey’ fusion proteins (e.g., the DNA binding domain of GAL4 fused to BCL-X_{L} and the transcriptional activation domain of VP16 fused to BIM) can be introduced to cells (e.g., HeLa cells) stably expressing a GAL4-luciferase reporter. A compound of Formula (I) or (II) (e.g., Formula (I-A), (I-B), or (I-C)) can be added to the cells in culture, incubated, and the luciferase activity can be measured. See, for example, Souers, Andrew J., et al. Nature Medicine 19.2 (2013): 202-208.

Potency of degradation by a compound of Formula (I) or (II) (e.g., Formula (I-A), (T-B), or (I-C)), or a pharmaceutically acceptable salt thereof, as provided herein can be determined by DC₅₀ value. As used herein, DC₅₀ refers to the concentration of the compound of Formula (I) or (II) (e.g., Formula (I-A), (I-B), or (I-C)) that results in a 50% decrease in the concentration of a protein (e.g., BCL-X_{L} protein) in a cell compared to the concentration of the protein before the cell is contacted with the compound of Formula (I) or (II) (e.g., Formula (I-A), (I-B), or (I-C)), or compared to the concentration of the protein in a cell not contacted with the compound of Formula (I) or (II) (e.g., Formula (I-A), (I-B), or (I-C)). A compound with a lower DC₅₀ value, as determined under substantially similar conditions, is a more efficient inducer of degradation relative to a compound with a higher DC₅₀ value. In some embodiments, a DC₅₀ value can be determined (e.g., using HiBiT detection) in vitro or in vivo (e.g., in tumor cells (e.g., cell lines such as MOLT4, RS4; 11, NCI-H146, EJM, HEK293T, HT1080, and/or H929) expressing a BCL-X_{L} protein).

Potency of degradation by a compound of Formula (I) or (II) (e.g., Formula (I-A), (T-B), or (I-C)), or a pharmaceutically acceptable salt thereof, as provided herein can be determined by EC₅₀ value. As used herein, EC₅₀ refers to the concentration of the compound of Formula (I) or (II) (e.g., Formula (I-A), (I-B), or (I-C)) that results in a 50% decrease in the concentration of a protein (e.g., BCL-X_{L} protein) relative to the trough concentration of the protein in a cell, when compared to the concentration of the protein before the cell is contacted with the compound of Formula (I) or (II) (e.g., Formula (I-A), (I-B), or (I-C)), or compared to the concentration of the protein in a cell not contacted with the compound of Formula (I) or (II) (e.g., Formula (I-A), (I-B), or (I-C)). A compound with a lower EC₅₀ value, as determined under substantially similar conditions, is a more potent compound relative to a compound with a higher EC₅₀ value. In some embodiments, an EC₅₀ value can be determined (e.g., using HiBiT detection) in vitro or in vivo (e.g., in tumor cells (e.g., cell lines such as MOLT4, RS4; 11, NCI-H146, EJM, HEK293T, HT1080, and/or H929) expressing a BCL-X_{L} protein).

Potency of degradation by a compound of Formula (I) or (II) (e.g., Formula (I-A), (I-B), or (I-C)), or a pharmaceutically acceptable salt thereof, as provided herein can be determined by Yₘᵢₙ value. As used herein, Yₘᵢₙ refers to the ratio of trough concentration of a protein (e.g., BCL-X_{L} protein) in a cell compared to the concentration of the protein before the cell is contacted with the compound of Formula (I) or (II) (e.g., Formula (I-A), (I-B), or (I-C)), or compared to the concentration of the protein in a cell not contacted with the compound of Formula (I) or (II) (e.g., Formula (I-A), (I-B), or (I-C)), expressed as a percentage. As used herein, Dₘₐₓ is 1−Yₘᵢₙ. Yₘᵢₙ can be measured by a HiBiT assay as described in Example B1. A compound with a lower Yₘᵢₙ value, as determined under substantially similar conditions, is a more potent inducer of degradation relative to a compound with a higher Yₘᵢₙ value. A compound with a lower Yₘᵢₙ value, as determined under substantially similar conditions, is a more potent compound relative to a compound with a higher Yₘᵢₙ value. In some embodiments, a Yₘᵢₙ value can be determined (e.g., using HiBiT detection) in vitro or in vivo (e.g., in tumor cells (e.g., cell lines such as MOLT4, RS4; 11, NCI-H146, EJM, HEK293T, HT1080, and/or H929) expressing a BCL-X_{L} protein).

An exemplary assay for determining the potency of a compound of Formula (I) or (II) (e.g., Formula (I-A), (I-B), or (I-C)), or a pharmaceutically acceptable salt thereof, includes measuring the effect of the compound of Formula (I) or (II) (e.g., Formula (I-A), (I-B), or (I-C)), or a pharmaceutically acceptable salt thereof, on cell proliferation and/or viability. Cell proliferation assays can be performed in a number of formats, including 2D and 3D. Similarly, a cell proliferation assay can be performed with any appropriate cell line, including, for example, MOLT4, RS4; 11, NCI-H146, EJM, HEK293T, HT1080, and/or H929. As an illustrative example, a 3D cell proliferation assay can include growing cells in a 3D medium, contacting the cells with a compound of Formula (I) or (II) (e.g., Formula (I-A), (I-B), or (I-C)), or a pharmaceutically acceptable salt thereof, measuring the cellular proliferation using an appropriate reagent (e.g., CELLTITERGLO® 3D), and then comparing the signal from an experiment with a compound of Formula (I) or (II) (e.g., Formula (I-A), (I-B), or (I-C)), or a pharmaceutically acceptable salt thereof, to the signal from a control experiment (e.g., lacking the compound of Formula (I) or (II) (e.g., Formula (I-A), (I-B), or (I-C)), or a pharmaceutically acceptable salt thereof). As another illustrative example, a 2D cell proliferation assay can include plating cells onto a growth surface, optionally letting the cells grow for a period of time, contacting the cells with a compound of Formula (I) or (II) (e.g., Formula (I-A), (I-B), or (I-C)), or a pharmaceutically acceptable salt thereof, measuring the cellular proliferation using an appropriate reagent (e.g., CELLTITERGLO®), and then comparing the signal from an experiment with compound of Formula (I) or (II) (e.g., Formula (I-A), (I-B), or (I-C)), or a pharmaceutically acceptable salt thereof, to the signal from a control experiment (e.g., lacking a compound of Formula (I) or (II) (e.g., Formula (I-A), (I-B), or (I-C)), or a pharmaceutically acceptable salt thereof). Additional cell viability assays include MTT assays, which are colorimetric assays based on the reduction of the tetrazolium dye MTT (3-(4,5-dimethylthiazol-2-yl)-2,5-diphenyltetrazolium bromide) to the insoluble purple formazan, and other similar assays based on related tetrazolium salts. See, for instance, Example B3 and Example B4.

A cell viability assay can be used to measure the effect of a compound of Formula (I) or (II) (e.g., Formula (I-A), (I-B), or (I-C)), or a pharmaceutically acceptable salt thereof, on cell death. For example, cells expressing BCL-X_{L} protein (e.g., MOLT-4 cells) can be incubated with various concentrations of a compound of Formula (I) or (II) (e.g., Formula (I-A), (I-B), or (I-C)), or a pharmaceutically acceptable salt thereof, then exposed to a detection reagent (e.g., a CELLTITER-GLO® Cell Viability Assay kit) to determine cell viability. An exemplary assay for evaluating the affinity of a compound of Formula (I) or (II) (e.g., Formula (I-A), (I-B), or (I-C)), or a pharmaceutically acceptable salt thereof, includes using a competition assay with recombinant BCL-X_{L} protein. For example, purified recombinant affinity-tagged (e.g., His-tagged) BCL-X_{L} protein can be incubated with various concentrations of a compound of Formula (I) or (II) (e.g., Formula (I-A), (I-B), or (I-C)), or a pharmaceutically acceptable salt thereof, and a fixed concentration of affinity-tagged (e.g., biotin-tagged) BAD protein. After a period of incubation, FRET acceptor beads with a complementary affinity tag (e.g., His-acceptor beads) and FRET donor beads (e.g., streptavidin-tagged donor beads) can be added to the mixture, and a FRET reaction can be used to determine an inhibition constant of the compound of Formula (I) or (II) (e.g., Formula (I-A), (I-B), or (I-C)), or pharmaceutically acceptable salt thereof. For example, an AlphaLISA competitive assay can be performed. See, e.g., International Publication No. WO 2019/144117.

An exemplary assay for determining the mechanism of cell death using of a compound of Formula (I) or (II) (e.g., Formula (I-A), (I-B), or (I-C)), or a pharmaceutically acceptable salt thereof, includes measuring the effect of the compound of Formula (I) or (II) (e.g., Formula (I-A), (I-B), or (I-C)), or a pharmaceutically acceptable salt thereof, on one or more markers of a mechanism of cell death (e.g., apoptosis). Exemplary markers of apoptosis include caspase induction (e.g., caspase 3/7 induction) and annexin V staining. Such assays may also be used as determinants of cell viability. For example, cells expressing BCL-X_{L} protein (e.g., MOLT-4 cells) can be incubated with various concentrations of a compound of Formula (I) or (II) (e.g., Formula (I-A), (I-B), or (I-C)), or a pharmaceutically acceptable salt thereof, and relative caspase activity can be assessed using a luciferase substrate that is activated by caspase 3/7 (e.g., using the CASPASE-GLO® 3/7 assay). As another example, cells expressing BCL-X_{L}protein (e.g., MOLT-4 cells) can be incubated with various concentrations of a compound of Formula (I) or (II) (e.g., Formula (I-A), (I-B), or (I-C)), or a pharmaceutically acceptable salt thereof, and relative caspase activity can be assessed using a dye that is coupled to an activated caspase motif (e.g., INCUCYTE® Caspase 3/7 Green Apoptosis Assay Reagent), followed by analysis using a platform for live cell imaging (e.g., an INCUCYTE® SX5 Live-Cell Analysis Instrument). As another example, cells expressing BCL-X_{L} protein (e.g., MOLT-4 cells) can be incubated with various concentrations of a compound of Formula (I) or (II) (e.g., Formula (I-A), (I-B), or (I-C)), or a pharmaceutically acceptable salt thereof, and annexin V positivity can be assessed using phosphatidylserine dye (e.g., an INCUCYTE® Annexin V dye), followed by analysis using a platform for live cell imaging (e.g., an INCUCYTE® SX5 Live-Cell Analysis Instrument). See, for instance, Example B5.

As another example, the potency and/or efficacy of a compound of Formula (I) or (II) (e.g., Formula (I-A), (I-B), or (I-C)), or a pharmaceutically acceptable salt thereof, can be evaluated in an animal model, for example, a xenograft model (e.g., using an established cancer cell line such as MOLT4, HEL, TF1, F36P, OCI-M1, OCI-M2, SET-2, CMK, M07E, or UKE-1, or a patient-derived xenograft (PDX) model). For example, a PDX model can be run in immunodeficient mice (e.g., athymic nude, outbred homozygous (e.g., Crl:NU(NCr)-Foxn1^{nu}) or Fox Chase SCID (CB17/Icr-Prkdc^{scid}/IcrIcoCrl), mice). The mice can be female, 6-12 weeks old at tumor implantation and have access to food and water ad libitum. Approximately 70 mg of a tumor can be implanted subcutaneously in the right flank of each mouse. Following implantation, tumors can be measured weekly and once the tumor volumes reach 150-300 mm³, the mice can be randomized into treatment and control groups. In some embodiments, one or more experimental arms can be added to evaluate pharmacokinetics and/or pharmacodynamics. The mice can be treated with a compound of Formula (I) or (II) (e.g., Formula (I-A), (I-B), or (I-C)), or a pharmaceutically acceptable salt thereof, (e.g., via IP or PO (oral) administration) and optionally an additional therapy or therapeutic agent (e.g., any of the additional therapies or therapeutic agents described herein). Throughout the study, health condition, body weight and tumor volumes of the mice can be recorded on a weekly basis. The mice can be sacrificed at 28 days or when the tumor reaches 1 cm³, and the tumors can be evaluated (e.g., by tumor weight, by tumor volume). At the end of each study, the Best Response can be calculated for each treatment arm. Best Response is defined as the minimum value of ΔVolumeₜ for t≥10 days. Best Responses between the control arm(s) and the treatment arm(s) can be compared to determine if the treatment(s) work better than the control(s). In some embodiments, tumor samples can also be collected at the end of each study and relevant proteins (e.g., BCL-X_{L}, BCL-2, MCL-1, BIM, BAX, and/or BAK) can be measured to determine if the treatment might have a better protein modulation profile compared to a control. In some embodiments, tumor samples can also be collected at the end of each study and analyzed for signaling pathway activity (e.g., via phosphoERK levels). For pharmacokinetic and pharmacodynamic studies, tumor and/or blood samples from the mice can be obtained at the same or different time points than efficacy studies. For example, for pharmacokinetic and pharmacodynamic studies, tumor and/or blood samples from the mice can be obtained at Day 5, 6 hours post dosing, and relevant proteins can be measured in the tumor samples and pharmacokinetic studies can be performed on the blood samples or a portion thereof (e.g., plasma).

In some embodiments, the PDX is a model of a myeloproliferative neoplasm (MPN) (e.g., CEL, CML, CNL, essential thrombocythemia (e.g., JAK2 mutant (e.g., JAK2 V617F mutant) essential thrombocythemia or JAK2 wild type essential thrombocythemia), polycythemia vera (e.g., JAK2 mutant (e.g., JAK2 V617F mutant) polycythemia vera or JAK2 wild type polycythemia vera), or myelofibrosis (e.g., primary myelofibrosis (e.g., JAK2 mutant (e.g., JAK2 V617F mutant) primary myelofibrosis or JAK2 wild type primary myelofibrosis), post-essential thrombocythemia myelofibrosis (e.g., JAK2 mutant (e.g., JAK2 V617F mutant) post-essential thrombocythemia myelofibrosis or JAK2 wild type post-essential thrombocythemia myelofibrosis), or post-polycythemia vera myelofibrosis (e.g., JAK2 mutant (e.g., JAK2 V617F mutant) post-polycythemia vera myelofibrosis or JAK2 wild type post-polycythemia vera myelofibrosis)))), a CRC (e.g., BRaf mutant CRC (e.g., Braf V600E CRC) or KRas mutant CRC (e.g., KRas G12C mutant CRC or KRas G12D CRC)), SCLC (e.g., ASCL1 subtype SCLC or NEUROD1 subtype SCLC), a NSCLC (e.g., BRaf mutant NSCLC (e.g., Braf V600E NSCLC), an EGFR mutant NSCLC (e.g., EGFR L858R NSCLC or EGFR exon 19 deletion NSCLC), MET mutant NSCLC (e.g., MET exon 14 deletion NSCLC, MET amplified NSCLC), a KRas mutant NSCLC (e.g., KRas G12C NSCLC)), a lung squamous cell carcinoma, a malignant pleural mesothelioma (e.g., a BAP1 mutant malignant pleural mesothelioma), a melanoma (e.g., Braf mutant melanoma (e.g., Braf V600E melanoma)), a breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), a lymphoma (e.g., a T cell lymphoma (e.g., anaplastic large T cell lymphoma, cutaneous T cell lymphoma, or peripheral T cell lymphoma), or a non-Hodgkin lymphoma (e.g., DLBCL, anaplastic large T cell lymphoma, cutaneous T cell lymphoma, or peripheral T cell lymphoma), a leukemia (e.g., T cell leukemia (e.g., a T-ALL (e.g., relapsed/refractory T-ALL)), post-MPN leukemia, M6-AML, M7-AML), a head and neck cancer, a pancreatic cancer, a bladder cancer, an ovarian cancer (e.g., BRCA1 mutant ovarian cancer or BRCA2 mutant ovarian cancer, HGSOC (e.g., BRCA1 mutant HGSOC or BRCA2 mutant HGSOC)), a cervical cancer, an intrahepatic cholangiocarcinoma, or a mesenchymal cancer (e.g., mesenchymal breast cancer or mesenchymal kidney cancer).

See, e.g., Khan, et al. Nature Medicine 25.12 (2019): 1938-1947; Balachander, et al. Clinical Cancer Research 26.24 (2020): 6535-6549.

The pharmacokinetic parameters of a compound of Formula (I) or (II) (e.g., Formula (I-A), (I-B), or (I-C)), or a pharmaceutically acceptable salt thereof, can be evaluated in an animal model, for instance, a mouse model, a rat model, a dog model, or a nonhuman primate (e.g., cynomolgus monkey) model. An exemplary assay includes the following. For example, pharmacokinetics (PK) studies can be conducted on animals (e.g., male or female CD-1 mice, Sprague Dawley rats, beagle dogs, or cynomolgus monkeys) by two delivery routes: intravenous (IV) injection and oral gavage (PO). Animals in both the IV and PO groups (e.g., n=3) are allowed free access to food and water. A compound of Formula (I) or (II) (e.g., Formula (I-A), (I-B), or (I-C)), or a pharmaceutically acceptable salt thereof, can be formulated in solution for the IV route and solution or suspension for the PO route. On the day of the experiment, the compound of Formula (I) or (II) (e.g., Formula (I-A), (I-B), or (I-C)), or a pharmaceutically acceptable salt thereof, can be administered via vein injection (e.g., at 1 mg/kg) for IV route or via oral gavage (e.g., at 5 to 10 mg/kg) for PO route. In some cases, the animals can be orally pre-dosed with a cytochrome P450 inhibitor (e.g., 1-aminobenzotriazole) prior to (e.g., 16 hours prior to) dosing the compound of Formula (I) or (II) (e.g., Formula (I-A), (I-B), or (I-C)), or a pharmaceutically acceptable salt thereof. Blood samples can be collected via serial bleeding (e.g., at 8 timepoints from 0.83 to 24 hours post dose). At each timepoint, blood can be collected (e.g., approximately 30 μL of blood/timepoint) in a K₂EDTA tube via a vein (e.g., the saphenous vein). Blood samples can be put on wet ice and centrifuged (e.g., at 4600 RPM for 4 minutes) to obtain plasma samples. Plasma samples can be diluted (e.g., with an equal volume of pH 3.0 phosphate buffer) and submitted to LC-MS/MS for sample analysis. Pharmacokinetic parameters, including clearance (IV or PO, depending on the mode of dosing), area under the curve (AUC), and oral bioavailability (% F) can be calculated using a non-compartmental model.

In some embodiments, the % F for a compound of Formula (I) or (II) (e.g., Formula (I-A), (I-B), or (I-C)), or a pharmaceutically acceptable salt thereof, is at least 4%. In some embodiments, the % F for a compound of Formula (I) or (II) (e.g., Formula (I-A), (I-B), or (I-C)), or a pharmaceutically acceptable salt thereof, is at least 10%. In some embodiments, the % F for a compound of Formula (I) or (II) (e.g., Formula (I-A), (I-B), or (I-C)), or a pharmaceutically acceptable salt thereof, is at least 20%. In some embodiments, the % F for a compound of Formula (I) or (II) (e.g., Formula (I-A), (I-B), or (I-C)), or a pharmaceutically acceptable salt thereof, is at least 30%. In some embodiments, the % F for a compound of Formula (I) or (II) (e.g., Formula (I-A), (I-B), or (I-C)), or a pharmaceutically acceptable salt thereof, is at least 40%. In some embodiments, the % F for a compound of Formula (I) or (II) (e.g., Formula (I-A), (I-B), or (I-C)), or a pharmaceutically acceptable salt thereof, is about 4% to about 80% (e.g., about 4% to about 60%, about 4% to about 40%, about 4% to about 20%, about 4% to about 10%, about 20% to about 40%, or about 20% to about 30%). In some embodiments, the % F for a compound of Formula (I) or (II) (e.g., Formula (I-A), (I-B), or (I-C)), or a pharmaceutically acceptable salt thereof, is about 4% to about 20%. In some embodiments, the % F for a compound of Formula (I) or (II) (e.g., Formula (I-A), (I-B), or (I-C)), or a pharmaceutically acceptable salt thereof, is about 20% to about 40%. In some embodiments, the % F for a compound of Formula (I) or (II) (e.g., Formula (I-A), (I-B), or (I-C)), or a pharmaceutically acceptable salt thereof, is about 40% to about 60%. In some embodiments, the % F for a compound of Formula (I) or (II) (e.g., Formula (I-A), (I-B), or (I-C)), or a pharmaceutically acceptable salt thereof, is about 60% to about 80%.

In some embodiments, the clearance for a compound of Formula (I) or (II) (e.g., Formula (I-A), (I-B), or (I-C)), or a pharmaceutically acceptable salt thereof, dosed PO in rats at 10 mg/kg, is less than 10 mL/min/kg (e.g., less than 5 mL/min/kg, less than 3 mL/min/kg, or less than 1 mL/min/kg). In some embodiments, clearance for a compound of Formula (I) or (II) (e.g., Formula (I-A), (I-B), or (I-C)), or a pharmaceutically acceptable salt thereof, dosed PO in rats at 10 mg/kg, is about 0.05 mL/min/kg to about 5 mL/min/kg (e.g., about 0.05 mL/min/kg to about 3 mL/min/kg, about 0.05 mL/min/kg to about 1 mL/min/kg, or about 0.05 mL/min/kg to about 0.5 mL/min/kg).

In some embodiments, the AUC for a compound of Formula (I) or (II) (e.g., Formula (I-A), (I-B), or (I-C)), or a pharmaceutically acceptable salt thereof, dosed PO in rats at 10 mg/kg, is about 10 μM·h to about 150 μM·h (e.g., about 10 μM·h to about 100 μM·h, about 10 μM·h to about 50 μM·h, or about 30 μM·h to about 80 μM·h).

Heterobifunctional degraders can, in some cases, induce the degradation of off-target proteins. For heterobifunctional degraders that utilize CRBN, common off-target proteins that can be degraded include GSPT1, IKZF1, IKZF2, IKZF3, and/or CK1α. This degradation is generally believed to be due to the E3 binding moiety of the heterobifunctional degrader facilitating ternary complex formation between the off-target protein and CRBN. GSPT1 is a translation termination factor, and CK1α is a kinase that is involved in many key cellular processes including cell cycle progression and chromosome segregation; these are both commonly essential genes, so undesired degradation of either or both may lead to nonspecific cytotoxicity. The IKZF proteins are zinc finger transcription factors that are involved with cell fate during hematopoiesis, and degradation of these proteins has been associated with hematotoxicity. See, e.g., Moreau, Kevin, et al. British Journal of Pharmacology 177.8 (2020): 1709-1718.

In some embodiments, the compounds of Formula (I) or (II) (e.g., Formula (I-A), (T-B), or (I-C)), or pharmaceutically acceptable salts thereof, can exhibit potent and selective induction of degradation of a BCL-X_{L} protein. In some embodiments, a compound of Formula (I) or (II) (e.g., Formula (I-A), (I-B), or (I-C)), or a pharmaceutically acceptable salt thereof, or a pharmaceutically acceptable salt thereof, can selectively target a BCL-X_{L} protein for degradation over an off-target protein, such as another BCL-2 family member (e.g., BCL-2 and/or MCL-1) or non-BCL-2 family member target (e.g., GSPT1, IKZF1, IKZF2, IKZF3, and/or CK1α).

As used herein, “selective” or “selectively”, when referring to a compound of Formula (I) or (II) (e.g., Formula (I-A), (I-B), or (I-C)), or a pharmaceutically acceptable salt thereof in a protein degradation assay, indicates at least a 5-fold (e.g., at least a 10-fold, at least a 25-fold, at least a 50-fold, or at least a 100-fold) superior performance in the protein degradation assay for a specified protein with reference to a comparator protein in the assay. In some embodiments, the specified protein is BCL-X_{L} protein and the comparator is BCL-2 protein. For example, if a compound of Formula (I) or (II) (e.g., Formula (I-A), (I-B), or (I-C)), or a pharmaceutically acceptable salt thereof, “selectively” induces degradation of BCL-X_{L} protein over BCL-2 protein as determined by a degradation assay, then the compound has at least a 5-fold (e.g., at least a 10-fold, at least a 25-fold, at least a 50-fold, or at least a 100-fold) smaller DC₅₀ value for BCL-X_{L} protein than for the BCL-2 protein when measured by the degradation assay.

In some embodiments, the compounds provided herein can exhibit potency (e.g., nanomolar potency) against a BCL-X_{L} protein with minimal activity (e.g., micromolar potency) against BCL-2 family members (e.g., BCL-2 or MCL-1 proteins). In some embodiments, a compound of Formula (I) or (II) (e.g., Formula (I-A), (I-B), or (I-C)), or a pharmaceutically acceptable salt thereof, can exhibit potent degradation of a BCL-X_{L} protein and have minimal potency in degrading (e.g., as measured by Yₘᵢₙ, DC₅₀, and/or Dₘₐₓ values) an off-target protein (e.g., a BCL-2 family member (e.g., BCL-2 and/or MCL-1), GSPT1, IKZF1, IKZF2, IKZF3 and/or CK1α). In some embodiments, a compound of Formula (I) or (II) (e.g., Formula (I-A), (I-B), or (I-C)), or a pharmaceutically acceptable salt thereof, can exhibit greater induction of degradation of a BCL-X_{L} protein relative to induction of degradation (e.g., as measured by Yₘᵢₙ, DC₅₀, and/or Dₘₐₓ values) of an off-target protein (e.g., a BCL-2 family member (e.g., BCL-2 and/or MCL-1), GSPT1, IKZF1, IKZF2, IKZF3, and/or CK1α). In some embodiments, a compound of Formula (I) or (II) (e.g., Formula (I-A), (I-B), or (I-C)), or a pharmaceutically acceptable salt thereof, can exhibit at least 2-fold, 3-fold, 5-fold, 10-fold, 25-fold, 50-fold, or 100-fold greater induction of degradation of a BCL-X_{L} protein relative to induction of degradation of an off-target protein. In some embodiments, a compound of Formula (I) or (II) (e.g., Formula (I-A), (I-B), or (I-C)), or a pharmaceutically acceptable salt thereof, can exhibit up to 1000-fold greater induction of degradation of a BCL-X_{L} protein relative to induction of degradation of an off-target protein. In some embodiments, a compound of Formula (I) or (II) (e.g., Formula (I-A), (I-B), or (I-C)), or a pharmaceutically acceptable salt thereof, can exhibit from about 2-fold to about 10-fold greater induction of degradation of a BCL-X_{L} protein relative to induction of degradation of an off-target protein (e.g., GSPT1, IKZF1, IKZF2, and/or IKZF3 and/or CK1α) (e.g., as measured by Yₘᵢₙ, DC₅₀, and/or Dₘₐₓ values). In some embodiments, a compound of Formula (I) or (II) (e.g., Formula (I-A), (I-B), or (I-C)), or a pharmaceutically acceptable salt thereof, can exhibit from about 10-fold to about 100-fold greater induction of degradation of a BCL-X_{L} protein relative to induction of degradation of an off-target protein. In some embodiments, a compound of Formula (I) or (II) (e.g., Formula (I-A), (I-B), or (I-C)), or a pharmaceutically acceptable salt thereof, can exhibit from about 100-fold to about 1000-fold greater induction of degradation of a BCL-X_{L} protein relative to induction of degradation of an off-target protein. In some embodiments, a compound of Formula (I) or (II) (e.g., Formula (I-A), (I-B), or (I-C)), or a pharmaceutically acceptable salt thereof, can exhibit from about 1000-fold to about 10000-fold greater induction of degradation of a BCL-X_{L} protein relative to induction of degradation of an off-target protein.

Certain agents that inhibit or induce degradation of BCL-X_{L} have demonstrated platelet toxicity, which resulted in dose-limiting toxicity (e.g., thrombocytopenia) in the clinic. See, e.g., Adams and Cory, Cell Death & Differentiation 25.1 (2018): 27-36; Campbell and Tait. Open Biology 8.5 (2018): 180002; Pullarkat et al., Cancer Discovery (2021) 10.1158/2159-8290.CD-20-1465; Negi and Voisin-Chiret. ChemBioChem (2022) (doi: 10.1002/cbic.202100689). The viability of platelets may be monitored with any appropriate assay, such as those described herein. See, for instance, Example B6.

In some embodiments, a therapeutically effective amount of a compound of Formula (I) or (II) (e.g., Formula (I-A), (I-B), or (I-C)), or a pharmaceutically acceptable salt thereof, shows at least about 30% platelet viability (e.g., at least about 50% platelet viability, or at least about 80% platelet viability) when administered to a subject. In some embodiments, a therapeutically effective amount of a compound of Formula (I) or (II) (e.g., Formula (I-A), (I-B), or (I-C)), or a pharmaceutically acceptable salt thereof, shows about 30% to about 100% platelet viability (e.g., about 50% to about 100% platelet viability, or about 80% to about 100% platelet viability) when administered to a subject.

In some embodiments, a therapeutically effective amount of a compound of Formula (I) or (II) (e.g., Formula (I-A), (I-B), or (I-C)), or a pharmaceutically acceptable salt thereof, shows at least about 30% platelet viability (e.g., at least about 50% platelet viability, or at least about 80% platelet viability) when administered to a subject and has a Yₘᵢₙ value of about 50% to about 70%. In some embodiments, a therapeutically effective amount of a compound of Formula (I) or (II) (e.g., Formula (I-A), (I-B), or (I-C)), or a pharmaceutically acceptable salt thereof, shows at least about 30% platelet viability (e.g., at least about 50% platelet viability, or at least about 80% platelet viability) when administered to a subject and has a Yₘᵢₙ value of less than about 50%. In some embodiments, a therapeutically effective amount of a compound of Formula (I) or (II) (e.g., Formula (I-A), (I-B), or (I-C)), or a pharmaceutically acceptable salt thereof, shows at least about 30% platelet viability (e.g., at least about 50% platelet viability, or at least about 80% platelet viability) when administered to a subject and has a Yₘᵢₙ value of about 0% to about 50%.

In some embodiments, a therapeutically effective amount of a compound of Formula (I) or (II) (e.g., Formula (I-A), (I-B), or (I-C)), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of about 50% to about 70%. In some embodiments, a therapeutically effective amount of a compound of Formula (I) or (II) (e.g., Formula (I-A), (I-B), or (I-C)), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of less than about 50%. In some embodiments, a therapeutically effective amount of a compound of Formula (I) or (II) (e.g., Formula (I-A), (I-B), or (I-C)), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of about 0% to about 50%.

In some embodiments, therapeutically effective amount of a compound of Formula (I) or (II) (e.g., Formula (I-A), (I-B), or (I-C)), or a pharmaceutically acceptable salt thereof, shows at least about 50% platelet viability (e.g., at least about 80% platelet viability) when administered to a subject and has a Yₘᵢₙ value of about 50% to about 70%. In some embodiments, a therapeutically effective amount of a compound of Formula (I) or (II) (e.g., Formula (I-A), (I-B), or (I-C)), or a pharmaceutically acceptable salt thereof, shows at least about 50% platelet viability (e.g., at least about 80% platelet viability) when administered to a subject and has a Yₘᵢₙ value of less than about 50%. In some embodiments, a therapeutically effective amount of a compound of Formula (I) or (II) (e.g., Formula (I-A), (I-B), or (I-C)), or a pharmaceutically acceptable salt thereof, shows at least about 50% platelet viability (e.g., at least about 80% platelet viability) when administered to a subject and has a Yₘᵢₙ value of about 0% to about 50%.

In some embodiments, a therapeutically effective amount of a compound of Formula (I) or (II) (e.g., Formula (I-A), (I-B), or (I-C)), or a pharmaceutically acceptable salt thereof, shows between about 50% and 100% platelet viability (e.g., about 80% to about 100% platelet viability) when administered to a subject and has a Yₘᵢₙ value of about 50% to about 70%. In some embodiments, a therapeutically effective amount of a compound of Formula (I) or (II) (e.g., Formula (I-A), (I-B), or (I-C)), or a pharmaceutically acceptable salt thereof, shows between about 50% and 100% platelet viability (e.g., about 80% to about 100% platelet viability) when administered to a subject and has a Yₘᵢₙ value of less than about 50%. In some embodiments, a therapeutically effective amount of a compound of Formula (I) or (II) (e.g., Formula (I-A), (I-B), or (I-C)), or a pharmaceutically acceptable salt thereof, shows between about 50% and 100% platelet viability (e.g., about 80% to about 100% platelet viability) when administered to a subject and has a Yₘᵢₙ value of about 0% to about 50%.

In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability). In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows about 30% to about 100% platelet viability (e.g., about 50% to about 100% platelet viability, or about 80% to about 100% platelet viability).

In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of about 50% to about 70% in the assay described in Example B1. In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of less than about 50% in the assay described in Example B1. In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of about 0% to about 50% in the assay described in Example B1.

In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of about 50% to about 70% in the assay described in Example B1. In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of less than about 50% in the assay described in Example B1. In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of about 0% to about 50% in the assay described in Example B1.

In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of about 50% to about 70% in the assay described in Example B1. In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of less than about 50% in the assay described in Example B1. In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of about 0% to about 50% in the assay described in Example B1.

In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of about 50% to about 70% in the assay described in Example B1. In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of less than about 50% in the assay described in Example B1. In some embodiments, a compound of Formula (I) or (II) (e.g., Formula (I-A), (I-B), or (I-C)), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of about 0% to about 50% in the assay described in Example B1.

Provided herein is a method of treating a cancer in a subject in need of such treatment, the method comprising administering to the subject a therapeutically effective amount of a compound of Formula (I) or (II) (e.g., Formula (I-A), (I-B), or (I-C)), or a pharmaceutically acceptable salt thereof, or a pharmaceutical composition thereof. In some embodiments, the subject is treatment naïve with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) or (II) (e.g., Formula (I-A), (I-B), or (I-C)), or a pharmaceutically acceptable salt thereof, or a pharmaceutical composition thereof, as a monotherapy. In some embodiments, the subject is treatment naïve with respect to the cancer. In some embodiments, the subject has received one or more lines of previous therapy for the cancer.

Provided herein is a method of treating a cancer in a subject in need of such treatment, the method comprising:
(a) detecting a biomarker (e.g., a mutation, an amplification, a copy number increase, and/or expression (optionally including level of expression) of the biomarker) associated with the cancer; and
(b) administering to the subject a therapeutically effective amount of a compound of Formula (I) or (II) (e.g., Formula (I-A), (I-B), or (I-C)), or a pharmaceutically acceptable salt thereof, or a pharmaceutical composition thereof, as a monotherapy or in combination with an additional therapy or therapeutic agent.

Also provided herein is a method of treating a cancer in a subject, wherein the subject has been determined (e.g., prior to administration of a compound of Formula (I) or (II) (e.g., Formula (I-A), (I-B), or (I-C))) to have a biomarker (e.g., a mutation, an amplification, a copy number increase, and/or expression (optionally including level of expression) of the biomarker) associated with the cancer, the method comprising administering to the subject a therapeutically effective amount of a compound of Formula (I) or (II) (e.g., Formula (I-A), (T-B), or (I-C)), or a pharmaceutically acceptable salt thereof, or a pharmaceutical composition thereof, as a monotherapy or in combination with an additional therapy or therapeutic agent.

Provided herein is a method of treating a cancer in a subject in need of such treatment, the method comprising:
(a) detecting a biomarker (e.g., a mutation, an amplification, a copy number increase, and/or expression (optionally including level of expression) of a marker of susceptibility to particular agents (e.g., HER2 expression, ER expression, PR expression, folate receptor expression)) associated with the cancer; and
(b) administering to the subject a therapeutically effective amount of a compound of Formula (I) or (II) (e.g., Formula (I-A), (I-B), or (I-C)), or a pharmaceutically acceptable salt thereof, or a pharmaceutical composition thereof, as a monotherapy or in combination with an additional therapy or therapeutic agent.

Also provided herein is a method of treating a cancer in a subject, wherein the subject has been determined (e.g., prior to administration of a compound of Formula (I) or (II) (e.g., Formula (I-A), (I-B), or (I-C))) to have a biomarker (e.g., a mutation, an amplification, a copy number increase, and/or expression (optionally including level of expression) of a marker of susceptibility to particular agents (e.g., HER2 expression, ER expression, PR expression, folate receptor expression)) associated with the cancer, the method comprising administering to the subject a therapeutically effective amount of a compound of Formula (I) or (II) (e.g., Formula (I-A), (I-B), or (I-C)), or a pharmaceutically acceptable salt thereof, or a pharmaceutical composition thereof, as a monotherapy or in combination with an additional therapy or therapeutic agent.

Provided herein is use of a compound of Formula (I) or (II) (e.g., Formula (I-A), (I-B), or (I-C)), or a pharmaceutically acceptable salt thereof, or a pharmaceutical composition thereof, for the treatment of cancer, for example, any of the cancers provided herein.

Provided herein is use of a compound of Formula (I) or (II) (e.g., Formula (I-A), (I-B), or (I-C)), or a pharmaceutically acceptable salt thereof, or a pharmaceutical composition thereof, as a medicament for the treatment of cancer, for example, any of the cancers provided herein.

Provided herein is use of a compound of Formula (I) or (II) (e.g., Formula (I-A), (I-B), or (I-C)), or a pharmaceutically acceptable salt thereof, in the manufacture of a medicament for the treatment of cancer, for example, any of the cancers provided herein.

Provided herein is a compound of Formula (I) or (II) (e.g., Formula (I-A), (I-B), or (I-C)), or a pharmaceutically acceptable salt thereof, or a pharmaceutical composition thereof, for use as a medicament. Also provided herein is a compound of Formula (I) or (II) (e.g., Formula (I-A), (I-B), or (I-C)), or a pharmaceutically acceptable salt thereof, or a pharmaceutical composition thereof, for use as a medicament for the treatment of cancer, for example, any of the cancers provided herein.

Provided herein is a compound of Formula (I) or (II) (e.g., Formula (I-A), (I-B), or (I-C)), or a pharmaceutically acceptable salt thereof, or a pharmaceutical composition thereof, for use in treating a cancer, for example, any of the cancers provided herein.

As used herein, “monotherapy”, when referring to a compound of Formula (I) or (II) (e.g., Formula (I-A), (I-B), or (I-C)), or a pharmaceutically acceptable salt thereof, means that the compound of Formula (I) or (II) (e.g., Formula (I-A), (I-B), or (I-C)), or a pharmaceutically acceptable salt thereof, is the only therapeutic agent or therapy (e.g., anticancer agent or therapy) administered to the subject during the treatment cycle (e.g., no additional targeted therapeutics, anticancer agents, chemotherapeutics, or checkpoint inhibitors are administered to the subject during the treatment cycle). As a person of ordinary skill in the art would understand, monotherapy does not exclude the co-administration of medicaments for the treatment of side effects or general symptoms associated with the cancer or treatment, such as pain, rash, edema, photosensitivity, pruritis, skin discoloration, hair brittleness, hair loss, brittle nails, cracked nails, discolored nails, swollen cuticles, fatigue, weight loss, general malaise, shortness of breath, infection, anemia, or gastrointestinal symptoms, including nausea, diarrhea, and lack of appetite.

As used herein, “the subject has previously received one or more therapeutic agents or therapies for the cancer” means that the subject has been previously administered one or more therapeutic agents or therapies (e.g., anticancer agent or therapy) for the cancer other than a compound of Formula (I) or (II) (e.g., Formula (I-A), (I-B), or (I-C)), or a pharmaceutically acceptable salt thereof, during a prior treatment cycle. In some embodiments, the subject cannot tolerate the one or more therapeutic agents or therapies previously administered for the cancer. In some embodiments, the subject did not respond to the one or more therapeutic agents or therapies previously administered for the cancer. In some embodiments, the subject did not adequately respond to one or more therapeutic agents or therapies previously administered for the cancer. In some embodiments, the subject has stopped responding to the one or more therapeutic agents or therapies previously administered for the cancer. In some embodiments, a lack of response, an inadequate response, or a discontinued response can be determined by objective criteria (e.g., tumor volume, or by criteria such as RECIST 1.1). In some embodiments, a lack of response, an inadequate response, or a discontinued response can be determined by the subject's physician.

As used herein, “the subject is treatment naïve with respect to the cancer” means that the subject has not been previously administered one or more therapeutic agents or therapies for the cancer.

For any of the solid tumors described herein, the solid tumor can be primary tumors or metastatic (or secondary) tumors. As used herein, “primary” tumors are those located at the site where the tumor began to grow (i.e., where it originated). As used herein, “metastatic” (or “secondary”) tumors are those that have spread to other parts of body from the original tumor site. In some embodiments, the metastatic or secondary tumors are the same type of cancer as the primary tumor. In some embodiments, the metastatic or secondary tumors are not genetically identical to the primary tumor.

In some embodiments of any of the methods or uses described herein, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, neuroblastoma, pheochromocytoma, paraganglioma), multiple neuroendocrine type I and type II tumors, parathyroid cancer, pituitary tumors, thyroid cancer (e.g., papillary thyroid cancer)), eye cancer (e.g., uveal cancer (e.g., uveal melanoma)), gastrointestinal cancer (e.g., anal cancer, bile duct cancer (e.g., cholangiocarcinoma (e.g., intrahepatic cholangiocarcinoma)), colorectal cancer (e.g., colon adenocarcinoma, rectal adenocarcinoma, mucinous adenocarcinoma, mucinous carcinoma), esophageal cancer (e.g., esophageal adenocarcinoma), gallbladder cancer, gastrointestinal carcinoid tumor, gastrointestinal stromal tumor (GIST), liver cancer (e.g., hepatocellular carcinoma, intrahepatic bile duct cancer), pancreatic cancer (e.g., pancreatic adenocarcinoma, pancreatic islet cell cancer), small intestine cancer, or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), testicular cancer (e.g., testicular germ cell tumors), or ureter cancer), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), uterine cancer (e.g., uterine carcinosarcoma, uterine endometrioid carcinoma, uterine serous carcinoma, uterine papillary serous carcinoma, uterine corpus endometrial carcinoma), or vulvar cancer), head and neck cancer (e.g., ear cancer (e.g., middle ear cancer), head and neck squamous cell carcinoma, nasal cavity cancer, oral cancer, pharynx cancer (e.g., hypopharynx cancer, nasopharynx cancer, oropharyngeal cancer), hematological cancer (e.g., leukemia (e.g., chronic eosinophilic leukemia (CEL), chronic lymphocytic leukemia (CLL), chronic neutrophilic leukemia (CNL), acute lymphocytic leukemia (ALL) (e.g., Philadelphia chromosome positive ALL or T cell ALL (T-ALL)), acute myeloid leukemia (AML) (e.g., acute promyelocytic leukemia (APL), post-MPN AML, post-myelodysplastic syndrome (post-MDS) AML, M6-AML (also known as pure erythroid leukemia (PEL)), or M7-AML (also known as acute megakaryoblastic leukemia (AKML))), chronic myeloid leukemia (CML)), lymphoma (e.g., Hodgkin lymphoma (e.g., nodular lymphocyte predominant Hodgkin lymphoma (NLPHL)), or non-Hodgkin lymphoma (e.g., Burkitt lymphoma (BL), diffuse large B cell lymphoma (DLBCL), diffuse histiocytic lymphoma (DHL), follicular lymphoma (FL), intravascular large B cell lymphoma (IVLBCL), mantle cell lymphoma (MCL), small lymphocytic lymphoma (SLL), a T cell lymphoma (e.g., anaplastic large T cell lymphoma, cutaneous T cell lymphoma, or peripheral T cell lymphoma))), essential thrombocythemia, polycythemia vera, myelofibrosis (e.g., primary myelofibrosis, post-essential thrombocythemia myelofibrosis, or post-polycythemia vera myelofibrosis), a myelodysplastic syndrome (MDS) (e.g., M6 MDS or M7 MDS), or myeloma (e.g., multiple myeloma)), Li-Fraumeni tumors, mesentery cancer (e.g., omentum cancer, peritoneal cancer), pleural cancer, respiratory cancer (e.g., larynx cancer, lung cancer (e.g., lung squamous cell carcinoma, lung adenocarcinoma, malignant pleural mesothelioma, non-small cell lung cancer (NSCLC), small cell lung cancer), tracheal cancer), sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), skin cancer (e.g., melanoma or Merkel cell carcinoma), thymus cancer (e.g., thymoma), or a combination thereof.

In some embodiments, the cancer is breast cancer, gastrointestinal cancer (e.g., bile duct cancer (e.g., cholangiocarcinoma (e.g., intrahepatic cholangiocarcinoma)), colorectal cancer (CRC), gastrointestinal stromal tumor, or pancreatic cancer), genitourinary cancer (e.g., bladder cancer (e.g., bladder urothelial carcinoma) or kidney cancer), gynecologic cancer (e.g., cervical cancer, ovarian cancer (e.g., high grade serous ovarian cancer (HGSOC), low grade serous ovarian cancer (LGSOC)), or uterine cancer), head and neck cancer (e.g., head and neck squamous cell carcinoma), hematological cancer (e.g., leukemia (e.g., acute lymphocytic leukemia (ALL) (e.g., T-ALL), acute myeloid leukemia (AML) (e.g., APL, post-MPN AML, post-MDS AML, M6-AML, or M7-AML), chronic lymphocytic leukemia (CLL)), lymphoma (e.g., follicular lymphoma (FL), small lymphocytic lymphoma (SLL), a T cell lymphoma (e.g., anaplastic large T cell lymphoma, cutaneous T cell lymphoma, or peripheral T cell lymphoma), or diffuse large B cell lymphoma (DLBCL)), essential thrombocythemia, polycythemia vera, myelofibrosis (e.g., primary myelofibrosis, post-essential thrombocythemia myelofibrosis, or post-polycythemia vera myelofibrosis), or a myelodysplastic syndrome (MDS) (e.g., M6 MDS or M7 MDS)), lung cancer (e.g., non small cell lung cancer (NSCLC), small cell lung cancer (SCLC), lung squamous cell carcinoma, or malignant pleural mesothelioma (MPM)), neuroblastoma, sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), or skin cancer (e.g., melanoma or Merkel cell carcinoma).

In some embodiments, the cancer is a myeloproliferative neoplasm (MPN). In some embodiments, the myeloproliferative neoplasm is CEL, CML, CNL, essential thrombocythemia, polycythemia vera, or myelofibrosis (e.g., primary myelofibrosis, post-essential thrombocythemia myelofibrosis, or post-polycythemia vera myelofibrosis). In some embodiments, the MPN has a JAK2 mutation (e.g., a JAK2 V617F mutation). In some embodiments, the MPN does not have a JAK2 mutation. In some embodiments, the cancer is low-risk myelofibrosis. In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis). In some embodiments, the cancer is intermediate or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation). In some embodiments, the cancer is intermediate or high-risk myelofibrosis (e.g., primary myelofibrosis, post-essential thrombocythemia myelofibrosis, or post-polycythemia vera myelofibrosis) without a JAK2 V617F mutation. In some embodiments, the subject has a platelet count below 50×10⁹/L.

In some embodiments, the cancer is polycythemia vera. In some embodiments, the cancer is polycythemia vera, and the subject has had an inadequate response to or is intolerant of hydroxyurea.

In some embodiments, the cancer is an MDS. In some embodiments, the cancer is M6 MDS. In some embodiments, the cancer is M7 MDS.

In some embodiments, the cancer is T-ALL. In some embodiments, the cancer is relapsed/refractory T-ALL.

In some embodiments, the cancer is CRC (e.g., Braf mutant CRC (e.g., Braf V600E CRC) or KRas mutant CRC (e.g., KRas G12C CRC or KRas G12D CRC)).

In some embodiments, the cancer is SCLC (e.g., ASCL1 subtype SCLC or NEUROD1 subtype SCLC).

In some embodiments, the cancer is NSCLC (e.g., Braf mutant NSCLC (e.g., Braf V600E NSCLC), EGFR mutant NSCLC (e.g., EGFR L858R NSCLC or EGFR exon 19 deletion NSCLC), MET mutant NSCLC (e.g., MET exon 14 deletion NSCLC, MET amplified NSCLC), KRas mutant NSCLC (e.g., KRas G12C NSCLC)).

In some embodiments, the cancer is lung squamous cell carcinoma.

In some embodiments, the cancer is malignant pleural mesothelioma (e.g., BAP1 mutant malignant pleural mesothelioma). In some embodiments, the cancer is malignant pleural mesothelioma, and a compound of Formula (I) or (II) (e.g., Formula (I-A), (I-B), or (I-C)), or a pharmaceutically acceptable salt thereof, is administered as a monotherapy.

In some embodiments, the cancer is melanoma (e.g., Braf mutant melanoma (e.g., Braf V600E melanoma)).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), HER2 low breast cancer, triple negative breast cancer, hormone receptor positive breast cancer (ER+ and/or PR+, with or without HER2 positivity)).

In some embodiments, the cancer is lymphoma. In some embodiments, the lymphoma is a T cell lymphoma (e.g., anaplastic large T cell lymphoma, cutaneous T cell lymphoma, or peripheral T cell lymphoma). In some embodiments, the lymphoma is a non-Hodgkin lymphoma (e.g., DLBCL, anaplastic large T cell lymphoma, cutaneous T cell lymphoma, or peripheral T cell lymphoma). In some embodiments, the lymphoma is peripheral T cell lymphoma.

In some embodiments, the cancer is leukemia. In some embodiments, the leukemia is a T cell leukemia (e.g., T cell ALL). In some embodiments, the cancer is post-MPN leukemia. In some embodiments, the cancer is a T cell leukemia (e.g., T cell ALL), and a compound of Formula (I) or (II) (e.g., Formula (I-A), (I-B), or (I-C)), or a pharmaceutically acceptable salt thereof, is administered as a monotherapy.

In some embodiments, the cancer is M6-AML. In some embodiments, the M6-AML is a post-MPN AML. In some embodiments, the M6-AML is a post-myelodysplastic syndrome (MDS) AML. In some embodiments, the cancer is M6-AML, and the compound of Formula (I) or (II) (e.g., Formula (I-A), (I-B), or (I-C)), or pharmaceutically acceptable salt thereof, is administered as a monotherapy.

In some embodiments, the cancer is M7-AML. In some embodiments, the M7-AML is a post-MPN AML. In some embodiments, the M7-AML is a post-MDS AML. In some embodiments, the cancer is M7-AML, and the compound of Formula (I) or (II) (e.g., Formula (I-A), (I-B), or (I-C)), or pharmaceutically acceptable salt thereof, is administered as a monotherapy.

In some embodiments, the cancer is head and neck cancer.

In some embodiments, the cancer is essential thrombocythemia.

In some embodiments, the cancer is polycythemia vera.

In some embodiments, the cancer is myelofibrosis. In some embodiments, the cancer is primary myelofibrosis. In some embodiments, the cancer is post-essential thrombocythemia myelofibrosis. In some embodiments, the cancer is post-polycythemia vera myelofibrosis.

In some embodiments, the cancer is an MDS. In some embodiments, the MDS is M6 MDS. IN some embodiments, the MDS is M7 MDS. In some embodiments, the cancer is an MDS, and a compound of Formula (I) or (II) (e.g., Formula (I-A), (I-B), or (I-C)), or a pharmaceutically acceptable salt thereof, is administered as a monotherapy.

In some embodiments, the cancer is pancreatic cancer.

In some embodiments, the cancer is bladder cancer (e.g., bladder urothelial carcinoma).

In some embodiments, the cancer is ovarian cancer (e.g., BRCA1 mutant ovarian cancer or BRCA2 mutant ovarian cancer). In some embodiments, the cancer is HGSOC (e.g., BRCA1 mutant HGSOC or BRCA2 mutant HGSOC).

In some embodiments, the cancer is cervical cancer.

In some embodiments, the cancer is colorectal cancer.

In some embodiments, the cancer is skin cancer. In some embodiments, the skin cancer is melanoma. In some embodiments, the cancer is Merkel cell carcinoma. In some embodiments, the cancer is Merkel cell carcinoma, and a compound of Formula (I) or (II) (e.g., Formula (I-A), (I-B), or (I-C)), or a pharmaceutically acceptable salt thereof, is administered as a monotherapy.

In some embodiments, the cancer is neuroblastoma.

In some embodiments, the cancer is intrahepatic cholangiocarcinoma.

In some embodiments, the cancer is a mesenchymal cancer. In some embodiments, the mesenchymal cancer is mesenchymal breast cancer or mesenchymal kidney cancer.

In some embodiments, a BCL-X_{L} copy number gain or a BCL-X_{L} amplification can be detected in a sample from the subject (e.g., detecting three or more copies of a BCL2L1 gene in the sample from the subject). In some embodiments, the subject was determined (e.g., prior to administration of a compound of Formula (I) or (II) (e.g., Formula (I-A), (I-B), or (I-C))) to have a cancer having a BCL-X_{L} copy number gain or a BCL-X_{L} amplification.

In some embodiments, the cancer has a BCL-X_{L} copy number gain.

In some embodiments, the cancer has a BCL-X_{L} amplification.

Non-limiting examples of BCL-X_{L} involvement in cancers can be found in: Wilson, Wyndham H., et al. The Lancet Oncology 11.12 (2010): 1149-1159; Keitel, Ulrike, et al. Oncotarget 5.23 (2014): 11778; Chonghaile, Triona Ni, et al. Cancer Discovery 4.9 (2014): 1074-1087; Zaanan, Aziz, et al. Journal of Biological Chemistry 290.39 (2015): 23838-23849; Zhang, Haichao, et al. Molecular Cancer 14.1 (2015): 1-9; Soderquist, Ryan S., et al. Nature Communications 9.1 (2018): 1-13; Stover, Elizabeth H., et al. Molecular Cancer Research 17.11 (2019): 2281-2293; Concoran, R. B., et al. Annals of Oncology (2019) 30 (suppl_5): v159-v193; Lakhani, Nehal J., et al. Journal of Clinical Oncology (2020): 3509-3509 doi: 10.1200/JCO.2020.38; He, Yonghan, et al. Journal of Hematology & Oncology 13.1 (2020): 1-13; Grubb, Treg, et al. Clinical Cancer Research (2022) doi: 10.1158/1078-0432.CCR-22-0669; Joly, Florence, et al. Gynecologic Oncology 165.1 (2022): 30-39; and Nanjo, Shigeki, et al. The Journal of Clinical Investigation (2022) doi: 10.1172/JCI145099.

In some embodiments, the subject has previously been treated with another anticancer agent, a chemotherapeutic agent, radiation, surgery, a multi-kinase inhibitor, or a combination thereof.

Provided herein is a method of treating an ocular disease or condition in a subject in need of such treatment, the method comprising administering (e.g., intravitreally or topically) to the subject a therapeutically effective amount of a compound of Formula (I) or (II) (e.g., Formula (I-A), (I-B), or (I-C)), or a pharmaceutically acceptable salt thereof, or a pharmaceutical composition thereof. In some embodiments, the ocular disease or condition is diabetic macular edema. In some embodiments, the ocular disease or condition is age-related macular degeneration. In some embodiments, the ocular disease or condition is diabetic retinopathy. See, e.g., Crespo-Garcia, Sergio, et al. Cell Metabolism 33.4 (2021): 818-832; Hassan, Jannah Waled, and Ashay D. Bhatwadekar, Frontiers in Pharmacology 13 (2022): 896907.

Provided herein is use of a compound of Formula (I) or (II) (e.g., Formula (I-A), (I-B), or (I-C)), or a pharmaceutically acceptable salt thereof, or a pharmaceutical composition thereof, for the treatment of an ocular disease or condition, for example, any of the ocular diseases or conditions provided herein.

Provided herein is use of a compound of Formula (I) or (II) (e.g., Formula (I-A), (I-B), or (I-C)), or a pharmaceutically acceptable salt thereof, or a pharmaceutical composition thereof, as a medicament for the treatment of an ocular disease or condition, for example, any of the ocular diseases or conditions provided herein.

Provided herein is use of a compound of Formula (I) or (II) (e.g., Formula (I-A), (I-B), or (I-C)), or a pharmaceutically acceptable salt thereof, in the manufacture of a medicament for the treatment of an ocular disease or condition, for example, any of the ocular diseases or conditions provided herein.

Also provided herein is a compound of Formula (I) or (II) (e.g., Formula (I-A), (I-B), or (I-C)), or a pharmaceutically acceptable salt thereof, or a pharmaceutical composition thereof, for use as a medicament for the treatment of an ocular disease or condition, for example, any of the ocular diseases or conditions provided herein.

Provided herein is a compound of Formula (I) or (II) (e.g., Formula (I-A), (I-B), or (I-C)), or a pharmaceutically acceptable salt thereof, or a pharmaceutical composition thereof, for use in treating an ocular disease or condition, for example, any of the ocular diseases or conditions provided herein.

Also provided herein is a method of treating a fibrotic disease or condition and/or a disease or condition associated with senescent cells in a subject in need of such treatment, the method comprising administering to the subject a therapeutically effective amount of a compound of Formula (I) or (II) (e.g., Formula (I-A), (I-B), or (I-C)), or a pharmaceutically acceptable salt thereof, or a pharmaceutical composition thereof. Non-limiting examples of fibrotic diseases or conditions and/or diseases or conditions associated with senescent cells include pulmonary fibrosis (e.g., idiopathic pulmonary fibrosis, systemic fibrosis-associated lung disease, radiation-associated pulmonary fibrosis), radiation-associated skin fibrosis, liver fibrosis, primary sclerosing cholangitis, diabetic macular edema, age-related macular degeneration, diabetic retinopathy, geographic atrophy, ischemia and reperfusion injury, heart failure, recovery from acute myocardial infarction, pulmonary hypertension, inflammatory bowel disease, colitis, Crohn's disease, diabetes, aging skin (including photoaging-related pigmentation), donor organ transplant survival and function, stem cell transplant survival and function, osteoarthritis, recovery from spinal cord injury, Alzheimer's disease, tau-opathies, progressive supranuclear palsy, and age-related neurological decline (e.g., related to impaired neurovascular coupling). See, e.g., Zhu, Y. I., et al. Aging Cell 14.4 (2015): 644-658; Zhu, Y. I., et al. Aging Cell 15.3 (2016): 428-435; Chang, Jianhui, et al. Nature Medicine 22.1 (2016): 78-83; Zhu, Yi, et al. Aging (Albany NY) 9.3 (2017): 955; Lagares, David, et al. Science Translational Medicine 9.420 (2017): eaa13765; Pan Jin et al. International Journal of Radiation Oncology Biology Physics 99.2 (2017): 353-361; Bussian, Tyler J., et al. Nature 562.7728 (2018): 578-582; Moncsek, Anja, et al. Hepatology 67.1 (2018): 247-259; van Willigenburg, Hester, Peter U de Keizer, and Ron W F de Bruin. Pharmacological Research 130 (2018): 322-330; Walaszczyk, Anna, et al. Aging Cell 18.3 (2019): e12945; Aguayo-Mazzucato, Cristina, et al. Cell Metabolism 30.1 (2019): 129-142; Sessions, Garrett A., et al. The FASEB Journal 33.11 (2019): 12364; Gerdes, Erin O. Wissler, et al. International Review of Neurobiology 155 (2020): 203-234; Sasaki, Motoko, Yasunori Sato, and Yasuni Nakanuma, Journal of Autoimmunity 107 (2020): 102377; Yabluchanskiy, Andriy, et al. Geroscience 42 (2020): 409-428; Dookun, Emily, et al. Aging Cell 19.10 (2020): e13249; Jia, Kangni, et al. Journal of Cardiovascular Pharmacology 76.4 (2020): 452-460; Sierra-Ramirez, Arantzazu, et al. Aging (Albany NY) 12.12 (2020): 11337; Yang, Hao, et al. Aging (Albany NY) 12.13 (2020): 12750; Lawrie, Allan, and Sheila F Francis. The Journal of Clinical Investigation 131.11 (2021): e149721; Paramos-de-Carvalho, Diogo, et al. Cell Reports 36.1 (2021): 109334; Tarantini, Stefano, et al. GeroScience 43.5 (2021): 2427-2440; Park, Ji Hee, et al. The British Journal of Dermatology (2021); Fielder, Edward, et al. ELife 11 (2022): e75492; Suzuki, Keiji, et al. Mutation Research/Genetic Toxicology and Environmental Mutagenesis 876 (2022): 503448; He, An, et al. American Journal of Transplantation 22.11 (2022): 2529-2547; Johnson, Laura A., et al. Inflammatory Bowel Diseases 28.2 (2022): 161-175; Miura, Yugo, et al. Stem Cell Research & Therapy 13.1 (2022): 222; Cooley, Joseph C., et al. JCI Insight 8.3 (2023): e163762. Watanabe, Yusuke, et al. Hepatology Research 53 (2023): 460-472; and Takaya, Kento, et al., Rejuvenation Research 26.1 (2023): 9-20.

Provided herein is use of a compound of Formula (I) or (II) (e.g., Formula (I-A), (I-B), or (I-C)), or a pharmaceutically acceptable salt thereof, or a pharmaceutical composition thereof, for the treatment of a fibrotic disease or condition and/or a disease or condition associated with senescent cells, for example, any of the fibrotic diseases or conditions and/or diseases or conditions associated with senescent cells provided herein.

Provided herein is use of a compound of Formula (I) or (II) (e.g., Formula (I-A), (I-B), or (I-C)), or a pharmaceutically acceptable salt thereof, or a pharmaceutical composition thereof, as a medicament for the treatment of a fibrotic disease or condition and/or a disease or condition associated with senescent cells, for example, any of the fibrotic diseases or conditions and/or diseases or conditions associated with senescent cells provided herein.

Provided herein is use of a compound of Formula (I) or (II) (e.g., Formula (I-A), (I-B), or (I-C)), or a pharmaceutically acceptable salt thereof, in the manufacture of a medicament for the treatment of a fibrotic disease or condition and/or a disease or condition associated with senescent cells, for example, any of the fibrotic diseases or conditions and/or diseases or conditions associated with senescent cells provided herein.

Also provided herein is a compound of Formula (I) or (II) (e.g., Formula (I-A), (I-B), or (I-C)), or a pharmaceutically acceptable salt thereof, or a pharmaceutical composition thereof, for use as a medicament for the treatment of a fibrotic disease or condition and/or a disease or condition associated with senescent cells, for example, any of the fibrotic diseases or conditions and/or diseases or conditions associated with senescent cells provided herein.

Provided herein is a compound of Formula (I) or (II) (e.g., Formula (I-A), (I-B), or (I-C)), or a pharmaceutically acceptable salt thereof, or a pharmaceutical composition thereof, for use in treating a fibrotic disease or condition and/or a disease or condition associated with senescent cells, for example, any of the fibrotic diseases or conditions and/or diseases or conditions associated with senescent cells provided herein.

Also provided is a method for modulating (e.g., decreasing) BCL-X_{L} protein activity in a cell, comprising contacting the cell with an effective compound of Formula (I) or (II) (e.g., Formula (I-A), (I-B), or (I-C)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) or (II) (e.g., Formula (I-A), (I-B), or (I-C)), or a pharmaceutically acceptable salt thereof, to a subject. In some embodiments, the cell is a cancer cell. In some embodiments, the cell is a mammalian cell. In some embodiments, the cell is a mammalian cancer cell. In some embodiments, the cancer cell is any cancer as described herein.

As used herein, the term “contacting” refers to the bringing together of indicated moieties in an in vitro system or an in vivo system. For example, “contacting” a cell with a compound provided herein includes the administration of a compound provided herein to the cell, in vitro or in vivo, including, for example, introducing a compound provided herein into a sample containing cells (e.g., grown in culture or derived from a patient), an organoid, or an organism (e.g., an animal (e.g., an animal bearing a tumor), or a human).

Also provided is a method of modulating (e.g., decreasing) the level of BCL-X_{L} protein in a cell, comprising contacting the cell with a compound of Formula (I) or (II) (e.g., Formula (I-A), (I-B), or (I-C)), or a pharmaceutically acceptable salt thereof. In some embodiments, the level of BCL-X_{L} protein is decreased by at least 30% (e.g., at least 40%, at least 50%, at least 60%, at least 70%, at least 80%, at least 90%, at least 95%, at least 97%, or at least 99%) compared to a cell not contacted with the compound of Formula (I) or (II) (e.g., Formula (I-A), (I-B), or (I-C)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) or (II) (e.g., Formula (I-A), (I-B), or (I-C)), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of inducing ubiquitination of a BCL-X_{L} protein in a cell, comprising contacting the cell with a compound of Formula (I) or (II) (e.g., Formula (I-A), (T-B), or (I-C)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) or (II) (e.g., Formula (I-A), (I-B), or (I-C)), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of forming a ternary complex comprising a BCL-X_{L} protein, a compound of Formula (I) or (II) (e.g., Formula (I-A), (I-B), or (I-C)), or a pharmaceutically acceptable salt thereof, and a CRBN protein or fragment thereof in a cell, comprising contacting the cell with a compound of Formula (I) or (II) (e.g., Formula (I-A), (I-B), or (I-C)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) or (II) (e.g., Formula (I-A), (I-B), or (I-C)), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided herein is a method of inhibiting cell proliferation, in vitro or in vivo, the method comprising contacting a cell with an effective amount of a compound of Formula (I) or (II) (e.g., Formula (I-A), (I-B), or (I-C)), or a pharmaceutically acceptable salt thereof, or a pharmaceutical composition thereof as defined herein.

Further provided herein is a method of increasing cell death, in vitro or in vivo, the method comprising contacting a cell with an effective amount of a compound of Formula (I) or (II) (e.g., Formula (I-A), (I-B), or (I-C)), or a pharmaceutically acceptable salt thereof, or a pharmaceutical composition thereof as defined herein. Also provided herein is a method of increasing tumor cell death in a subject. The method comprises administering to the subject a compound of Formula (I) or (II) (e.g., Formula (I-A), (I-B), or (I-C)), or a pharmaceutically acceptable salt thereof, in an amount effective to increase tumor cell death.

When employed as pharmaceuticals, the compounds of Formula (I) or (II) (e.g., Formula (I-A), (I-B), or (I-C)), or pharmaceutically acceptable salts thereof, can be administered in the form of pharmaceutical compositions as described herein.

Also provided herein is a method for inducing degradation of a BCL-X_{L} protein in a mammalian cell, the method comprising contacting the mammalian cell with an effective amount of a compound of Formula (I) or (II) (e.g., Formula (I-A), (I-B), or (I-C)), or a pharmaceutically acceptable salt thereof.

Also provided herein is a method of treating a subject having a cancer, wherein the method comprises:
administering a therapeutically effective amount of a compound of Formula (I) or (II) (e.g., Formula (I-A), (I-B), or (I-C)), or a pharmaceutically acceptable salt thereof, as a monotherapy or in conjunction with a first anticancer agent to the subject who has been administered one or more doses of the first anticancer agent to the subject for a period of time.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) or (II) (e.g., Formula (I-A), (I-B), or (I-C)), or a pharmaceutically acceptable salt thereof, as a monotherapy or in conjunction with the first anticancer agent to the subject.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) or (II) (e.g., Formula (I-A), (I-B), or (I-C)), or a pharmaceutically acceptable salt thereof, as a monotherapy or in conjunction with a second anticancer agent to the subject.

### Combinations

In any of the indications described herein, a compound of Formula (I) or (II) (e.g., Formula (I-A), (I-B), or (I-C)), or a pharmaceutically acceptable salt thereof, can be used as a monotherapy. In some embodiments, a compound of Formula (I) or (II) (e.g., Formula (I-A), (I-B), or (I-C)), or a pharmaceutically acceptable salt thereof, can be used prior to administration of an additional therapeutic agent or additional therapy. For example, a subject in need thereof can be administered one or more doses of a compound of Formula (I) or (II) (e.g., Formula (I-A), (I-B), or (I-C)), or a pharmaceutically acceptable salt thereof, for a period of time and then undergo at least partial resection of the tumor. In some embodiments, the treatment with one or more doses of a compound of Formula (I) or (II) (e.g., Formula (I-A), (I-B), or (I-C)), or a pharmaceutically acceptable salt thereof, reduces the size of the tumor (e.g., the tumor burden) prior to the at least partial resection of the tumor.

In some embodiments, a subject in need thereof can be administered one or more doses of a compound of Formula (I) or (II) (e.g., Formula (I-A), (I-B), or (I-C)), or a pharmaceutically acceptable salt thereof, for a period of time and under one or more rounds of radiation therapy. In some embodiments, the treatment with one or more doses of a compound of Formula (I) or (II) (e.g., Formula (I-A), (I-B), or (I-C)), or a pharmaceutically acceptable salt thereof, reduces the size of the tumor (e.g., the tumor burden) prior to the one or more rounds of radiation therapy.

In some embodiments of any the methods described herein, the compound of Formula (I) or (II) (e.g., Formula (I-A), (I-B), or (I-C)), or a pharmaceutically acceptable salt thereof, is administered in combination with a therapeutically effective amount of at least one additional therapeutic agent selected from one or more additional therapies or therapeutic (e.g., chemotherapeutic) agents.

Non-limiting examples of additional therapies and therapeutic agents include: RAS pathway targeted therapeutic agents (e.g., Ras/RAF/MEK/PI3K pathway inhibitors, (e.g., Ras inhibitors (e.g., KRas inhibitors), KRas-targeted therapeutic agents, SOS1 inhibitors, SOS1/Ras protein-protein interaction inhibitors, SHP2 inhibitors, PI3K-AKT-mTOR pathway inhibitors)), kinase-targeted therapeutics (e.g., MEK inhibitors, ERK inhibitors, Raf inhibitors (e.g., BRaf inhibitors), PI3K inhibitors, Abl inhibitors (e.g., BCR-Abl inhibitors), ALK inhibitors, AKT inhibitors, AURKA inhibitors, mTOR inhibitors, CDK2 inhibitors, CDK4/5 inhibitors, CDK4/6 inhibitors, CDK7 inhibitors, CDK9 inhibitors, MET (also known as cMET) inhibitors, FAK inhibitors, FGFR1 inhibitors, FGFR2 inhibitors, FGFR3 inhibitors, FGFR4 inhibitors, ErbB family inhibitors (e.g., EGFR inhibitors, anti-EGFR antibodies or anti-EGFR antibody-drug conjugates, HER2 inhibitors, anti-HER2 antibodies or anti-HER2 antibody-drug conjugates), JAK inhibitors (e.g., JAK inhibitors having activity against JAK1, JAK2, and/or JAK1/2), Src inhibitors, VEGFR inhibitors), LSD1 inhibitors, EZH2 inhibitors, BET inhibitors, STING agonists, telomerase inhibitors, mTORC1 inhibitors, YAP inhibitors, proteasome inhibitors, farnesyl transferase inhibitors, Hif2α inhibitors, HSP90 inhibitors, PTEN inhibitors, PARP inhibitors, signal transduction pathway inhibitors, checkpoint inhibitors, modulators of the apoptosis pathway (e.g., BCL-2 inhibitors), chemotherapeutics, angiogenesis-targeted therapies, immune-targeted agents, including immunomodulatory imide drugs (sometimes called “IMiDs” or “CELMoDs”) and immunotherapy (e.g., anti-PD1 therapy or anti-PD-L1 therapy), and radiotherapy.

As used herein, a biosimilar antibody refers to an antibody or antigen-binding fragment that has the same primary amino acid sequence as compared to a reference antibody and optionally, may have detectable differences in post-translation modifications (e.g., glycosylation and/or phosphorylation) as compared to the reference antibody (e.g., a different glycoform).

In some embodiments, the additional therapy or therapeutic agent is an Abl inhibitor (e.g., a BCR-Abl inhibitor), an ALK inhibitor, an AURKA inhibitor, a BCL-2 inhibitor, a Braf inhibitor, a CDK2 inhibitor, a CDK4/6 inhibitor, a CDK7 inhibitor, a CDK9 inhibitor, an EGFR inhibitor, an anti-EGFR antibody or anti-EGFR antibody-utilizing biologic, an ERK inhibitor, an EZH2 inhibitor, a FGFR1 inhibitor, a FGFR2 inhibitor, a FGFR3 inhibitor, a FGFR4 inhibitor, a HER2 inhibitor, an anti-HER2 antibody or anti-HER2 antibody-utilizing biologic, a JAK inhibitor, a KRas inhibitor, a MEK inhibitor, a MET inhibitor, a Hif2α inhibitor, a PARP inhibitor, a VEGFR inhibitor, an LSD1 inhibitor, a BET inhibitor, a STING agonist, a telomerase inhibitor, a TORC1/2 inhibitor, an immunomodulatory imide drug, immunotherapy (e.g., a PD-1 inhibitor (e.g., anti-PD1 therapy), a PD-L1 inhibitor (e.g., anti-PD-L1 therapy)), chemotherapy, radiotherapy, or a combination thereof.

In some embodiments, the additional therapy or therapeutic agent is an Abl degrader (e.g., a BCR-Abl degrader), an ALK degrader, an AURKA degrader, a BCL-2 degrader, a BRaf degrader, a CDK2 degrader, a CDK4/6 degrader, a CDK7 degrader, a CDK9 degrader, an EGFR degrader, an ERK degrader, an EZH2 degrader, a FGFR1 degrader, a FGFR2 degrader, a FGFR3 degrader, a FGFR4 degrader, a HER2 degrader, a JAK2 degrader, a KRas degrader, a MEK degrader, a MET degrader, a Hif2α degrader, a PARP degrader, a VEGFR degrader, an LSD1 degrader, a BET degrader, a telomerase degrader, a TORC1/2 degrader, an immunomodulatory imide drug, immunotherapy (e.g., anti-PD1 therapy or anti-PD-L1 therapy), chemotherapy, radiotherapy, or a combination thereof.

In some embodiments, the Abl inhibitor (e.g., BCR-Abl inhibitor) is asciminib (e.g., asciminib hydrochloride), bafetinib, bosutinib (e.g., bosutinib monohydrate), danusertib, dasatinib (e.g., dasatinib monohydrate), flumatinib (e.g., flumatinib mesylate), imatinib (e.g., imatinib mesylate), nilotinib (e.g., nilotinib monochloride monohydrate), olverembatinib (e.g., olverembatinib mesylate), ponatinib (e.g., ponatinib hydrochloride), radotinib (e.g., radotinib dihydrochloride), ruserontinib, vandetanib, AN-019, AT-9283, IkT-148009, NPB-001-056, or a combination thereof.

In some embodiments, the ALK inhibitor is alectinib (e.g., alectinib hydrochloride), brigatinib, ceritinib, crizotinib, ensartinib (e.g., ensartinib hydrochloride), entrectinib, fidrisertib, lorlatinib, TQ-B-3101, TQ-B-3139, or a combination thereof. In some embodiments, the ALK inhibitor is alectinib (e.g., alectinib hydrochloride), brigatinib, ceritinib, crizotinib, ensartinib (e.g., ensartinib hydrochloride), fidrisertib, lorlatinib, TQ-B-3101, TQ-B-3139, or a combination thereof.

In some embodiments, the AURKA inhibitor is alisertib, danusertib, ilorasertib, tinengotinib, AT-9283, BI-811283, ENMD-2076, or a combination thereof.

In some embodiments, the BCL-2 inhibitor is lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), ZN-d5, or a combination thereof.

In some embodiments, the cancer is a lung cancer (e.g., SCLC), and the additional therapy or therapeutic agent is a BCL-2 inhibitor (e.g., lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), or ZN-d5). In some embodiments, the cancer is a lung cancer (e.g., SCLC), and the additional therapy or therapeutic agent is venetoclax.

In some embodiments, the cancer is a non-Hodgkin lymphoma, and the additional therapy or therapeutic agent is a BCL-2 inhibitor (e.g., lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), or ZN-d5). In some embodiments, the cancer is a non-Hodgkin lymphoma, and the additional therapy or therapeutic agent is venetoclax.

In some embodiments, the BRaf inhibitor is avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, PLX-4720, or a combination thereof.

In some embodiments, a BRaf V600E mutation can be detected in a sample from the subject (e.g., detecting a BRAF gene having a mutation corresponding to a V600E mutation in BRaf protein and/or detecting a BRaf protein having a V600E mutation). In some embodiments, the subject was determined (e.g., prior to administration of a compound of Formula (I) or (II) (e.g., Formula (I-A), (I-B), or (I-C))) to have a cancer having a BRaf V600E mutation.

In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720). In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and an anti-EGFR antibody or anti-EGFR antibody-drug conjugate (e.g., amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI-B-001, TAS-0313, or biosimilars thereof). In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and cetuximab (e.g., ERBITUX® (cetuximab), or a biosimilar thereof (e.g., CMAB-009, CPGJ-602, or KL-140)). In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and panitumumab (e.g., VECTIBIX® (panitumumab), or a biosimilar thereof). In some embodiments, the cancer is a BRaf mutant CRC (e.g., BRaf V600E mutant CRC), and the additional therapy or therapeutic agent is a dabrafenib (e.g., dabrafenib mesylate, GSK2118436), encorafenib (e.g., BRAFTOVI™ LGX818), or vemurafenib (e.g., ZELBORAF®, RO5185426) and cetuximab (e.g., ERBITUX® (cetuximab), or a biosimilar thereof (e.g., CMAB-009, CPGJ-602, or KL-140)). In some embodiments, the cancer is a BRaf mutant CRC (e.g., BRaf V600E mutant CRC), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426) and panitumumab (e.g., VECTIBIX® (panitumumab), or a biosimilar thereof). In some embodiments, the cancer is a BRaf mutant CRC (e.g., BRaf V600E mutant CRC), and the additional therapy or therapeutic agent is a dabrafenib (e.g., dabrafenib mesylate, GSK2118436) and cetuximab (e.g., ERBITUX® (cetuximab), or a biosimilar thereof (e.g., CMAB-009, CPGJ-602, or KL-140)). In some embodiments, the cancer is a BRaf mutant CRC (e.g., BRaf V600E mutant CRC), and the additional therapy or therapeutic agent is dabrafenib (e.g., dabrafenib mesylate, GSK2118436) and panitumumab (e.g., VECTIBIX® (panitumumab), or a biosimilar thereof).

In some embodiments, the cancer is a BRaf mutant NSCLC (e.g., BRaf V600E mutant NSCLC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and a MEK inhibitor (e.g., avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, or TAK-733). In some embodiments, the cancer is a BRaf mutant NSCLC (e.g., BRaf V600E mutant NSCLC), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426), and binimetinib, cobimetinib (e.g., cobimetinib fumarate), selumetinib (e.g., selumetinib sulfate), or trametinib (e.g., trametinib dimethyl sulfoxide). In some embodiments, the cancer is a BRaf mutant NSCLC (e.g., BRaf V600E mutant NSCLC), and the additional therapy or therapeutic agent is dabrafenib (e.g., dabrafenib mesylate, GSK2118436) and trametinib (e.g., trametinib dimethyl sulfoxide). In some embodiments, the cancer is a BRaf mutant NSCLC (e.g., BRaf V600E mutant NSCLC), and the additional therapy or therapeutic agent is vemurafenib (e.g., ZELBORAF®, RO5185426), and cobimetinib (e.g., cobimetinib fumarate). In some embodiments, the cancer is a BRaf mutant NSCLC (e.g., BRaf V600E mutant NSCLC), and the additional therapy or therapeutic agent is encorafenib (e.g., BRAFTOVI™, LGX818) and binimetinib.

In some embodiments, the cancer is a BRaf mutant melanoma (e.g., BRaf V600E mutant melanoma), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720). In some embodiments, the cancer is a BRaf mutant melanoma (e.g., BRaf V600E mutant melanoma), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and a MEK inhibitor (e.g., avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, or TAK-733). In some embodiments, the cancer is a BRaf mutant melanoma (e.g., BRaf V600E mutant melanoma), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426), and binimetinib, cobimetinib (e.g., cobimetinib fumarate), selumetinib (e.g., selumetinib sulfate), or trametinib (e.g., trametinib dimethyl sulfoxide). In some embodiments, the cancer is a BRaf mutant melanoma (e.g., BRaf V600E mutant melanoma), and the additional therapy or therapeutic agent is dabrafenib (e.g., dabrafenib mesylate, GSK2118436) and trametinib (e.g., trametinib dimethyl sulfoxide). In some embodiments, the cancer is a BRaf mutant melanoma (e.g., BRaf V600E mutant melanoma), and the additional therapy or therapeutic agent is vemurafenib (e.g., ZELBORAF®, RO5185426), and cobimetinib (e.g., cobimetinib fumarate). In some embodiments, the cancer is a BRaf mutant melanoma (e.g., BRaf V600E mutant melanoma), and the additional therapy or therapeutic agent is encorafenib (e.g., BRAFTOVI™ LGX818) and binimetinib. In some such embodiments, the subject has previously been treated with an immunotherapy.

In some embodiments, the CDK2 inhibitor is ebvaciclib, fadraciclib, milciclib, pacritinib (e.g., pacritinib citrate), roniciclib, roscovitine, BLU-222, NUV-422, PF-07104091, TQB-3616, or a combination thereof.

In some embodiments, the CDK4/6 inhibitor is abemaciclib, birociclib, dalpiciclib, lerociclib, milciclib, palbociclib, ribociclib (e.g., ribociclib succinate), riviciclib, roniciclib, trilaciclib (e.g., trilaciclib dihydrochloride), FCN-437, TQB-3616, or a combination thereof.

In some embodiments, the CDK7 inhibitor is milciclib, roscovitine, samuraciclib, or a combination thereof.

In some embodiments, the CDK9 inhibitor is fadraciclib, riviciclib, roniciclib, roscovitine, zotiraciclib, AZD-4573, KB-0742, or a combination thereof.

In some embodiments, the EGFR inhibitor is abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, an anti-EGFR antibody or anti-EGFR antibody-drug conjugate, or a combination thereof.

In some embodiments, the anti-EGFR antibody or anti-EGFR antibody-drug conjugate is amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI-B-001, TAS-0313, biosimilars thereof, or a combination thereof. In some embodiments, the anti-EGFR antibody or anti-EGFR antibody-drug conjugate is amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI-B-001, biosimilars thereof, or a combination thereof.

In some embodiments, an EGFR mutation (e.g., an EGFR exon 19 deletion or an EGFR L858R mutation (with or without an EGFR T790M mutation)) can be detected in a sample from the subject (e.g., detecting an EGFR gene having a mutation (e.g., a mutation corresponding to an EGFR exon 19 deletion or an EGFR L858R mutation (with or without an EGFR T790M mutation) in an EGFR protein) and/or detecting an EGFR protein having a mutation (e.g., an EGFR exon 19 deletion or an EGFR L858R mutation (with or without an EGFR T790M mutation))). In some embodiments, the subject was determined (e.g., prior to administration of a compound of Formula (I) or (II) (e.g., Formula (I-A), (I-B), or (I-C))) to have a cancer having an EGFR mutation (e.g., an EGFR exon 19 deletion or an EGFR L858R mutation (with or without an EGFR T790M mutation)).

In some embodiments, the cancer is an EGFR mutant NSCLC (e.g., EGFR exon 19 deletion NSCLC or EGFR L858R (with or without T790M) mutant NCLC), and the additional therapy or therapeutic agent is an EGFR inhibitor (e.g., abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, or an anti-EGFR antibody or anti-EGFR antibody-drug conjugate). In some embodiments, the cancer is an EGFR mutant NSCLC (e.g., EGFR exon 19 deletion NSCLC or EGFR L858R (with or without T790M) mutant NCLC), and the additional therapy or therapeutic agent is osimertinib (e.g., osimertinib mesylate).

In some embodiments, the ERK inhibitor is rineterkib, temuterkib, ulixertinib, ASN-0007, ASTX-029, ATG-017, BPI-27336, HH-2710, JSI-1187, MK-8353, or a combination thereof.

In some embodiments, the EZH2 inhibitor is lirametostat, tazemetostat (e.g., tazemetostat hydrobromide), valemetostat (e.g., valemetostat tosylate), tulmimetostat (CPI-0209), EBI-2511, HH-2853, HM-97662, PF-6821497, SHR-2554, XNW-5004, or a combination thereof. In some embodiments, the EZH2 inhibitor also inhibits EZH1 (also referred to as an EZH1/2 inhibitor).

In some embodiments, the cancer is peripheral T cell lymphoma, and the additional therapy or therapeutic agent is an EZH2 inhibitor (e.g., lirametostat, tazemetostat (e.g., tazemetostat hydrobromide), valemetostat (e.g., valemetostat tosylate), tulmimetostat (CPI-0209), EBI-2511, HH-2853, HM-97662, PF-6821497, SHR-2554, or XNW-5004).

In some embodiments, the FGFR1 inhibitor is danusertib, dovitinib, erdafitinib, futibatinib, infigratinib (e.g., infigratinib phosphate), lenvatinib (e.g., lenvatinib mesylate), lucitanib, nintedanib (e.g., nintedanib esylate), pemigatinib, surufatinib, tasurgratinib, tinengotinib, zoligratinib, FH-2001, HMPL-453, LY-2874455, or a combination thereof.

In some embodiments, the FGFR2 inhibitor is erdafitinib, futibatinib, infigratinib (e.g., infigratinib phosphate), lucitanib, pemigatinib, tasurgratinib, zoligratinib, bemarituzumab (or biosimilars thereof), FH-2001, HMPL-453, LY-2874455, or a combination thereof.

In some embodiments, the FGFR3 inhibitor is dovitinib, erdafitinib, futibatinib, infigratinib (e.g., infigratinib phosphate), lucitanib, masitinib, nintedanib, pemigatinib, tasurgratinib, zoligratinib, vofatamab (or biosimilars thereof), EXEL-0999, FH-2001, HMPL-453, LY-2874455, or a combination thereof.

In some embodiments, the FGFR4 inhibitor is axitinib, erdafitinib, futibatinib, infigratinib (e.g., infigratinib phosphate), irpagratinib, nintedanib, pemigatinib, FH-2001, H3B-6527, LY-2874455, or a combination thereof.

In some embodiments, the HER2 inhibitor is afatinib (e.g., afatinib dimaleate), dacomitinib (e.g., dacomitinib monohydrate), lapatinib (e.g., lapatinib ditosylate monohydrate), mobocertinib (e.g., mobocertinib succinate), neratinib (e.g., neratinib maleate), poziotinib, pyrotinib (e.g., pyrotinib maleate), sunvozertinib, tesevatinib, tucatinib, varlitinib, an anti-HER2 antibody or anti-HER2 antibody-drug conjugate, or a combination thereof.

In some embodiments, the anti-HER2 antibody or anti-HER2 antibody-drug conjugate is anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, AVX-901, IDM-1, TPIV-100, TAA-013, SHR-A1811, BAT-8001, MDX-210, Alpha-Her2-pAF1-AS-269, MRG-002, DF-1001, AC-101, MM-111, biosimilars thereof, or a combination thereof. In some embodiments, the anti-HER2 antibody or anti-HER2 antibody-drug conjugate is anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, TAA-013, SHR-A1811, BAT-8001, MDX-210, Alpha-Her2-pAF1-AS-269, MRG-002, DF-1001, AC-101, MM-111, biosimilars thereof, or a combination thereof.

In some embodiments, HER2+ status can be detected in a sample from the subject (e.g., via immunohistochemistry (IHC) and/or fluorescent in situ hybridization (FISH)). In some embodiments, the subject was determined (e.g., prior to administration of a compound of Formula (I) or (II) (e.g., Formula (I-A), (I-B), or (I-C))) to have a cancer with HER2+ status.

In some embodiments, HER2 low status can be detected in a sample from the subject (e.g., via IHC and/or FISH). In some embodiments, the subject was determined (e.g., prior to administration of a compound of Formula (I) or (II) (e.g., Formula (I-A), (I-B), or (I-C))) to have a cancer with HER2 low status.

In some embodiments, HER2− status can be detected in a sample from the subject (e.g., via IHC and/or FISH). In some embodiments, the subject was determined (e.g., prior to administration of a compound of Formula (I) or (II) (e.g., Formula (I-A), (I-B), or (I-C))) to have a cancer with HER2− status.

In some embodiments, ER expression status can be detected in a sample from the subject (e.g., via IHC and/or FISH). In some embodiments, the subject was determined (e.g., prior to administration of a compound of Formula (I) or (II) (e.g., Formula (I-A), (I-B), or (I-C))) to have a cancer with ER expression. In some embodiments, the subject was determined (e.g., prior to administration of a compound of Formula (I) or (II) (e.g., Formula (I-A), (I-B), or (I-C))) to have a cancer without ER expression.

In some embodiments, PR expression status can be detected in a sample from the subject (e.g., via IHC, and/or FISH). In some embodiments, the subject was determined (e.g., prior to administration of a compound of Formula (I) or (II) (e.g., Formula (I-A), (I-B), or (I-C))) to have a cancer with PR expression. In some embodiments, the subject was determined (e.g., prior to administration of a compound of Formula (I) or (II) (e.g., Formula (I-A), (I-B), or (I-C))) to have a cancer without PR expression.

In some embodiments, the cancer is a HER2+ breast cancer (e.g., HER2+ breast cancer with ER expression, HER2+ breast cancer without ER expression), and the additional therapy is a HER2 inhibitor (e.g., afatinib (e.g., afatinib dimaleate), dacomitinib (e.g., dacomitinib monohydrate), lapatinib (e.g., lapatinib ditosylate monohydrate), mobocertinib (e.g., mobocertinib succinate), neratinib (e.g., neratinib maleate), poziotinib, pyrotinib (e.g., pyrotinib maleate), sunvozertinib, tesevatinib, tucatinib, varlitinib, or an anti-HER2 antibody or anti-HER2 antibody-drug conjugate). In some embodiments, the cancer is a HER2+ breast cancer (e.g., HER2+ breast cancer with ER expression, HER2+ breast cancer without ER expression), and the additional therapy is tucatinib. In some embodiments, the cancer is a HER2+ breast cancer (e.g., HER2+ breast cancer with ER expression, HER2+ breast cancer without ER expression), and the additional therapy is an anti-HER2 antibody or anti-HER2 antibody-drug conjugate (e.g., anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, AVX-901, IDM-1, TPIV-100, TAA-013, SHR-A1811, BAT-8001, MDX-210, Alpha-Her2-pAF1-AS-269, MRG-002, DF-1001, AC-101, MM-111, or biosimilars thereof). In some embodiments, the cancer is a HER2+ breast cancer (e.g., HER2+ breast cancer with ER expression, HER2+ breast cancer without ER expression), and the additional therapy is trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)). In some embodiments, the cancer is a HER2+ breast cancer (e.g., HER2+ breast cancer with ER expression, HER2+ breast cancer without ER expression), and the additional therapy is trastuzumab deruxtecan (e.g., fam-trastuzumab deruxtecan-nxki, or a biosimilar thereof). In some embodiments, the cancer is a HER2+ breast cancer (e.g., HER2+ breast cancer with ER expression, HER2+ breast cancer without ER expression), and the additional therapy is trastuzumab emtansine, or a biosimilar thereof. In some embodiments, the cancer is a HER2 low breast cancer, and the additional therapy is an anti-HER2 antibody or anti-HER2 antibody-drug conjugate. In some embodiments, the cancer is a HER2 low breast cancer, and the additional therapy is trastuzumab deruxtecan (e.g., fam-trastuzumab deruxtecan-nxki, or a biosimilar thereof).

In some embodiments, the JAK inhibitor is adelatinib, baricitinib, brepocitinib, deuruxolitinib, fedratinib (e.g., fedratinib dihydrochloride monohydrate), filgotinib (e.g., filgotinib maleate), gandotinib, gusacitinib, ilginatinib, izencitinib, jaktinib, momelotinib (e.g., momelotinib dihydrochloride), nezulcitinib, pacritinib (e.g., pacritinib citrate), peficitinib (e.g., peficitinib hydrobromide), ropsacitinib, ruxolitinib (e.g., ruxolitinib phosphate), tasocitinib (e.g., tofacitinib citrate), AT-9283, TQ-05105, or a combination thereof. In some embodiments, the JAK inhibitor is fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), or a combination thereof. In some embodiments, the JAK inhibitor is fedratinib (e.g., fedratinib dihydrochloride monohydrate). In some embodiments, the JAK inhibitor is momelotinib (e.g., momelotinib dihydrochloride). In some embodiments, the JAK inhibitor is pacritinib (e.g., pacritinib citrate). In some embodiments, the JAK inhibitor is ruxolitinib (e.g., ruxolitinib phosphate).

In some embodiments, a JAK V617F mutation can be detected in a sample from the subject (e.g., detecting a JAK2 gene having a mutation corresponding to a V617F mutation in JAK2 protein and/or detecting a JAK2 protein having a V617F mutation). In some embodiments, the subject was determined (e.g., prior to administration of a compound of Formula (I) or (II) (e.g., Formula (I-A), (I-B), or (I-C))) to have a cancer having a JAK2 V617F mutation. In some embodiments, the subject was determined (e.g., prior to administration of a compound of Formula (I) or (II) (e.g., Formula (I-A), (I-B), or (I-C))) to have a cancer lacking a JAK2 V617F mutation.

In some embodiments, the cancer is post-MPN AML, M6-AML, or M7-AML, and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the patient has received a previous line of therapy including a JAK inhibitor. In some embodiments, the patient has not received a previous line of therapy including a JAK inhibitor.

In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)) and a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)) and a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694). In some embodiments, the JAK inhibitor is fedratinib (e.g., fedratinib dihydrochloride monohydrate). In some embodiments, the JAK inhibitor is momelotinib (e.g., momelotinib dihydrochloride). In some embodiments, the JAK inhibitor is pacritinib (e.g., pacritinib citrate). In some embodiments, the JAK inhibitor is ruxolitinib (e.g., ruxolitinib phosphate). In some embodiments, the patient has received a previous line of therapy including a JAK inhibitor. In some embodiments, the patient has not received a previous line of therapy including a JAK inhibitor. In some embodiments, treatment effect can be measured by Spleen Volume Reduction (e.g., Spleen Volume Reduction of greater than or equal to 35% (SVR₃₅), for example, measured by MRI or CT), Total Symptom Score (e.g., Total Symptom Score reduction of greater than or equal to 50% (TSS₅₀), for example, measured by the Myelofibrosis Symptom Assessment Form (MFSAF) version 4.0), or both, such as at 24 weeks after beginning of treatment; see, e.g., Harrison, Claire, et al., New England Journal of Medicine 366.9 (2012): 787-798; and Verstovsek, Srdan, et al. New England Journal of Medicine 366.9 (2012): 799-807. In some embodiments, treatment effect can be measured (e.g., in addition to or instead of SVR₃₅ and/or TSS₅₀) by anemia response (e.g., measured by current International Working Group-Myeloproliferative Neoplasms Research and European LeukemiaNet (IWG-MRT/ELN) criteria), bone marrow fibrosis (e.g., according to the European Consensus Grading System through bone marrow biopsy, such as at 24 or 96 weeks after beginning of treatment), variant allele fraction (e.g., JAK2 V617F variant allele fraction), transfusion independence, overall survival, leukemia-free survival, change in physical functioning (e.g., measured by the physical functioning domain of the European Organization for Research and Treatment of Cancer (EORTC) Quality of Life Questionnaire (QLQ)-C30 or death), change in fatigue (e.g., assessed using the Patient-Reported Outcomes Measurement Information System (PROMIS) Fatigue SF 7a), or a combination thereof.

In some embodiments, the KRas inhibitor is adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, RSC-1255, or a combination thereof.

In some embodiments, a KRas mutation (e.g., a KRas G12C mutation or a KRas G12D mutation) can be detected in a sample from the subject (e.g., detecting a KRAS gene having a mutation corresponding to a G12C mutation or a G12D mutation in KRas protein and/or detecting a KRas protein having a G12C mutation or a G12D mutation). In some embodiments, the subject was determined (e.g., prior to administration of a compound of Formula (I) or (II) (e.g., Formula (I-A), (I-B), or (I-C))) to have a cancer having a KRas G12C mutation. In some embodiments, the subject was determined (e.g., prior to administration of a compound of Formula (I) or (II) (e.g., Formula (I-A), (I-B), or (I-C))) to have a cancer having a KRas G12D mutation.

In some embodiments, the cancer is a KRas mutant lung cancer (e.g., a KRas mutant NSCLC), a KRas mutant CRC, or a KRas mutant pancreatic cancer, and the additional therapy or therapeutic agent is a KRas inhibitor (e.g., adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, or RSC-1255). In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is adagrasib. In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is divarasib. In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is sotorasib. In some embodiments, the cancer is a KRas mutant lung cancer (e.g., a KRas mutant NSCLC (e.g., a KRas G12D mutant NSCLC)), a KRas mutant CRC (e.g., a KRas G12D mutant CRC), or a KRas mutant pancreatic cancer (e.g., a KRas G12D mutant pancreatic cancer), and the additional therapy or therapeutic agent is MRTX1133.

In some embodiments, the MEK inhibitor is avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, TAK-733, or a combination thereof.

In some embodiments, a BRCA1 mutation can be detected in a sample from the subject (e.g., detecting a BRCA1 gene having a mutation and/or detecting a BRCA1 protein having a mutation). In some embodiments, the subject was determined (e.g., prior to administration of a compound of Formula (I) or (II) (e.g., Formula (I-A), (I-B), or (I-C))) to have a cancer having a BRCA1 mutation.

In some embodiments, a BRCA2 mutation can be detected in a sample from the subject (e.g., detecting a BRCA2 gene having a mutation and/or detecting a BRCA2 protein having a mutation). In some embodiments, the subject was determined (e.g., prior to administration of a compound of Formula (I) or (II) (e.g., Formula (I-A), (I-B), or (I-C))) to have a cancer having a BRCA2 mutation.

In some embodiments, the cancer is ovarian cancer (e.g., BRCA1 mutant ovarian cancer or BRCA2 mutant ovarian cancer, HGSOC (e.g., BRCA1 mutant HGSOC or BRCA2 mutant HGSOC), or LGSOC), and the additional therapy or therapeutic agent is a MEK inhibitor (e.g., binimetinib, cobimetinib, selumetinib, or trametinib). In some embodiments, the cancer is BRCA1 mutant ovarian cancer, and the additional therapy or therapeutic agent is a MEK inhibitor (e.g., binimetinib, cobimetinib, selumetinib, or trametinib). In some embodiments, the cancer is BRCA2 mutant ovarian cancer, and the additional therapy or therapeutic agent is a MEK inhibitor (e.g., binimetinib, cobimetinib, selumetinib, or trametinib). In some embodiments, the cancer is HGSOC (e.g., BRCA1 mutant HGSOC or BRCA2 mutant HGSOC), and the additional therapy or therapeutic agent is a MEK inhibitor (e.g., binimetinib, cobimetinib, selumetinib, or trametinib). In some embodiments, the cancer is LGSOC (e.g., BRCA1 mutant HGSOC or BRCA2 mutant HGSOC), and the additional therapy or therapeutic agent is a MEK inhibitor (e.g., binimetinib, cobimetinib, selumetinib, or trametinib).

In some embodiments, the cancer is a KRas mutant CRC (e.g., a KRas G12C mutant NSCLC), and the additional therapy or therapeutic agent is a MEK inhibitor (e.g., binimetinib, cobimetinib, selumetinib, or trametinib).

In some embodiments, the MET inhibitor is cabozantinib (e.g., cabozantinib S-malate), capmatinib (e.g., capmatinib hydrochloride), crizotinib, foritinib, glesatinib, gumarontinib, merestinib, pamufetinib, savolitinib, sitravatinib, tepotinib (e.g., tepotinib hydrochloride hydrate), vebreltinib, zanzalintinib (XL-092), amivantamab (e.g., amivantamab-vmjw, or a biosimilar thereof), emibetuzumab (or biosimilars thereof), RC-108, telisotuzumab vedotin (or biosimilars thereof), ABBV-400, ABN-401, AL-2846, AMG-337, SAR-125844, TQ-B-3139, or a combination thereof.

In some embodiments, a MET alteration can be detected in a sample from the subject (e.g., detecting a MET gene having an alteration (e.g., gene amplification or exon14 skipping) and/or detecting a MET protein having a mutation (e.g., exon14 skipping)). In some embodiments, the subject was determined (e.g., prior to administration of a compound of Formula (I) or (II) (e.g., Formula (I-A), (I-B), or (I-C))) to have a cancer having a MET alteration.

In some embodiments, the cancer is a MET-altered NCLC (e.g., MET amplified NSCLC or MET exon14 skipping NSCLC), and the additional therapy or therapeutic agent is a MET inhibitor (e.g., cabozantinib (e.g., cabozantinib S-malate), capmatinib (e.g., capmatinib hydrochloride), crizotinib, foritinib, glesatinib, gumarontinib, merestinib, pamufetinib, savolitinib, sitravatinib, tepotinib (e.g., tepotinib hydrochloride hydrate), vebreltinib, zanzalintinib (XL-092), amivantamab (e.g., amivantamab-vmjw, or a biosimilar thereof), emibetuzumab (or biosimilars thereof), RC-108, telisotuzumab vedotin (or biosimilars thereof), ABBV-400, ABN-401, AL-2846, AMG-337, SAR-125844, or TQ-B-3139). In some embodiments, the cancer is a MET-altered NCLC (e.g., MET amplified NSCLC or MET exon14 skipping NSCLC), and the additional therapy or therapeutic agent is capmatinib (e.g., capmatinib hydrochloride) or tepotinib (e.g., tepotinib hydrochloride hydrate). In some embodiments, the cancer is a MET-altered NCLC (e.g., MET amplified NSCLC or MET exon14 skipping NSCLC), and the additional therapy or therapeutic agent is telisotuzumab vedotin (or biosimilars thereof).

In some embodiments, the Hif2α inhibitor is belzutifan, AB-521, DFF-332, NKT-2152, PT-2399, or a combination thereof.

In some embodiments, the PARP inhibitor is fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, NMS-03305293, or a combination thereof. In some embodiments, the PARP inhibitor is a PARP1 inhibitor. In some embodiments, the PARP1 inhibitor is saruparib (AZD5305), NMS-03305293, or a combination thereof.

In some embodiments, the cancer is BRCA1 mutant breast cancer or BRCA2 mutant breast cancer, and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is BRCA1 mutant breast cancer or BRCA2 mutant breast cancer, and the additional therapy or therapeutic agent is saruparib.

In some embodiments, the cancer is triple negative breast cancer, and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is triple negative breast cancer, and the additional therapy or therapeutic agent is saruparib.

In some embodiments, the cancer is HGSOC (e.g., BRCA1 mutant HGSOC or BRCA2 mutant HGSOC), and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is HGSOC (e.g., BRCA1 mutant HGSOC or BRCA2 mutant HGSOC), and the additional therapy or therapeutic agent is saruparib.

In some embodiments, the LSD1 inhibitor is bomedemstat, iadademstat, pulrodemstat, seclidemstat (HCl-2577), vafidemstat, GSK-2879552, INCB-059872, JBI-802, or a combination thereof.

In some embodiments, the BET inhibitor is alobresib, apabetalone, mivebresib, pelabresib, trotabresib, ABBV-744, BI-2536, BMS-986158, INCB-057643, JAB-8263, ODM-207, PLX-2853, ZEN-003694, or a combination thereof.

In some embodiments, the cancer is myelofibrosis (e.g., primary myelofibrosis, post-essential thrombocythemia myelofibrosis, or post-polycythemia vera myelofibrosis), and the additional therapy or therapeutic agent is a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694).

In some embodiments, the STING agonist is ulevostinag, ADU-S100, or a combination thereof.

In some embodiments, the telomerase inhibitor is imetelstat (e.g., imetelstat sodium).

In some embodiments, the TORC1/2 inhibitor is apitolisib, bimiralisib, dactolisib, deforolimus, everolimus, fosciclopirox (e.g., fosciclopirox sodium), gedatolisib, onatasertib, paxalisib, sapanisertib, sirolimus, sodium 2-hydroxylinoleate, temsirolimus, umirolimus, vistusertib, zandelisib, zotarolimus, BI-860585, CC-115, CLL-442, PF-04691502, or a combination thereof.

In some embodiments, the VEGFR inhibitor is apatinib, axitinib, cabozantinib (e.g., cabozantinib S-malate), catequentinib (alontinib), cediranib, dovitinib, famitinib, fruquintinib, glesatinib, ibcasertib, ilorasertib, lenvatinib (e.g., lenvatinib mesylate), lucitanib, nintedanib (e.g., nintedanib esylate), pamufetinib, pazopanib (e.g., pazopanib hydrochloride), regorafenib (e.g., regorafenib monohydrate), sitravatinib, sorafenib (e.g., sorafenib tosylate), sunitinib (e.g., sunitinib malate), surufatinib (sulfatinib), telatinib, tinengotinib, tivozanib (e.g., tivozanib hydrochloride monohydrate), vandetanib, vorolanib, zanzalintinib, olinvacimab (or biosimilars thereof), ramucirumab (or biosimilars thereof), CEP-11981, ENMD-2076, ODM-203, or a combination thereof.

In some embodiments, the chemotherapy is a platinum complex, a microtubule inhibitor (e.g., a microtubule destabilizer or a microtubule stabilizer), a topoisomerase inhibitor, a hypomethylating agent, or an antibody-drug conjugate including any thereof. In some embodiments, the platinum complex is carboplatin, cisplatin, lobaplatin, miriplatin, oxaliplatin, or a combination thereof. In some embodiments, the microtubule inhibitor is cabazitaxel, colchicine, desoxyepothilone B, docetaxel, eribulin, ixabepilone, nab-paclitaxel, paclitaxel, plinabulin, sabizabulin, tirbanibulin, vinblastine, vinflunine, vinorelbine, or a combination thereof. In some embodiments, the microtubule inhibitor is cabazitaxel, docetaxel, nab-paclitaxel, paclitaxel, or a combination thereof. In some embodiments, the topoisomerase inhibitor is aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, SN-38, or a combination thereof. In some embodiments, the hypomethylating agent is azacitidine, decitabine, or a combination thereof. In some embodiments, the chemotherapy is a platinum complex and a topoisomerase inhibitor (e.g., cisplatin and etoposide). In some embodiments, the antibody-drug conjugate including the microtubule inhibitor is belantamab mafodotin, brentuximab vedotin, cofetuzumab pelidotin, disitamab vedotin, enfortumab vedotin (e.g., enfortumab vedotin-ejfv, or a biosimilar thereof), mirvetuximab soravtansine (e.g., mirvetuximab soravtansine-gynx, or a biosimilar thereof), polatuzumab vedotin, telisotuzumab vedotin, tisotumab vedotin, trastuzumab emtansine (e.g., ado-trastuzumab emtansine, or a biosimilar thereof), tusamitamab ravtansine, upifitamab rilsodotin, zilovertamab vedotin, Alpha-Her2-pAF1-AS-269, BAT-8001, TAA-013, biosimilars thereof, or a combination thereof. In some embodiments, the antibody-drug conjugate including the microtubule inhibitor is enfortumab vedotin (e.g., enfortumab vedotin-ejfv, or a biosimilar thereof). In some embodiments, the antibody-drug conjugate including the microtubule inhibitor is mirvetuximab soravtansine (e.g., mirvetuximab soravtansine-gynx, or a biosimilar thereof). In some embodiments, the antibody-drug conjugate including the microtubule inhibitor is trastuzumab emtansine (e.g., ado-trastuzumab emtansine, or a biosimilar thereof). In some embodiments, the antibody-drug conjugate including the topoisomerase inhibitor is datopotamab deruxtecan, patritumab deruxtecan, sacituzumab govitecan (e.g., sacituzumab govitecan-hziy, or a biosimilar thereof), trastuzumab deruxtecan (fam-trastuzumab deruxtecan-nxki, or a biosimilar thereof), or a combination thereof. In some embodiments, the antibody-drug conjugate including the topoisomerase inhibitor is sacituzumab govitecan (e.g., sacituzumab govitecan-hziy, or a biosimilar thereof). In some embodiments, the antibody-drug conjugate including the topoisomerase inhibitor is trastuzumab deruxtecan (e.g., fam-trastuzumab deruxtecan-nxki, or a biosimilar thereof).

In some embodiments, the cancer is a lung cancer (e.g., NSCLC (e.g., squamous cell carcinoma)), and the additional therapy or therapeutic agent is a microtubule inhibitor (e.g., cabazitaxel, docetaxel, nab-paclitaxel, or paclitaxel). In some embodiments, the cancer is a lung cancer (e.g., NSCLC (e.g., squamous cell carcinoma)), and the additional therapy or therapeutic agent is docetaxel.

In some embodiments, the cancer is NSCLC (e.g., NSCLC with a MET amplification), and the additional therapy or therapeutic agent is telisotuzumab vedotin.

In some embodiments, the cancer is a lung cancer (e.g., NSCLC), and the additional therapy or therapeutic agent is a platinum complex (e.g., carboplatin, cisplatin, lobaplatin, miriplatin, or oxaliplatin) and anti-PD1 therapy.

In some embodiments, the cancer is a lung cancer (e.g., NSCLC), and the additional therapy or therapeutic agent is a platinum complex (e.g., carboplatin, cisplatin, lobaplatin, miriplatin, or oxaliplatin) and anti-PD-L1 therapy.

In some embodiments, the cancer is a lung cancer (e.g., NSCLC), and the additional therapy or therapeutic agent is a pemetrexed and anti-PD1 therapy.

In some embodiments, the cancer is a lung cancer (e.g., NSCLC), and the additional therapy or therapeutic agent is a pemetrexed and anti-PD-L1 therapy.

In some embodiments, the cancer is a lung cancer (e.g., SCLC), and the additional therapy or therapeutic agent is a platinum complex (e.g., carboplatin, cisplatin, lobaplatin, miriplatin, or oxaliplatin) and a topoisomerase inhibitor (e.g., aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, or SN-38). In some embodiments, the cancer is a lung cancer (e.g., SCLC), and the additional therapy or therapeutic agent is carboplatin and etoposide. In some embodiments, the cancer is a lung cancer (e.g., SCLC), and the additional therapy or therapeutic agent is cisplatin and etoposide.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel). In some embodiments, the cancer is triple negative breast cancer, and the additional therapy or therapeutic agent is a microtubule inhibitor (e.g., cabazitaxel, docetaxel, nab-paclitaxel, or paclitaxel). In some embodiments, the cancer is triple negative breast cancer, and the additional therapy or therapeutic agent is nab-paclitaxel or paclitaxel.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel) and anti-PD1 therapy.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel) and anti-PD-L1 therapy.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is capecitabine.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is sacituzumab govitecan (e.g., sacituzumab govitecan-hziy, or a biosimilar thereof).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is trastuzumab deruxtecan (e.g., fam-trastuzumab deruxtecan-nxki, or a biosimilar thereof).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)).

In some embodiments, the cancer is hormone receptor positive breast cancer, and the additional therapy or therapeutic agent is hormone therapy (e.g., tamoxifen, toremifene, or a combination thereof).

In some embodiments, the cancer is hormone receptor positive breast cancer, and the additional therapy or therapeutic agent is a selective estrogen receptor degrader (SERD) (e.g., fulvestrant, elacestrant, or a combination thereof).

In some embodiments, the cancer is a HER2+ breast cancer (e.g., HER2+ breast cancer with ER expression, HER2+ breast cancer without ER expression), and the additional therapy or therapeutic agent is trastuzumab deruxtecan (e.g., fam-trastuzumab deruxtecan-nxki, or a biosimilar thereof) or trastuzumab emtansine (e.g., KADCYLA® (trastuzumab emtansine), or a biosimilar thereof (e.g., UJVIRA® (trastuzumab emtansine))).

In some embodiments, the cancer is a HER2 low breast cancer, and the additional therapy or therapeutic agent is trastuzumab deruxtecan (e.g., fam-trastuzumab deruxtecan-nxki, or a biosimilar thereof).

In some embodiments, the cancer is a head and neck cancer, and the additional therapy or therapeutic agent is a microtubule inhibitor (e.g., cabazitaxel, docetaxel, nab-paclitaxel, or paclitaxel). In some embodiments, the cancer is a head and neck cancer, and the additional therapy or therapeutic agent is docetaxel.

In some embodiments, the cancer is a cervical cancer, and the additional therapy or therapeutic agent is a microtubule inhibitor (e.g., cabazitaxel, docetaxel, nab-paclitaxel, or paclitaxel).

In some embodiments, the cancer is an endometrial cancer, and the additional therapy or therapeutic agent is a microtubule inhibitor (e.g., cabazitaxel, docetaxel, nab-paclitaxel, or paclitaxel).

In some embodiments, the cancer is a prostate cancer, and the additional therapy or therapeutic agent is a microtubule inhibitor (e.g., cabazitaxel, docetaxel, nab-paclitaxel, or paclitaxel).

In some embodiments, the cancer is an ovarian cancer (e.g., HGSOC) and the additional therapy or therapeutic agent is a microtubule inhibitor (e.g., cabazitaxel, docetaxel, nab-paclitaxel, or paclitaxel). In some embodiments, the cancer is an ovarian cancer (e.g., HGSOC) and the additional therapy or therapeutic agent is a microtubule inhibitor (e.g., cabazitaxel, docetaxel, nab-paclitaxel, or paclitaxel) and a platinum complex (e.g., carboplatin, cisplatin, lobaplatin, miriplatin, or oxaliplatin). In some embodiments, the cancer is an ovarian cancer (e.g., HGSOC) and the additional therapy or therapeutic agent is nab-paclitaxel or paclitaxel and carboplatin.

In some embodiments, the cancer is a pancreatic cancer, and the additional therapy or therapeutic agent is a microtubule inhibitor (e.g., cabazitaxel, docetaxel, nab-paclitaxel, or paclitaxel). In some embodiments, the cancer is a pancreatic cancer, and the additional therapy or therapeutic agent is a microtubule inhibitor (e.g., cabazitaxel, docetaxel, nab-paclitaxel, or paclitaxel) and gemcitabine.

In some embodiments, the cancer is a bladder cancer (e.g., bladder urothelial carcinoma), and the additional therapy or therapeutic agent is enfortumab vedotin (e.g., enfortumab vedotin-ejfv, or a biosimilar thereof).

In some embodiments, the cancer is a bladder cancer (e.g., bladder urothelial carcinoma), and the additional therapy or therapeutic agent is sacituzumab govitecan (e.g., sacituzumab govitecan-hziy, or a biosimilar thereof).

In some embodiments, the cancer is triple negative breast cancer, and the additional therapy or therapeutic agent is sacituzumab govitecan (e.g., sacituzumab govitecan-hziy, or a biosimilar thereof).

In some embodiments, folate receptor positivity status can be detected in a sample from the subject (e.g., via immunohistochemistry (HC) and/or fluorescent in situ hybridization (FISH)). In some embodiments, the subject was determined (e.g., prior to administration of a compound of Formula (I) or (II) (e.g., Formula (I-A), (I-B), or (I-C))) to have a cancer that is folate receptor positive.

In some embodiments, the cancer is folate receptor positive ovarian cancer (e.g., folate receptor positive HGSOC), and the additional therapy or therapeutic agent is mirvetuximab soravtansine (e.g., mirvetuximab soravtansine-gynx, or a biosimilar thereof).

In some embodiments the immunomodulatory imide drug is thalidomide, lenalidomide, pomalidomide, iberdomide, avadomide, CC-99282, or a combination thereof.

In some embodiments, the anti-PD1 therapy is balstilimab, budigalimab, cadonilimab, camrelizumab, cemiplimab (e.g., cemiplimab-rwlc, or a biosimilar thereof), cetrelimab, dostarlimab (e.g., dostarlimab-gxly, or a biosimilar thereof), ezabenlimab, geptanolimab, ivonescimab, nivolumab (e.g., OPDIVO® (nivolumab), or a biosimilar thereof), nofazinlimab, pembrolizumab (e.g., KEYTRUDA® (pembrolizumab), or a biosimilar thereof), penpulimab, pidilizumab, pimivalimab, prolgolimab, pucotenlimab, retifanlimab (e.g., retifanlimab-dlwr, or a biosimilar thereof), rilvegostomig, rosnilimab, rulonilimab, sasanlimab, serplulimab, sintilimab (e.g., TYVYT® (sintilimab), or a biosimilar thereof), spartalizumab, tebotelimab, tislelizumab, toripalimab, volrustomig, vudalimab, zimberelimab, QL-1604, HX-009, INCB-086550, RG-6139, BAT-1306, SG-001, biosimilars thereof, or a combination thereof.

In some embodiments, the PD-L1 inhibitor is INCB-086550.

In some embodiments, the anti-PD-L1 therapy is adebrelimab, atezolizumab (e.g., TECENTRIQ® (atezolizumab), or a biosimilar thereof), avelumab (e.g., BAVENCIO® (avelumab), or a biosimilar thereof), bintrafusp alfa, cosibelimab, danburstotug, durvalumab (e.g., TIFINZI® (durvalumab), or a biosimilar thereof), envafolimab (e.g., ENWEIDA® (envafolimab), or a biosimilar thereof), erfonrilimab, pacmilimab, socazolimab, sugemalimab (e.g., CEJEMLY® (sugemalimab), or a biosimilar thereof), A-167, APL-502, AUPM-170, BNT-311, SHR-1701, biosimilars thereof, or a combination thereof.

In some embodiments, the additional therapy is radiotherapy.

In some embodiments, the cancer is head and neck cancer (e.g., head and neck squamous cell carcinoma), and the additional therapy is radiotherapy.

In some embodiments, the additional therapy includes a BRaf inhibitor and a MEK inhibitor. For example, the additional therapy can include dabrafenib and trametinib, vemurafenib and cobimetinib, or encorafenib and binimetinib.

Exemplary descriptions of agents in combination with BCL-2 family inhibitors can be found in: Hikita, Hayato, et al. Hepatology 52.4 (2010): 1310-1321; Chen, Jun, et al. Molecular Cancer Therapeutics 10.12 (2011): 2340-2349; Inuzuka, Hiroyuki, et al. Nature 471.7336 (2011): 104-109; Wertz, Ingrid E., et al. Nature 471.7336 (2011): 110-114; Tan, Nguyen, et al. Clinical Cancer Research 17.6 (2011): 1394-1404; Wong, Maureen, et al. Molecular Cancer Therapeutics 11.4 (2012): 1026-1035; Corcoran, Ryan B., et al. Cancer Cell 23.1 (2013): 121-128; Waibel, Michaela, et al. Cell Reports 5.4 (2013): 1047-1059; Frederick, Dennie T., et al. PloS One 9.7 (2014): e101286; Vlahovic, Gordana, et al. Investigational New Drugs 32.5 (2014): 976-984; Faber, Anthony C., et al. Cancer Discovery 4.1 (2014): 42-52; Leverson, Joel D., et al. Science Translational Medicine 7.279 (2015): 279ra40-279ra40; Guo, Jun, et al. PLoS One 10.3 (2015): e0114363; Lheureux, Stephanie, et al. International Journal of Cancer 136.5 (2015): E340-E350; Zoeller, Jason J., et al. Cancer Research 76.14_Supplement (2016): 4358-4358; Weeden, Clare E., et al. Oncogene 37.32 (2018): 4475-4488; Lucantoni, Federico, et al. Cell Death & Disease 9.2 (2018): 1-13; Iavarone, Claudia, et al. Molecular Cancer Therapeutics 18.3 (2019): 642-655; Fleury, Hubert, et al. Nature Communications 10.1 (2019): 1-15; Lohard, Steven, et al. Nature Communications 11.1 (2020): 1-16; Bertino, Erin M., et al. Clinical Cancer Research 27.6 (2021): 1604-1611; Guo, Ting, et al. Aging 13.15 (2021): 19750; Puglisi, Martina, et al. Future Oncology 17.21 (2021): 2747-2758; Harrison, Claire N., et al. Journal of Clinical Oncology 40.15 (2022): 1671; Kohler, Jens, et al. Molecular Cancer Therapeutics 20.4 (2021): 641-654; Jaaks, Patricia, et al. Nature 603.7899 (2022): 166-173; Sobol, Benjamin, et al. International Journal of Molecular Sciences 23.14 (2022): 7850; Passamonti, J Clin Oncol 40, (2022) (suppl 16; abstr 7015); Qin, J Clin Oncol 40, (2022) (suppl 16; abstr e20612); Potter, Danielle S., et al. Cancer Research 82.12_Supplement (2022): 3691-3691; and Shebl, Bassem, et al. Cancer Research 82.12_Supplement (2022): 3888-3888.

Also provided herein is a method of treating cancer, comprising administering to a subject in need thereof (a) a compound of Formula (I) or (II) (e.g., Formula (I-A), (I-B), or (I-C)), or a pharmaceutically acceptable salt thereof, and (b) an additional therapeutic agent, for simultaneous, separate or sequential use for the treatment of cancer, wherein the amounts of the compound of Formula (I) or (II) (e.g., Formula (I-A), (I-B), or (I-C)), or a pharmaceutically acceptable salt thereof, and the additional therapeutic agent are together effective in treating the cancer. In some embodiments, the method comprises administering (c) at least one pharmaceutically acceptable carrier.

These additional therapeutic agents may be administered with one or more doses of the compound of Formula (I) or (II) (e.g., Formula (I-A), (I-B), or (I-C)), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art. In some embodiments, the compound of Formula (I) or (II) (e.g., Formula (I-A), (I-B), or (I-C)), or a pharmaceutically acceptable salt thereof, and the additional therapeutic agent are administered simultaneously as separate dosages. In some embodiments, a compound of Formula (I) or (II) (e.g., Formula (I-A), (I-B), or (I-C)), or a pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (I) or (II) (e.g., Formula (I-A), (T-B), or (I-C)), or a pharmaceutically acceptable salt thereof, and the additional therapeutic agent are administered simultaneously as a combined dosage.

Also provided herein is (i) a pharmaceutical combination for treating a cancer in a subject in need thereof, which comprises (a) a compound of Formula (I) or (II) (e.g., Formula (I-A), (I-B), or (I-C)), or a pharmaceutically acceptable salt thereof, and (b) at least one additional therapeutic agent (e.g., any of the exemplary additional therapeutic agents described herein or known in the art), for simultaneous, separate or sequential use for the treatment of cancer, wherein the amounts of the compound of Formula (I) or (II) (e.g., Formula (I-A), (T-B), or (I-C)), or a pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the pharmaceutical combination comprises (c) at least one pharmaceutically acceptable carrier.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease, disorder, or condition to be treated and/or prevented.

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The term “preventing” as used herein means to delay the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The phrase “therapeutically effective amount” means an amount of compound that, when administered to a subject in need thereof, is sufficient to (i) treat a disease, disorder, or condition, (ii) attenuate, ameliorate, or eliminate one or more symptoms of the particular disease, disorder, or condition, or (iii) delay the onset of one or more symptoms of the particular disease, disorder, or condition described herein. The amount of a compound of Formula (I) or (II) (e.g., Formula (I-A), (I-B), or (I-C)), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

The phrase “effective amount” of a compound of Formula (I) or (II) (e.g., Formula (I-A), (I-B), or (I-C)), as used herein, means an amount of the compound that, when administered to a cell, in vitro or in vivo, is sufficient to reduce proliferation of the cell or to kill the cell. The amount of a compound of Formula (I) or (II) (e.g., Formula (I-A), (I-B), or (I-C)), or a pharmaceutically acceptable salt thereof, that will correspond to such an amount will vary depending upon factors such as the particular compound and genetics of the cell to be treated, but can nevertheless be routinely determined by one skilled in the art.

### Pharmaceutical Compositions and Administration

### General

In some embodiments, a compound of Formula (I) or (II) (e.g., Formula (I-A), (I-B), or (I-C)), or a pharmaceutically acceptable salt thereof, is administered as a pharmaceutical composition that includes the compound, or a pharmaceutically acceptable salt thereof, and one or more pharmaceutically acceptable excipients, and optionally one or more additional therapeutic agents as described herein.

In some embodiments, the compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic excipient may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, a compound of Formula (I) or (II) (e.g., Formula (I-A), (I-B), or (I-C)), or a pharmaceutically acceptable salt thereof, as described herein, or a pharmaceutical composition thereof, can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, intravitreal, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

In some embodiments, a compound of Formula (I) or (II) (e.g., Formula (I-A), (I-B), or (I-C)), or a pharmaceutically acceptable salt thereof, as described herein, or a pharmaceutical composition thereof, can be administered orally to a subject in need thereof. Without being bound by any particular theory, it is believed that oral dosing (e.g., versus IV dosing) can be preferred by patients for convenience, perception of efficacy, and/or past experience.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the compound described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein, or a pharmaceutical composition thereof, are suitable for local delivery to the digestive or GI tract by way of oral administration (e.g., solid or liquid dosage forms).

Solid dosage forms for oral administration include capsules, tablets, pills, powders, and granules. In such solid dosage forms, the compound is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a compound, or pharmaceutically acceptable salt thereof, as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEGs, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the compounds to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. As with other carriers or vehicles, an ointment base should be inert, stable, nonirritating, and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated, and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The term “acceptable” with respect to a formulation, composition, or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid, and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The term “pharmacologically acceptable salts” is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to a subject. Multiple techniques of administering a compound exist in the art including, but not limited to: rectal, oral, intravenous, aerosol, parenteral, ophthalmic, pulmonary, and topical administration.

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

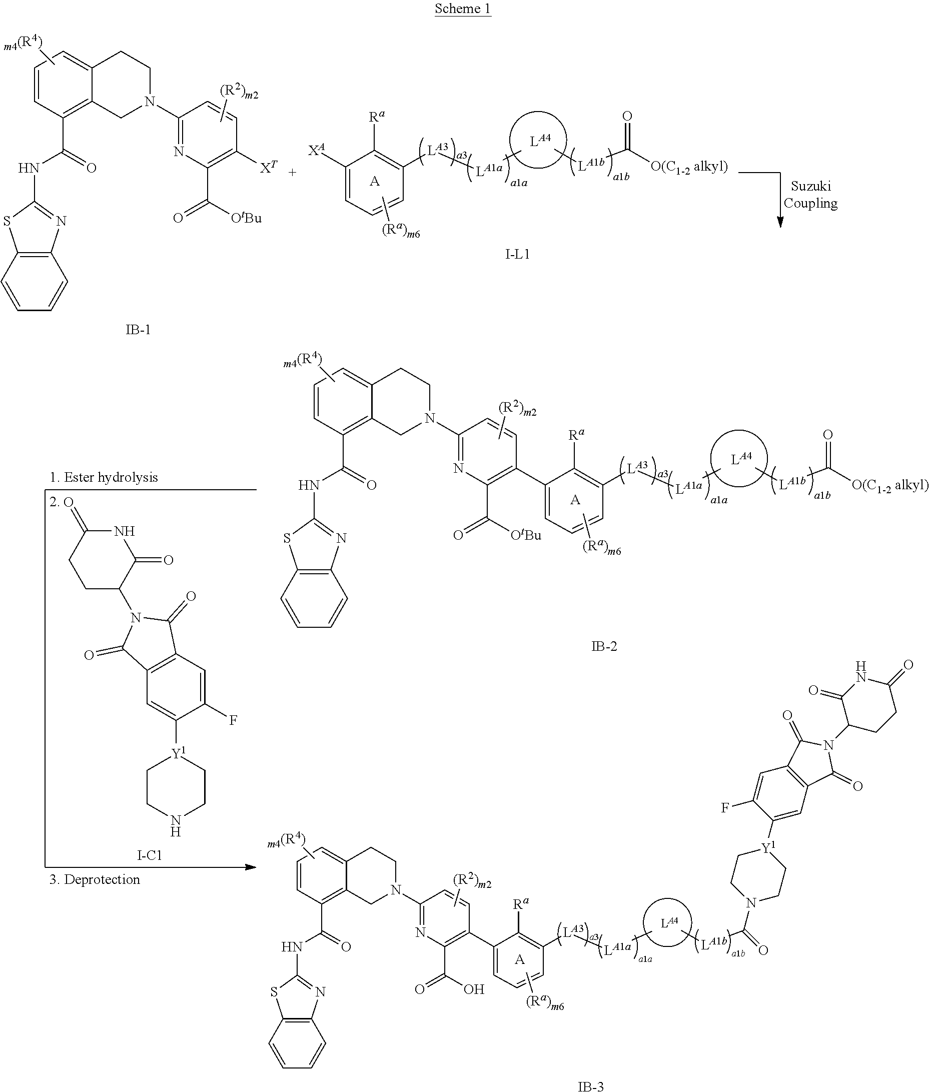

Scheme 1 depicts the synthesis of Compound IB-3, which is a compound of Formula (I-B), wherein R¹ is C(O)OH; and each remaining variable is defined in accordance with Formula (I-B). Compound IB-1 is reacted with Compound I-L1 under standard conditions for Suzuki coupling to provide Compound IB-2, wherein one of X^{A} and X^{T} is B(OH)₂ or Bpin; the other of X^{A} and X^{T} is —Br; and each remaining variable in IB-1 and I-L1 is defined in accordance with Formula (I-B). Hydrolysis of the C₁₋₂ alkyl ester in IB-2 provides the corresponding carboxylic acid which is then reacted with I-C1 under standard conditions for amide bond formation, wherein each variable in I-C1 is defined in accordance with Formula (I-B). Removal of the tert-butyl group under standard conditions for deprotection then provides Compound IB-3.

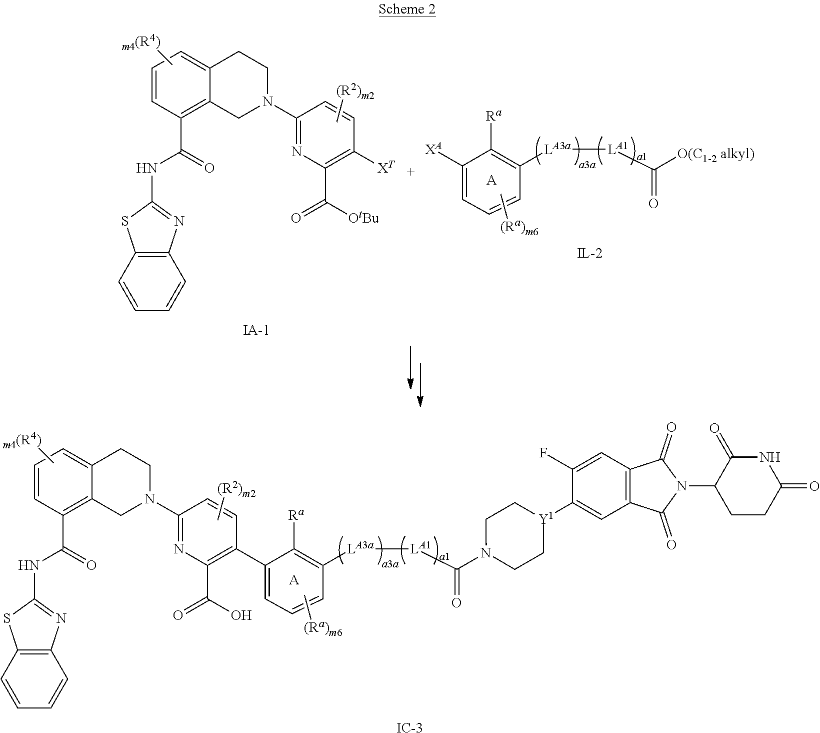

Compound IC-3 is a compound of Formula (I-C), wherein R¹ is C(O)OH; a3c is 1; and each remaining variable is defined in accordance with Formula (I-C), is prepared from IA-1 and I-L2 using methods depicted for Compound IB-3 in Scheme 1, above. One of X^{A} and X^{T} is B(OH)₂ or Bpin; the other of X^{A} and X^{T} is —Br; and each remaining variable in IA-1 and I-L2 is defined in accordance with Formula (I-C).

### EXAMPLES

### Example 1. Preparation of Compound 111a

### 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperazin-1-yl)-3-oxopropyl)cyclohexyl)oxy)-2-methylphenyl)picolinic acid

### Step A. Ethyl (1r,4r)-4-(3-bromo-2-methylphenoxy)cyclohexane-1-carboxylate

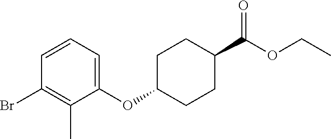 [SMILES: CCOC(=O)[C@H]1CC[C@H](Oc2cccc(Br)c2C)CC1]

To a solution of ethyl 4-hydroxycyclohexanecarboxylate (10 g, 58.06 mmol, 1 eq.), 3-bromo-2-methyl-phenol (11.95 g, 63.87 mmol, 1.1 eq.), PPh₃ (24.37 g, 92.90 mmol, 1.6 eq.) in THE (100 mL) was added DIAD (18.79 g, 92.90 mmol, 18.06 mL, 1.6 eq.) under 0° C. Then the mixture was stirred at 25° C. for 16 h. The reaction mixture was concentrated under reduced pressure to give a residue that was purified by silica gel chromatography (ethyl acetate/petroleum ether gradient) to afford the title compound (3.6 g, 10.55 mmol, 18.17% yield). ¹H NMR (400 MHz, CDCl₃) δ 7.14 (d, J=8.0 Hz, 1H), 6.97 (t, J=8.0 Hz, 1H), 6.80 (d, J=8.0 Hz, 1H), 4.20-4.11 (m, 3H), 2.39-2.35 (m, 2H), 2.30 (s, 3H), 2.19-2.14 (m, 2H), 2.09-2.04 (m, 2H), 1.62-1.52 (m, 5H), 1.26 (t, J=6.8 Hz, 3H).

### Step B. ((1r,4r)-4-(3-Bromo-2-methylphenoxy)cyclohexyl)methanol

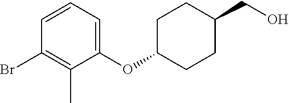 [SMILES: Cc1c(Br)cccc1O[C@H]1CC[C@H](CO)CC1]

To a solution of ethyl 4-(3-bromo-2-methyl-phenoxy)cyclohexanecarboxylate (3.6 g, 10.55 mmol, 1 eq.) in THF (15 mL) was added LAH (480.49 mg, 12.66 mmol, 1.2 eq.) under 0° C. The resulting mixture was then stirred at 25° C. for 1.5 h. The reaction was quenched by addition of a saturated aqueous solution of sodium sulfate (10 mL), and then extracted with ethyl acetate (10 mL×3). The combined organic layers were dried over anhydrous sodium sulfate, filtered, and concentrated under reduced pressure to afford the title compound (2.96 g, crude) that was used without further purification. ¹H NMR (400 MHz, CDCl₃) δ 7.13 (d, J=8.0 Hz, 1H), 6.97 (t, J=8.0 Hz, 1H), 6.81 (d, J=8.4 Hz, 1H), 4.13-4.08 (m, 1H), 3.50 (d, J=6.4 Hz, 2H), 2.30 (s, 3H), 2.20-2.14 (m, 2H), 1.95-1.86 (m, 2H), 1.53-1.45 (m, 3H), 1.14-1.04 (m, 2H).

### Step C. (1r,4r)-4-(3-Bromo-2-methylphenoxy)cyclohexane-1-carbaldehyde

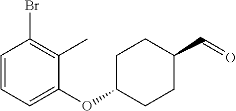 [SMILES: Cc1c(Br)cccc1O[C@H]1CC[C@H](C=O)CC1]

To a solution of DMSO (3.09 g, 39.57 mmol, 3.09 mL, 4 eq.) in DCM (10 mL) was added dropwise a solution of oxalyl chloride (2.51 g, 19.79 mmol, 1.73 mL, 2 eq.) in DCM (2 mL) at −70° C. under a nitrogen atmosphere. The mixture was stirred at −70° C. for 1 h. [4-(3-bromo-2-methyl-phenoxy)cyclohexyl]methanol (2.96 g, 9.89 mmol, 1 eq.) in DCM (10 mL) was then added dropwise at −70° C. The solution was stirred for 1 h at −70° C. TEA (6.01 g, 59.36 mmol, 8.26 mL, 6 eq.) was added and the mixture was stirred at −70° C. for 0.5 h under a nitrogen atmosphere. The reaction mixture was quenched by addition water (10 mL) and then diluted with DCM 10 mL and extracted with DCM (10 mL×3). The combined organic layers were washed with water (5 mL×3), dried over anhydrous sodium sulfate, filtered, and concentrated under reduced pressure to afford the title compound (3 g, crude) that was used without further purification. ¹H NMR (400 MHz, DMSO-d6) δ 9.62-9.59 (m, 1H), 7.16-7.01 (m, 3H), 4.36-4.23 (m, 1H), 2.39-2.31 (m, 1H), 2.25-2.18 (m, 3H), 2.03-1.92 (m, 4H), 1.50-1.38 (m, 4H).

### Step D. Ethyl (E)-3-((1r,4r)-4-(3-bromo-2-methylphenoxy)cyclohexyl)acrylate

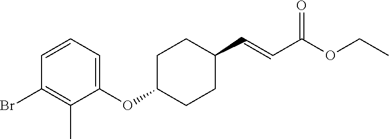 [SMILES: CCOC(=O)/C=C/[C@H]1CC[C@H](Oc2cccc(Br)c2C)CC1]

To a solution of NaH (56.52 mg, 1.41 mmol, 60% purity, 2.1 eq.) in THF (3 mL) at 0° C. was added ethyl 2-diethoxyphosphorylacetate (301.75 mg, 1.35 mmol, 267.04 μL, 2 eq.). 4-(3-bromo-2-methyl-phenoxy)cyclohexanecarbaldehyde (200 mg, 672.98 μmol, 1 eq.) was then added and the mixture was stirred at 25° C. for 6 h. The reaction mixture was quenched by addition of a saturated aqueous solution of ammonium chloride (5 mL) at 0° C., and then extracted with ethyl acetate (10 mL×3). The combined organic layers were washed with water (10 mL), dried over anhydrous sodium sulfate, filtered, and concentrated under reduced pressure to give a residue which was purified by silica gel chromatography (ethyl acetate/petroleum ether gradient) to afford the title compound (140 mg, 362.12 μmol, 53.81% yield). ¹H NMR (400 MHz, CDCl3) δ 7.14 (d, J=8.0 Hz, 1H), 7.01-6.87 (m, 2H), 6.80 (d, J=8.4 Hz, 1H), 5.82 (dd, J=1.2, 15.6 Hz, 1H), 4.21-4.15 (m, 2H), 4.16-4.07 (m, 1H), 2.30 (s, 3H), 2.26-2.16 (m, 3H), 1.97-1.89 (m, 2H), 1.58-1.48 (m, 2H), 1.33-1.26 (m, 5H).

### Step E. Ethyl 3-((1r,4r)-4-(3-bromo-2-methylphenoxy)cyclohexyl)propanoate

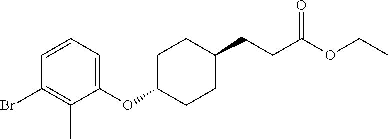 [SMILES: CCOC(=O)CC[C@H]1CC[C@H](Oc2cccc(Br)c2C)CC1]

A mixture of ethyl (E)-3-[4-(3-bromo-2-methyl-phenoxy)cyclohexyl]prop-2-enoate (1.6 g, 4.36 mmol, 1 eq.), PtO₂ (98.92 mg, 435.64 μmol, 0.1 eq.) in EtOH (15 mL) was degassed and purged with hydrogen 3 times. The mixture was stirred at 25° C. for 3 hours under a hydrogen atmosphere (balloon, ˜15 psi. The reaction mixture was filtered and concentrated under reduced pressure to give a residue that was purified by preparative RP-HPLC (water/acetonitrile with 0.1% formic acid in each). Combined and lyophilized pure product containing fractions to afford the title compound (1.4 g, 3.79 mmol, 87.02% yield). ¹H NMR (400 MHz, DMSO-d6) δ 7.17-7.10 (m, 1H), 7.06 (t, J=8.0 Hz, 1H), 7.03-6.99 (m, 1H), 4.27-4.17 (m, 1H), 4.11-4.04 (m, 2H), 2.33-2.26 (m, 2H), 2.20 (s, 3H), 2.03 (d, J=10.0 Hz, 2H), 1.75 (d, J=12.0 Hz, 2H), 1.55-1.42 (m, 2H), 1.39-1.21 (m, 3H), 1.17 (t, J=7.2 Hz, 3H), 1.11-0.97 (m, 2H).

### Step F. tert-Butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate

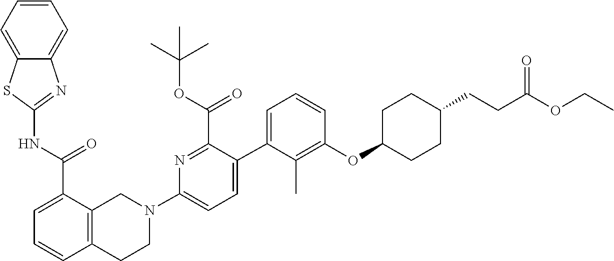 [SMILES: CCOC(=O)CC[C@H]1CC[C@H](Oc2cccc(-c3ccc(N4CCc5cccc(C(=O)Nc6nc7ccccc7s6)c5C4)nc3C(=O)OC(C)(C)C)c2C)CC1]

A mixture of ethyl 3-[4-(3-bromo-2-methyl-phenoxy)cyclohexyl]propanoate (200 mg, 541.58 μmol, 1 eq.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (546.40 mg, 758.21 μmol, 1.4 eq.), [2-(2-aminophenyl)phenyl]palladium(1+); bis(1-adamantyl)-butyl-phosphane; methanesulfonate (78.88 mg, 108.32 μmol, 0.2 eq.), an aqueous solution of potassium carbonate (1.5 M, 541.58 μL, 1.5 eq.) in dioxane (2.5 mL) was degassed and purged with nitrogen 3 times. The mixture was stirred at 100° C. for 1 h under microwave heating. The reaction mixture was filtered and concentrated under reduced pressure to provide a residue that was purified by silica gel chromatography (ethyl acetate/petroleum ether gradient) to afford the title compound (440 mg, crude). ¹H NMR (400 MHz, DMSO-d6) δ 8.02 (d, J=7.6 Hz, 1H), 7.78 (d, J=8.0 Hz, 1H), 7.60 (d, J=7.5 Hz, 1H), 7.50-7.40 (m, 3H), 7.40-7.30 (m, 2H), 7.12-7.03 (m, 1H), 6.99-6.89 (m, 2H), 6.59-6.49 (m, 1H), 5.03-4.90 (m, 2H), 4.27-4.14 (m, 1H), 4.03 (d, J=6.8 Hz, 2H), 3.87 (t, J=5.6 Hz, 2H), 3.29 (s, 2H), 3.03 (t, J=5.6 Hz, 2H), 2.29 (t, J=7.6 Hz, 2H), 2.05 (d, J=11.2 Hz, 1H), 1.84 (s, 3H), 1.79-1.70 (m, 2H), 1.50-1.42 (m, 2H), 1.23 (d, J=8.4 Hz, 2H), 1.19-1.17 (m, 3H), 1.07 (s, 2H), 1.00 (s, 9H).

### Step G. 3-((1r,4r)-4-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid

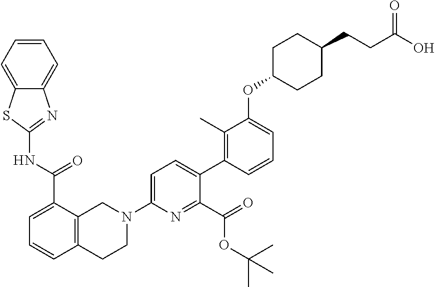 [SMILES: Cc1c(O[C@H]2CC[C@H](CCC(=O)O)CC2)cccc1-c1ccc(N2CCc3cccc(C(=O)Nc4nc5ccccc5s4)c3C2)nc1C(=O)OC(C)(C)C]

A mixture of tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (440 mg, 567.77 μmol, 1 eq.), LiOH monohydrate (71.47 mg, 1.70 mmol, 3 eq.) in THF (1.2 mL) and water (0.4 mL) was stirred at 25° C. for 1.5 h. The mixture was concentrated and redissolved in water (20 mL) and an aqueous solution of 1M HCl was added to adjust the pH to 2. The resulting solids were filtered and dried under reduced pressure to afford the title compound (380 mg, 508.76 μmol, 89.61% yield).

### Step H. tert-Butyl 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperazin-1-yl)-3-oxopropyl)cyclohexyl)oxy)-2-methylphenyl)picolinate

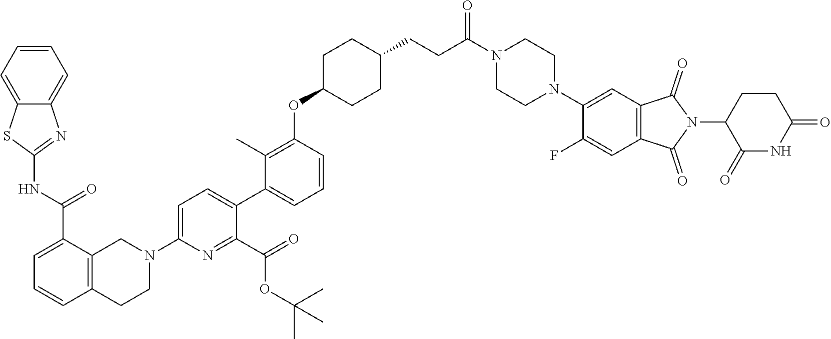 [SMILES: Cc1c(O[C@H]2CC[C@H](CCC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)OC(C)(C)C]

A mixture of 3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]cyclohexyl]propanoic acid (150 mg, 200.83 μmol, 1 eq.), 2-(2,6-dioxo-3-piperidyl)-5-fluoro-6-piperazin-1-yl-isoindoline-1,3-dione (72.37 mg, 200.83 μmol, 1 eq.), HATU (114.54 mg, 301.24 μmol, 1.5 eq.), DIPEA (77.86 mg, 602.48 μmol, 104.94 μL, 3 eq.) in DMF (1.5 mL) was stirred at 25° C. for 12 h. The mixture was and concentrated under reduced pressure to afford the title compound (120 mg, crude).

### Step I. 6-(8-(Benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperazin-1-yl)-3-oxopropyl)cyclohexyl)oxy)-2-methylphenyl)picolinic acid

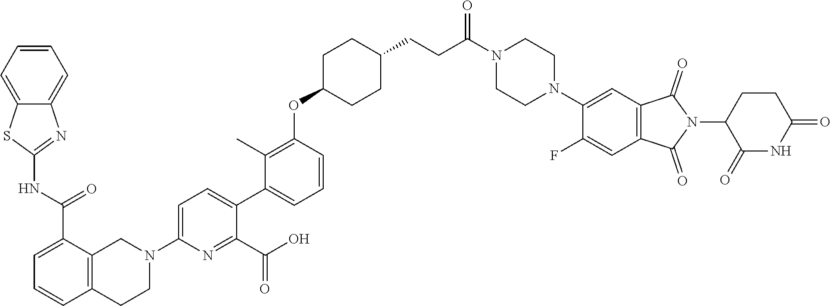 [SMILES: Cc1c(O[C@H]2CC[C@H](CCC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

tert-Butyl 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperazin-1-yl)-3-oxopropyl)cyclohexyl)oxy)-2-methylphenyl)picolinate (120 mg, 110.17 μmol, 1 eq.) in TFA (0.8 mL) and DCM (0.8 mL) was stirred at 40° C. for 1.5 h. The reaction mixture was filtered and concentrated under reduced pressure to provide a residue that was purified by preparative RP-HPLC (water/acetonitrile with 0.1% formic acid in each). Combined and lyophilized pure product containing fractions to afford the title compound (54.27 mg, 50.87 μmol, 46.17% yield, 96.84% purity). MS (ESI) m/z: 1033.2 [M+H]⁺; ¹H NMR (400 MHz, DMSO-d6) δ=13.00-12.72 (m, 1H), 11.11 (s, 1H), 8.03 (d, J=8.0 Hz, 1H), 8.00-8.00 (m, 1H), 7.82-7.73 (m, 2H), 7.62 (d, J=7.6 Hz, 1H), 7.57-7.12 (m, 7H), 7.11-7.04 (m, 1H), 6.94 (dd, J=8.4, 17.6 Hz, 2H), 6.61 (d, J=7.6 Hz, 1H), 5.11 (dd, J=5.2, 12.8 Hz, 1H), 4.98 (s, 2H), 4.25-4.14 (m, 1H), 3.91 (t, J=5.6 Hz, 2H), 3.76 (s, 3H), 3.28-3.19 (m, 4H), 3.02 (t, J=5.6 Hz, 2H), 2.93-2.84 (m, 1H), 2.64-2.53 (m, 2H), 2.38 (t, J=7.6 Hz, 2H), 2.13-1.99 (m, 3H), 1.87 (s, 3H), 1.81 (d, J=11.2 Hz, 2H), 1.52-1.24 (m, 6H), 1.15-1.02 (m, 2H).

### Example 2. Preparation of Compound 104

### 6-(8-(benzo[d]thiazol-2-ylcarbamoyl)-3,4-dihydroisoquinolin-2(1H)-yl)-3-(3-((7-(4-(2-(2,6-dioxopiperidin-3-yl)-6-fluoro-1,3-dioxoisoindolin-5-yl)piperazin-1-yl)heptyl)oxy)-2-methylphenyl)picolinic acid

### Step A. tert-Butyl 8-(benzo[d]thiazol-2-ylcarbamoyl)-3,4-dihydroisoquinoline-2(1H)-carboxylate

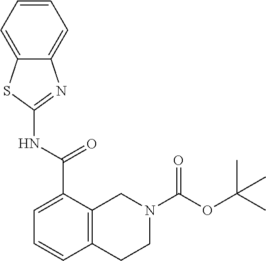 [SMILES: CC(C)(C)OC(=O)N1CCc2cccc(C(=O)Nc3nc4ccccc4s3)c2C1]

2-(tert-Butoxycarbonyl)-1,2,3,4-tetrahydroisoquinoline-8-carboxylic acid (6.5 g, 1 Eq, 23 mmol) was dissolved in dimethylformamide (30 mL). To the mixture was added HATU (11 g, 1.2 Eq, 28 mmol), followed by benzo[d]thiazol-2-amine (4.2 g, 1.2 Eq, 28 mmol) and N,N-diisopropylethylamine (4.5 g, 6.1 mL, 1.5 Eq, 35 mmol). The mixture was stirred at room temperature for overnight. The mixture was concentrated. The crude was extracted with ethyl acetate and water. The organic layer was separated and washed with brine, dried over anhydrous sodium sulfate, filtered, and evaporated to dryness. The crude was then purified by silica gel chromatography (ethyl acetate/hexane) to afford the title compound (8.1 g, 20 mmol, 84% yield). MS (ESI) m/z: 410.2 [M+H]⁺

### Step B. N-(Benzo[d]thiazol-2-yl)-1,2,3,4-tetrahydroisoquinoline-8-carboxamide hydrochloride

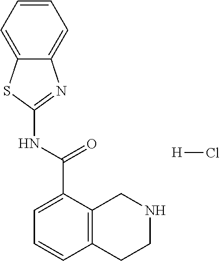 [SMILES: O=C(Nc1nc2ccccc2s1)c1cccc2c1CNCC2 ; Cl]

tert-Butyl 8-(benzo[d]thiazol-2-ylcarbamoyl)-3,4-dihydroisoquinoline-2(1H)-carboxylate (8 g, 0.02 mol) was suspended in 4M HCl in 1,4-dioxane (80 mL). The mixture was then stirred room temperature for overnight. The precipitate was collected via filtration. The solid was washed with ethyl acetate 3 times, and then dried under vacuum to afford the title compound (8 g, 0.02 mol, 100% yield). MS (ESI) m/z: 310.1 [M+H]⁺

### Step C. tert-Butyl 6-(8-(benzo[d]thiazol-2-ylcarbamoyl)-3,4-dihydroisoquinolin-2(1H)-yl)-3-bromopicolinate

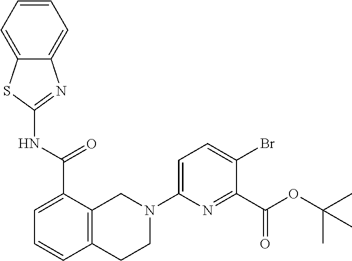 [SMILES: CC(C)(C)OC(=O)c1nc(N2CCc3cccc(C(=O)Nc4nc5ccccc5s4)c3C2)ccc1Br]

N-(Benzo[d]thiazol-2-yl)-1,2,3,4-tetrahydroisoquinoline-8-carboxamide, HCl (6.0 g, 1 Eq, 17 mmol) was suspended in dimethylacetamide (60 mL). To the mixture was added tert-butyl 3-bromo-6-chloropicolinate (6.6 g, 1.3 Eq, 23 mmol), followed by cesium carbonate (28 g, 5 Eq, 87 mmol). The mixture was then heated at 120° C. for 20 h. The mixture was cooled down to room temperature. To the mixture was then added 180 mL water. The mixture was then stirred at room temperature for 10 min. The mixture was then filtered and rinsed with water. The product was then recrystallized from 40 mL ethanol. The precipitate was collected via filtration, rinsed with ethanol 2×. The solid was then dried under vacuum overnight to afford the title compound (5.2 g, 9.2 mmol, 53% yield). MS (ESI) m/z: 565.1 [M+H]⁺

### Step D. Methyl-3-(4,4,5,5-tetramethyl-1,3,2-dioxaborolan-2-yl)phenol

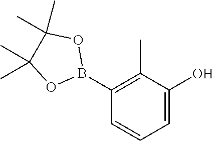 [SMILES: Cc1c(O)cccc1B1OC(C)(C)C(C)(C)O1]

3-bromo-2-methylphenol (2.0 g, 1 Eq, 11 mmol) was dissolved 1,4-dioxane (20 mL). To the mixture was added 4,4,4′,4′,5,5,5′,5′-octamethyl-2,2′-bi(1,3,2-dioxaborolane) (4.1 g, 1.5 Eq, 16 mmol), followed by potassium acetate (3.1 g, 3 Eq, 32 mmol) and PdCl₂(dppf)-CH₂Cl₂ adduct (0.44 g, 0.05 Eq, 0.53 mmol). The mixture was then heated at 85° C. After overnight, the mixture was cooled down to room temperature. The mixture was then filtered through Celite, and the filtrate was extracted with ethyl acetate and water. The organic layer was separated, washed with brine, dried over Na₂SO₄, filtered, and evaporated to dryness. The crude was then purified by silica gel chromatography (ethyl acetate/hexane gradient). The combined fractions were evaporated to dryness. The product was then recrystallized with cold ether to afford the title compound (2.1 g, 9.0 mmol, 84% yield). MS (ESI) m/z: 235.2 [M+H]⁺

### Step E. 7-(2-Methyl-3-(4,4,5,5-tetramethyl-1,3,2-dioxaborolan-2-yl)phenoxy)heptan-1-ol

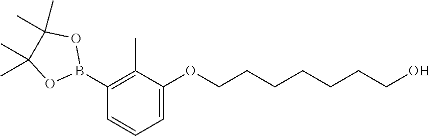 [SMILES: Cc1c(OCCCCCCCO)cccc1B1OC(C)(C)C(C)(C)O1]

2-Methyl-3-(4,4,5,5-tetramethyl-1,3,2-dioxaborolan-2-yl)phenol (100 mg, 1 Eq, 427 μmol), 7-bromoheptan-1-ol (91.7 mg, 1.1 Eq, 470 μmol) and K₂CO₃ (177 mg, 3 Eq, 1.28 mmol) were suspended in acetonitrile (4 mL). The mixture was then heated at 70° C. for overnight. After completion, the mixture was evaporated. The crude was then purified by silica gel chromatography (ethyl acetate/hexane gradient) to afford the title product 7-(2-methyl-3-(4,4,5,5-tetramethyl-1,3,2-dioxaborolan-2-yl)phenoxy)heptan-1-ol (108 mg, 310 μmol, 72.6% yield). MS (ESI) m/z: 349.3 [M+H]⁺

### Step F. tert-Butyl 6-(8-(benzo[d]thiazol-2-ylcarbamoyl)-3,4-dihydroisoquinolin-2(1H)-yl)-3-(3-((7-hydroxyheptyl)oxy)-2-methylphenyl)picolinate

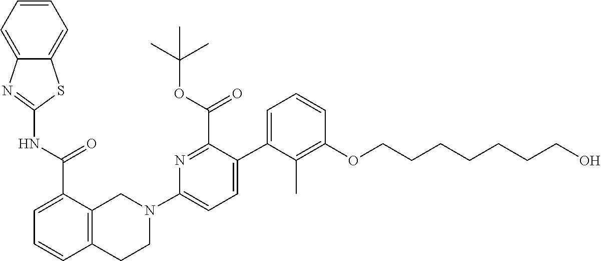 [SMILES: Cc1c(OCCCCCCCO)cccc1-c1ccc(N2CCc3cccc(C(=O)Nc4nc5ccccc5s4)c3C2)nc1C(=O)OC(C)(C)C]

tert-Butyl 6-(8-(benzo[d]thiazol-2-ylcarbamoyl)-3,4-dihydroisoquinolin-2(1H)-yl)-3-bromopicolinate (150 mg, 1 Eq, 265 μmol), 7-(2-methyl-3-(4,4,5,5-tetramethyl-1,3,2-dioxaborolan-2-yl)phenoxy)heptan-1-ol (111 mg, 1.2 Eq, 318 μmol) and mesylate[(di(1-adamantyl)-n-butylphosphine)-2-(2′-amino-1,1′-biphenyl)]palladium(II) (9.66 mg, 0.05 Eq, 13.3 μmol) were suspended in 1,4-dioxane (2.4 mL) and 0.6 mL 1.5 M K₃PO₄ (aq.). The mixture was heated at 100° C. for 30 min using microwave. After completion, the mixture was concentrated and then purified by preparative RP-HPLC (water/acetonitrile with 0.1% formic acid in each). Combined and lyophilized pure product containing fractions to afford the title compound (67 mg, 95 μmol, 36%). MS (ESI) m/z: 707.3 [M+H]⁺

### Step G. tert-Butyl 6-(8-(benzo[d]thiazol-2-ylcarbamoyl)-3,4-dihydroisoquinolin-2(1H)-yl)-3-(2-methyl-3-((7-oxoheptyl)oxy)phenyl)picolinate

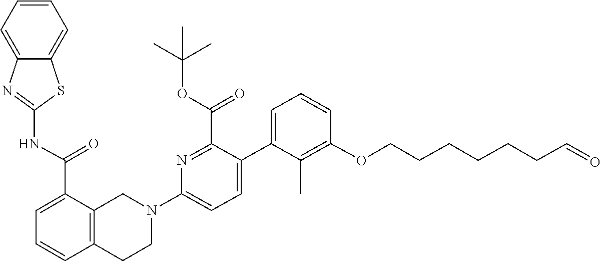 [SMILES: Cc1c(OCCCCCCC=O)cccc1-c1ccc(N2CCc3cccc(C(=O)Nc4nc5ccccc5s4)c3C2)nc1C(=O)OC(C)(C)C]

tert-Butyl 6-(8-(benzo[d]thiazol-2-ylcarbamoyl)-3,4-dihydroisoquinolin-2(1H)-yl)-3-(3-((7-hydroxyheptyl)oxy)-2-methylphenyl)picolinate (50 mg, 1 Eq, 71 μmol) was dissolved in dichloromethane (4 mL). To the mixture was added Dess-Martin periodinane (36 mg, 26 μL, 1.2 Eq, 85 μmol). The mixture was then stirred at room temperature for 2 h. A few drops of methanol were added. The mixture was then concentrated under reduced pressure to afford the title compound that was used without further purification. MS (ESI) m/z: 705.3 [M+H]⁺

### Step H. tert-Butyl 6-(8-(benzo[d]thiazol-2-ylcarbamoyl)-3,4-dihydroisoquinolin-2(1H)-yl)-3-(3-((7-(4-(2-(2,6-dioxopiperidin-3-yl)-6-fluoro-1,3-dioxoisoindolin-5-yl)piperazin-1-yl)heptyl)oxy)-2-methylphenyl)picolinate

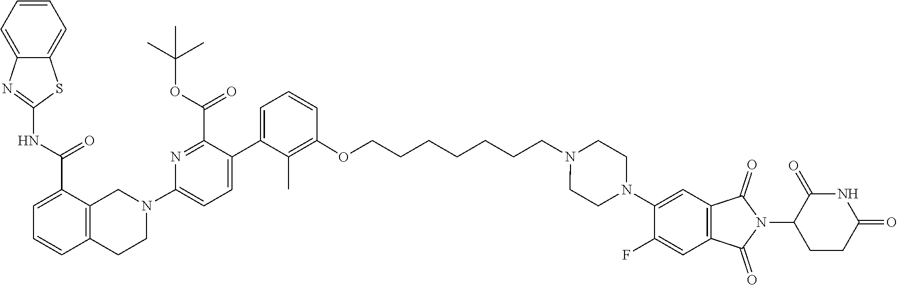 [SMILES: Cc1c(OCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)OC(C)(C)C]

tert-Butyl 6-(8-(benzo[d]thiazol-2-ylcarbamoyl)-3,4-dihydroisoquinolin-2(1H)-yl)-3-(2-methyl-3-((7-oxoheptyl)oxy)phenyl)picolinate (crude, 50 mg, 1 Eq, 71 μmol) was resuspended in ethanol (5 mL). To the mixture was added 2-(2,6-dioxopiperidin-3-yl)-5-fluoro-6-(piperazin-1-yl)isoindoline-1,3-dione (38 mg, 1.5 Eq, 0.11 mmol), followed by sodium cyanoborohydride (22 mg, 5 Eq, 0.35 mmol) and acetic acid (4.3 mg, 4.1 μL, 1 Eq, 71 μmol). The mixture was then stirred at room temperature for overnight. After completion, the solvents were removed. The crude was then purified by preparative RP-HPLC (water/acetonitrile with 0.1% formic acid in each). Combined and lyophilized pure product containing fractions to afford the title compound (30 mg, 29 μmol, 40%). MS (ESI) m/z: 1049.3 [M+H]⁺

### Step I. 6-(8-(Benzo[d]thiazol-2-ylcarbamoyl)-3,4-dihydroisoquinolin-2(1H)-yl)-3-(3-((7-(4-(2-(2,6-dioxopiperidin-3-yl)-6-fluoro-1,3-dioxoisoindolin-5-yl)piperazin-1-yl)heptyl)oxy)-2-methylphenyl)picolinic acid

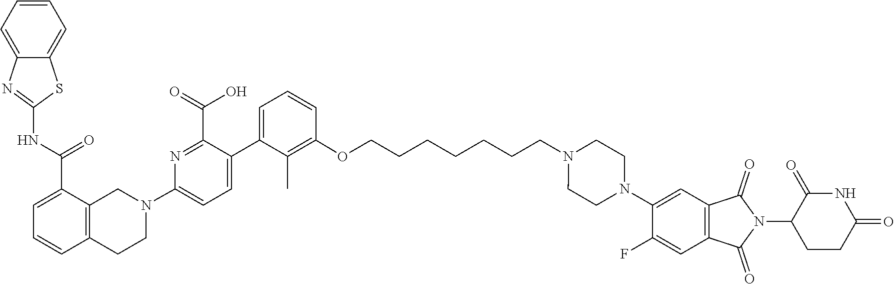 [SMILES: Cc1c(OCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)O]

tert-Butyl 6-(8-(benzo[d]thiazol-2-ylcarbamoyl)-3,4-dihydroisoquinolin-2(1H)-yl)-3-(3-((7-(4-(2-(2,6-dioxopiperidin-3-yl)-6-fluoro-1,3-dioxoisoindolin-5-yl)piperazin-1-yl)heptyl)oxy)-2-methylphenyl)picolinate (30 mg, 1 Eq, 29 μmol) was dissolved in dichloromethane (2.4 mL). To the mixture was added 0.6 mL TFA. The mixture was then stirred at room temperature for 24 hrs. The solvents were removed. The residue was then purified by preparative RP-HPLC (water/acetonitrile with 0.1% formic acid in each). Combined and lyophilized pure product containing fractions to afford the title compound (15 mg, 15 μmol, 53%) as a yellow solid. MS (ESI) m/z: 993.2 [M+H]⁺

### Example 3. Preparation of Compound 106

### 6-[8-(1,3-Benzothiazol-2-ylcarbamoyl)-3,4-dihydro-1H-isoquinolin-2-yl]-3-[3-[2-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]ethoxy]-2-methyl-phenyl]pyridine-2-carboxylic acid

### Step A. tert-Butyl 4-[2-(3-bromo-2-methyl-phenoxy) ethyl]piperidine-1-carboxylate

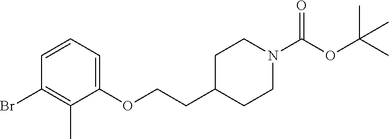 [SMILES: Cc1c(Br)cccc1OCCC1CCN(C(=O)OC(C)(C)C)CC1]

A mixture of tert-butyl 4-(2-hydroxyethyl)piperidine-1-carboxylate (5 g, 21.80 mmol, 1 equiv.), 3-bromo-2-methyl-phenol (4.08 g, 21.80 mmol, 1 equiv.), PPh₃ (7.43 g, 28.35 mmol, 1.3 equiv.) in THF (40 mL) was degassed and purged with nitrogen 3 times and then DIAD (5.29 g, 26.16 mmol, 5.09 mL, 1.2 equiv.) was added dropwise at 0° C., the mixture was stirred at 25° C. for 12 h. The reaction was diluted with H₂O (100 mL) and extracted with EtOAC (60 mL×3). The combined organic layers were washed with H₂O (60 mL×3), dried over anhydrous sodium sulfate, filtered and concentrated under reduced pressure to give a residue that was then purified by silica gel chromatography (ethyl acetate/hexane) to afford the title compound (8.2 g, 18.53 mmol, 84.97% yield). MS (ESI) m/z: 344.0 [M−tBu]⁺.

### Step B. 4-[2-(3-Bromo-2-methyl-phenoxy)ethyl]piperidine

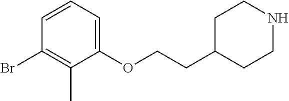 [SMILES: Cc1c(Br)cccc1OCCC1CCNCC1]

To a solution of tert-butyl 4-[2-(3-bromo-2-methyl-phenoxy)ethyl]piperidine-1-carboxylate (8.2 g, 20.59 mmol, 1 equiv.) was added 4.0N HCl in dioxane (100 mL). The mixture was stirred at 25° C. for 2 hours and then concentrated under reduced pressure to afford the title compound that was used without further purification (6 g, crude).

### Step C. Ethyl 2-[4-[2-(3-bromo-2-methyl-phenoxy)ethyl]-1-piperidyl]acetate

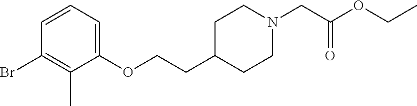 [SMILES: CCOC(=O)CN1CCC(CCOc2cccc(Br)c2C)CC1]

To a solution of 4-[2-(3-bromo-2-methyl-phenoxy)ethyl]piperidine (3 g, 10.06 mmol, 1 equiv.) in ACN (30 mL) was added ethyl 2-bromoacetate (2.02 g, 12.07 mmol, 1.34 mL, 1.2 equiv.) and TEA (3.05 g, 30.18 mmol, 4.20 mL, 3 equiv.). The mixture was stirred at 20° C. for 12 h. The then diluted with H₂O (30 mL) and extracted with ethyl acetate (60 mL×3). The combined organic layers were washed with H₂O (10 mL×3), dried over anhydrous sodium sulfate, filtered, and concentrated under reduced pressure to give a residue that was then purified by silica gel chromatography (ethyl acetate/hexane) to afford the title compound (3 g, 7.73 mmol, 76.82% yield). MS (ESI) m/z: 385.8 [M+H]⁺.

### Step D. tert-Butyl 6-[8-(1,3-benzothiazol-2-ylcarbamoyl)-3,4-dihydro-1H-isoquinolin-2-yl]-3-[3-[2-[1-(2-ethoxy-2-oxo-ethyl)-4-piperidyl]ethoxy]-2-methyl-phenyl]pyridine-2-carboxylate

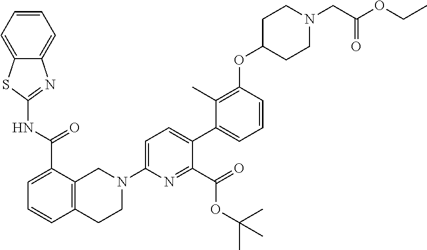 [SMILES: CCOC(=O)CN1CCC(Oc2cccc(-c3ccc(N4CCc5cccc(C(=O)Nc6nc7ccccc7s6)c5C4)nc3C(=O)OC(C)(C)C)c2C)CC1]

To a solution of ethyl 2-[4-[2-(3-bromo-2-methyl-phenoxy)ethyl]-1-piperidyl]acetate (700 mg, 1.82 mmol, 1 equiv.) in 1,4-dioxane (8 mL) was added ditert-butyl(cyclopentyl)phosphane; dichloropalladium; iron (237.43 mg, 364.29 μmol, 0.2 equiv.), KF (1.5 M, 1.82 mL, 1.5 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate (1.23 g, 2.00 mmol, 1.1 equiv.), The mixture was stirred at 90° C. for 2 hours then diluted with H₂O 30 mL and extracted with ethyl acetate (60 mL×3). The combined organic layers were washed with H₂O (10 mL×3), dried over anhydrous sodium sulfate, filtered, and concentrated under reduced pressure to give a residue that was then purified by silica gel chromatography (ethyl acetate/hexane) to afford the title compound (600 mg, 721.54 μmol, 39.61% yield). MS (ESI) m/z: 790.5 [M+H]⁺.

### Step E. 2-[4-[2-[3-[6-[8-(1,3-Benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid

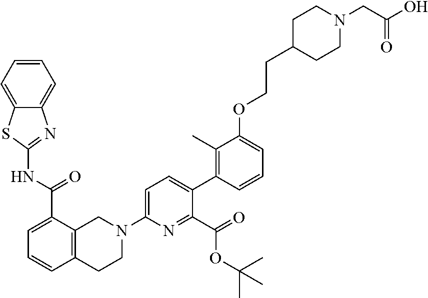 [SMILES: Cc1c(OCCC2CCN(CC(=O)O)CC2)cccc1-c1ccc(N2CCc3cccc(C(=O)Nc4nc5ccccc5s4)c3C2)nc1C(=O)OC(C)(C)C]

To a solution of tert-butyl 6-[8-(1,3-benzothiazol-2-ylcarbamoyl)-3,4-dihydro-1H-isoquinolin-2-yl]-3-[3-[2-[1-(2-ethoxy-2-oxo-ethyl)-4-piperidyl]ethoxy]-2-methyl-phenyl]pyridine-2-carboxylate (600 mg, 759.51 μmol, 1 equiv.) in THF (3 mL) was added LiOH·H2O (95.62 mg, 2.28 mmol, 3 equiv.) and H₂O (1 mL). The mixture was stirred at 20° C. for 1 h. The pH of the reaction mixture was adjusted to 5 with citric acid, diluted with H₂O (10 mL) and extracted with DCM (10 mL×3). The combined organic layers were washed with H₂O (10 mL×3), dried over anhydrous sodium sulfate, filtered, and concentrated under reduced pressure to afford the title compound that was used without further purification (410 mg, crude). MS (ESI) m/z: 762.3 [M+H]⁺.

### Step F. tert-Butyl 6-[8-(1,3-benzothiazol-2-ylcarbamoyl)-3,4-dihydro-1H-isoquinolin-2-yl]-3-[3-[2-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]ethoxy]-2-methyl-phenyl]pyridine-2-carboxylate

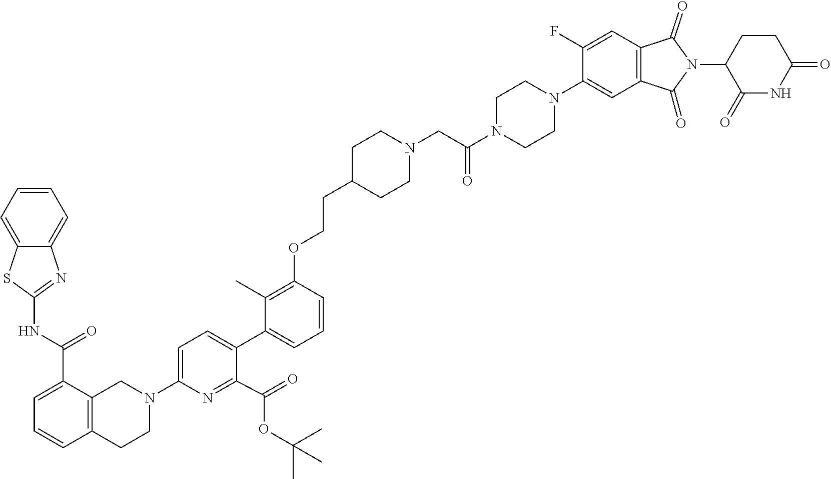 [SMILES: Cc1c(OCCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)OC(C)(C)C]

To a solution of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid (70 mg, 91.87 μmol, 1 equiv.) in DMF (1 mL) was added 2-(2,6-dioxo-3-piperidyl)-5-fluoro-6-piperazin-1-yl-isoindoline-1,3-dione (36.42 mg, 101.06 μmol, 1.1 equiv.), HATU (38.43 mg, 101.06 μmol, 1.1 equiv.) and DIPEA (35.62 mg, 275.62 μmol, 48.01 μL, 3 equiv.) at 25° C. The reaction mixture was stirred at 25° C. for 2 h then the reaction mixture was diluted with water 5 mL and filtered to afford the title compound that was used without further purification (166 mg, crude). MS (ESI) m/z: 553.1 [M+2H]⁺/2.

### Step G. 6-[8-(1,3-Benzothiazol-2-ylcarbamoyl)-3,4-dihydro-1H-isoquinolin-2-yl]-3-[3-[2-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]ethoxy]-2-methyl-phenyl]pyridine-2-carboxylic acid

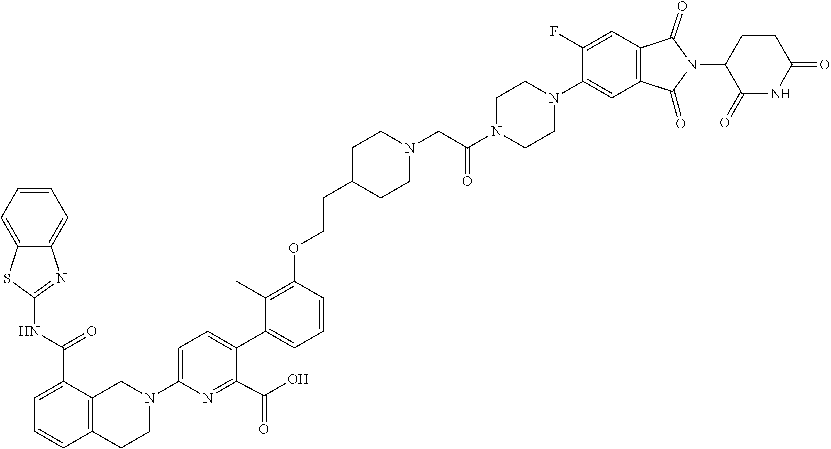 [SMILES: Cc1c(OCCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

A solution of tert-butyl 6-[8-(1,3-benzothiazol-2-ylcarbamoyl)-3,4-dihydro-1H-isoquinolin-2-yl]-3-[3-[2-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]ethoxy]-2-methyl-phenyl]pyridine-2-carboxylate (166 mg, 150.33 μmol, 1 equiv.) in DCM (1 mL) and TFA (1 mL) was stirred at 40° C. for 2 h. The reaction mixture was concentrated under reduced pressure to give a residue that was purified by preparative RP-HPLC (water/acetonitrile with 0.1% formic acid in each). Combined and lyophilized pure product containing fractions to afford the title compound (32.33 mg, 29.18 μmol, 19.41% yield, 94.60% purity). MS (ESI) m/z: 1048.3 [M+H]⁺; ¹H NMR (400 MHz, DMSO-d₆) δ 11.11 (s, 1H), 8.03 (d, J=8.0 Hz, 1H), 7.81-7.73 (m, 2H), 7.62 (d, J=7.6 Hz, 1H), 7.55-7.28 (m, 7H), 7.09 (t, J=8.0 Hz, 1H), 6.96 (d, J=8.8 Hz, 1H), 6.89 (d, J=8.4 Hz, 1H), 6.62 (d, J=7.6 Hz, 1H), 5.11 (dd, J=5.4, 12.8 Hz, 1H), 4.98 (s, 2H), 4.04-3.96 (m, 2H), 3.92 (t, J=5.6 Hz, 2H), 3.77-3.70 (m, 2H), 3.62 (s, 2H), 3.21 (s, 4H), 3.03 (t, J=5.6 Hz, 2H), 2.91-2.81 (m, 3H), 2.63-2.57 (m, 2H), 2.11-1.98 (m, 3H), 1.88 (s, 3H), 1.79-1.64 (m, 5H), 1.50 (d, J=3.2 Hz, 1H), 1.25 (d, J=11.6 Hz, 3H).

### Example 4. Preparation of Compound 110

### 6-[8-(1,3-Benzothiazol-2-ylcarbamoyl)-3,4-dihydro-1H-isoquinolin-2-yl]-3-[3-[4-[3-[4-[2-(2,6-dioxo-3-piperidyl)-6-fluoro-1,3-dioxo-isoindolin-5-yl]piperazin-1-yl]propyl]phenoxy]-2-methyl-phenyl]pyridine-2-carboxylic acid

### Step A. Ethyl 3-[4-(3-bromo-2-methyl-phenoxy)phenyl]propanoate

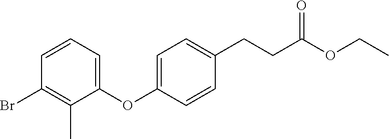 [SMILES: CCOC(=O)CCc1ccc(Oc2cccc(Br)c2C)cc1]

A mixture of ethyl 3-(4-hydroxyphenyl)propanoate (5 g, 25.74 mmol, 1 equiv.), 1,3-dibromo-2-methyl-benzene (12.87 g, 51.49 mmol, 2 equiv.), Cs₂CO₃ (10.07 g, 30.89 mmol, 1.2 equiv.) and 2,2,6,6-tetramethylheptane-3,5-dione (1.19 g, 6.44 mmol, 1.33 mL, 0.25 equiv.) in NMP (50 mL) was degassed and purged with nitrogen 3 times. CuI (2.45 g, 12.87 mmol, 0.5 equiv.) was then added and the mixture was stirred at 120° C. for 12 h under a nitrogen atmosphere. The reaction mixture was filtered, the filtrate was diluted with water 30 mL and extracted with ethyl acetate (40 mL×2). The combined organic layers were washed with brine (20 mL×2), dried over anhydrous sodium sulfate, filtered, and concentrated under reduced pressure to give a residue that was then purified by silica gel chromatography (ethyl acetate/hexane) to afford the title compound (7.6 g, 20.92 mmol, 81.27% yield). ¹H NMR (400 MHz, CDCl₃) δ 7.35 (d, J=8.0 Hz, 1H), 7.15 (d, J=8.4 Hz, 2H), 7.01 (t, J=8.0 Hz, 1H), 6.86-6.80 (m, 3H), 4.14 (q, J=7.2 Hz, 2H), 2.93 (t, J=8.0 Hz, 2H), 2.61 (t, J=8.0 Hz, 2H), 2.34 (s, 3H), 1.25 (t, J=7.2 Hz, 3H).

### Step B. 3-[4-(3-Bromo-2-methyl-phenoxy)phenyl]propan-1-ol

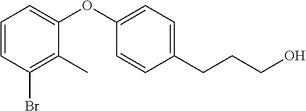 [SMILES: Cc1c(Br)cccc1Oc1ccc(CCCO)cc1]

To a mixture of ethyl 3-[4-(3-bromo-2-methyl-phenoxy)phenyl]propanoate (7.6 g, 20.92 mmol, 1 equiv.) in THF (70 mL) was slowly added LiAlH₄ (794.01 mg, 20.92 mmol, 1 equiv.) at 0° C., then the mixture was stirred at 25° C. for 2 hours under a nitrogen atmosphere. The mixture was slowly poured into ice-water (150 mL). The pH was adjusted to pH 4-5, extracted with EtOAc (60 mL×3), washed with brine (30 mL×2), dried over anhydrous sodium sulfate, filtered, and concentrated under reduced pressure to afford the title compound which was used without further purification (6 g, 18.68 mmol, 89.28% yield). ¹H NMR (400 MHz, CDCl₃) δ=7.32 (d, J=8.0 Hz, 1H), 7.13 (d, J=8.4 Hz, 2H), 6.98 (t, J=8.0 Hz, 1H), 6.85-6.79 (m, 3H), 3.67 (t, J=6.4 Hz, 2H), 2.70-2.66 (m, 2H), 2.33 (s, 3H), 1.91-1.83 (m, 2H).

### Step C. 3-[4-(3-Bromo-2-methyl-phenoxy)phenyl]propanal

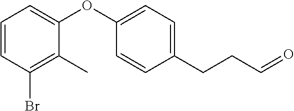 [SMILES: Cc1c(Br)cccc1Oc1ccc(CCC=O)cc1]

To a solution of oxalyl dichloride (2.37 g, 18.68 mmol, 1.64 mL, 2 equiv.) in DCM (5 mL) was added a mixture of DMSO (2.92 g, 37.36 mmol, 2.92 mL, 4 equiv.) in DCM (5 mL) at −70° C. and stirred for 0.5 h. 3-[4-(3-bromo-2-methyl-phenoxy)phenyl]propan-1-ol (3 g, 9.34 mmol, 1 equiv.) in DCM (5 mL) was added to the mixture over 20 min. TEA (5.67 g, 56.04 mmol, 7.80 mL, 6 equiv.) was added into the mixture and the reaction was then stirred at −70° C. for 2 h.

The reaction mixture was quenched by addition water (60 mL) at 0° C., and then extracted with DCM (30 mL×2). The combined organic layers were washed with brine (20 mL×2), dried over anhydrous sodium sulfate, filtered, and concentrated under reduced pressure to give a residue that was purified by silica gel chromatography (ethyl acetate/hexane) to afford the title compound (2.6 g, 8.15 mmol, 87.21% yield). ¹H NMR (400 MHz, CDCl₃) δ 9.84 (s, 1H), 7.35 (d, J=8.0 Hz, 1H), 7.15 (d, J=8.4 Hz, 2H), 7.02 (t, J=8.0 Hz, 1H), 6.86-6.81 (m, 3H), 2.97-2.91 (m, 2H), 2.82-2.76 (m, 2H), 2.34 (s, 3H).

### Step D. tert-Butyl 6-[8-(1,3-benzothiazol-2-ylcarbamoyl)-3,4-dihydro-1H-isoquinolin-2-yl]-3-[2-methyl-3-[4-(3-oxopropyl)phenoxy]phenyl]pyridine-2-carboxylate

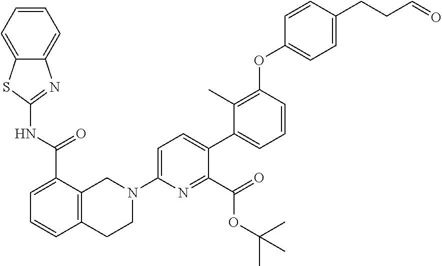 [SMILES: Cc1c(Oc2ccc(CCC=O)cc2)cccc1-c1ccc(N2CCc3cccc(C(=O)Nc4nc5ccccc5s4)c3C2)nc1C(=O)OC(C)(C)C]

A mixture of 3-[4-(3-bromo-2-methyl-phenoxy)phenyl]propanal (100 mg, 313.29 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (153.52 mg, 250.63 μmol, 0.8 equiv.), Ad2nBuP Pd G3(cataCXium® A Pd G3, 45.63 mg, 62.66 μmol, 0.2 equiv.), K₂CO₃ (1.5 M aq., 313.29 μL, 1.5 equiv.) in 1,4-dioxane (1 mL) was degassed and purged with nitrogen 3 times, and then the mixture was stirred at 80° C. for 2 h under a nitrogen atmosphere. The reaction mixture was concentrated under reduced pressure to give a residue that was purified by silica gel chromatography (methanol/DCM) to afford the title compound (100 mg, 107.30 μmol, 11.42% yield). MS (ESI) m/z: 725.2 [M+H]⁺.

### Step E. tert-Butyl 6-[8-(1,3-benzothiazol-2-ylcarbamoyl)-3,4-dihydro-1H-isoquinolin-2-yl]-3-[3-[4-[3-[4-[2-(2,6-dioxo-3-piperidyl)-6-fluoro-1,3-dioxo-isoindolin-5-yl]piperazin-1-yl]propyl]phenoxy]-2-methyl-phenyl]pyridine-2-carboxylate

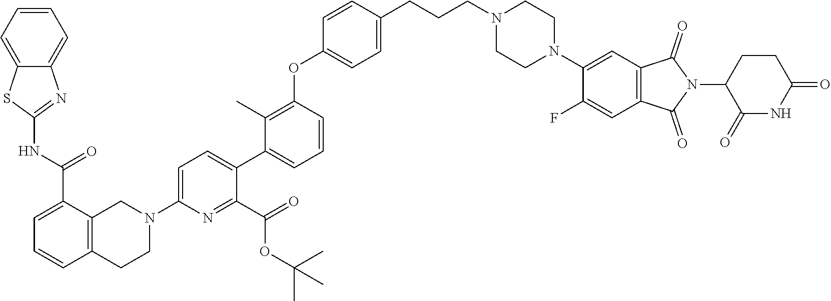 [SMILES: Cc1c(Oc2ccc(CCCN3CCN(c4cc5c(cc4F)C(=O)N(C4CCC(=O)NC4=O)C5=O)CC3)cc2)cccc1-c1ccc(N2CCc3cccc(C(=O)Nc4nc5ccccc5s4)c3C2)nc1C(=O)OC(C)(C)C]

A mixture of tert-butyl 6-[8-(1,3-benzothiazol-2-ylcarbamoyl)-3,4-dihydro-1H-isoquinolin-2-yl]-3-[2-methyl-3-[4-(3-oxopropyl)phenoxy]phenyl]pyridine-2-carboxylate (100 mg, 137.96 μmol, 1 equiv.), 2-(2,6-dioxo-3-piperidyl)-5-fluoro-6-piperazin-1-yl-isoindoline-1,3-dione (49.71 mg, 137.96 μmol, 1 equiv.), acetic acid (8.28 mg, 137.96 μmol, 7.89 μL, 1 equiv.), NaBH(OAc)₃ (43.86 mg, 206.94 μmol, 1.5 equiv.) in DCM (1 mL) was degassed and purged with nitrogen 3 times. The mixture was stirred at 25° C. for 20 h under a nitrogen atmosphere. The reaction mixture was concentrated under reduced pressure to give a residue that was purified by silica gel chromatography (ethyl acetate/hexane) to afford the title compound (150 mg, 104.10 μmol, 75.46% yield). MS (ESI) m/z: 1071.3 [M+H]⁺.

### Step F. 6-[8-(1,3-Benzothiazol-2-ylcarbamoyl)-3,4-dihydro-1H-isoquinolin-2-yl]-3-[3-[4-[3-[4-[2-(2,6-dioxo-3-piperidyl)-6-fluoro-1,3-dioxo-isoindolin-5-yl]piperazin-1-yl]propyl]phenoxy]-2-methyl-phenyl]pyridine-2-carboxylic acid

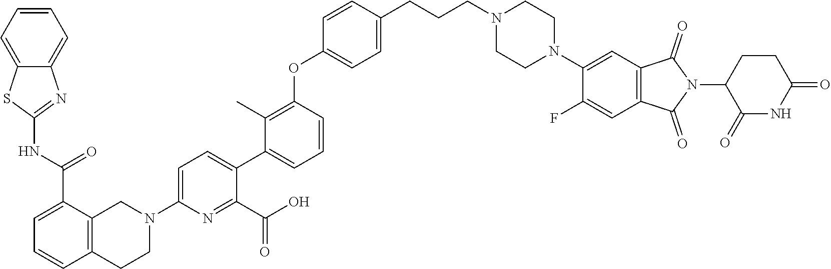 [SMILES: Cc1c(Oc2ccc(CCCN3CCN(c4cc5c(cc4F)C(=O)N(C4CCC(=O)NC4=O)C5=O)CC3)cc2)cccc1-c1ccc(N2CCc3cccc(C(=O)Nc4nc5ccccc5s4)c3C2)nc1C(=O)O]

A mixture of tert-butyl 6-[8-(1,3-benzothiazol-2-ylcarbamoyl)-3,4-dihydro-1H-isoquinolin-2-yl]-3-[3-[4-[3-[4-[2-(2,6-dioxo-3-piperidyl)-6-fluoro-1,3-dioxo-isoindolin-5-yl]piperazin-1-yl]propyl]phenoxy]-2-methyl-phenyl]pyridine-2-carboxylate (120 mg, 112.23 μmol, 1 equiv.), TFA (12.80 mg, 112.23 μmol, 8.31 μL, 1 equiv.) in DCM (1 mL) was stirred at 25° C. for 3 h. The reaction mixture was concentrated under reduced pressure to give a residue that was purified by preparative RP-HPLC (water/acetonitrile with 0.1% formic acid in each). Combined and lyophilized pure product containing fractions to afford the title compound (38.86 mg, 36.59 μmol, 32.60% yield, 95.4% purity). MS (ESI) m/z: 1070.3 [M+H]⁺; ¹H NMR (400 MHz, CDCl₃) δ 8.40 (d, J=7.6 Hz, 1H), 7.89 (d, J=8.0 Hz, 1H), 7.81 (d, J=8.8 Hz, 1H), 7.70 (d, J=7.2 Hz, 1H), 7.56-7.45 (m, 4H), 7.43-7.38 (m, 3H), 7.23 (s, 1H), 7.11-7.06 (m, 3H), 6.96 (d, J=9.2 Hz, 1H), 6.91 (d, J=8.0 Hz, 2H), 5.24 (s, 2H), 5.00-4.91 (m, 1H), 3.94 (t, J=6.4 Hz, 2H), 3.60 (s, 4H), 3.17 (t, J=6.0 Hz, 2H), 3.07-2.96 (m, 4H), 2.92-2.79 (m, 3H), 2.76-2.68 (m, 3H), 2.20-2.12 (m, 4H), 1.92 (s, 3H).

### Example 5. Preparation of Compound 118

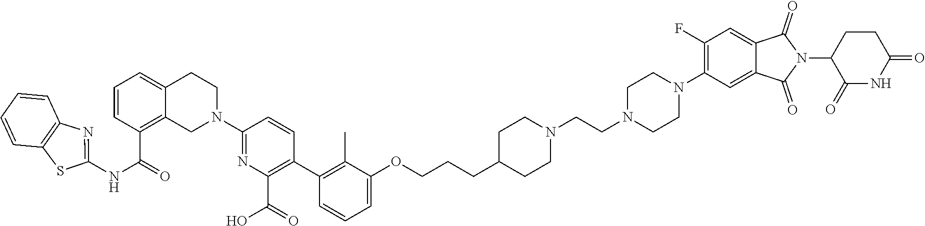 [SMILES: Cc1c(OCCCC2CCN(CCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)ethyl)piperidin-4-yl)propoxy)-2-methylphenyl)picolinic acid

### Step A. tert-Butyl 4-[3-(3-bromo-2-methyl-phenoxy)propyl]piperidine-1-carboxylate

To a solution of 3-bromo-2-methyl-phenol (1 g, 5.35 mmol, 1 eq.), tert-butyl 4-(3-bromopropyl)piperidine-1-carboxylate (1.64 g, 5.35 mmol, 1 equiv.) in MeCN (4 mL) was added K₂CO₃ (2.22 g, 16.04 mmol, 3 eq.) and the mixture was stirred at 60° C. After 2 h the reaction solution was concentrated under reduced pressure purified by column chromatography (SiO₂, Petroleum ether/Ethyl acetate=1/0 to 10/1) to give tert-butyl 4-[3-(3-bromo-2-methyl-phenoxy)propyl]piperidine-1-carboxylate (2.2 g, 5.34 mmol, 99.8% yield) as a colorless oil. MS(ESI) m/z: 312.4 [M+H]⁺; ¹H NMR (400 MHz, CDCl₃) δ=7.14 (d, J=8.0 Hz, 1H), 6.99 (t, J=8.0 Hz, 1H), 6.76 (d, J=8.4 Hz, 1H), 4.14-4.06 (m, 2H), 3.94 (t, J=6.4 Hz, 2H), 2.69 (t, J=2.4, 12.8 Hz, 2H), 2.32 (s, 3H), 1.88-1.79 (m, 2H), 1.70 (d, J=12.8 Hz, 2H), 1.48-1.43 (m, 12H), 1.18-1.08 (m, 2H)

### Step B. 4-[3-(3-bromo-2-methyl-phenoxy)propyl]piperidine

To a solution of tert-butyl 4-[3-(3-bromo-2-methyl-phenoxy)propyl]piperidine-1-carboxylate (2.2 g, 5.34 mmol, 1 equiv.) in 4 M HCl in EtOAc (10 mL) was stirred at 25° C. After 1 h the reaction mixture was filtered and concentrated under reduced pressure to give 4-[3-(3-bromo-2-methyl-phenoxy)propyl]piperidine (1.6 g, 5.12 mmol, 96.05% yield) as a white solid which was carried forward without further purification. MS(ESI) m/z: 314.4 [M+H]⁺; ¹HNMR (400 MHz, CDCl₃) δ=7.14 (d, J=8.0 Hz, 1H), 6.99 (t, J=8.0 Hz, 1H), 6.76 (d, J=8.4 Hz, 1H), 4.14-4.06 (m, 2H), 3.94 (t, J=6.4 Hz, 2H), 2.69 (t, J=2.4, 12.8 Hz, 2H), 2.32 (s, 3H), 1.88-1.79 (m, 2H), 1.70 (d, J=12.8 Hz, 2H), 1.48-1.43 (m, 12H), 1.18-1.08 (m, 2H).

### Step C. 4-[3-(3-bromo-2-methyl-phenoxy)propyl]-1-(2,2-diethoxyethyl)piperidine

To a solution of 4-[3-(3-bromo-2-methyl-phenoxy)propyl]piperidine (1.5 g, 4.80 mmol, 1 equiv.) and 2-bromo-1,1-diethoxy-ethane (946.71 mg, 4.80 mmol, 722.68 uL, 1 equiv.) in MeCN (5 mL) was added K₂CO₃ (1.99 g, 14.41 mmol, 3 equiv.) and potassium iodide (797.46 mg, 4.80 mmol, 1 equiv.). The mixture was stirred at 80° C. for 8 hours. The reaction mixture was concentrated under reduced pressure and purified by column chromatography (SiO₂, Ethyl acetate/MeOH=1/0 to 20/1) to give 4-[3-(3-bromo-2-methyl-phenoxy)propyl]-1-(2,2-diethoxyethyl)piperidine (1.7 g, 3.97 mmol, 82.6% yield) as a yellow oil. MS(ESI) m/z: 430.5 [M+H]⁺.

### Step D. tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate

A mixture of 4-[3-(3-bromo-2-methyl-phenoxy)propyl]-1-(2,2-diethoxyethyl)piperidine (800 mg, 1.87 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.14 g, 1.87 mmol, 1 equiv.) and [2-(2-aminophenyl)phenyl]palladium(1+); bis(1-adamantyl)-butyl-phosphane; methanesulfonate (136.00 mg, 186.74 umol, 0.1 eq.), KF (1.5 M, 3.73 mL, 3 equiv.) in dioxane (5 mL) and H₂O (1 mL) was degassed and purged with N₂ 3 times and then stirred at 100° C. in a microwave reactor. After 1 h the reaction solution was concentrated under reduced pressure and purified by column chromatography (SiO₂, Ethyl acetate/MeOH=1/0 to 10/1) to give compound tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate (1.5 g, 1.72 mmol, 91.99% yield, 95.52% purity) as a yellow oil, which was carried forward without further purification. MS(ESI) m/z: 834.8 [M+H]⁺; ¹H NMR (400 MHz, CDCl₃) δ=7.85 (d, J=8.0 Hz, 1H), 7.59 (dd, J=7.2, 15.6 Hz, 2H), 7.40-7.30 (m, 5H), 7.14-7.06 (m, 1H), 6.90 (d, J=8.8 Hz, 1H), 6.80 (d, J=8.4 Hz, 1H), 6.69 (d, J=7.6 Hz, 1H), 5.12-4.95 (m, 2H), 4.81-4.60 (m, 1H), 4.12-4.08 (m, 2H), 3.97 (t, J=6.4 Hz, 2H), 3.72-3.68 (m, 2H), 3.61-3.54 (m, 2H), 3.07 (t, J=5.2 Hz, 4H), 2.64-2.52 (m, 2H), 2.05 (s, 3H), 1.84-1.80 (m, 2H), 1.69-1.55 (m, 9H), 1.24-1.20 (m, 6H), 1.15 (s, 9H).

### Step E. 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate (100 mg, 119.89 umol, 1 equiv.) in formic acid (2 mL). The mixture was stirred at 90° C. for 2 hours. The reaction mixture was concentrated under reduced pressure to give 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid hydrochloride (70 mg, 99.5 umol, 83% yield) as a dark green oil, which was carried forward without further purification. MS(ESI) m/z: 722.5 [M+H]⁺.

### Step F. 6-[8-(1,3-benzothiazol-2-ylcarbamoyl)-3,4-dihydro-1H-isoquinolin-2-yl]-3-[3-[3-[1-[2-[4-[2-(2,6-dioxo-3-piperidyl)-6-fluoro-1,3-dioxo-isoindolin-5-yl]piperazin-1-yl]ethyl]-4-piperidyl]propoxy]-2-methyl-phenyl]pyridine-2-carboxylic acid

To a solution of 2-(2,6-dioxo-3-piperidyl)-5-fluoro-6-piperazin-1-yl-isoindoline-1,3-dione hydrochloride (47.36 mg, 119.34 umol, 1.2 equiv.), 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid (70 mg, 99.45 umol, 1 equiv.) in dichloromethane (2 mL) at 0° C. and the mixture warmed to room temperature while stirring. After 15 min NaBH(OAc)₃ (63.23 mg, 298.36 umol, 3 eq.) was added, the mixture was stirred at 25° C. for 15 min. The reaction mixture was and concentrated under reduced pressure and purified by prep-HPLC (column: Phenomenex luna C18 150×25 mm×10 um; mobile phase: [water (1% formic acid)-MeCN]; B %: 28%-58%, 8 min) to give 6-[8-(1,3-benzothiazol-2-ylcarbamoyl)-3,4-dihydro-1H-isoquinolin-2-yl]-3-[3-[3-[1-[2-[4-[2-(2,6-dioxo-3-piperidyl)-6-fluoro-1,3-dioxo-isoindolin-5-yl]piperazin-1-yl]ethyl]-4-piperidyl]propoxy]-2-methyl-phenyl]pyridine-2-carboxylic acid (11.8 mg, 10.8 umol, 10.8% yield, 95.9% purity) as a yellow solid. MS(ESI) m/z: 1048.5 [M+H]⁺; ¹HNMR (400 MHz, DMSO-d₆) δ=11.10 (s, 1H), 8.18 (s, 1H), 8.01 (d, J=8.0 Hz, 1H), 7.77 (d, J=8.0 Hz, 1H), 7.71 (d, J=11.2 Hz, 1H), 7.61 (d, J=7.6 Hz, 1H), 7.48-7.40 (m, 3H), 7.39-7.31 (m, 3H), 7.07 (t, J=7.6 Hz, 1H), 6.87 (dd, J=6.0, 7.8 Hz, 2H), 6.65 (d, J=7.6 Hz, 1H), 5.10 (dd, J=5.6, 12.8 Hz, 1H), 4.96 (s, 2H), 3.98 (dd, J=2.0, 6.0 Hz, 2H), 3.89 (t, J=5.6 Hz, 2H), 3.23 (s, 6H), 3.01-2.97 (m, 4H), 2.61 (d, J=1.6 Hz, 2H), 2.56 (s, 4H), 2.21 (s, 1H), 2.13-1.99 (m, 4H), 1.90 (s, 3H), 1.80-1.55 (m, 6H), 1.41-1.18 (m, 6H).

### Example 6. Preparation of Compound 112a

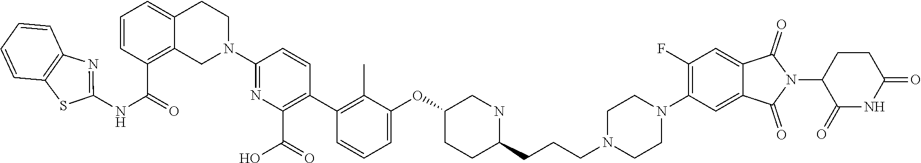 [SMILES: Cc1c(O[C@H]2CC[C@H](CCCN3CCN(c4cc5c(cc4F)C(=O)N(C4CCC(=O)NC4=O)C5=O)CC3)NC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(2-(2,6-dioxopiperidin-3-yl)-6-fluoro-1,3-dioxoisoindolin-5-yl)piperazin-1-yl)propyl)cyclohexyl)oxy)-2-methylphenyl)picolinic acid

### Step A. Procedure for Preparation of 3-((1r, 4s)-4-(3-bromo-2-methylphenoxy)cyclohexyl)propan-1-ol

To a solution of ethyl 3-((1r, 4r)-4-(3-bromo-2-methylphenoxy)cyclohexyl)propanoate (300 mg, 812.36 μmol, 1 equiv.) in THF (5 mL) was added LiAlH₄ (37.00 mg, 974.84 μmol, 1.2 equiv.) under N₂ at 0° C. Then the mixture was stirred at 25° C. for 1.5 hours. The reaction mixture was quenched by addition H₂O 0.5 mL, 15% NaOH 0.5 mL, H₂O 1.5 mL, and then extracted with EtOAc 3 mL (1 mL×3). The combined organic layers were dried over Na₂SO₄, filtered, and concentrated under reduced pressure to give 3-((1r, 4s)-4-(3-bromo-2-methylphenoxy)cyclohexyl)propan-1-ol (277 mg, crude) as a white solid.

### Step B. Procedure for Preparation of 3-((1r,4r)-4-(3-bromo-2-methylphenoxy)cyclohexyl)propanal

To a solution of DMSO (264.53 mg, 3.39 mmol, 264.53 μL, 4 equiv.) in DCM (2 mL) was added dropwise a solution of (COCl)₂ (214.88 mg, 1.69 mmol, 148.19 μL, 2 equiv.) in DCM (2 mL) at −70° C. under N₂ atmosphere. The mixture was stirred at −70° C. for 1 hour. 3-((1r, 4s)-4-(3-bromo-2-methylphenoxy)cyclohexyl)propan-1-ol (277 mg, 846.43 μmol, 1 equiv.) in DCM (2 mL) was then added dropwise at −70° C. The solution was stirred for 1 hour at −70° C. Then TEA (513.90 mg, 5.08 mmol, 706.88 μL, 6 equiv.) was added into the solution. The solution was stirred at −70° C. for 0.5 hour under N₂ atmosphere. The reaction mixture was quenched by addition H₂O 10 mL, and then diluted with DCM 10 mL and extracted with DCM 30 mL (10 mL×3). The combined organic layers were washed with H₂O 15 mL (5 mL×3), dried over anhydrous sodium sulfate, filtered, and concentrated under reduced pressure to give a yellow residue. The residue was purified by flash silica gel chromatography (eluent of 0˜10% ethyl acetate/petroleum ether) to give 3-((1r,4r)-4-(3-bromo-2-methylphenoxy)cyclohexyl)propanal (220 mg, 676.4 μmol, 79.9% yield) as a yellow solid.

### Step C. Procedure for Preparation of tert-butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate

A mixture of 3-((1r,4r)-4-(3-bromo-2-methylphenoxy)cyclohexyl)propanal (220 mg, 676.42 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (497.21 mg, 811.71 μmol, 1.2 equiv.), [2-(2-aminophenyl)phenyl]palladium(1+); bis(1-adamantyl)-butyl-phosphane; methanesulfonate (98.52 mg, 135.28 μmol, 0.2 equiv.), K₂CO₃ (1.5 M, 676.42 μL, 1.5 equiv.) in dioxane (5 mL) was degassed and purged with N₂ 3 times, and then the mixture was stirred at 100° C. for 1 hour under microwave. The reaction mixture was filtered and then concentrated under reduced pressure to give a residue. The residue was purified by flash silica gel chromatography (eluent of 0˜10% ethyl acetate/petroleum ether) to give tert-butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (280 mg, 383.0 μmol, 56.6% yield) as a white solid.

MS (ESI) m/z: 731.5 [M+H]⁺.

¹H NMR (400 MHz, DMSO-d₆) δ=12.86-12.82 (m, 1H), 9.94 (s, 1H), 8.03 (d, J=7.6 Hz, 1H), 7.79 (d, J=8.0 Hz, 1H), 7.69-7.64 (m, 1H), 7.57 (d, J=8.0 Hz, 1H), 7.50-7.44 (m, 2H), 7.41-7.32 (m, 4H), 7.20 (d, J=7.2 Hz, 1H), 7.06 (d, J=8.4 Hz, 1H), 5.07-4.89 (m, 3H), 2.98 (t, J=6.0 Hz, 2H), 1.99 (s, 3H), 1.36 (s, 2H), 1.33 (s, 4H), 1.24 (s, 9H), 1.16 (s, 9H).

### Step D. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(2-(2,6-dioxopiperidin-3-yl)-6-fluoro-1,3-dioxoisoindolin-5-yl)piperazin-1-yl)propyl)cyclohexyl)oxy)-2-methylphenyl)picolinate

A mixture of tert-butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (20 mg, 27.36 μmol, 1 equiv.), 2-(2,6-dioxopiperidin-3-yl)-5-fluoro-6-(piperazin-1-yl)isoindoline-1,3-dione (11.83 mg, 32.83 μmol, 1.2 equiv.), 4 Å MS (10 mg) and NaBH(OAc)₃ (17.40 mg, 82.08 μmol, 3 equiv.) in EtOH (0.8 mL) was degassed and purged with N₂ 3 times, and then the mixture was stirred at 25° C. for 8 hours under N₂ atmosphere. The mixture was filtered and then concentrated under reduced pressure to give tert-butyl 6-(8-(benzo[d]thiazol-2-ylcarbamoyl)-3,4-dihydroisoquinolin-2(1H)-yl)-3-(3-(((1s,4r)-4-(3-(4-(2-(2,6-dioxopiperidin-3-yl)-6-fluoro-1,3-dioxoisoindolin-5-yl)piperazin-1-yl)propyl)cyclohexyl)oxy)-2-methylphenyl)picolinate (90 mg, crude) as a white solid.

MS (ESI) m/z: 1075.5 [M+H]⁺.

### Step E. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(2-(2,6-dioxopiperidin-3-yl)-6-fluoro-1,3-dioxoisoindolin-5-yl)piperazin-1-yl)propyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(3-(4-(2-(2,6-dioxopiperidin-3-yl)-6-fluoro-1,3-dioxoisoindolin-5-yl)piperazin-1-yl)propyl)cyclohexyl)oxy)-2-methylphenyl)picolinate (90 mg, 83.70 μmol, 1 equiv.) in TFA (2 mL) was added DCM (2 mL). The mixture was stirred at 40° C. for 2 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-(4-(2-(2,6-dioxopiperidin-3-yl)-6-fluoro-1,3-dioxoisoindolin-5-yl)piperazin-1-yl)propyl)cyclohexyl)oxy)-2-methylphenyl)picolinic acid (30 mg, 28.7 μmol, 33.4% yield, 95% purity) as a yellow solid.

MS (ESI) m/z: 1019.5 [M+H]⁺.

¹H NMR (400 MHz, DMSO-d₆) δ=12.98-12.68 (m, 2H), 11.07 (d, J=4.8 Hz, 1H), 8.14 (s, 1H), 8.04 (d, J=8.0 Hz, 1H), 7.79 (d, J=8.0 Hz, 1H), 7.73 (d, J=11.2 Hz, 1H), 7.63 (d, J=7.6 Hz, 1H), 7.49-7.45 (m, 4H), 7.40-7.34 (m, 2H), 7.08 (t, J=8.0 Hz, 1H), 6.98-6.92 (m, 2H), 6.62 (d, J=7.2 Hz, 1H), 5.11 (d, J=12.8 Hz, 1H), 5.04-4.98 (m, 2H), 4.27-4.16 (m, 1H), 3.97-3.91 (m, 2H), 3.03 (t, J=5.6 Hz, 2H), 2.94-2.85 (m, 2H), 2.71-2.61 (m, 10H), 2.39-2.29 (m, 4H), 2.10-2.01 (m, 4H), 1.80 (d, J=11.5 Hz, 2H), 1.51-1.48 (m, 2H), 1.39-1.33 (m, 2H), 1.25-1.22 (m, 2H), 1.12-1.06 (m, 2H).

¹⁹F NMR (376 MHz, DMSO-d₆) δ=−73.41 (s, 1F), −111.94 (s, 1F)

### Example 7. Preparation of Compound 113

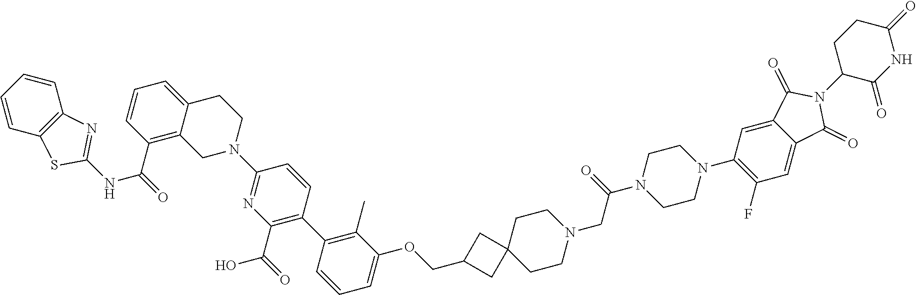

### 6-[8-(1,3-benzothiazol-2-ylcarbamoyl)-3,4-dihydro-1H-isoquinolin-2-yl]-3-[3-[[7-[2-[4-[2-(2,6-dioxo-3-piperidyl)-6-fluoro-1,3-dioxo-isoindolin-5-yl]piperazin-1-yl]-2-oxo-ethyl]-7-azaspiro[3.5]nonan-2-yl]methoxy]-2-methyl-phenyl]pyridine-2-carboxylic acid

### Step A. Procedure for Preparation of tert-butyl 2-[(3-bromo-2-methyl-phenoxy)methyl]-7-azaspiro[3.5]nonane-7-carboxylate

A mixture of 3-bromo-2-methyl-phenol (1 g, 5.35 mmol, 1 equiv.), tert-butyl 2-(hydroxymethyl)-7-azaspiro[3.5]nonane-7-carboxylate (1.50 g, 5.88 mmol, 1.1 equiv.), 2-(tributyl-λ5-phosphanylidene) acetonitrile (1.55 g, 6.42 mmol, 1.2 equiv.) in toluene (10 mL) was degassed and purged with N₂ and stirred at 120° C. for 2 hours under N₂ atmosphere. The reaction mixture was concentrated under reduced pressure to give a residue. The residue was purified by column chromatography (SiO₂, petroleum ether/ethyl acetate=0/1 to 10/1) to give tert-butyl 2-[(3-bromo-2-methyl-phenoxy)methyl]-7-azaspiro[3.5]nonane-7-carboxylate (2.35 g, crude) as a green oil.

¹H NMR (400 MHz, CDCl₃) δ=7.15 (d, J=8.0 Hz, 1H), 6.99 (t, J=8.0 Hz, 1H), 6.76 (d, J=8.4 Hz, 1H), 3.91 (d, J=6.0 Hz, 2H), 3.42-3.35 (m, 2H), 3.34-3.26 (m, 2H), 2.83-2.67 (m, 1H), 2.33 (s, 3H), 2.03-1.94 (m, 2H), 1.78-1.69 (m, 2H), 1.66-1.60 (m, 2H), 1.52-1.49 (m, 2H), 1.46 (s, 9H).

### Step B. Procedure for Preparation of 2-[(3-bromo-2-methyl-phenoxy)methyl]-7-azaspiro[3.5]nonane

To a solution of tert-butyl 2-[(3-bromo-2-methyl-phenoxy)methyl]-7-azaspiro[3.5]nonane-7-carboxylate (1.85, 4.36 mmol, 1 equiv.) in DCM (10 mL) was added HCl/1,4-dioxane (4 M, 10 mL, 9.18 equiv.). The mixture was stirred at 25° C. for 1 hour. The reaction mixture was concentrated under reduced pressure to give a residue. The residue was purified by column chromatography (SiO₂, DCM:MeOH=0/1 to 10/1) to give 2-[(3-bromo-2-methyl-phenoxy)methyl]-7-azaspiro[3.5]nonane (1.4 g, 4.3 mmol, 99.0% yield) as a white solid.

¹H NMR (400 MHz, DMSO-d₆) δ=7.18-7.14 (m, 1H), 7.09 (t, J=8.0 Hz, 1H), 6.96 (d, J=8.0 Hz, 1H), 3.96 (d, J=6.0 Hz, 2H), 3.02-2.97 (m, 2H), 2.93-2.88 (m, 2H), 2.74-2.66 (m, 1H), 2.24 (s, 3H), 2.01-1.92 (m, 2H), 1.81-1.76 (m, 2H), 1.75-1.70 (m, 2H), 1.70-1.67 (m, 2H).

### Step C. Procedure for Preparation of ethyl 2-[2-[(3-bromo-2-methyl-phenoxy)methyl]-7-azaspiro[3.5]nonan-7-yl]acetate

To a solution of 2-[(3-bromo-2-methyl-phenoxy)methyl]-7-azaspiro[3.5]nonane (500 mg, 1.54 mmol, 1 equiv.) and ethyl 2-bromoacetate (257.51 mg, 1.54 mmol, 170.54 μL, 1 equiv.) in MeCN (5 mL) was added K₂CO₃ (1.07 g, 7.71 mmol, 5 equiv.). The mixture was stirred at 60° C. for 12 hours. The reaction mixture was filtered and then concentrated under reduced pressure to give a residue. The residue was purified by prep-TLC (SiO₂, DCM:MeOH=10:1) to give ethyl 2-[2-[(3-bromo-2-methyl-phenoxy)methyl]-7-azaspiro[3.5]nonan-7-yl]acetate (240 mg, 584.7 μmol, 37.9% yield) as a colorless oil.

¹H NMR (400 MHz, CDCl₃) δ=7.15 (d, J=8.0 Hz, 1H), 6.99 (t, J=8.0 Hz, 1H), 6.76 (d, J=8.4 Hz, 1H), 4.25-4.15 (m, 2H), 3.90 (d, J=6.4 Hz, 2H), 3.20 (s, 2H), 2.80-2.66 (m, 1H), 2.62-2.39 (m, 4H), 2.32 (s, 3H), 2.04-1.90 (m, 2H), 1.76 (t, J=4.4 Hz, 2H), 1.58 (s, 4H), 1.28 (t, J=7.2 Hz, 3H).

### Step D. Procedure for Preparation of tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]methoxy]-2-methyl-phenyl]pyridine-2-carboxylate

Ethyl 2-[2-[(3-bromo-2-methyl-phenoxy)methyl]-7-azaspiro[3.5]nonan-7-yl]acetate (240 mg, 584.87 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (358.26 mg, 584.87 μmol, 1 equiv.) K₂CO₃ (1.5 M, 1.17 mL, 3 equiv.) and Ad₂nBuP Pd G₃(cataCXium® APd G₃) (85.19 mg, 116.97 μmol, 0.2 equiv.) were taken up into a microwave tube in 1,4-dioxane (3 mL). The sealed tube was heated at 100° C. for 1 hour under microwave. The reaction mixture was filtered and then concentrated under reduced pressure to give a residue. The residue was purified by column chromatography (SiO₂, petroleum ether/ethyl acetate=0/1 to 1/1) to give tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]methoxy]-2-methyl-phenyl]pyridine-2-carboxylate (240 mg, 294.1 μmol, 50.2% yield) as a yellow solid.

¹H NMR (400 MHz, CDCl₃) δ=7.86 (d, J=7.6 Hz, 1H), 7.72 (d, J=8.0 Hz, 1H), 7.57 (d, J=7.2 Hz, 1H), 7.47-7.41 (m, 1H), 7.38 (d, J=8.4 Hz, 2H), 7.35-7.30 (m, 2H), 7.14-7.06 (m, 1H), 6.90 (d, J=8.8 Hz, 1H), 6.80 (d, J=8.0 Hz, 1H), 6.70 (d, J=7.6 Hz, 1H), 5.16-4.90 (m, 2H), 4.23-4.16 (m, 2H), 4.11 (t, J=6.0 Hz, 2H), 3.94 (d, J=6.0 Hz, 2H), 3.32-3.14 (m, 2H), 3.07 (t, J=5.6 Hz, 2H), 2.77-2.68 (m, 1H), 2.64-2.36 (m, 4H), 2.00 (s, 3H), 1.95 (d, J=9.6 Hz, 2H), 1.82-1.66 (m, 6H), 1.28 (s, 3H), 1.15 (s, 9H).

### Step E. Procedure for Preparation of 2-[2-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-7-azaspiro[3.5]nonan-7-yl]acetic acid

To a solution of tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]methoxy]-2-methyl-phenyl]pyridine-2-carboxylate (240 mg, 294.11 μmol, 1 equiv.) in THF (0.5 mL) and H₂O (0.5 mL) was added LiOH·H₂O (61.71 mg, 1.47 mmol, 5 equiv.). The mixture was stirred at 25° C. for 12 hours. The reaction mixture concentrated under reduced pressure to remove THE and adjusted pH to 5 with HCl (1M), and then filtered, and the filter cake was concentrated under reduced pressure to give 2-[2-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-7-azaspiro[3.5]nonan-7-yl]acetic acid (270 mg, crude) as a yellow solid.

MS (ESI) m/z: 788.9 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[[7-[2-[4-[2-(2,6-dioxo-3-piperidyl)-6-fluoro-1,3-dioxo-isoindolin-5-yl]piperazin-1-yl]-2-oxo-ethyl]-7-azaspiro[3.5]nonan-2-yl]methoxy]-2-methyl-phenyl]pyridine-2-carboxylate

To a solution of 2-[2-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-7-azaspiro[3.5]nonan-7-yl]acetic acid (80 mg, 101.53 μmol, 1 equiv.) and 2-(2,6-dioxo-3-piperidyl)-5-fluoro-6-piperazin-1-yl-isoindoline-1,3-dione (43.90 mg, 121.83 μmol, 1.2 equiv.) in pyridine (0.5 mL) was added EDCI (29.19 mg, 152.29 μmol, 1.5 equiv.). The mixture was stirred at 25° C. for 2 hours. The reaction mixture was quenched by addition water (2 mL), and then diluted with EtOAc (2 mL) and extracted with EtOAc (2 mL×3). The combined organic layers were washed with brine (2 mL×3), dried over anhydrous sodium sulfate, filtered, and concentrated under reduced pressure to give tert-butyl 6-[8-(1,3-benzothiazol-2-ylcarbamoyl)-3,4-dihydro-1H-isoquinolin-2-yl]-3-[3-[[7-[2-[4-[2-(2,6-dioxo-3-piperidyl)-6-fluoro-1,3-dioxo-isoindolin-5-yl]piperazin-1-yl]-2-oxo-ethyl]-7-azaspiro[3.5]nonan-2-yl]methoxy]-2-methyl-phenyl]pyridine-2-carboxylate (80 mg, 70.8 μmol, 69.7% yield) as a brown oil.

MS (ESI) m/z: 1130.8 [M+H]⁺

### Step G. Procedure for Preparation of 6-[8-(1,3-benzothiazol-2-ylcarbamoyl)-3,4-dihydro-1H-isoquinolin-2-yl]-3-[3-[[7-[2-[4-[2-(2,6-dioxo-3-piperidyl)-6-fluoro-1,3-dioxo-isoindolin-5-yl]piperazin-1-yl]-2-oxo-ethyl]-7-azaspiro[3.5]nonan-2-yl]methoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[[7-[2-[4-[2-(2,6-dioxo-3-piperidyl)-6-fluoro-1,3-dioxo-isoindolin-5-yl]piperazin-1-yl]-2-oxo-ethyl]-7-azaspiro[3.5]nonan-2-yl]methoxy]-2-methyl-phenyl]pyridine-2-carboxylate (80 mg, 70.78 μmol, 1 equiv.) in DCM (1 mL) was added TFA (8.07 mg, 70.78 μmol, 5.24 μL, 1 equiv.). The mixture was stirred at 25° C. for 12 hours. The reaction mixture was filtered and then concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[[7-[2-[4-[2-(2,6-dioxo-3-piperidyl)-6-fluoro-1,3-dioxo-isoindolin-5-yl]piperazin-1-yl]-2-oxo-ethyl]-7-azaspiro[3.5]nonan-2-yl]methoxy]-2-methyl-phenyl]pyridine-2-carboxylic acid (34 mg, 29.0 μmol, 41.1% yield, 91.9% purity) as a yellow solid.

MS (ESI) m/z: 1074.7 [M+H]⁺

¹H NMR (400 MHz, DMSO-d₆) δ=13.27-11.96 (m, 2H), 11.11 (s, 1H), 8.03 (d, J=7.6 Hz, 1H), 7.82-7.73 (m, 2H), 7.62 (d, J=7.6 Hz, 1H), 7.51-7.42 (m, 4H), 7.41-7.31 (m, 2H), 7.13-7.05 (m, 1H), 6.97 (d, J=8.8 Hz, 1H), 6.87 (d, J=8.4 Hz, 1H), 6.63 (d, J=7.6 Hz, 1H), 5.15-5.07 (m, 1H), 4.98 (s, 2H), 3.92 (d, J=6.0 Hz, 4H), 3.74-3.58 (m, 4H), 3.22 (s, 10H), 3.03 (t, J=5.6 Hz, 2H), 2.97-2.80 (m, 2H), 2.63-2.55 (m, 2H), 2.09-1.98 (m, 1H), 1.90 (s, 5H), 1.74-1.50 (m, 6H).

### Example 8. Preparation of Compound 116

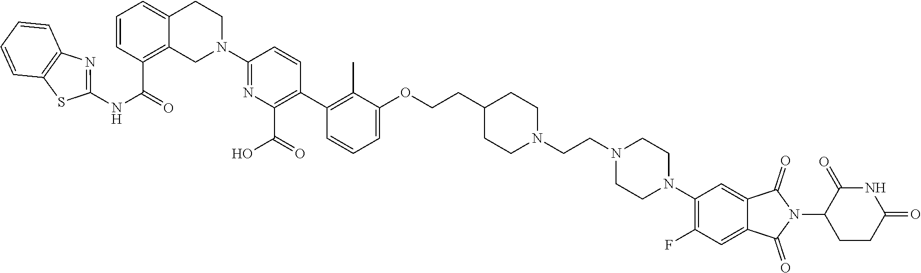 [SMILES: Cc1c(OCCC2CCN(CCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[2-[4-[2-(2,6-dioxo-3-piperidyl)-6-fluoro-1,3-dioxo-isoindolin-5-yl]piperazin-1-yl]ethyl]-4-piperidyl]ethoxy]-2-methyl-phenyl]pyridine-2-carboxylic acid

### Step A. Procedure for Preparation of tert-butyl 4-[2-(3-bromo-2-methyl-phenoxy)ethyl]piperidine-1-carboxylate

To a mixture of tert-butyl 4-(2-hydroxyethyl)piperidine-1-carboxylate (5 g, 21.80 mmol, 1 equiv.), PPh₃ (7.43 g, 28.35 mmol, 1.3 equiv.) and 3-bromo-2-methyl-phenol (4.08 g, 21.80 mmol, 1 equiv.) in THF (40 mL) was added DIAD (5.29 g, 26.16 mmol, 5.09 mL, 1.2 equiv.) under N₂ at 0° C. The solution was stirred at 0° C. for 30 minutes, then the solution was warmed to 20° C. and stirred for 12 hours. The reaction mixture was concentrated under reduced pressure to give a residue. The residue was then purified by column chromatography (silica gel, petroleum ether/ethyl acetate=500/1 to 150/1) to give tert-butyl 4-[2-(3-bromo-2-methyl-phenoxy)ethyl]piperidine-1-carboxylate (8 g, 20.08 mmol, 92.11% yield) as a yellow solid.

¹H NMR (400 MHz, CDCl₃) δ=7.15 (d, J=7.6 Hz, 1H), 7.00 (t, J=8.0 Hz, 1H), 6.76 (d, J=8.0 Hz, 1H), 4.21-4.05 (m, 2H), 4.00 (t, J=6.0 Hz, 2H), 2.72 (t, J=12.4 Hz, 2H), 2.32 (s, 3H), 1.80-1.70 (m, 5H), 1.47 (s, 9H), 1.26-1.12 (m, 2H)

### Step B. Procedure for Preparation of 4-[2-(3-bromo-2-methyl-phenoxy)ethyl]piperidine

A mixture of tert-butyl 4-[2-(3-bromo-2-methyl-phenoxy)ethyl]piperidine-1-carboxylate (8 g, 20.08 mmol, 1 equiv.) in HCl/dioxane (4 M, 50 mL, 9.96 equiv.) was stirred at 20° C. for 0.5 hours. The reaction mixture was concentrated under reduced pressure to give a residue. The residue was then purified by re-crystallization from DCM (20 mL) at 20° C. to give 4-[2-(3-bromo-2-methyl-phenoxy)ethyl]piperidine (6 g, 17.9 mmol, 89.2% yield) as a white solid.

¹H NMR (400 MHz, CD₃OD) δ=7.13 (dd, J=0.8, 8.0 Hz, 1H), 7.04 (t, J=8.0 Hz, 1H), 6.91 (d, J=8.0 Hz, 1H), 4.08 (t, J=6.0 Hz, 2H), 3.40 (d, J=12.8 Hz, 2H), 3.07-2.93 (m, 2H), 2.29 (s, 3H), 2.09-1.98 (m, 2H), 1.98-1.89 (m, 1H), 1.84 (q, J=6.4 Hz, 2H), 1.56-1.43 (m, 2H)

### Step C. Procedure for Preparation of 4-[2-(3-bromo-2-methyl-phenoxy)ethyl]-1-(2,2-diethoxyethyl)piperidine

To a solution of 4-[2-(3-bromo-2-methyl-phenoxy)ethyl]piperidine (1 g, 2.99 mmol, 1 equiv.) and K₂CO₃ (1.24 g, 8.97 mmol, 3 equiv.) in DMF (10 mL) was added 2-bromo-1,1-diethoxy-ethane (589.24 mg, 2.99 mmol, 449.80 uL, 1 equiv.). The solution was stirred at 60° C. for 12 hours. The reaction mixture was filtered then and concentrated under reduced pressure to give a residue. The residue was purified by column chromatography (silica gel, DCM:MeOH=100/1 to 12/1) to give 4-[2-(3-bromo-2-methyl-phenoxy)ethyl]-1-(2,2-diethoxyethyl)piperidine (800 mg, 1.9 mmol, 64.5% yield) as a colorless oil.

MS (ESI) m/z: 416.0 [M+H]⁺

¹H NMR (400 MHz, CDCl₃) δ=7.14 (d, J=8.0 Hz, 1H), 6.99 (t, J=8.0 Hz, 1H), 6.76 (d, J=8.0 Hz, 1H), 4.65 (t, J=5.2 Hz, 1H), 3.98 (t, J=6.4 Hz, 2H), 3.74-3.63 (m, 2H), 3.60-3.48 (m, 2H), 3.03-2.96 (m, 2H), 2.54 (d, J=5.2 Hz, 2H), 2.31 (s, 3H), 2.13-2.03 (m, 2H), 1.80-1.66 (m, 5H), 1.60-1.46 (m, 1H), 1.41-1.28 (m, 2H), 1.22 (t, J=7.2 Hz, 6H)

### Step D. Procedure for Preparation of tert-butyl 6-[8-(1,3-benzothiazol-2-ylcarbamoyl)-3,4-dihydro-1H-isoquinolin-2-yl]-3-[3-[2-[1-(2,2-diethoxyethyl)-4-piperidyl]ethoxy]-2-methyl-phenyl]pyridine-2-carboxylate

A mixture of 4-[2-(3-bromo-2-methyl-phenoxy)ethyl]-1-(2,2-diethoxyethyl)piperidine (427 mg, 1.03 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (504.97 mg, 824.37 umol, 0.8 equiv.), [2-(2-aminophenyl)phenyl]palladium(1+); bis(1-adamantyl)-butyl-phosphane; methanesulfonate (150.09 mg, 206.09 umol, 0.2 equiv.) K₂CO₃ (1.5 M, 2.06 mL, 3 equiv.) in dioxane (3 mL) was degassed and purged with N₂ 3 times, and then the mixture was stirred at 60° C. for 12 hours under N₂ atmosphere. The reaction mixture was extracted with DCM 15 mL (5 mL×3). The combined organic layers were washed with brine 10 mL (5 mL×2), dried over Na₂SO₄, filtered, and concentrated under reduced pressure to give a residue. The residue was purified by flash silica gel chromatography (eluent of 0-10% methanol/dichloromethane) to give tert-butyl 6-[8-(1,3-benzothiazol-2-ylcarbamoyl)-3,4-dihydro-1H-isoquinolin-2-yl]-3-[3-[2-[1-(2,2-diethoxyethyl)-4-piperidyl]ethoxy]-2-methyl-phenyl]pyridine-2-carboxylate (200 mg, 243.9 umol, 23.7% yield) as a brown solid.

MS (ESI) m/z: 820.3 [M+H]⁺

¹H NMR (400 MHz, DMSO-d₆) δ=8.01 (d, J=8.0 Hz, 1H), 7.76 (d, J=8.4 Hz, 1H), 7.64-7.57 (m, 1H), 7.50-7.41 (m, 3H), 7.40-7.29 (m, 2H), 7.13-7.06 (m, 1H), 6.99-6.87 (m, 2H), 6.63-6.53 (m, 1H), 4.98 (s, 2H), 4.54 (t, J=5.2 Hz, 1H), 4.00 (t, J=5.6 Hz, 2H), 3.87 (t, J=5.6 Hz, 2H), 3.59-3.52 (m, 2H), 3.47-3.42 (m, 2H), 3.03 (t, J=5.6 Hz, 2H), 2.85 (d, J=10.4 Hz, 2H), 2.36 (d, J=5.2 Hz, 2H), 2.01-1.90 (m, 2H), 1.86 (s, 3H), 1.72-1.57 (m, 4H), 1.50-1.38 (m, 1H), 1.25-1.14 (m, 3H), 1.09 (t, J=7.2 Hz, 6H), 1.00 (s, 9H)

### Step E. Procedure for Preparation of 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[2-[1-(2,2-diethoxyethyl)-4-piperidyl]ethoxy]-2-methyl-phenyl]pyridine-2-carboxylate (100 mg, 121.94 μmol, 1 equiv.) in HCOOH (5.86 mg, 121.94 μmol, 1 mL, 1 equiv.) was degassed and purged with N₂ 3 times, and then the mixture was stirred at 90° C. for 0.5 hours under N₂ atmosphere. The reaction mixture was quenched by saturated NaHCO₃ solution (5 mL), and then diluted with H₂O 5 mL and extracted with EtOAc 15 mL (5 mL×3). The combined organic layers were washed with brine (5 mL) and dried over Na₂SO₄, filtered, and concentrated under reduced pressure to give a residue. The residue was purified by prep-TLC (silica gel, DCM:MeOH=10:1) to give 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid (50 mg, 72.4 μmol, 59.4% yield) as a yellow oil.

MS (ESI) m/z: 708.2 [M+18+H]⁺

### Step F. Procedure for Preparation of 6-[8-(1,3-benzothiazol-2-ylcarbamoyl)-3,4-dihydro-1H-isoquinolin-2-yl]-3-[3-[2-[1-[2-[4-[2-(2,6-dioxo-3-piperidyl)-6-fluoro-1,3-dioxo-isoindolin-5-yl]piperazin-1-yl]ethyl]-4-piperidyl]ethoxy]-2-methyl-phenyl]pyridine-2-carboxylic acid

To a solution of 2-(2,6-dioxo-3-piperidyl)-5-fluoro-6-piperazin-1-yl-isoindoline-1,3-dione (62.12 mg, 156.56 μmol, 1.2 equiv, HCl) and NaBH(OAc)₃ (82.95 mg, 391.41 μmol, 3 equiv.) in DCM (1.5 mL) and IPA (1.5 mL) was added 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid (90 mg, 130.47 μmol, 1 equiv.) at 0° C. slowly. The mixture was stirred at 0° C. for 1 hour, then the mixture was warmed to 25° C. and stirred at 25° C. for 12 hours. The mixture was concentrated under reduced pressure to remove DCM and IPA, and the mixture was diluted with DMF (2 mL). The mixture was purified by prep-HPLC to give 6-[8-(1,3-benzothiazol-2-ylcarbamoyl)-3,4-dihydro-1H-isoquinolin-2-yl]-3-[3-[2-[1-[2-[4-[2-(2,6-dioxo-3-piperidyl)-6-fluoro-1,3-dioxo-isoindolin-5-yl]piperazin-1-yl]ethyl]-4-piperidyl]ethoxy]-2-methyl-phenyl]pyridine-2-carboxylic acid (25.6 mg, 22.8 μmol, 17.4% yield, 96.2% purity) as a yellow solid.

MS (ESI) m/z: 1035.4 [M+2H]⁺

¹H NMR (400 MHz, DMSO-d₆) δ=11.11 (s, 1H), 8.02 (d, J=8.0 Hz, 1H), 7.78 (d, J=8.0 Hz, 1H), 7.72 (d, J=11.2 Hz, 1H), 7.62 (d, J=7.6 Hz, 1H), 7.50-7.24 (m, 6H), 7.12-7.04 (m, 1H), 6.96-6.84 (m, 2H), 6.64 (d, J=8.0 Hz, 1H), 5.14-5.07 (m, 1H), 4.97 (s, 2H), 3.99 (t, J=6.0 Hz, 2H), 3.91 (t, J=5.6 Hz, 2H), 3.24-3.21 (m, 6H), 3.02 (t, J=6.0 Hz, 2H), 2.97-2.92 (m, 2H), 2.90-2.79 (m, 2H), 2.63-2.54 (m, 5H), 2.54-2.52 (m, 2H), 2.10-1.99 (m, 3H), 1.93-1.86 (m, 3H), 1.74-1.62 (m, 4H), 1.55-1.46 (m, 1H), 1.26-1.17 (m, 2H)

### Example 9. Preparation of Compound 117

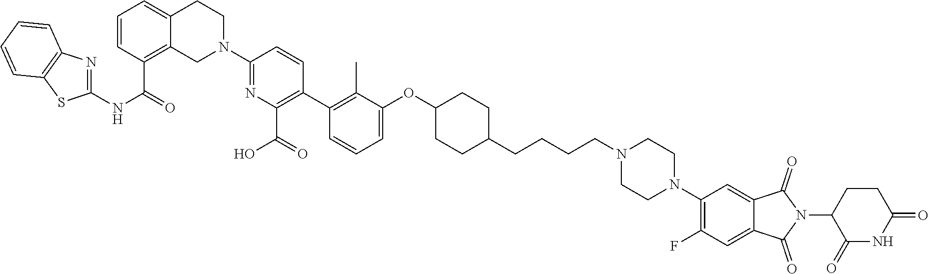 [SMILES: Cc1c(OC2CCC(CCCCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)butyl)phenoxy)-2-methylphenyl)picolinic acid

### Step A. Procedure for Preparation of methyl 4-(4-(3-bromo-2-methylphenoxy)phenyl)butanoate

A mixture of methyl 4-(4-hydroxyphenyl)butanoate (700 mg, 3.60 mmol, 1 equiv.), 1,3-dibromo-2-methylbenzene (2.70 g, 10.81 mmol, 3 equiv.), CuI (343.19 mg, 1.80 mmol, 0.5 equiv.), Cs₂CO₃ (1.41 g, 4.32 mmol, 1.2 equiv.) and 2,2,6,6-tetramethylheptane-3,5-dione (166.03 mg, 901.01 μmol, 185.51 μL, 0.25 equiv.) in NMP (20 mL) was degassed and purged with N₂ and stirred at 120° C. for 12 hours under N₂ atmosphere. The reaction mixture was diluted with H₂O (30 mL) and extracted with ethyl acetate 80 mL (20 mL×4). The combined organic layers were washed with brine 30 mL (15 mL×2), dried over Na₂SO₄, filtered, and concentrated under reduced pressure to give a residue. The residue was purified by column chromatography (eluent of 0˜7% ethyl acetate/petroleum ether) to give methyl 4-(4-(3-bromo-2-methylphenoxy)phenyl)butanoate (975 mg, 2.6 mmol, 74.4% yield) as a yellow solid.

¹H NMR (400 MHz, DMSO-d₆) δ=7.41 (d, J=8.0 Hz, 1H), 7.20-7.12 (m, 3H), 6.90-6.81 (m, 3H), 3.59 (s, 3H), 2.56 (t, J=7.6 Hz, 2H), 2.33-2.26 (m, 5H), 1.81 (quin, J=7.6 Hz, 2H)

### Step B. Procedure for Preparation of 4-(4-(3-bromo-2-methylphenoxy)phenyl)butan-1-ol

To a solution of methyl 4-(4-(3-bromo-2-methylphenoxy)phenyl)butanoate (700 mg, 1.93 mmol, 1 equiv.) in THF (8 mL) was added LiAlH₄ (73.13 mg, 1.93 mmol, 1 equiv.) at 0° C. The mixture was stirred at 0° C. for 2 hours. The reaction mixture was quenched by addition of water (0.1 mL) and 15% NaOH (0.1 mL) and water (0.3 mL). The combined mixture was mixed with Na₂SO₄, filtered, and concentrated under reduced pressure to give 4-(4-(3-bromo-2-methylphenoxy)phenyl)butan-1-ol (640 mg, 1.9 mmol, 99.0% yield) was obtained as a yellow oil.

¹H NMR (400 MHz, CDCl₃) δ=7.26 (d, J=8.0 Hz, 1H), 7.08-7.01 (m, 2H), 6.93 (t, J=8.0 Hz, 1H), 6.79-6.72 (m, 3H), 3.68 (s, 1H), 3.60 (t, J=6.4 Hz, 2H), 2.55 (t, J=7.6 Hz, 2H), 2.27 (s, 3H), 1.78 (d, J=3.2, 6.4 Hz, 2H), 1.65-1.60 (m, 2H)

### Step C. Procedure for Preparation of 4-(4-(3-bromo-2-methylphenoxy)phenyl)butanal

A solution of oxalyl dichloride (454.34 mg, 3.58 mmol, 313.34 μL, 2 equiv.) in DCM (1 mL) was added dropwise to a solution of DMSO (559.37 mg, 7.16 mmol, 559.37 μL, 4 equiv.) in DCM (1 mL) at −70° C. under N₂ atmosphere. The mixture was stirred at −70° C. for 1 hour. After that, 4-(4-(3-bromo-2-methylphenoxy)phenyl)butan-1-ol (600 mg, 1.79 mmol, 1 equiv.) in DCM (5 mL) was added dropwise at −70° C. The solution was stirred for 1 hour at −70° C. Then TEA (1.09 g, 10.74 mmol, 1.49 mL, 6 equiv.) was added into solution and stirred at −70° C. for 1 hour under N₂ atmosphere. The reaction was diluted with water (50 mL) and extracted with DCM 200 mL (100 mL×2). The combined organic layers were washed with brine 100 mL (100 mL×1), dried over Na₂SO₄, filtered, and concentrated under reduced pressure to give 4-(4-(3-bromo-2-methylphenoxy)phenyl)butanal (550 mg, 1.6 mmol, 92.2% yield) as a white solid. It was used in next step without further purification.

¹H NMR (400 MHz, DMSO-d₆) δ=9.67 (t, J=1.6 Hz, 1H), 7.42 (d, J=8.0 Hz, 1H), 7.23-7.17 (m, 3H), 6.85 (br s, 3H), 2.58 (d, J=7.6 Hz, 2H), 2.46-2.43 (m, 2H), 2.27 (s, 3H), 1.86 (s, 2H)

### Step D. Procedure for Preparation of tert-butyl 6-(8-(benzo[d]thiazol-2-ylcarbamoyl)-3,4-dihydroisoquinolin-2(1H)-yl)-3-(2-methyl-3-(4-(4-oxobutyl)phenoxy)phenyl)picolinate

To a solution of 4-(4-(3-bromo-2-methylphenoxy)phenyl)butanal (522.23 mg, 1.57 mmol, 1.2 equiv.) in dioxane (8 mL) and H₂O (2 mL) was added KF (227.63 mg, 3.92 mmol, 91.79 μL, 3 equiv.) and Ad₂nBuP Pd G₃ (95.11 mg, 130.60 μmol, 0.1 equiv.) and tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (800 mg, 1.31 mmol, 1 equiv.). The residue was diluted with EtOAc (30 mL) and water (30 mL). The layers were separated, and the aqueous phase was extracted with EtOAc 150 mL (50×3 mL). The organic layer was dried over Na₂SO₄, filtered, and concentrated in vacuum. The residue was purified by TLC (petroleum ether/ethyl acetate=2/1) to give tert-butyl 6-(8-(benzo[d]thiazol-2-ylcarbamoyl)-3,4-dihydroisoquinolin-2(1H)-yl)-3-(2-methyl-3-(4-(4-oxobutyl)phenoxy)phenyl)picolinate (430 mg, 581.9 μmol, 44.5% yield) as a yellow solid.

¹H NMR (400 MHz, DMSO-d₆) δ=12.88 (s, 1H), 9.66 (s, 1H), 8.03 (d, J=7.6 Hz, 1H), 7.78 (d, J=8.0 Hz, 1H), 7.61 (d, J=7.6 Hz, 1H), 7.54-7.43 (m, 3H), 7.41-7.32 (m, 2H), 7.72-7.26 (m, 1H), 7.17 (d, J=8.4 Hz, 4H), 6.98 (d, J=8.8 Hz, 1H), 6.93-6.61 (m, 5H), 5.00 (s, 2H), 3.91-3.86 (m, 2H), 3.61-3.35 (m, 1H), 3.33-3.15 (m, 1H), 3.04 (t, J=5.6 Hz, 2H), 2.54 (d, J=8.4 Hz, 5H), 2.43 (dt, J=1.2, 7.2 Hz, 3H), 1.91 (s, 3H), 1.80 (quin, J=7.6 Hz, 2H), 1.27-1.10 (m, 1H), 1.05-0.80 (m, 1H)

### Step E. Procedure for Preparation of tert-butyl 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)butyl)phenoxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(4-(4-oxobutyl)phenoxy)phenyl)picolinate (70 mg, 94.74 μmol, 1 equiv.) in DCM (1 mL) was added 2-(2,6-dioxopiperidin-3-yl)-5-fluoro-6-(piperazin-1-yl)isoindoline-1,3-dione (37.55 mg, 104.21 μmol, 1.1 equiv.) and AcOH (11.38 mg, 189.47 μmol, 10.84 μL, 2 equiv.). The mixture was stirred at 25° C. for 12 hours. The reaction was diluted with water (30 mL) and extracted with DCM (50 mL×2). The combined organic layers were washed with brine 50 mL (50 mL×1), dried over Na₂SO₄, filtered, and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)butyl)phenoxy)-2-methylphenyl)picolinate (80 mg, 73.8 μmol, 77.9% yield) as a white solid.

MS (ESI) m/z: 1083.6 [M+H]⁺.

### Step F. Procedure for Preparation of 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)butyl)phenoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)butyl)phenoxy)-2-methylphenyl)picolinate (80 mg, 73.85 μmol, 1 equiv.) in DCM (1 mL) was added TFA (8.42 mg, 73.85 μmol, 5.47 μL, 1 equiv.). The mixture was stirred at 25° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)butyl)phenoxy)-2-methylphenyl)picolinic acid (22.17 mg, 20.0 μmol, 27.0% yield, 96.8% purity) as a yellow solid.

MS (ESI) m/z: 1027.7 [M+H]⁺.

¹H NMR (400 MHz, DMSO-d₆) δ=11.13-11.09 (m, 1H), 8.24 (s, 1H), 8.02 (d, J=7.6 Hz, 1H), 7.80-7.70 (m, 2H), 7.64 (d, J=7.6 Hz, 1H), 7.50-7.41 (m, 4H), 7.39-7.31 (m, 2H), 7.21-7.11 (m, 3H), 6.92 (d, J=7.6 Hz, 2H), 6.85-6.78 (m, 3H), 5.11 (dd, J=5.6, 12.8 Hz, 1H), 5.00 (s, 2H), 3.92 (t, J=5.6 Hz, 2H), 3.26-3.19 (m, 8H), 3.03 (t, J=5.6 Hz, 2H), 2.69-2.55 (m, 5H), 2.37-2.33 (m, 2H), 2.09-1.99 (m, 1H), 1.93 (s, 3H), 1.62-1.54 (m, 2H), 1.52-1.43 (m, 2H)

### Example 10. Preparation of Compound 119

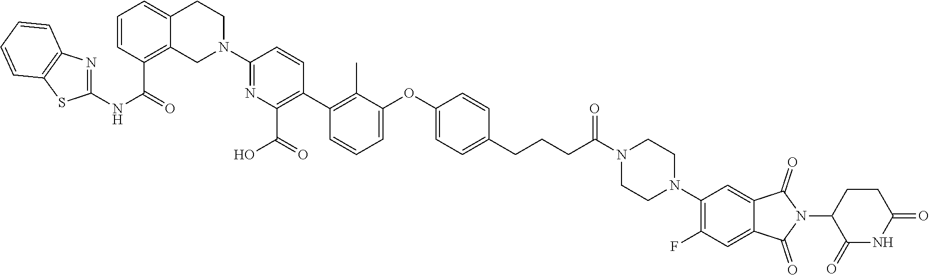 [SMILES: Cc1c(Oc2ccc(CCCC(=O)N3CCN(c4cc5c(cc4F)C(=O)N(C4CCC(=O)NC4=O)C5=O)CC3)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)-4-oxobutyl) phenoxy)-2-methylphenyl)picolinic acid

### Step A. Procedure for Preparation of methyl 4-(4-hydroxyphenyl)butanoate

To a solution of 4-(4-hydroxyphenyl)butanoic acid (2 g, 11.10 mmol, 1 equiv.) in MeOH (25 mL) was added H₂SO₄ (23.55 g, 240.13 mmol, 12.8 mL, 21.64 equiv.). The mixture was stirred at 25° C. for 12 hours. The reaction mixture was diluted with H₂O (50 mL) and extracted with ethyl acetate 100 mL (50 mL×2). The combined organic layers were washed with brine 40 mL (20 mL×2), dried over Na₂SO₄, filtered, and concentrated under reduced pressure to give a residue. The residue was purified by flash silica gel chromatography (eluent of 0-20% ethyl acetate/petroleum ether) to give methyl 4-(4-hydroxyphenyl)butanoate (1.7 g, 7.8 mmol, 70.9% yield, 90% purity) as a colorless oil.

¹H NMR (400 MHz, CDCl₃) δ=7.04 (d, J=6.8 Hz, 2H), 6.78-6.75 (m, 2H), 3.69-3.67 (m, 3H), 2.58 (t, J=7.2 Hz, 2H), 2.33 (dt, J=1.6, 6.0 Hz, 2H), 1.98-1.91 (m, 2H).

### Step B. Procedure for Preparation of methyl 4-(4-(3-bromo-2-methylphenoxy)phenyl)butanoate

A mixture of methyl 4-(4-hydroxyphenyl)butanoate (500 mg, 2.57 mmol, 1 equiv.), 1,3-dibromo-2-methylbenzene (1.93 g, 7.72 mmol, 3 equiv.), CuI (245.14 mg, 1.29 mmol, 0.5 equiv.), Cs₂CO₃ (1.01 g, 3.09 mmol, 1.2 equiv.) and 2,2,6,6-tetramethylheptane-3,5-dione (118.60 mg, 643.58 μmol, 132.51 μL, 0.25 equiv.) in NMP (20 mL) was degassed and purged with N₂ and then stirred at 120° C. for 12 hours under N₂ atmosphere. The reaction mixture was diluted with H₂O (30 mL) and extracted with ethyl acetate 80 mL (20 mL×4). The combined organic layers were washed with brine 30 mL (15 mL×2), dried over Na₂SO₄, filtered, and concentrated under reduced pressure to give a residue. The residue was purified by flash silica gel chromatography (eluent of 0-7% ethyl acetate/petroleum ether) to give methyl 4-(4-(3-bromo-2-methylphenoxy)phenyl)butanoate (900 mg, 2.2 mmol, 86.6% yield, 90% purity) as a yellow oil.

¹H NMR (400 MHz, CDCl₃) δ=7.36-7.32 (m, 1H), 7.13 (d, J=8.4 Hz, 2H), 7.01 (t, J=8.4 Hz, 1H), 6.88-6.82 (m, 3H), 3.68 (s, 3H), 2.63 (t, J=7.6 Hz, 2H), 2.38-2.33 (m, 5H), 1.95 (q, J=7.6 Hz, 2H).

### Step C. Procedure for Preparation of tert-butyl 6-(8-(benzo[d]thiazol-2-ylcarbamoyl)-3,4-dihydroisoquinolin-2(1H)-yl)-3-(3-(4-(4-methoxy-4-oxobutyl)phenoxy)-2-methylphenyl)picolinate

To a mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (850 mg, 1.39 mmol, 1 equiv.), methyl 4-(4-(3-bromo-2-methylphenoxy)phenyl)butanoate (672.08 mg, 1.67 mmol, 90% purity, 1.2 equiv.) in 1,4-dioxane (10 mL) was added Ad₂nBuP Pd G₃(cataCXium® A Pd G₃) (101.06 mg, 138.77 μmol, 0.1 equiv.) and KF (1.5 M, 2.78 mL, 3 equiv.). After addition, the mixture was degassed and purged with N₂ and stirred at 100° C. for 2 hours under microwave. The reaction mixture was filtered and then concentrated under reduced pressure to give a residue. The residue was purified by flash silica gel chromatography (eluent of 0-30% dichloromethane/methanol) to give tert-butyl 6-(8-(benzo[d]thiazol-2-ylcarbamoyl)-3,4-dihydroisoquinolin-2(1H)-yl)-3-(3-(4-(4-methoxy-4-oxobutyl)phenoxy)-2-methylphenyl)picolinate (520 mg, 635.7 μmol, 45.8% yield, 94% purity) as a yellow solid.

MS (ESI) m/z: 769.9 [M+H]⁺.

### Step D. Procedure for Preparation of 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy) phenyl)butanoic acid

To a solution of tert-butyl 6-(8-(benzo[d]thiazol-2-ylcarbamoyl)-3,4-dihydroisoquinolin-2(1H)-yl)-3-(3-(4-(4-methoxy-4-oxobutyl)phenoxy)-2-methylphenyl)picolinate (520 mg, 635.70 μmol, 94% purity, 1 equiv.) in THF (5 mL) was added LiOH·H₂O (80.03 mg, 1.91 mmol, 3 equiv.) and H₂O (1.25 mL) at 25° C. The mixture was stirred at 25° C. for 12 hours.

The reaction mixture was concentrated under reduced pressure to remove THF. The aqueous phase was adjusted to pH=4-5 with 1M HCl. The reaction mixture was filtered. After filtration, the filter cake was diluted in ethyl acetate. The combined organic layers were dried over Na₂SO₄, filtered, and concentrated under reduced pressure to give 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy) phenyl)butanoic acid (500 mg, crude) as a yellow solid.

MS (ESI) m/z: 755.5 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)-4-oxobutyl)phenoxy)-2-methylphenyl)picolinate

To a solution of 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy) phenyl)butanoic acid (100 mg, 132.47 μmol, 1 equiv.) in DMF (1 mL) was added HATU (60.44 mg, 158.96 μmol, 1.2 equiv.), DIPEA (51.36 mg, 397.41 μmol, 69.22 μL, 3 equiv.), and 2-(2,6-dioxopiperidin-3-yl)-5-fluoro-6-(piperazin-1-yl)isoindoline-1,3-dione (47.73 mg, 132.47 μmol, 1 equiv.). The mixture was stirred at 25° C. for 3 hours. The reaction mixture was concentrated under reduced pressure to give tert-butyl 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)-4-oxobutyl)phenoxy)-2-methylphenyl)picolinate (130 mg, crude) as a yellow solid.

MS (ESI) m/z: 1097.7 [M+H]⁺.

### Step F. Procedure for Preparation of 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)-4-oxobutyl) phenoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)-4-oxobutyl)phenoxy)-2-methylphenyl)picolinate (130 mg, 118.48 μmol, 1 equiv.) in DCM (1 mL) was added TFA (1.34 g, 11.77 mmol, 871.40 μL, 99.34 equiv.). The mixture was stirred at 25° C. for 1 hour. The reaction mixture was concentrated under reduced pressure to give a residue. The crude product was purified by prep-HPLC to give 6-(8-(benzo[d]thiazol-2-ylcarbamoyl)-3,4-dihydroisoquinolin-2(1H)-yl)-3-(3-(4-(4-(4-(2-(2,6-dioxopiperidin-3-yl)-6-fluoro-1,3-dioxoisoindolin-5-yl)piperazin-1-yl)-4-oxobutyl) phenoxy)-2-methylphenyl)picolinic acid (31.1 mg, 28.4 μmol, 23.9% yield, 95.1% purity) as a yellow solid.

MS (ESI) m/z: 1041.6 [M+H]⁺.

¹H NMR (400 MHz, DMSO-d₆) δ=12.94-12.58 (m, 2H), 11.11 (s, 1H), 8.03 (d, J=8.0 Hz, 1H), 7.80-7.74 (m, 2H), 7.62 (d, J=7.2 Hz, 1H), 7.53 (d, J=8.4 Hz, 1H), 7.49-7.33 (m, 5H), 7.19-7.15 (m, 3H), 7.00 (d, J=8.8 Hz, 1H), 6.89-6.81 (m, 4H), 5.14-5.09 (m, 1H), 4.99 (s, 2H), 3.93 (t, J=5.6 Hz, 2H), 3.63-3.59 (m, 4H), 3.22 (d, J=15.6 Hz, 4H), 3.03 (t, J=5.6 Hz, 2H), 2.95-2.80 (m, 1H), 2.69-2.53 (m, 4H), 2.37 (t, J=7.2 Hz, 2H), 2.10-1.98 (m, 1H), 1.90 (s, 3H), 1.84-1.76 (m, 2H).

### Example 11. Preparation of Compound 120a

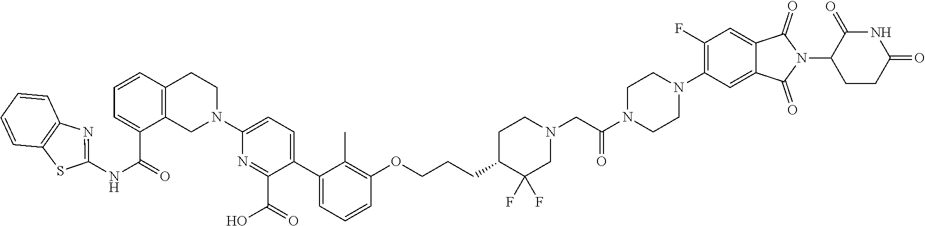 [SMILES: Cc1c(OCCC[C@@H]2CCN(CC(=O)N3CCN(c4cc5c(cc4F)C(=O)N(C4CCC(=O)NC4=O)C5=O)CC3)CC2(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((4R)-1-(2-(4-(2-(2,6-dioxopiperidin-3-yl)-6-fluoro-1,3-dioxoisoindolin-5-yl)piperazin-1-yl)-2-oxoethyl)-3,3-difluoropiperidin-4-yl)propoxy)-2-methylphenyl)picolinic acid

### Step A. Procedure for Preparation of (R)-4-(3-(3-bromo-2-methylphenoxy)propyl)-3,3-difluoropiperidine

To a solution of (R)-tert-butyl 4-(3-(3-bromo-2-methylphenoxy)propyl)-3,3-difluoropiperidine-1-carboxylate (500.00 mg, 1.12 mmol, 1 equiv.) was added HCl/dioxane (4 M, 278.81 μL, 1 equiv.). The mixture was stirred at 25° C. for 2 hours. The reaction mixture was concentrated under reduced pressure to remove solvent. The compound (R)-4-(3-(3-bromo-2-methylphenoxy)propyl)-3,3-difluoropiperidine (350 mg, crude, HCl) was obtained as a white solid.

### Step B. Procedure for Preparation of (R)-ethyl 2-(4-(3-(3-bromo-2-methylphenoxy)propyl)-3,3-difluoropiperidin-1-yl)acetate

To a solution of (R)-4-(3-(3-bromo-2-methylphenoxy)propyl)-3,3-difluoropiperidine (700.00 mg, 2.01 mmol, 1 equiv.) in CH₃CN (10 mL) was added K₂CO₃ (833.46 mg, 6.03 mmol, 3 equiv.) and ethyl 2-bromoacetate (302.13 mg, 1.81 mmol, 200.09 μL, 0.9 equiv.). The mixture was stirred at 60° C. for 1 hour. The reaction mixture was concentrated under reduced pressure to give a residue. The residue was purified by column chromatography (SiO₂, petroleum ether/ethyl acetate 1/0 to 2/1). The compound (R)-ethyl 2-(4-(3-(3-bromo-2-methylphenoxy)propyl)-3,3-difluoropiperidin-1-yl)acetate (600 mg, 1.3 mmol, 68.7% yield, 100% purity) was obtained as a white solid.

MS (ESI) m/z: 436.0 [M+H]⁺

### Step C. Procedure for Preparation of (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate

To a solution of (R)-ethyl 2-(4-(3-(3-bromo-2-methylphenoxy)propyl)-3,3-difluoropiperidin-1-yl)acetate (400 mg, 920.99 μmol, 1 equiv.) in dioxane (10 mL) was added [2-(2-aminophenyl)phenyl]palladium(1+); bis(1-adamantyl)-butyl-phosphane; methanesulfonate (67.07 mg, 92.10 μmol, 0.2 equiv.), K₂CO₃ (1 M, 2.76 mL, 3 equiv.) and tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (564.15 mg, 920.99 μmol, 1 equiv.). The mixture was stirred at 100° C. for 1 hour. The reaction mixture was concentrated under reduced pressure to give a residue. The residue was purified by column chromatography (SiO₂, petroleum ether/ethyl acetate=10/1 to 2/1). The compound (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate (600 mg, 707.1 μmol, 76.8% yield, 99% purity) was obtained as a yellow solid.

MS (ESI) m/z: 840.3 [M+H]⁺

### Step D. Procedure for Preparation of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid

To a solution of (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate (600 mg, 714.30 μmol, 1 equiv.) in THF (10 mL) and H₂O (4 mL) was added LiOH·H₂O (51.32 mg, 2.14 mmol, 3 equiv.). The mixture was stirred at 25° C. for 1 hour. The pH was adjusted to 6 with 1 M HCl. The residue was diluted with H₂O (10 mL) and extracted with (10 mL×3). The combined organic layers were washed with aqueous NaCl (10 mL×3), dried over Na₂SO₄, filtered, and concentrated under reduced pressure to give a residue. The compound (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid (500 mg, crude) was obtained as a yellow solid.

MS (ESI) m/z: 812.8 [M+H]⁺

### Step E. Procedure for Preparation of tert-butyl 6-(8-(benzo[d]thiazol-2-ylcarbamoyl)-3,4-dihydroisoquinolin-2(1H)-yl)-3-(3-(3-((4R)-1-(2-(4-(2-(2,6-dioxopiperidin-3-yl)-6-fluoro-1,3-dioxoisoindolin-5-yl)piperazin-1-yl)-2-oxoethyl)-3,3-difluoropiperidin-4-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid (100.00 mg, 123.16 μmol, 1 equiv.) in DMF (2 mL) was added HATU (56.19 mg, 147.79 μmol, 1.2 equiv.), DIEA (47.75 mg, 369.48 μmol, 64.36 μL, 3 equiv.), and 2-(2,6-dioxo-3-piperidyl)-5-fluoro-6-piperazin-1-yl-isoindoline-1,3-dione (53.26 mg, 147.79 μmol, 1.2 equiv.). The mixture was stirred at 25° C. for 1 hour. The residue was diluted with H₂O (10 mL) and extracted with DCM 30 mL (10 mL×3). The combined organic layers were washed with aqueous NaCl 30 mL (10 mL×3), dried over Na₂SO₄, filtered, and concentrated under reduced pressure to give a residue. Tert-butyl 6-(8-(benzo[d]thiazol-2-ylcarbamoyl)-3,4-dihydroisoquinolin-2(1H)-yl)-3-(3-(3-((4R)-1-(2-(4-(2-(2,6-dioxopiperidin-3-yl)-6-fluoro-1,3-dioxoisoindolin-5-yl)piperazin-1-yl)-2-oxoethyl)-3,3-difluoropiperidin-4-yl)propoxy)-2-methylphenyl)picolinate (120 mg, crude) was obtained as a brown oil.

MS (ESI) m/z: 578.2 [(M+2)/2]⁺

### Step F. Procedure for Preparation of 6-(8-(benzo[d]thiazol-2-ylcarbamoyl)-3,4-dihydroisoquinolin-2(1H)-yl)-3-(3-(3-((4R)-1-(2-(4-(2-(2,6-dioxopiperidin-3-yl)-6-fluoro-1,3-dioxoisoindolin-5-yl)piperazin-1-yl)-2-oxoethyl)-3,3-difluoropiperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-((4R)-1-(2-(4-(2-(2,6-dioxopiperidin-3-yl)-6-fluoro-1,3-dioxoisoindolin-5-yl)piperazin-1-yl)-2-oxoethyl)-3,3-difluoropiperidin-4-yl)propoxy)-2-methylphenyl)picolinate (120.00 mg, 103.96 μmol, 1 equiv.) in DCM (1 mL) was added TFA (1.54 g, 13.51 mmol, 1 mL, 129.91 equiv.). The mixture was stirred at 25° C. for 2 hours. The reaction mixture was concentrated under reduced pressure to give a residue. The residue was diluted with DMF (1.5 mL). The residue was purified by prep-HPLC. 6-(8-(Benzo[d]thiazol-2-ylcarbamoyl)-3,4-dihydroisoquinolin-2(1H)-yl)-3-(3-(3-((4R)-1-(2-(4-(2-(2,6-dioxopiperidin-3-yl)-6-fluoro-1,3-dioxoisoindolin-5-yl)piperazin-1-yl)-2-oxoethyl)-3,3-difluoropiperidin-4-yl)propoxy)-2-methylphenyl)picolinic acid (50.34 mg, 43.6 μmol, 41.9% yield, 95.1% purity) was obtained as a yellow solid.

MS (ESI) m/z: 1098.7 [M+H]⁺

¹H NMR (400 MHz, DMSO-d₆) δ=12.86 (d, J=1.2 Hz, 1H), 12.74-12.25 (m, 1H), 11.11 (s, 1H), 8.03 (d, J=8.0 Hz, 1H), 7.81-7.72 (m, 2H), 7.62 (d, J=7.2 Hz, 1H), 7.50-7.41 (m, 4H), 7.40-7.31 (m, 2H), 7.13-7.05 (m, 1H), 6.97 (d, J=8.8 Hz, 1H), 6.88 (d, J=8.4 Hz, 1H), 6.63 (d, J=7.6 Hz, 1H), 5.10 (dd, J=5.6, 12.8 Hz, 1H), 4.98 (s, 2H), 3.98 (s, 2H), 3.91 (t, J=5.6 Hz, 2H), 3.63 (d, J=2.0 Hz, 4H), 3.28-3.20 (m, 6H), 3.02 (t, J=5.6 Hz, 2H), 2.94-2.82 (m, 2H), 2.70-2.56 (m, 2H), 2.40-2.30 (m, 1H), 2.09-1.97 (m, 2H), 1.96-1.61 (m, 9H), 1.41-1.22 (m, 2H).

### Example 12. Preparation of Compound 121

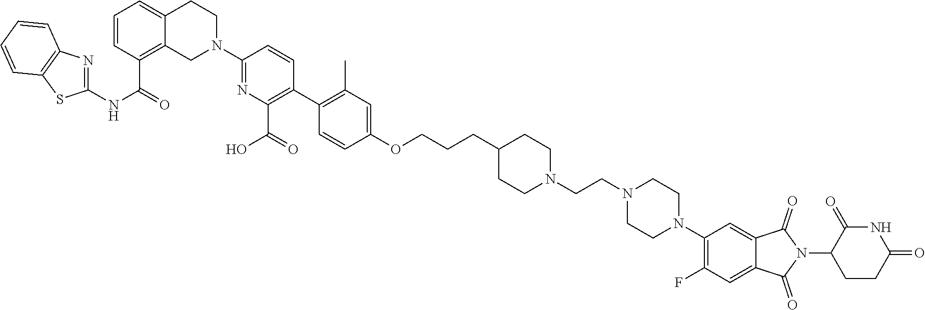 [SMILES: Cc1cc(OCCCC2CCN(CCN3CCN(c4cc5c(cc4F)C(=O)N(C4CCC(=O)NC4=O)C5=O)CC3)CC2)ccc1-c1ccc(N2CCc3cccc(C(=O)Nc4nc5ccccc5s4)c3C2)nc1C(=O)O]

### 6-[8-benzothiazol-2-ylcarbamoyl)-3,4-dihydro-1H-isoquinolin-2-yl]-3-[4-[3-[1-[2-[4-[2-(2,6-dioxo-3-piperidyl)-6-fluoro-1,3-dioxo-isoindolin-5-yl]piperazin-1-yl]ethyl]-4-piperidyl]propoxy]-2-methyl-phenyl]pyridine-2-carboxylic acid

### Step A. Procedure for Preparation of tert-butyl 4-(3-(4-bromo-3-methylphenoxy)propyl)piperidine-1-carboxylate

To a solution of tert-butyl 4-(3-bromopropyl)piperidine-1-carboxylate (2.0 g, 6.53 mmol, 1.0 equiv.) in ACN (20 mL) was added K₂CO₃ (2.71 g, 19.59 mmol, 3.0 equiv.) and 4-bromo-3-methyl-phenol (1.83 g, 9.80 mmol, 1.5 equiv.). The mixture was stirred at 60° C. for 16 hours. The reaction mixture was concentrated under reduced pressure to give tert-butyl 4-(3-(4-bromo-3-methylphenoxy)propyl)piperidine-1-carboxylate (2.5 g, crude) as a yellow solid.

MS (ESI) m/z: 313.8 [M−100+H]⁺.

### Step B. Procedure for Preparation of 4-(3-(4-bromo-3-methylphenoxy)propyl)piperidine

A mixture of tert-butyl 4-(3-(4-bromo-3-methylphenoxy)propyl)piperidine-1-carboxylate (2.5 g, 6.06 mmol, 1.0 equiv.) in HCl/EtOAc (15 mL) was stirred at 25° C. for 1 hour. The mixture was filtered and then concentrated under reduced pressure to give 4-(3-(4-bromo-3-methylphenoxy)propyl)piperidine (1.2 g, 3.4 mmol, 56.7% yield) as an off-white solid.

### Step C. Procedure for Preparation of 4-[3-(4-bromo-3-methyl-phenoxy)propyl]-1-(2,2-diethoxyethyl)piperidine

To a solution of 4-(3-(4-bromo-3-methylphenoxy)propyl)piperidine (500 mg, 1.43 mmol, 1.0 equiv., HCl) in MeCN (5 mL) was added K₂CO₃ (594.51 mg, 4.30 mmol, 3.0 equiv.) and 2-bromo-1,1-diethoxyethane (282.57 mg, 1.43 mmol, 215.70 μL, 1.0 equiv.). The mixture was stirred at 80° C. for 5 hours. The reaction mixture was concentrated under reduced pressure to remove solvent. The residue was purified by flash silica gel chromatography (eluent of 0-5% DCM/MeOH) to give 4-[3-(4-bromo-3-methyl-phenoxy)propyl]-1-(2,2-diethoxyethyl)piperidine (400 mg, 924.3 μmol, 64.4% yield, 99% purity) as a white solid.

MS (ESI) m/z: 430.0 [M+H]⁺.

¹H NMR (400 MHz, DMSO-d₆) δ=7.42 (d, J=8.8 Hz, 1H), 6.94 (d, J=2.8 Hz, 1H), 6.73-6.68 (m, 1H), 4.55 (t, J=5.2 Hz, 1H), 3.91 (t, J=6.4 Hz, 2H), 3.62-3.52 (m, 2H), 3.50-3.39 (m, 3H), 2.85 (d, J=11.2 Hz, 2H), 2.36 (d, J=5.2 Hz, 2H), 2.29 (s, 3H), 1.95 (t, J=11.2 Hz, 2H), 1.73-1.64 (m, 2H), 1.60 (d, J=11.6 Hz, 2H), 1.36-1.25 (m, 2H), 1.10 (t, J=7.2 Hz, 8H)

### Step D. Procedure for Preparation of tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate

A mixture of 4-[3-(4-bromo-3-methyl-phenoxy)propyl]-1-(2,2-diethoxyethyl)piperidine (300 mg, 700.28 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (514.74 mg, 840.33 μmol, 1.2 eq.) and KF (1.5 M, 1.40 mL, 3.0 equiv.) in dioxane (3 mL) was degassed and purged with N₂, and then [2-(2-aminophenyl)phenyl]palladium(1+); bis(1-adamantyl)-butyl-phosphane; methanesulfonate (51.00 mg, 70.03 μmol, 0.1 equiv.) was added into the mixture and stirred at 100° C. for 1 hour under N₂ atmosphere. The reaction mixture was concentrated under reduced pressure to remove solvent. The residue was purified by flash silica gel chromatography (eluent of 0˜100% ethyl acetate/petroleum ether) to give tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate (400 mg, 479.5 μmol, 68.4% yield) as a yellow oil.

MS (ESI) m/z: 834.2 [M+H]⁺.

### Step E. Procedure for Preparation of 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid

A solution of tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate (200 mg, 239.79 μmol, 1 equiv.) in HCOOH (1.5 mL) was stirred at 105° C. for 1 hour. The reaction mixture was concentrated under reduced pressure to give 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid (150 mg, crude) as a yellow solid.

MS (ESI) m/z: 704.4 [M+H]⁺

### Step F. Procedure for Preparation of 6-[8-(1,3-benzothiazol-2-ylcarbamoyl)-3,4-dihydro-1H-isoquinolin-2-yl]-3-[4-[3-[1-[2-[4-[2-(2,6-dioxo-3-piperidyl)-6-fluoro-1,3-dioxo-isoindolin-5-yl]piperazin-1-yl]ethyl]-4-piperidyl]propoxy]-2-methyl-phenyl]pyridine-2-carboxylic acid

To a solution of 2-(2,6-dioxo-3-piperidyl)-5-fluoro-6-piperazin-1-yl-isoindoline-1,3-dione (101.48 mg, 255.74 μmol, 0.9 equiv., HCl) and NaBH(OAc)₃ (180.67 mg, 852.46 μmol, 3.0 equiv.) in IPA (2 mL) and DCM (2 mL) was added 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid (200 mg, 284.15 μmol, 1.0 equiv.) at 0° C. slowly. The mixture was stirred at 0° C. for 1 hour, then the mixture was warmed to 25° C. and stirred at 25° C. for 12 hours. The mixture was concentrated under reduced pressure to remove DCM and IPA, and the mixture was diluted with DMF (3 mL). The mixture was purified by prep-HPLC to give 6-[8-(1,3-benzothiazol-2-ylcarbamoyl)-3,4-dihydro-1H-isoquinolin-2-yl]-3-[4-[3-[1-[2-[4-[2-(2,6-dioxo-3-piperidyl)-6-fluoro-1,3-dioxo-isoindolin-5-yl]piperazin-1-yl]ethyl]-4-piperidyl]propoxy]-2-methyl-phenyl]pyridine-2-carboxylic acid (24.0 mg, 21.2 μmol, 7.4% yield, 96.4% purity) as a yellow solid.

MS (ESI) m/z: 1048.6 [M+H]⁺

¹H NMR (400 MHz, DMSO-d₆) δ=11.10 (s, 1H), 8.01 (d, J=7.6 Hz, 1H), 7.77 (d, J=8.0 Hz, 1H), 7.71 (d, J=11.6 Hz, 1H), 7.61 (d, J=7.6 Hz, 1H), 7.52-7.27 (m, 6H), 6.94 (d, J=8.4 Hz, 1H), 6.86 (d, J=9.2 Hz, 1H), 6.80-6.40 (m, 1H), 6.72-6.65 (m, 1H), 5.15-5.05 (m, 1H), 4.95 (s, 2H), 3.95-3.86 (m, 5H), 3.25-3.19 (m, 6H), 3.02-2.95 (m, 4H), 2.92-2.83 (m, 2H), 2.63-2.60 (m, 1H), 2.58-2.53 (m, 6H), 2.11-2.05 (m, 2H), 2.04 (s, 3H), 1.73-1.65 (m, 2H), 1.65-1.57 (m, 2H), 1.34-1.22 (m, 3H), 1.16-1.06 (m, 2H)

¹⁹F NMR (377 MHz, DMSO-d₆) δ=−111.92 (s, 1F)

### Example 13. Preparation of Compound 122a

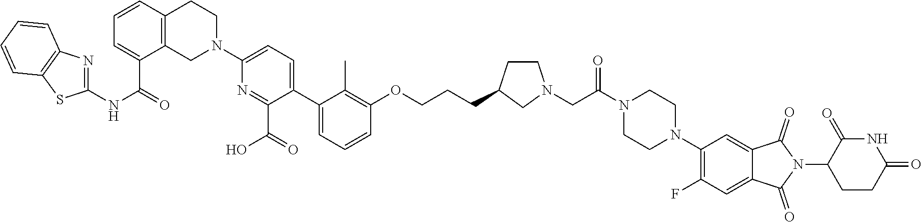 [SMILES: Cc1c(OCCC[C@H]2CCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3S)-1-(2-(4-(2-(2,6-dioxopiperidin-3-yl)-6-fluoro-1,3-dioxoisoindolin-5-yl)piperazin-1-yl)-2-oxoethyl)pyrrolidin-3-yl)propoxy)-2-methylphenyl)picolinic acid

### Step A. Procedure for Preparation of (R,E)-tert-butyl 3-(3-ethoxy-3-oxoprop-1-en-1-yl)pyrrolidine-1-carboxylate

A mixture of NaH (1.69 g, 42.16 mmol, 60% purity, 2.1 equiv.) in THF (40 mL) was purged with N₂ for three times. To the mixture was slowly added ethyl 2-(diethoxyphosphoryl)acetate (9.00 g, 40.15 mmol, 7.97 mL, 2 equiv.) at 0° C., and then the mixture was stirred for 0.5 hours under N₂ atmosphere. To the resulting solution was added (S)-tert-butyl 3-formylpyrrolidine-1-carboxylate (4 g, 20.08 mmol, 1 equiv.) at 0° C. The solution was warm to 25° C. and stirred for 16 hours under N₂ atmosphere. The reaction mixture was quenched by addition saturated NH₄Cl (40 mL) under 0° C. The mixture was extracted with ethyl acetate (20 mL×3). The combined organic layers were washed with brine (20 mL×2), dried over Na₂SO₄, filtered, and concentrated under reduced pressure to give (R,E)-tert-butyl 3-(3-ethoxy-3-oxoprop-1-en-1-yl)pyrrolidine-1-carboxylate (2.4 g, 9.0 mmol, 45.1% yield) as a white oil.

¹H NMR (400 MHz, DMSO-d₆) δ=6.85 (dd, J=7.6, 15.6 Hz, 1H), 5.93 (dd, J=0.8, 15.6 Hz, 1H), 4.11 (q, J=7.2 Hz, 2H), 3.45 (dd, J=7.2, 10.0 Hz, 1H), 3.39-3.31 (m, 3H), 3.26-3.15 (m, 1H), 2.07-1.96 (m, 1H), 1.82-1.67 (m, 1H), 1.39 (s, 9H), 1.24-1.17 (m, 3H)

### Step B. Procedure for Preparation of (S)-tert-butyl 3-(3-ethoxy-3-oxopropyl)pyrrolidine-1-carboxylate

A mixture of (R,E)-tert-butyl 3-(3-ethoxy-3-oxoprop-1-en-1-yl)pyrrolidine-1-carboxylate (2.44 g, 9.06 mmol, 1 equiv.) PtO₂ (1.44 g, 6.34 mmol, 0.7 equiv.) in EtOH (25 mL) was degassed and purged with H₂ three times, and then the mixture was stirred at 25° C. for 15 hours under H₂ (15 Psi) atmosphere. The mixture was filtered by EtOH (30 mL), and the filtrate was concentrated under reduced pressure to give (S)-tert-butyl 3-(3-ethoxy-3-oxopropyl)pyrrolidine-1-carboxylate (1.41 g, crude) as a black oil (The filter cake was immersed in EtOH).

¹H NMR (400 MHz, DMSO-d₆) δ=4.05 (q, J=7.2 Hz, 2H), 3.44-3.28 (m, 4H), 2.34-2.27 (m, 2H), 2.11-1.99 (m, 1H), 1.97-1.86 (m, 1H), 1.59 (dq, J=3.2, 7.6 Hz, 2H), 1.49-1.41 (m, 1H), 1.38 (s, 9H), 1.18 (t, J=7.2 Hz, 3H)

### Step C. Procedure for Preparation of (S)-tert-butyl 3-(3-hydroxypropyl)pyrrolidine-1-carboxylate

A mixture of (S)-tert-butyl 3-(3-ethoxy-3-oxopropyl)pyrrolidine-1-carboxylate (1.41 g, 5.20 mmol, 1 equiv.) in THF (25 mL) was slowly added LiAlH₄ (197.20 mg, 5.20 mmol, 1 equiv.) at 0° C., then the mixture was stirred at 25° C. for 5 hours. The mixture was quenched by 6.5 g Na₂SO₄·10 H₂O. The solution was poured onto ice-water (30 mL) slowly and acidified to pH=4-5 with 1 M HCl. The resulting mixture was extracted with EtOAc 60 mL (20 mL×3), washed with brine 40 mL (20 mL×2), dried by sodium sulfate, filtered, and concentrated under reduced pressure to give (S)-tert-butyl 3-(3-hydroxypropyl)pyrrolidine-1-carboxylate (1.08 g, 4.07 mmol, 78.31% yield, 86.4% purity) as a white oil.

### Step D. Procedure for Preparation of (S)-tert-butyl 3-(3-(3-bromo-2-methylphenoxy)propyl)pyrrolidine-1-carboxylate

A mixture of (S)-tert-butyl 3-(3-hydroxypropyl)pyrrolidine-1-carboxylate (1.08 g, 4.71 mmol, 1 equiv.), 3-bromo-2-methylphenol (1.06 g, 5.65 mmol, 1.2 equiv.), 2-(tributyl-λ⁵-phosphanylidene)acetonitrile (1.36 g, 5.65 mmol, 1.2 equiv.) in toluene (10 mL) was degassed and purged with N₂ three times, and then the mixture was stirred at 120° C. for 18 hours under N₂ atmosphere. The mixture concentrated under reduced pressure to give a residue. The residue was purified by flash silica gel chromatography (Eluent of 0˜7% ethyl acetate/petroleum ether) to give (S)-tert-butyl 3-(3-(3-bromo-2-methylphenoxy)propyl)pyrrolidine-1-carboxylate (1.05 g, 2.48 mmol, 52.61% yield, 94.0% purity) as a yellow oil.

### Step E. Procedure for Preparation of (S)-3-(3-(3-bromo-2-methylphenoxy)propyl)pyrrolidine

A mixture of (S)-tert-butyl 3-(3-(3-bromo-2-methylphenoxy)propyl)pyrrolidine-1-carboxylate (905 mg, 2.27 mmol, 1 equiv.) in HCl/EtOAc (10 mL) was stirred at 25° C. for 16 hours. The mixture was concentrated under the reduced pressure to give (S)-3-(3-(3-bromo-2-methylphenoxy)propyl)pyrrolidine (685 mg, 2.2 mmol, 99.2% yield, 98.2% purity) a colorless oil.

MS (ESI) m/z: 300.1 [M+H]⁺

### Step F. Procedure for Preparation of (S)-ethyl 2-(3-(3-(3-bromo-2-methylphenoxy)propyl)pyrrolidin-1-yl)acetate

A mixture of (S)-3-(3-(3-bromo-2-methylphenoxy)propyl)pyrrolidine (685 mg, 2.30 mmol, 1 equiv.), ethyl 2-bromoacetate (383.60 mg, 2.30 mmol, 254.04 μL, 1 equiv.), K₂CO₃ (952.40 mg, 6.89 mmol, 3 equiv.) in CH₃CN (1 mL) was degassed and purged with N₂ three times, and then the mixture was stirred at 60° C. for 6 hours under N₂ atmosphere. The mixture concentrated under the reduced pressure to give a residue. The residue was purified by flash silica gel chromatography (Eluent of 0˜75% ethyl acetate/petroleum ether) to give (S)-ethyl 2-(3-(3-(3-bromo-2-methylphenoxy)propyl)pyrrolidin-1-yl)acetate (903 mg, 2.30 mmol, 99.94% yield, 97.7% purity) as a blue oil.

MS (ESI) m/z: 384.1 [M+H]⁺

¹H NMR (400 MHz, DMSO-d₆) δ=7.16-7.12 (m, 1H), 7.11-7.06 (m, 1H), 6.95 (d, J=8.0 Hz, 1H), 4.10 (q, J=7.2 Hz, 2H), 3.96 (t, J=6.4 Hz, 2H), 3.47 (d, J=4.4 Hz, 2H), 2.96 (t, J=8.4 Hz, 1H), 2.81 (q, J=7.6 Hz, 1H), 2.73-2.66 (m, 1H), 2.40-2.31 (m, 1H), 2.23 (s, 3H), 2.20-2.11 (m, 1H), 2.01-1.91 (m, 1H), 1.77-1.65 (m, 2H), 1.54-1.45 (m, 2H), 1.44-1.34 (m, 1H), 1.19 (t, J=7.2 Hz, 3H)

### Step H. Procedure for Preparation of (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate

(S)-ethyl 2-(3-(3-(3-bromo-2-methylphenoxy)propyl)pyrrolidin-1-yl)acetate (314 mg, 817.06 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (550.53 mg, 898.76 μmol, 1.1 equiv.), Ad₂nBuP Pd G₃ (119.01 mg, 163.41 μmol, 0.2 equiv.), KF (1.5 M, 817.06 μL, 1.5 equiv.) in dioxane (10 mL) and were taken up into a microwave tube in dioxane (10 mL), The sealed tube was heated at 100° C. for 2 hours under microwave. The reaction mixture was partitioned between H₂O (15 mL) and ethyl acetate 60 mL (20 mL×3). The organic phase was separated, washed with aqueous NaCl 45 mL (15 mL×3), dried over (Na₂SO₄), filtered, and concentrated under reduced pressure to give a residue. The residue was purified by flash silica gel chromatography (Eluent of 0˜74% ethyl acetate/petroleum ether) to give (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate (368 mg, 465.83 μmol, 57.01% yield) as a yellow solid.

MS (ESI) m/z: 790.3 [M+H]⁺

### Step I. Procedure for Preparation of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid

A mixture of (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate (368 mg, 465.83 μmol, 1 equiv.), LiOH·H₂O (1 M, 1.40 mL, 3 equiv.) in THF (6 mL) was stirred at 25° C. for 6 hours. The mixture was concentrated and diluted with H₂O (2 mL), then the pH of the mixture was adjusted to 3 with 1 M HCl. Then the mixture was filtered and concentrated to give (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid (313 mg, 390.67 μmol, 83.86% yield, 95.1% purity) as a white solid.

MS (ESI) m/z: 762.6 [M+H]⁺

### Step J. Procedure for Preparation tert-butyl 6-(8-(benzo[d]thiazol-2-ylcarbamoyl)-3,4-dihydroisoquinolin-2(1H)-yl)-3-(3-(3-((3S)-1-(2-(4-(2-(2,6-dioxopiperidin-3-yl)-6-fluoro-1,3-dioxoisoindolin-5-yl)piperazin-1-yl)-2-oxoethyl)pyrrolidin-3-yl)propoxy)-2-methylphenyl)picolinate

To a mixture of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid (93 mg, 122.06 μmol, 1 equiv.) and 2-(2,6-dioxopiperidin-3-yl)-5-fluoro-6-(piperazin-1-yl)isoindoline-1,3-dione (52.78 mg, 146.47 μmol, 1.2 equiv.) in pyridine (2 mL) was added EDCI (35.10 mg, 183.09 μmol, 1.5 equiv.), and then the mixture was stirred at 25° C. for 15 hours. The reaction mixture was partitioned between H₂O (10 mL) and DCM 30 mL (10 mL×3). The organic phase was separated, washed with aqueous NaCl 20 mL (10 mL×2), dried over (Na₂SO₄), filtered, and concentrated under reduced pressure to give tert-butyl 6-(8-(benzo[d]thiazol-2-ylcarbamoyl)-3,4-dihydroisoquinolin-2(1H)-yl)-3-(3-(3-((3S)-1-(2-(4-(2-(2,6-dioxopiperidin-3-yl)-6-fluoro-1,3-dioxoisoindolin-5-yl)piperazin-1-yl)-2-oxoethyl)pyrrolidin-3-yl)propoxy)-2-methylphenyl)picolinate (118 mg, crude) as a yellow solid.

MS (ESI) m/z: 1105.8 [M+H]⁺

### Step K. Procedure for Preparation of 6-(8-(benzo[d]thiazol-2-ylcarbamoyl)-3,4-dihydroisoquinolin-2(1H)-yl)-3-(3-(3-((3S)-1-(2-(4-(2-(2,6-dioxopiperidin-3-yl)-6-fluoro-1,3-dioxoisoindolin-5-yl)piperazin-1-yl)-2-oxoethyl)pyrrolidin-3-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((3S)-1-(2-(4-(2-(2,6-dioxopiperidin-3-yl)-6-fluoro-1,3-dioxoisoindolin-5-yl)piperazin-1-yl)-2-oxoethyl)pyrrolidin-3-yl)propoxy)-2-methylphenyl)picolinate (93 mg, 84.22 μmol, 1 equiv.) in DCM (3 mL) and TFA (1 mL) was stirred at 40° C. for 39 hours. The reaction mixture was concentrated under the reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((3S)-1-(2-(4-(2-(2,6-dioxopiperidin-3-yl)-6-fluoro-1,3-dioxoisoindolin-5-yl)piperazin-1-yl)-2-oxoethyl)pyrrolidin-3-yl)propoxy)-2-methylphenyl)picolinic acid (60.29 mg, 53.9 μmol, 64.0% yield, 93.7% purity) as a yellow solid.

MS (ESI) m/z: 1048.9 [M+H]⁺.

¹H NMR (400 MHz, DMSO-d₆) δ=12.93-12.80 (m, 1H), 12.76-12.25 (m, 1H), 11.11 (s, 1H), 8.03 (d, J=8.0 Hz, 1H), 7.79 (d, J=10.8 Hz, 2H), 7.63 (d, J=7.2 Hz, 1H), 7.50 (d, J=7.6 Hz, 1H), 7.48-7.41 (m, 3H), 7.40-7.33 (m, 2H), 7.13-7.07 (m, 1H), 6.97 (d, J=8.8 Hz, 1H), 6.89 (d, J=8.4 Hz, 1H), 6.64 (d, J=7.6 Hz, 1H), 5.11 (dd, J=5.2, 12.8 Hz, 1H), 4.98 (s, 2H), 4.46-4.39 (m, 2H), 3.98 (t, J=5.6 Hz, 2H), 3.91 (t, J=5.6 Hz, 2H), 3.68 (d, J=2.4 Hz, 4H), 3.35-3.25 (m, 6H), 3.24-3.13 (m, 2H), 3.13-3.06 (m, 1H), 3.03 (t, J=5.6 Hz, 2H), 2.92-2.84 (m, 1H), 2.81-2.69 (m, 1H), 2.64-2.53 (m, 2H), 2.23-2.11 (m, 1H), 2.04 (dd, J=4.8, 10.8 Hz, 1H), 1.91 (s, 3H), 1.81-1.73 (m, 2H), 1.63-1.54 (m, 2H)

### Example 14. Preparation of Compound 122b

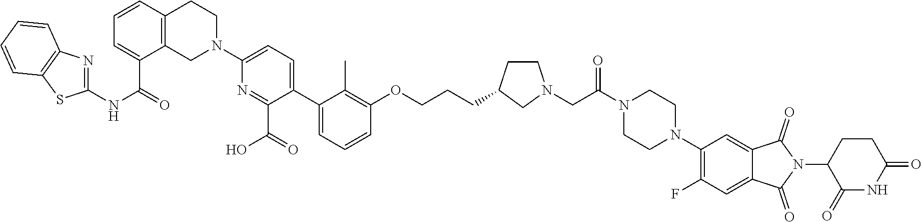 [SMILES: Cc1c(OCCC[C@@H]2CCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3R)-1-[2-[4-[2-(2,6-dioxo-3-piperidyl)-6-fluoro-1,3-dioxo-isoindolin-5-yl]piperazin-1-yl]-2-oxo-ethyl]pyrrolidin-3-yl]propoxy]-2-methyl-phenyl]pyridine-2-carboxylic acid

### Step A. Procedure for Preparation of tert-butyl (3S)-3-[(E)-3-ethoxy-3-oxo-prop-1-enyl]pyrrolidine-1-carboxylate

To a solution of tert-butyl (3R)-3-formylpyrrolidine-1-carboxylate (4 g, 20.08 mmol, 1 equiv.) in THF (60 mL) was added NaH (1.20 g, 30.11 mmol, 60% purity, 1.5 equiv.) stirred at 0° C. for 10 min under N₂ atmosphere, then ethyl 2-diethoxyphosphorylacetate (9.00 g, 40.15 mmol, 7.97 mL, 2 equiv.) was added, and the mixture was stirred at 0° C. for 2 hours under N₂ atmosphere. The reaction mixture was quenched by slow addition of water 30 mL at 0° C., and then it was extracted with ethyl acetate 90 mL (30 mL×3). The combined organic layers were washed with water 90 mL (30 mL×3), filtered, and concentrated under reduced pressure to give a residue. The residue was purified by flash silica gel chromatography (eluent of 0˜28% ethyl acetate/petroleum ether) to give tert-butyl (3S)-3-[(E)-3-ethoxy-3-oxo-prop-1-enyl]pyrrolidine-1-carboxylate (4.5 g, 16.7 mmol, 83.2% yield) as a white solid.

¹H NMR (400 MHz, DMSO-d₆) δ=6.88-6.82 (m, 1H), 5.95-5.91 (m, 1H), 5.77-5.74 (m, 1H), 4.14-4.09 (m, 2H), 3.48-3.43 (m, 1H), 3.38-3.29 (m, 2H), 3.25-3.16 (m, 1H), 3.08-2.94 (m, 2H), 1.80-1.70 (m, 1H), 1.39 (s, 9H), 1.22-1.19 (t, J=7.1 Hz, 3H).

### Step B. Procedure for Preparation of tert-butyl (3R)-3-(3-ethoxy-3-oxo-propyl) pyrrolidine-1-carboxylate

To a solution of tert-butyl (3S)-3-[(E)-3-ethoxy-3-oxo-prop-1-enyl]pyrrolidine-1-carboxylate (4.5 g, 16.71 mmol, 1 equiv.) in EtOH (60 mL) was added PtO₂ (1.14 g, 5.01 mmol, 0.3 equiv.) in EtOH (5 mL) under H₂ atmosphere (15 psi). The mixture was stirred at 25° C. for 12 hours. The mixture was filtered and washed with EtOH 15 mL (5 mL×3), and then concentrated under reduced pressure to give a residue. The residue was used in the next step without purification. Tert-butyl (3R)-3-(3-ethoxy-3-oxo-propyl) pyrrolidine-1-carboxylate (3.8 g, crude) was obtained as a white solid.

¹H NMR (400 MHz, DMSO-d₆) δ=4.08-4.03 (m, 2H), 3.47-3.38 (m, 2H), 3.30-3.29 (m, 1H), 3.18-3.08 (m, 1H), 2.80-2.74 (m, 1H), 2.34-2.30 (m, 2H), 2.11-2.00 (m, 1H), 1.95-1.90 (m, 1H), 1.66-1.53 (m, 2H), 1.39 (s, 9H), 1.24-1.16 (m, 3H).

### Step C. Procedure for Preparation of tert-butyl (3R)-3-(3-hydroxypropyl)pyrrolidine-1-carboxylate

To a solution of tert-butyl (3R)-3-(3-ethoxy-3-oxo-propyl) pyrrolidine-1-carboxylate (3.5 g, 12.90 mmol, 1 equiv.) in THF (40 mL) was added LiAlH₄ (489.55 mg, 12.90 mmol, 1 equiv.). The mixture was stirred at 0° C. for 2 hours. The reaction mixture was quenched by addition of water (0.49 mL) and 0.49 mL 15% NaOH at 0° C., and then diluted with water (5 mL) and extracted with ethyl acetate 45 mL (15 mL×3). The combined organic layers were washed with water 45 mL (15 mL×3), and concentrated under reduced pressure to give a residue. The residue was purified by flash silica gel chromatography (eluent of 0˜40% ethyl acetate/petroleum ether) to give tert-butyl (3R)-3-(3-hydroxypropyl)pyrrolidine-1-carboxylate (2.6 g, 11.3 mmol, 87.9% yield) as a white solid.

MS (ESI) m/z: 130.5 [M+H−100]⁺

### Step D. Procedure for Preparation of tert-butyl (3R)-3-[3-(3-bromo-2-methyl-phenoxy)propyl]pyrrolidine-1-carboxylate

A mixture of tert-butyl (3R)-3-(3-hydroxypropyl)pyrrolidine-1-carboxylate (2.6 g, 11.34 mmol, 1 equiv.), 3-bromo-2-methyl-phenol (2.54 g, 13.61 mmol, 1.2 equiv.), and 2-(tributyl-λ5-phosphanylidene)acetonitrile (3.28 g, 13.61 mmol, 1.2 equiv.) in toluene (30 mL) was degassed and purged with N₂ 3 times, and then the mixture was stirred at 120° C. for 12 hours under N₂ atmosphere. The reaction mixture was concentrated under reduced pressure to give a residue. The residue was purified by flash silica gel chromatography (eluent of 0˜17% ethyl acetate/petroleum ether) to give tert-butyl (3R)-3-[3-(3-bromo-2-methyl-phenoxy)propyl]pyrrolidine-1-carboxylate (3.4 g, 8.0 mmol, 70.7% yield, 94% purity) as a white solid.

MS (ESI) m/z: 300.2 [M+H−100]⁺

### Step E. Procedure for Preparation of (3R)-3-[3-(3-bromo-2-methyl-phenoxy) propyl]pyrrolidine

A solution of tert-butyl (3R)-3-[3-(3-bromo-2-methyl-phenoxy)propyl]pyrrolidine-1-carboxylate (3.4 g, 8.54 mmol, 1 equiv.) in HCl/dioxane (4 M) (10 mL) was stirred at 25° C. for 1 hour. The reaction mixture was concentrated under reduced pressure to give a residue. The residue was used in the next step without purification. (3R)-3-[3-(3-Bromo-2-methyl-phenoxy) propyl]pyrrolidine (2.8 g, crude) was obtained as a white solid.

### Step F. Procedure for Preparation of ethyl 2-[(3R)-3-[3-(3-bromo-2-methyl-phenoxy)propyl]pyrrolidin-1-yl]acetate

A mixture of (3R)-3-[3-(3-bromo-2-methyl-phenoxy) propyl]pyrrolidine (2.8 g, 8.37 mmol, 1 equiv., HCl), ethyl 2-bromoacetate (1.12 g, 6.69 mmol, 740.22 μL, 0.8 equiv.), and K₂CO₃ (3.47 g, 25.10 mmol, 3 equiv.) in MeCN (40 mL) was degassed and purged with N₂ 3 times, and then the mixture was stirred at 40° C. for 1 hour under N₂ atmosphere. The reaction mixture was concentrated under reduced pressure to give a residue. The residue was purified by flash silica gel chromatography (eluent of 0˜17% ethyl acetate/petroleum ether) to give a crude. The crude was purified by prep-SFC to give ethyl 2-[(3R)-3-[3-(3-bromo-2-methyl-phenoxy)propyl]pyrrolidin-1-yl]acetate (2 g, 4.8 mmol, 57.8% yield, 93% purity) as a white solid.

MS (ESI) m/z: 384.2 [M+H]⁺

### Step G. Procedure for Preparation of tert-butyl 6-[8-(1,3-benzothiazol-2-ylcarbamoyl)-3,4-dihydro-1H-isoquinolin-2-yl]-3-[3-[3-[(3R)-1-(2-ethoxy-2-oxo-ethyl)pyrrolidin-3-yl]propoxy]-2-methyl-phenyl]pyridine-2-carboxylate

Ethyl 2-[(3R)-3-[3-(3-bromo-2-methyl-phenoxy)propyl]pyrrolidin-1-yl]acetate (300 mg, 780.63 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (525.99 mg, 858.69 μmol, 1.1 equiv.), Ad₂nBuP Pd G₃(cataCXium® A Pd G₃) (113.70 mg, 156.13 μmol, 0.2 equiv.), and K₂CO₃ (1.5 M, 780.63 μL, 1.5 equiv.) were taken up into a microwave tube in 1,4-dioxane (4 mL). The sealed tube was heated at 100° C. for 1 hour under microwave. The reaction mixture was concentrated under reduced pressure to give a residue. The residue was purified by flash silica gel chromatography (eluent of 0˜55% ethyl acetate/petroleum ether) to give tert-butyl 6-[8-(1,3-benzothiazol-2-ylcarbamoyl)-3,4-dihydro-1H-isoquinolin-2-yl]-3-[3-[3-[(3R)-1-(2-ethoxy-2-oxo-ethyl)pyrrolidin-3-yl]propoxy]-2-methyl-phenyl]pyridine-2-carboxylate (450 mg, 569.6 μmol, 72.9% yield) as a white solid.

MS (ESI) m/z: 790.4 [M+H]⁺

### Step H. Procedure for Preparation of 2-[(3R)-3-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]pyrrolidin-1-yl]acetic acid

A mixture of tert-butyl 6-[8-(1,3-benzothiazol-2-ylcarbamoyl)-3,4-dihydro-1H-isoquinolin-2-yl]-3-[3-[3-[(3R)-1-(2-ethoxy-2-oxo-ethyl)pyrrolidin-3-yl]propoxy]-2-methyl-phenyl]pyridine-2-carboxylate (450 mg, 569.64 μmol, 1 equiv.) and LiOH·H₂O (71.71 mg, 1.71 mmol, 3 equiv.) in THF (8 mL) and H₂O (2 mL) was degassed and purged with N₂ 3 times, and then the mixture was stirred at 25° C. for 3 hours under N₂ atmosphere. The reaction mixture was concentrated under reduced pressure to remove THF, then HCl (1 mol/L) was added to adjust to pH to 5, and the mixture was filtered to give a filter cake. The filter cake was used into next step without purification. 2-[(3R)-3-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]pyrrolidin-1-yl]acetic acid (360 mg, crude) was obtained as a white solid.

MS (ESI) m/z: 762.8 [M+H]⁺

### Step I. Procedure for preparation of tert-butyl 6-[8-(1,3-benzothiazol-2-ylcarbamoyl)-3,4-dihydro-1H-isoquinolin-2-yl]-3-[3-[3-[(3R)-1-[2-[4-[2-(2,6-dioxo-3-piperidyl)-6-fluoro-1,3-dioxo-isoindolin-5-yl]piperazin-1-yl]-2-oxo-ethyl]pyrrolidin-3-yl]propoxy]-2-methyl-phenyl]pyridine-2-carboxylate

A mixture of 2-[(3R)-3-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]pyrrolidin-1-yl]acetic acid (120 mg, 157.50 μmol, 1 equiv.), 2-(2,6-dioxo-3-piperidyl)-5-fluoro-6-piperazin-1-yl-isoindoline-1,3-dione (68.10 mg, 188.99 μmol, 1.2 equiv.), DIPEA (61.06 mg, 472.49 μmol, 82.30 μL, 3 equiv.), and HATU (59.88 mg, 157.50 μmol, 1.5 equiv.) in DMF (3 mL) was degassed and purged with N₂ and then stirred at 40° C. for 6 hours under N₂ atmosphere. The reaction mixture was quenched by slow addition of water (30 mL) at 0° C., and then extracted with ethyl acetate 90 mL (30 mL×3). The combined organic layers were washed with water 90 mL (30 mL×3), filtered, and concentrated under reduced pressure to give a residue. The residue was used in the next step without purification. Tert-butyl 6-[8-(1,3-benzothiazol-2-ylcarbamoyl)-3,4-dihydro-1H-isoquinolin-2-yl]-3-[3-[3-[(3R)-1-[2-[4-[2-(2,6-dioxo-3-piperidyl)-6-fluoro-1,3-dioxo-isoindolin-5-yl]piperazin-1-yl]-2-oxo-ethyl]pyrrolidin-3-yl]propoxy]-2-methyl-phenyl]pyridine-2-carboxylate (150 mg, crude) was obtained as a white solid.

MS (ESI) m/z: 1104.8 [M+H]⁺

### Step J. Procedure for Preparation of 6-[8-(1,3-benzothiazol-2-ylcarbamoyl)-3,4-dihydro-1H-isoquinolin-2-yl]-3-[3-[3-[(3R)-1-[2-[4-[2-(2,6-dioxo-3-piperidyl)-6-fluoro-1,3-dioxo-isoindolin-5-yl]piperazin-1-yl]-2-oxo-ethyl]pyrrolidin-3-yl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[(3R)-1-[2-[4-[2-(2,6-dioxo-3-piperidyl)-6-fluoro-1,3-dioxo-isoindolin-5-yl]piperazin-1-yl]-2-oxo-ethyl]pyrrolidin-3-yl]propoxy]-2-methyl-phenyl]pyridine-2-carboxylate (150 mg, 135.84 μmol, 1 equiv.) in TFA (3 mL) and DCM (3 mL) was degassed and purged with N₂ and stirred at 25° C. for 3 hours under N₂ atmosphere. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[(3R)-1-[2-[4-[2-(2,6-dioxo-3-piperidyl)-6-fluoro-1,3-dioxo-isoindolin-5-yl]piperazin-1-yl]-2-oxo-ethyl]pyrrolidin-3-yl]propoxy]-2-methyl-phenyl]pyridine-2-carboxylic acid (95.9 mg, 84.8 μmol, 62.4% yield, 92.6% purity) was obtained as a yellow solid.

MS (ESI) m/z: 1048.7 [M+H]⁺

¹H NMR (400 MHz, DMSO-d₆) δ=11.11 (s, 1H), 8.17 (s, 1H), 8.02 (d, J=8.0 Hz, 1H), 7.80-7.71 (m, 2H), 7.62 (d, J=7.6 Hz, 1H), 7.48-7.41 (m, 3H), 7.41-7.31 (m, 3H), 7.07 (t, J=7.6 Hz, 1H), 6.92 (d, J=8.8 Hz, 1H), 6.85 (d, J=8.0 Hz, 1H), 6.62 (d, J=7.2 Hz, 1H), 5.13-5.08 (m, 1H), 4.97-4.95 (m, 2H), 3.96-3.95 (m, 2H), 3.92-3.90 (m, 2H), 3.69-3.67 (m, 2H), 3.62-3.58 (m, 2H), 3.26-3.25 (m, 4H), 3.22-3.21 (m, 2H), 3.03-3.00 (m, 2H), 2.88-2.79 (m, 2H), 2.69-2.56 (m, 4H), 2.23-2.12 (m, 2H), 2.06-2.00 (m, 1H), 1.97-1.92 (m, 1H), 1.88 (s, 3H), 1.75-1.67 (m, 2H), 1.53-1.45 (m, 2H), 1.40-1.31 (m, 1H).

### Example 15. Preparation of Compound 123

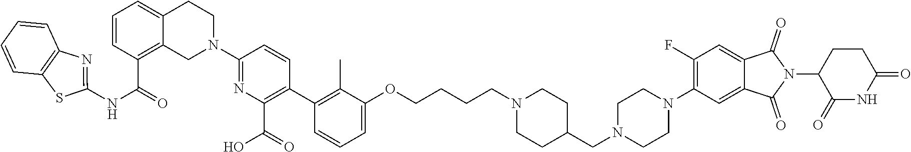 [SMILES: Cc1c(OCCCCN2CCC(CN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[4-[[4-[2-(2,6-dioxo-3-piperidyl)-6-fluoro-1,3-dioxo-isoindolin-5-yl]piperazin-1-yl]methyl]-1-piperidyl]butoxy]-2-methyl-phenyl]pyridine-2-carboxylic acid

### Step A. Procedure for Preparation of 5-[4-[[1-[4-(3-bromo-2-methyl-phenoxy)butyl]-4-piperidyl]methyl]piperazin-1-yl]-2-(2,6-dioxo-3-piperidyl)-6-fluoro-isoindoline-1,3-dione

To mixture of 1-[4-(3-bromo-2-methyl-phenoxy)butyl]piperidine-4-carbaldehyde (150 mg, 423.39 μmol, 1.0 equiv.), 2-(2,6-dioxo-3-piperidyl)-5-fluoro-6-piperazin-1-yl-isoindoline-1,3-dione (183.08 mg, 508.07 μmol, 1.2 equiv.), and CH₃COOH (254.26 μg, 4.23 μmol, 24.2 μL, 0.01 equiv.) in DCM (3 mL) and t-BuOH (3 mL), was added NaBH(OAc)₃ (269.20 mg, 1.27 mmol, 3.0 equiv.), and then the mixture was stirred at 25° C. for 2.5 hours. The reaction mixture was concentrated under reduced pressure to give a residue, the residue was purified by prep-TLC (SiO₂, DCM:MeOH=10:1) to give the 5-[4-[[1-[4-(3-bromo-2-methyl-phenoxy)butyl]-4-piperidyl]methyl]piperazin-1-yl]-2-(2,6-dioxo-3-piperidyl)-6-fluoro-isoindoline-1,3-dione (120 mg, 166.5 μmol, 39.3% yield, 96.9% purity) as brown oil.

MS (ESI) m/z: 698.2 [M+H]⁺.

### Step B. Procedure for Preparation of tert-butyl 6-[8-(1,3-benzothiazol-2-ylcarbamoyl)-3,4-dihydro-1H-isoquinolin-2-yl]-3-[3-[4-[4-[[4-[2-(2,6-dioxo-3-piperidyl)-6-fluoro-1,3-dioxo-isoindolin-5-yl]piperazin-1-yl]methyl]-1-piperidyl]butoxy]-2-methyl-phenyl]pyridine-2-carboxylate

A mixture of 5-[4-[[1-[4-(3-bromo-2-methyl-phenoxy)butyl]-4-piperidyl]methyl]piperazin-1-yl]-2-(2,6-dioxo-3-piperidyl)-6-fluoro-isoindoline-1,3-dione (100 mg, 143.14 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (113.98 mg, 186.08 μmol, 1.3 equiv.), [2-(2-aminophenyl)phenyl]palladium(1+); bis(1-adamantyl)-butyl-phosphane; methanesulfonate (10.42 mg, 14.31 μmol, 0.1 equiv.), and KF (41.58 mg, 715.70 μmol, 16.77 μL, 5.0 equiv.) in a solvent of dioxane (2 mL) and H₂O (0.2 mL) was degassed and purged with N₂ and then stirred at 100° C. for 1 hour under microwave. The reaction mixture was concentrated under reduced pressure to give a residue, and the residue was purified by prep-TLC (SiO₂, DCM:MeOH=10:1) to give tert-butyl 6-[8-(1,3-benzothiazol-2-ylcarbamoyl)-3,4-dihydro-1H-isoquinolin-2-yl]-3-[3-[4-[4-[[4-[2-(2,6-dioxo-3-piperidyl)-6-fluoro-1,3-dioxo-isoindolin-5-yl]piperazin-1-yl]methyl]-1-piperidyl]butoxy]-2-methyl-phenyl]pyridine-2-carboxylate (70 mg, 53.0 μmol, 37.0% yield, 83.7% purity) as yellow oil.

MS (ESI) m/z: 1105.8 [M+H]⁺.

### Step C. Procedure for Preparation of 6-[8-(1,3-benzothiazol-2-ylcarbamoyl)-3,4-dihydro-1H-isoquinolin-2-yl]-3-[3-[4-[4-[[4-[2-(2,6-dioxo-3-piperidyl)-6-fluoro-1,3-dioxo-isoindolin-5-yl]piperazin-1-yl]methyl]-1-piperidyl]butoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[4-[[4-[2-(2,6-dioxo-3-piperidyl)-6-fluoro-1,3-dioxo-isoindolin-5-yl]piperazin-1-yl]methyl]-1-piperidyl]butoxy]-2-methyl-phenyl]pyridine-2-carboxylate (70 mg, 63.39 μmol, 1.0 equiv.) in a solvent of DCM (0.8 mL) and TFA (0.8 mL) was stirred at 25° C. for 12 hours. The reaction mixture was concentrated under reduced pressure to give a residue, and the residue was purified by reverse-phase HPLC to give 6-[8-(1,3-benzothiazol-2-ylcarbamoyl)-3,4-dihydro-1H-isoquinolin-2-yl]-3-[3-[4-[4-[[4-[2-(2,6-dioxo-3-piperidyl)-6-fluoro-1,3-dioxo-isoindolin-5-yl]piperazin-1-yl]methyl]-1-piperidyl]butoxy]-2-methyl-phenyl]pyridine-2-carboxylic acid (33 mg, 29.7 μmol, 46.9% yield, 98.6% purity) as a yellow solid.

MS (ESI) m/z: 1048.6 [M+H]⁺.

¹H NMR (400 MHz, DMSO-d6) δ=11.10 (s, 1H), 8.13 (s, 1H), 8.02 (d, J=8.4 Hz, 1H), 7.79 (d, J=8.0 Hz, 1H), 7.75-7.70 (m, 1H), 7.63 (d, J=8.0 Hz, 1H), 7.50-7.41 (m, 4H), 7.40-7.32 (m, 2H), 7.14-7.08 (m, 1H), 6.96 (d, J=8.8 Hz, 1H), 6.92-6.88 (m, 1H), 6.65 (d, J=7.6 Hz, 1H), 5.13-5.06 (m, 1H), 4.98 (s, 2H), 4.02 (s, 2H), 3.92 (t, J=5.6 Hz, 2H), 3.24 (s, 4H), 3.06-2.94 (m, 5H), 2.93-2.82 (m, 2H), 2.80-2.70 (m, 2H), 2.63-2.57 (m, 3H), 2.54 (s, 2H), 2.24-2.16 (m, 2H), 2.07-2.01 (m, 1H), 1.92 (s, 3H), 1.90-1.71 (m, 8H), 1.34-1.22 (m, 2H).

### Example 16. Preparation of Compound 124a

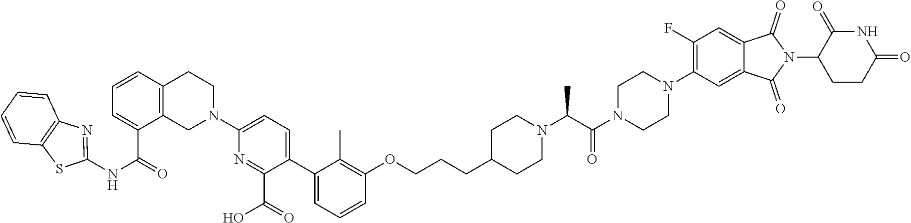 [SMILES: Cc1c(OCCCC2CCN([C@@H](C)C(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-((2S)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinic acid

### Step A. Procedure for Preparation of (S)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate

To a mixture of (S)-ethyl 2-(4-(3-(3-bromo-2-methylphenoxy)propyl)piperidin-1-yl)propanoate (450 mg, 1.09 mmol, 1 equiv.) and tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (735.30 mg, 1.20 mmol, 1.1 equiv.) in dioxane (10 mL) was added KF (1.5 M, 2.18 mL, 3 equiv.) and Ad₂nBuP Pd G₃(cataCXium® A Pd G₃) (79.47 mg, 109.13 μmol, 0.1 equiv.). After addition, the mixture was degassed and purged with N₂ and stirred at 100° C. for 2 hours under N₂ atmosphere. The reaction mixture was filtered and then concentrated under reduced pressure to give a residue. The residue was purified by flash silica gel chromatography (eluent of 0˜45% ethyl acetate/petroleum ether) to give (S)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate (600 mg, 690.7 μmol, 63.2% yield, 94.1% purity) as a yellow solid.

MS (ESI) m/z: 818.5 [M+H]⁺

### Step B. Procedure for Preparation of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid

To a solution of (S)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate (600 mg, 733.47 μmol, 1 equiv.) in THF (6 mL) was added LiOH·H₂O (92.34 mg, 2.20 mmol, 3 equiv.) and H₂O (1.5 mL). The mixture was stirred at 40° C. for 16 hours. The reaction mixture was concentrated under reduced pressure to remove THF. The aqueous phase was adjusted to pH=4-5 with 1M HCl. The reaction mixture was filtered. After filtration, the filter cake was diluted in ethyl acetate. The combined organic layers were dried over Na₂SO₄, filtered, and concentrated under reduced pressure to give (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (600 mg, crude) as a yellow solid.

MS (ESI) m/z: 790.8 [M+H]⁺

### Step C. Procedure for Preparation of tert-butyl 6-(8-(benzo[d]thiazol-2-ylcarbamoyl)-3,4-dihydroisoquinolin-2(1H)-yl)-3-(3-(3-(1-((2S)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinate

To a solution of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (100 mg, 126.59 μmol, 1 equiv.) and 2-(2,6-dioxopiperidin-3-yl)-5-fluoro-6-(piperazin-1-yl)isoindoline-1,3-dione (54.74 mg, 151.91 μmol, 1.2 equiv.) in pyridine (1 mL) was added EDCI (36.40 mg, 189.89 μmol, 1.5 equiv.). The mixture was stirred at 25° C. for 2 hours. The reaction mixture was quenched by addition H₂O (2 mL). The reaction mixture was filtered, and the filter cake was washed with 5 mL of water. After washing, the filter cake was diluted in DCM (10 mL). The combined organic layers were dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-((2S)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinate (100 mg, crude) as a yellow solid.

MS (ESI) m/z: 1134.8 [M+H]⁺

### Step D. Procedure for Preparation of 6-(8-(benzo[d]thiazol-2-ylcarbamoyl)-3,4-dihydroisoquinolin-2(1H)-yl)-3-(3-(3-(1-((2S)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-((2S)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinate (100 mg, 81.49 μmol, 92.27% purity, 1 equiv.) in DCM (1 mL) was added TFA (1.54 g, 13.51 mmol, 1 mL, 165.74 equiv.). The mixture was stirred at 25° C. for 12 hours. The reaction mixture was filtered and then concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-((2S)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinic acid (72 mg, 65.9 μmol, 80.8% yield, 98.4% purity) as a yellow solid.

MS (ESI) m/z: 1076.8 [M+H]⁺.

¹H NMR (400 MHz, DMSO-d₆) δ=13.30-12.38 (m, 1H), 11.10 (s, 1H), 8.14 (s, 1H), 8.03 (d, J=8.0 Hz, 1H), 7.83-7.68 (m, 2H), 7.62 (d, J=7.6 Hz, 1H), 7.51-7.31 (m, 6H), 7.13-7.02 (m, 1H), 6.95 (d, J=9.2 Hz, 1H), 6.86 (d, J=8.0 Hz, 1H), 6.62 (d, J=7.6 Hz, 1H), 5.10 (dd, J=5.2, 7.6 Hz, 1H), 4.97 (s, 2H), 4.08-3.76 (m, 7H), 3.65-3.60 (m, 2H), 3.23-3.18 (m, 2H), 3.13-2.98 (m, 4H), 2.94-2.73 (m, 3H), 2.60-2.56 (m, 2H), 2.38-2.29 (m, 1H), 2.17-1.99 (m, 2H), 1.88 (s, 3H), 1.77-1.61 (m, 4H), 1.42-1.29 (m, 2H), 1.25-0.99 (m, 6H).

### Example 17. Preparation of Compound 124b

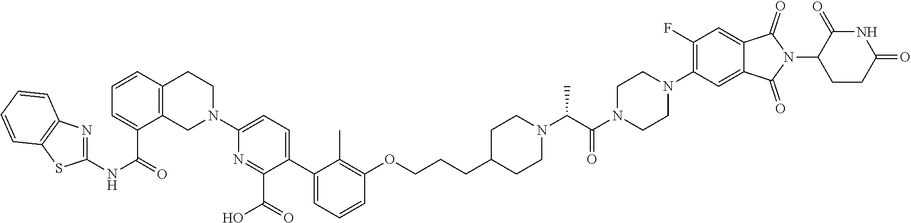 [SMILES: Cc1c(OCCCC2CCN([C@H](C)C(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-((2R)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinic acid

### Step A. Procedure for Preparation of tert-butyl 4-(3-(3-bromo-2-methylphenoxy)propyl)piperidine-1-carboxylate

A mixture of 3-bromo-2-methylphenol (1 g, 5.35 mmol, 1 equiv.), tert-butyl 4-(3-hydroxypropyl)piperidine-1-carboxylate (1.30 g, 5.35 mmol, 1 equiv.), and 2-(tributyl-λ5-phosphanylidene)acetonitrile (1.55 g, 6.42 mmol, 1.2 equiv.) in toluene (20 mL) was degassed and purged with N₂ and then stirred at 120° C. for 2 hours under N₂ atmosphere. The reaction mixture was filtered and then concentrated under reduced pressure to give a residue. The residue was purified by flash silica gel chromatography (eluent of 0˜6% ethyl acetate/petroleum ether) to give tert-butyl 4-(3-(3-bromo-2-methylphenoxy)propyl)piperidine-1-carboxylate (2.2 g, 4.8 mmol, 89.8% yield, 90% purity) as a white solid.

¹H NMR (400 MHz, DMSO-d₆) δ=7.18-7.12 (m, 1H), 7.12-7.06 (m, 1H), 6.96 (d, J=8.0 Hz, 1H), 4.02-3.86 (m, 4H), 2.67 (s, 2H), 2.23 (s, 3H), 1.81-1.71 (m, 2H), 1.65 (d, J=12.4 Hz, 2H), 1.49-1.32 (m, 11H), 1.05-0.89 (m, 2H).

### Step B. Procedure for Preparation of 4-(3-(3-bromo-2-methylphenoxy)propyl)piperidine

To a solution of tert-butyl 4-(3-(3-bromo-2-methylphenoxy)propyl)piperidine-1-carboxylate (1.7 g, 3.71 mmol, 90% purity, 1 equiv.) was added HCl/dioxane (4 M, 14.17 mL, 15.27 equiv.).

The mixture was stirred at 25° C. for 1 hour. The reaction mixture was filtered and then concentrated under reduced pressure to give 4-(3-(3-bromo-2-methylphenoxy)propyl)piperidine (1.5 g, crude, HCl) as a white solid.

¹H NMR (400 MHz, CDCl₃) δ=9.94-9.02 (m, 2H), 7.14 (d, J=8.0 Hz, 1H), 6.99 (t, J=8.0 Hz, 1H), 6.74 (d, J=8.0 Hz, 1H), 3.93 (t, J=6.4 Hz, 2H), 3.50 (d, J=12.4 Hz, 2H), 2.87 (q, J=11.6 Hz, 2H), 2.30 (s, 3H), 1.94 (d, J=13.6 Hz, 2H), 1.86-1.78 (m, 2H), 1.75-1.62 (m, 2H), 1.56-1.49 (m, 2H).

### Step C. Procedure for Preparation of ethyl 2-(4-(3-(3-bromo-2-methylphenoxy)propyl)piperidin-1-yl)propanoate

To a solution of 4-(3-(3-bromo-2-methylphenoxy)propyl)piperidine (1.3 g, 3.73 mmol, 1 eq, HCl) in MeCN (25 mL) was added K₂CO₃ (1.55 g, 11.18 mmol, 3 equiv.) and ethyl 2-bromopropanoate (1.01 g, 5.59 mmol, 728.29 μL, 1.5 equiv.). The mixture was stirred at 80° C. for 5 hours. The reaction mixture was filtered and then concentrated under reduced pressure to give a residue. The residue was purified by flash silica gel chromatography (eluent of 0-25% ethyl acetate/petroleum ether) to give ethyl 2-(4-(3-(3-bromo-2-methylphenoxy)propyl)piperidin-1-yl)propanoate (1.2 g, 2.8 mmol, 76.4% yield, 97.8% purity) as a yellow oil.

MS (ESI) m/z: 413.9 [M+H]⁺.

¹H NMR (400 MHz, CDCl₃) δ=7.14 (d, J=8.0 Hz, 1H), 6.99 (t, J=8.0 Hz, 1H), 6.75 (d, J=8.0 Hz, 1H), 4.21-4.15 (m, 2H), 3.93 (t, J=6.4 Hz, 2H), 3.28 (q, J=6.8 Hz, 1H), 2.93 (d, J=10.8 Hz, 2H), 2.34-2.17 (m, 5H), 1.86-1.77 (m, 2H), 1.73 (d, J=9.2 Hz, 2H), 1.46-1.38 (m, 2H), 1.35-1.26 (m, 9H).

### Step D. Procedure for Preparation of (R)-ethyl 2-(4-(3-(3-bromo-2-methylphenoxy)propyl)piperidin-1-yl)propanoate

The ethyl 2-(4-(3-(3-bromo-2-methylphenoxy)propyl)piperidin-1-yl)propanoate residue was further separated by SFC to give (R)-ethyl 2-(4-(3-(3-bromo-2-methylphenoxy)propyl)piperidin-1-yl)propanoate (250 mg, 597.78 μmol, 20.54% yield, 98.6% purity) as a yellow oil and (S)-ethyl 2-(4-(3-(3-bromo-2-methylphenoxy)propyl)piperidin-1-yl)propanoate (250 mg, 555.0 μmol, 19.0% yield, 91.5% purity) as a yellow oil.

¹H NMR (400 MHz, CDCl₃) δ=7.14 (d, J=8.0 Hz, 1H), 6.99 (t, J=8.0 Hz, 1H), 6.75 (d, J=8.4 Hz, 1H), 4.31-4.10 (m, 2H), 3.93 (t, J=6.4 Hz, 2H), 3.33-3.32 (m, 1H), 3.04-2.95 (m, 2H), 2.39-2.15 (m, 5H), 1.87-1.79 (m, 2H), 1.74 (d, J=9.2 Hz, 2H), 1.45-1.39 (m, 2H), 1.37-1.26 (m, 9H).

### Step E. Procedure for Preparation of (R)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate

To a mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (408.50 mg, 666.89 μmol, 1.1 equiv.) and (R)-ethyl 2-(4-(3-(3-bromo-2-methylphenoxy)propyl)piperidin-1-yl)propanoate (250 mg, 606.27 μmol, 1 equiv.) in dioxane (5 mL) was added Ad₂nBuP Pd G3(cataCXium® A Pd G₃) (44.15 mg, 60.63 μmol, 0.1 equiv.) and KF (1.5 M, 1.21 mL, 3 equiv.). After addition, the mixture was degassed and purged with N₂ and then stirred at 100° C. for 2 hours under N₂ atmosphere. The reaction mixture was filtered and then concentrated under reduced pressure to give a residue. The residue was purified by flash silica gel chromatography (eluent of 0˜60% ethyl acetate/petroleum ether) to give (R)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate (320 mg, 361.1 μmol, 59.5% yield, 92.3% purity) as a yellow solid.

MS (ESI) m/z: 818.8 [M+H]⁺.

### Step F. Procedure for Preparation of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid

To a solution of (R)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate (320 mg, 391.18 μmol, 1 equiv.) in THF (3 mL) was added LiOH·H₂O (49.25 mg, 1.17 mmol, 3 equiv.) and H₂O (0.6 mL). The mixture was stirred at 40° C. for 16 hours. The reaction mixture was concentrated under reduced pressure to remove THF. The aqueous phase was adjusted to pH=4-5 with 1M HCl. The reaction mixture was filtered. After filtration, the filter cake was diluted in ethyl acetate. The combined organic layers were dried over Na₂SO₄, filtered, and concentrated under reduced pressure to give (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (350 mg, crude) as a yellow solid.

MS (ESI) m/z: 790.7 [M+H]⁺

### Step G. Procedure for Preparation of tert-butyl 6-(8-(benzo[d]thiazol-2-ylcarbamoyl)-3,4-dihydroisoquinolin-2(1H)-yl)-3-(3-(3-(1-((2R)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (60 mg, 67.76 μmol, 89.21% purity, 1 equiv.), and 2-(2,6-dioxopiperidin-3-yl)-5-fluoro-6-(piperazin-1-yl)isoindoline-1,3-dione (29.30 mg, 81.31 μmol, 1.2 equiv.) in pyridine (1 mL) was added EDCI (19.48 mg, 101.63 μmol, 1.5 equiv.). The mixture was stirred at 25° C. for 3 hours. The reaction mixture was quenched by addition H₂O (2 mL). The reaction mixture was filtered, and the filter cake was washed with 5 mL of water. After washing, the filter cake was diluted in DCM 10 mL. The combined organic layers were dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-((2R)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinate (100 mg, crude) as a yellow solid.

MS (ESI) m/z: 1132.8 [M+H]⁺

### Step H. Procedure for Preparation of 6-(8-(benzo[d]thiazol-2-ylcarbamoyl)-3,4-dihydroisoquinolin-2(1H)-yl)-3-(3-(3-(1-((2R)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-((2R)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinate (100 mg, 88.32 μmol, 1 equiv.) in DCM (1 mL) was added TFA (1.54 g, 13.51 mmol, 1 mL, 152.93 equiv.). The mixture was stirred at 25° C. for 12 hours. The reaction mixture was filtered and then concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-((2R)-1-(4-(2-(2,6-dioxopiperidin-3-yl)-6-fluoro-1,3-dioxoisoindolin-5-yl)piperazin-1-yl)-1-oxopropan-2-yl)piperidin-4-yl)propoxy)-2-methylphenyl)picolinic acid (83.3 mg, 76.0 μmol, 86.1% yield, 98.2% purity) as a yellow solid.

MS (ESI) m/z: 1076.8 [M+H]⁺.

¹H NMR (400 MHz, DMSO-d₆) δ=13.07-12.65 (m, 1H), 11.11 (s, 1H), 8.03 (d, J=8.0 Hz, 1H), 7.99-7.93 (m, 2H), 7.80-7.77 (m, 1H), 7.64-7.61 (m, 2H), 7.52-7.32 (m, 5H), 7.10 (t, J=8.0 Hz, 1H), 7.01-6.93 (m, 1H), 6.88 (d, J=8.4 Hz, 1H), 6.64 (d, J=7.2 Hz, 1H), 5.11 (dd, J=5.6, 7.6 Hz, 1H), 4.98 (s, 2H), 4.64-4.49 (m, 1H), 4.19 (s, 2H), 4.11 (q, J=6.8 Hz, 2H), 4.00-3.88 (m, 4H), 3.84-3.81 (m, 2H), 3.36-3.23 (m, 4H), 3.03 (t, J=6.0 Hz, 2H), 2.93-2.82 (m, 2H), 2.65-2.55 (m, 2H), 2.08-2.00 (m, 1H), 1.90 (s, 4H), 1.81-1.72 (m, 2H), 1.55 (s, 2H), 1.49-1.33 (m, 5H), 1.17 (t, J=6.8 Hz, 3H).

### Example 18. Preparation of Compound 125

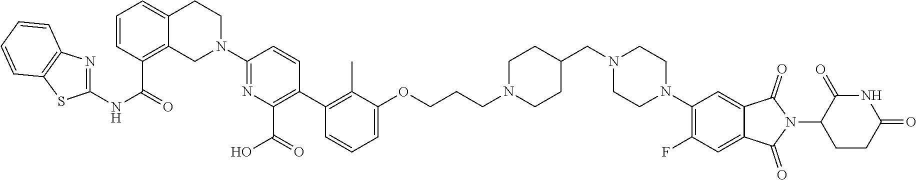 [SMILES: Cc1c(OCCCN2CCC(CN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(4-((4-(2-(2,6-dioxopiperidin-3-yl)-6-fluoro-1,3-dioxoisoindolin-5-yl)piperazin-1-yl)methyl)piperidin-1-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-(4-((4-(2-(2,6-dioxopiperidin-3-yl)-6-fluoro-1,3-dioxoisoindolin-5-yl)piperazin-1-yl)methyl)piperidin-1-yl)propoxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (62.63 mg, 102.25 μmol, 1 equiv.), 5-(4-((1-(3-(3-bromo-2-methylphenoxy)propyl)piperidin-4-yl)methyl)piperazin-1-yl)-2-(2,6-dioxopiperidin-3-yl)-6-fluoroisoindoline-1,3-dione (70 mg, 102.25 μmol, 1 equiv.), Ad₂nBuP Pd G₃(cataCXium® A Pd G₃) (14.89 mg, 20.45 μmol, 0.2 equiv.), and KF (1.5 M, 102.25 μL, 1.5 equiv.) in dioxane (0.8 mL) was degassed and purged with N₂ and stirred at 100° C. for 1 hour under N₂ atmosphere. The reaction mixture was concentrated under reduced pressure to give a residue. The residue was purified by flash silica gel chromatography (eluent of 20% ethyl acetate/petroleum ether) to give tert-butyl 6-(8-(benzo[d]thiazol-2-ylcarbamoyl)-3,4-dihydroisoquinolin-2(1H)-yl)-3-(3-(3-(4-((4-(2-(2,6-dioxopiperidin-3-yl)-6-fluoro-1,3-dioxoisoindolin-5-yl)piperazin-1-yl)methyl)piperidin-1-yl)propoxy)-2-methylphenyl)picolinate (100 mg, 91.7 μmol, 89.7% yield) as a white solid.

MS (ESI) m/z: 545.9 [M/2+H]⁺.

### Step B. Procedure for Preparation of 6-(8-(benzo[d]thiazol-2-ylcarbamoyl)-3,4-dihydroisoquinolin-2(1H)-yl)-3-(3-(3-(4-((4-(2-(2,6-dioxopiperidin-3-yl)-6-fluoro-1,3-dioxoisoindolin-5-yl)piperazin-1-yl)methyl)piperidin-1-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-(4-((4-(2-(2,6-dioxopiperidin-3-yl)-6-fluoro-1,3-dioxoisoindolin-5-yl)piperazin-1-yl)methyl)piperidin-1-yl)propoxy)-2-methylphenyl)picolinate (100 mg, 91.72 μmol, 1 equiv.) in TFA (0.5 mL) and DCM (0.5 mL) was degassed and purged with N₂ 3 times, and then the mixture was stirred at 25° C. for 10 hours under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(4-((4-(2-(2,6-dioxopiperidin-3-yl)-6-fluoro-1,3-dioxoisoindolin-5-yl)piperazin-1-yl)methyl)piperidin-1-yl)propoxy)-2-methylphenyl)picolinic acid (21.0 mg, 16.5 μmol, 17.9% yield, 90.1% purity) as a yellow solid.

MS (ESI) m/z: 1034.4 [M−H]⁺.

¹H NMR (400 MHz, DMSO-d₆) δ=11.10 (s, 1H), 8.17 (s, 1H), 8.05-7.99 (m, 1H), 7.78 (d, J=8.0 Hz, 1H), 7.72 (d, J=12.0 Hz, 1H), 7.66-7.58 (m, 1H), 7.48-7.41 (m, 3H), 7.39-7.31 (m, 2H), 7.14-7.04 (m, 1H), 6.94 (d, J=9.2 Hz, 1H), 6.90-6.82 (m, 1H), 6.68-6.60 (m, 1H), 5.10 (dd, J=5.2, 12.8 Hz, 1H), 4.98 (s, 2H), 4.01-3.95 (m, 2H), 3.91 (t, J=4.9 Hz, 2H), 3.23 (s, 6H), 3.04-3.01 (m, 2H), 2.91 (d, J=12.4 Hz, 4H), 2.69-2.58 (m, 2H), 2.18 (d, J=7.4 Hz, 2H), 2.10-1.95 (m, 4H), 1.70 (d, J=12.4 Hz, 2H), 1.53 (s, 1H), 1.25-1.05 (m, 3H)

### Example 19. Preparation of Compound 126

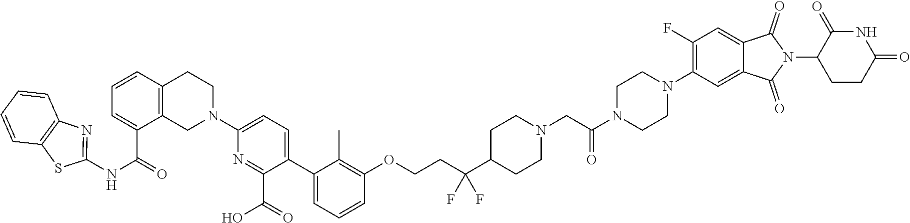 [SMILES: Cc1c(OCCC(F)(F)C2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-3,3-difluoropropoxy)-2-methylphenyl)picolinic acid

### Step A. Procedure for Preparation of tert-butyl 4-(methoxy(methyl)carbamoyl)piperidine-1-carboxylate

A mixture of 1-(tert-butoxycarbonyl)piperidine-4-carboxylic acid (50.0 g, 218 mmol, 1.0 equiv.), N,O-dimethylhydroxylamine (27.7 g, 283 mmol, 1.3 equiv.), EDCI (100 g, 523 mmol, 2.4 equiv.), NMM (33.1 g, 327 mmol, 36.0 mL, 1.5 equiv.), and HOBt (14.7 g, 109 mmol, 0.50 equiv.) in CH₂Cl₂ (500 mL) was stirred at 20° C. for 5 hours. After completion, the reaction mixture was diluted with H₂O (500 mL) and extracted with CH₂Cl₂ (400 mL×2). The combined organic layers were dried over anhydrous sodium sulfate, filtered, and concentrated under reduced pressure. The residue was purified by column chromatography (SiO₂, dichloromethane/methanol=100/1 to 20/1) to give tert-butyl 4-(methoxy(methyl)carbamoyl)piperidine-1-carboxylate (52.3 g, 192 mmol, 88.1% yield) as a yellow oil.

1H NMR (400 MHz) δ=4.30-4.00 (m, 2H), 3.71 (s, 3H), 3.18 (s, 3H), 2.90-2.67 (m, 3H), 1.75-1.62 (m, 4H), 1.45 (s, 9H).

### Step B. Procedure for Preparation of tert-butyl 4-(but-3-enoyl)piperidine-1-carboxylate

To a solution of tert-butyl 4-(methoxy(methyl)carbamoyl)piperidine-1-carboxylate (15.0 g, 55.1 mmol, 1.0 equiv.) in THF (200 mL) was added dropwise allylmagnesium bromide (1 M, 66.1 mL, 1.2 equiv.) at 0° C. After addition, the mixture was stirred 20° C. for 3 hours. The reaction mixture was quenched by addition saturated NH₄Cl aqueous solution (200 mL) at 0° C. and then extracted with CH₂Cl₂ (200 mL×2). The combined organic layers were dried over anhydrous sodium sulfate, filtered, and concentrated under reduced pressure. The residue was purified by column chromatography (SiO₂, dichloromethane/methanol=100/1 to 10/1) to give tert-butyl 4-(but-3-enoyl)piperidine-1-carboxylate (14.0 g, 49.7 mmol, 90.3% yield, 90% purity) as a brown oil.

¹H NMR (400 MHz, CDCl₃) δ=5.99-5.81 (m, 1H), 5.22-5.06 (m, 2H), 4.11 (d, J=7.2 Hz, 2H), 3.22 (d, J=6.8 Hz, 2H), 2.77 (t, J=12.0 Hz, 2H), 2.58-2.47 (m, 1H), 1.84-1.75 (m, 2H), 1.58-1.48 (m, 2H), 1.44 (s, 9H).

### Step C. Procedure for Preparation of tert-butyl 4-(1,1-difluorobut-3-en-1-yl)piperidine-1-carboxylate

To a solution of tert-butyl 4-(but-3-enoyl)piperidine-1-carboxylate (14.0 g, 55.3 mmol, 1.0 equiv.) in CH₂Cl₂ (200 mL) was added DAST (44.5 g, 276 mmol, 36.5 mL, 5.0 equiv.) dropwise at 0° C. After that, the mixture was stirred at 20° C. for 5 hours. The reaction mixture was quenched by addition of saturated NaHCO₃ aqueous solution (200 mL) at 0° C. and then extracted with CH₂Cl₂ (200 mL×3). The combined organic layers were dried over anhydrous sodium sulfate, filtered, and concentrated under reduced pressure. The residue was purified by column chromatography (SiO₂, petroleum ether/ethyl acetate=50/1 to 5/1) to give tert-butyl 4-(1,1-difluorobut-3-en-1-yl)piperidine-1-carboxylate (4.40 g, 16.0 mmol, 28.9% yield) as a colorless oil.

¹H NMR (400 MHz, CDCl₃) δ=5.90-5.70 (m, 1H), 5.35-5.01 (m, 2H), 4.32-4.14 (m, 2H), 2.80-2.49 (m, 4H), 1.96-1.85 (m, 1H), 1.76 (d, J=12.8 Hz, 2H), 1.46 (s, 9H), 1.44-1.37 (m, 2H).

### Step D. Procedure for Preparation of tert-butyl 4-(1,1-difluoro-3-hydroxypropyl)piperidine-1-carboxylate

Ozone was bubbled into a solution of tert-butyl 4-(1,1-difluorobut-3-en-1-yl)piperidine-1-carboxylate (4.40 g, 16.0 mmol, 1.0 equiv.) in CH₂Cl₂ (30.0 mL) and ethanol (10.0 mL) at −78° C. for 1 hour until the solution became blue. After excess ozone was degassed and purged by nitrogen, NaBH₄ (1.81 g, 47.9 mmol, 3.0 equiv.) was added in portions at 0° C. and the reaction was stirred at 0° C. for 1 hour. The reaction mixture was quenched by addition saturated ammonium chloride aqueous solution (80 mL) at 0° C., and then extracted with CH₂Cl₂ (40 mL×2). The combined organic layers were dried over anhydrous sodium sulfate, filtered, and concentrated under reduced pressure to give a residue. The residue was purified by column chromatography (SiO₂, petroleum ether/ethyl acetate=40/1 to 1/1) to give tert-butyl 4-(1,1-difluoro-3-hydroxypropyl)piperidine-1-carboxylate (3.50 g, 11.3 mmol, 70.6% yield, 90.0% purity) as a yellow oil.

¹H NMR (400 MHz, CDCl₃) δ=4.25-4.04 (m, 2H), 3.90-3.77 (m, 2H), 2.71-2.48 (m, 2H), 2.10-1.96 (m, 2H), 1.92-1.82 (m, 1H), 1.71 (d, J=13.2 Hz, 2H), 1.39 (s, 9H), 1.37-1.27 (m, 2H).

### Step E. Procedure for Preparation of tert-butyl 4-(3-(3-bromo-2-methylphenoxy)-1,1-difluoropropyl)piperidine-1-carboxylate

To a solution of tert-butyl 4-(1,1-difluoro-3-hydroxypropyl)piperidine-1-carboxylate (3.50 g, 12.5 mmol, 1.0 equiv.) in toluene (40.0 mL) were added 3-bromo-2-methylphenol (2.58 g, 13.8 mmol, 1.1 equiv.) and CMPB (6.05 g, 25.1 mmol, 2.0 equiv.). The reaction was stirred at 120° C. for 2 hours. After completion, the mixture was concentrated under reduced pressure, and the residue was purified by column chromatography (SiO₂, petroleum ether/ethyl acetate=30/1 to 3/1) to give tert-butyl 4-(3-(3-bromo-2-methylphenoxy)-1,1-difluoropropyl)piperidine-1-carboxylate (2.4 g, 3.5 mmol, 28.3% yield, 63.6% purity) as a yellow oil.

MS (ESI) m/z: 448.3 [M+H]⁺

¹H NMR (400 MHz, CDCl₃) δ=7.10 (d, J=8.0 Hz, 1H), 6.94 (t, J=8.4 Hz, 1H), 6.71 (d, J=8.4 Hz, 1H), 4.19-4.06 (m, 4H), 2.64-2.54 (m, 2H), 2.38-2.27 (m, 2H), 2.23 (s, 3H), 1.97-1.89 (m, 1H), 1.76 (d, J=13.2 Hz, 2H), 1.40-1.39 (m, 11H).

### Step F. Procedure for Preparation of 4-(3-(3-bromo-2-methylphenoxy)-1,1-difluoropropyl)piperidine

A mixture of tert-butyl 4-(3-(3-bromo-2-methylphenoxy)-1,1-difluoropropyl)piperidine-1-carboxylate (2.26 g, 5.04 mmol, 1.0 equiv.) in HCl/1,4-dioxane (20.0 mL) was stirred at 25° C. for 1 hour. After completion, the reaction mixture was concentrated under reduced pressure to give 4-(3-(3-bromo-2-methylphenoxy)-1,1-difluoropropyl)piperidine (1.76 g, crude) as a yellow oil.

MS (ESI) m/z: 347.8 [M+H]⁺

### Step G. Procedure for Preparation of ethyl 2-(4-(3-(3-bromo-2-methylphenoxy)-1,1-difluoropropyl)piperidin-1-yl)acetate

To a solution of 4-(3-(3-bromo-2-methylphenoxy)-1,1-difluoropropyl)piperidine (1.76 g, 5.05 mmol, 1.0 equiv.) and ethyl 2-bromoacetate (802 mg, 4.80 mmol, 531 μL, 0.95 equiv.) in MeCN (20 mL) was added K₂CO₃ (1.40 g, 10.1 mmol, 2.0 equiv.). The mixture was stirred at 60° C. for 2 hours. After completion, the reaction mixture was diluted with H₂O (30 mL) and extracted with CH₂Cl₂ (30 mL×2). The combined organic layers were dried over anhydrous sodium sulfate, filtered, and concentrated under reduced pressure to give a residue. The residue was purified by column chromatography (SiO₂, petroleum ether/ethyl acetate=30/1 to 3/1) to give ethyl 2-(4-(3-(3-bromo-2-methylphenoxy)-1,1-difluoropropyl)piperidin-1-yl)acetate (1.10 g, 2.45 mmol, 48.5% yield, 96.8% purity) as a yellow oil.

MS (ESI) m/z: 435.9 [M+H]⁺

¹H NMR (400 MHz, CDCl₃) δ=7.10 (d, J=8.0 Hz, 1H), 6.94 (t, J=8.0 Hz, 1H), 6.71 (d, J=8.0 Hz, 1H), 4.18-4.05 (m, 4H), 3.15 (s, 2H), 2.97 (d, J=11.2 Hz, 2H), 2.38-2.25 (m, 2H), 2.23 (s, 3H), 2.11 (t, J=11.2 Hz, 2H), 1.85-1.72 (m, 3H), 1.69-1.58 (m, 2H), 1.21 (t, J=7.2 Hz, 3H).

### Step H. Procedure for Preparation of tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)-3,3-difluoropropoxy)-2-methylphenyl)picolinate

A mixture of ethyl 2-(4-(3-(3-bromo-2-methylphenoxy)-1,1-difluoropropyl)piperidin-1-yl)acetate (313 mg, 684 μmol, 95% purity, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (440 mg, 718. μmol, 1.05 equiv.), Ad₂nBuP Pd G₃ (cataCXium® A Pd G₃) (99.6 mg, 137 μmol, 0.20 equiv.), K₃PO₄ (436 mg, 2.05 mmol, 3.0 equiv.), and H₂O (0.30 mL) in 1,4-dioxane (3.00 mL) was degassed and purged with nitrogen three times, and then the mixture was stirred at 90° C. for 2 hours under nitrogen atmosphere. The reaction mixture was diluted with H₂O (10 mL) and extracted with CH₂Cl₂ (10 mL×3). The combined organic layers were dried over anhydrous sodium sulfate, filtered, and concentrated under reduced pressure to give a residue. The residue was purified by column chromatography (SiO₂, petroleum ether/ethyl acetate=30/1 to 1/1) to give tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)-3,3-difluoropropoxy)-2-methylphenyl)picolinate (357 mg, 372 μmol, 54.4% yield, 87.6% purity) as a yellow solid.

MS (ESI) m/z: 840.7 [M+H]⁺

¹H NMR (400 MHz, CDCl₃) δ=7.86 (d, J=7.6 Hz, 1H), 7.66 (d, J=8.0 Hz, 1H), 7.57 (d, J=8.0 Hz, 1H), 7.44-7.28 (m, 5H), 7.12 (t, J=8.0 Hz, 1H), 6.90 (d, J=8.8 Hz, 1H), 6.83 (d, J=8.0 Hz, 1H), 6.73 (d, J=8.0 Hz, 1H), 5.16-4.93 (m, 2H), 4.26-4.14 (m, 4H), 4.13-4.08 (m, 2H), 3.24 (s, 2H), 3.11-3.01 (m, 4H), 2.45-2.33 (m, 2H), 2.29-2.12 (m, 2H), 1.98 (s, 3H), 1.89-1.83 (m, 2H), 1.79-1.70 (m, 2H), 1.28-1.25 (m, 4H), 1.15 (s, 9H).

### Step I. Procedure for preparation of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-1,1-difluoropropyl)piperidin-1-yl)acetic acid

To a solution of tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)-3,3-difluoropropoxy)-2-methylphenyl)picolinate (307 mg, 365 μmol, 1.0 equiv.) in THF (2.00 mL) and H₂O (2.00 mL) was added LiOH·H₂O (43.8 mg, 1.83 mmol, 5.0 equiv.). The mixture was stirred at 20° C. for 2 hours. The reaction mixture was concentrated under reduced pressure to give 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-1,1-difluoropropyl)piperidin-1-yl)acetic acid (190 mg, crude) as a yellow solid.

MS (ESI) m/z: 812.4 [M+H]⁺

### Step J. Procedure for Preparation of tert-butyl 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-3,3-difluoropropoxy)-2-methylphenyl)picolinate

To a solution of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-1,1-difluoropropyl)piperidin-1-yl)acetic acid (70.0 mg, 86.2 μmol, 1.0 equiv.) in DMF (1.00 mL) were added 2-(2,6-dioxopiperidin-3-yl)-5-fluoro-6-(piperazin-1-yl)isoindoline-1,3-dione (34.2 mg, 94.8 μmol, 1.1 equiv.), HATU (39.3 mg, 103 μmol, 1.2 equiv.), and DIEA (33.4 mg, 259 μmol, 45.1 μL, 3.0 equiv.). The mixture was stirred at 20° C. for 1 hour. The reaction mixture was filtered to give a filtrate. The filtrate was purified by prep-HPLC to give tert-butyl 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-3,3-difluoropropoxy)-2-methylphenyl)picolinate (40.0 mg, 34.7 μmol, 40.2% yield) as a yellow solid.

MS (ESI) m/z: 1154.6 [M+H]⁺

### Step K. Procedure for Preparation of 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-3,3-difluoropropoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-3,3-difluoropropoxy)-2-methylphenyl)picolinate (40.0 mg, 34.7 μmol, 1.0 equiv.) in CH₂Cl₂ (1.00 mL) was added TFA (770 mg, 6.75 mmol, 0.5 mL, 195 equiv.). The mixture was stirred at 20° C. for 6 hours. After completion, the reaction mixture was concentrated under reduced pressure and the residue was purified by prep-HPLC to give 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-3,3-difluoropropoxy)-2-methylphenyl)picolinic acid (16.7 mg, 14.0 μmol, 40.5% yield, 91.9% purity) as a yellow solid.

MS (ESI) m/z: 1098.5 [M+H]⁺

¹H NMR (400 MHz, DMSO-d₆) δ=13.04-12.65 (m, 1H), 11.12 (s, 1H), 9.75-9.29 (m, 1H), 8.02 (d, J=8.0 Hz, 1H), 7.84-7.74 (m, 2H), 7.63 (d, J=8.0 Hz, 1H), 7.51-7.42 (m, 4H), 7.40-7.31 (m, 2H), 7.13 (t, J=7.6 Hz, 1H), 7.01-6.90 (m, 2H), 6.67 (d, J=7.6 Hz, 1H), 5.16-5.07 (m, 1H), 4.98 (s, 2H), 4.44-4.27 (m, 2H), 4.22-4.11 (m, 2H), 3.92 (t, J=5.6 Hz, 2H), 3.69 (s, 2H), 3.62-3.50 (m, 4H), 3.29-3.27 (m, 2H), 3.09-2.99 (m, 3H), 2.99-2.81 (m, 3H), 2.62-2.57 (m, 2H), 2.46-2.39 (m, 3H), 2.09-1.98 (m, 3H), 1.90 (s, 3H), 1.88-1.75 (m, 2H).

### Example 20. Preparation of Compound 127

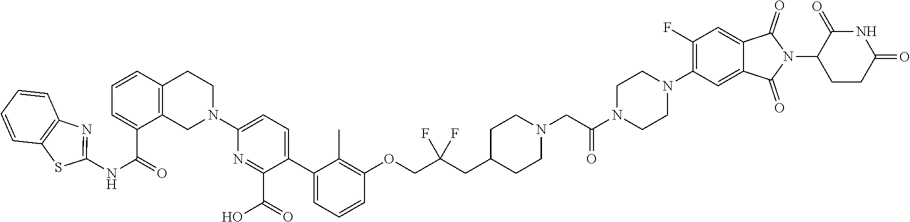 [SMILES: Cc1c(OCC(F)(F)CC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-2,2-difluoropropoxy)-2-methylphenyl)picolinic acid

### Step A. Procedure for Preparation of tert-butyl 4-(3-ethoxy-2,2-difluoro-1-hydroxy-3-oxo-propyl)piperidine-1-carboxylate

A mixture of Zn (5.21 g, 79.71 mmol, 2 equiv.) in THF (100 mL) was degassed and purged with N₂ 3 times and 1,2-dibromoethane (748.72 mg, 3.99 mmol, 300.69 μL, 0.1 equiv.) was added dropwise, and then the mixture was stirred at 60° C. for 0.5 hours under N₂ atmosphere. Then, a solution of tert-butyl 4-formylpiperidine-1-carboxylate (8.5 g, 39.86 mmol, 1 equiv.) and ethyl 2-bromo-2,2-difluoro-acetate (11.33 g, 55.80 mmol, 7.17 mL, 1.4 equiv.) in THF (100 mL) was added dropwise to the solution, and the resulting solution was stirred at 60° C. for 3 hours. The mixture was cooled to 20° C. and then quenched by 1N HCl (200 mL), and then extracted with EtOAc (100 mL×3). The combined organic layers were washed with brine (100 mL×2), dried over Na₂SO₄, filtered, and concentrated under reduced pressure to give a residue. The residue was purified by flash silica gel chromatography (eluent of 0˜20% ethyl acetate/petroleum ether) to give tert-butyl 4-(3-ethoxy-2,2-difluoro-1-hydroxy-3-oxo-propyl)piperidine-1-carboxylate (19.6 g, crude) as a colorless oil.

¹H NMR (400 MHz, DMSO-d₆,) 6=4.35-4.21 (m, 2H), 3.94 (s, 2H), 3.80-3.64 (m, 1H), 3.18-3.16 (m, 1H), 2.67 (s, 2H), 1.82-1.68 (m, 2H), 1.59 (d, J=12.4 Hz, 1H), 1.39 (s, 9H), 1.34-1.12 (m, 5H).

### Step B. Procedure for Preparation of tert-butyl 4-(3-ethoxy-2,2-difluoro-1-methylsulfanylcarbothioyloxy-3-oxo-propyl)piperidine-1-carboxylate

To a solution of tert-butyl 4-(3-ethoxy-2,2-difluoro-1-hydroxy-3-oxo-propyl)piperidine-1-carboxylate (10 g, 29.64 mmol, 1 equiv.) in DMF (100 mL) was added DBU (18.05 g, 118.57 mmol, 17.87 mL, 4 equiv.) and CS₂ (22.57 g, 296.42 mmol, 17.91 mL, 10 equiv.). The mixture was stirred at 20° C. for 1 hours, then Mel (42.07 g, 296.42 mmol, 18.45 mL, 10 equiv.) was added to the mixture, and the mixture was stirred at 20° C. for 12 hours. The mixture was diluted with water (200 mL) and extracted with EtOAc (100 mL×3). The combined organic layers were washed with brine (100 mL×3), dried over Na₂SO₄, filtered, and concentrated under reduced pressure to give a residue. The residue was purified by flash silica gel chromatography (eluent of 0˜8% ethyl acetate/petroleum ether) to give tert-butyl 4-(3-ethoxy-2,2-difluoro-1-methylsulfanylcarbothioyloxy-3-oxo-propyl)piperidine-1-carboxylate (11 g, 25.7 mmol, 86.8% yield) as a yellow oil.

¹H NMR (400 MHz, CDCl₃) δ=6.40-6.15 (m, 1H), 4.29 (q, J=7.2 Hz, 2H), 4.18-4.10 (m, 2H), 2.80-2.62 (m, 2H), 2.59 (s, 3H), 2.30-2.15 (m, 1H), 1.78 (t, J=11.2 Hz, 2H), 1.45 (s, 10H), 1.33 (t, J=7.2 Hz, 3H).

### Step C. Procedure for Preparation of tert-butyl 4-(3-ethoxy-2,2-difluoro-3-oxo-propyl)piperidine-1-carboxylate

A mixture of tert-butyl 4-(3-ethoxy-2,2-difluoro-1-methylsulfanylcarbothioyloxy-3-oxo-propyl)piperidine-1-carboxylate (10.5 g, 24.56 mmol, 1 equiv.), 2-tert-butylperoxy-2-methyl-propane (3.95 g, 27.02 mmol, 4.98 mL, 1.1 equiv.), and phenylphosphonoylbenzene (5.96 g, 29.47 mmol, 1.2 equiv.) in dioxane (100 mL) was degassed and purged with N₂ 3 times, and then the mixture was stirred at 100° C. for 24 hours under N₂ atmosphere. The reaction mixture was diluted with H₂O (50 mL) and extracted with ethyl acetate 200 mL (100 mL×2). The combined organic layers were washed with brine 50 mL (25 mL×2), dried over Na₂SO₄, filtered, and concentrated under reduced pressure to give a residue. The residue was purified by flash silica gel chromatography (eluent of 0˜16% ethyl acetate/petroleum ether) to give tert-butyl 4-(3-ethoxy-2,2-difluoro-3-oxo-propyl)piperidine-1-carboxylate (5.1 g, 15.8 mmol, 64.6% yield) as a colorless oil.

¹H NMR (400 MHz, CDCl₃) δ=4.37-4.31 (m, 2H), 4.07 (d, J=12.8 Hz, 2H), 2.71 (t, J=12.8 Hz, 2H), 2.12-1.93 (m, 2H), 1.81-1.71 (m, 3H), 1.46 (s, 9H), 1.37 (t, J=7.2 Hz, 3H), 1.28-1.15 (m, 2H).

### Step D. Procedure for Preparation of tert-butyl 4-(2,2-difluoro-3-hydroxy-propyl)piperidine-1-carboxylate

To a mixture of tert-butyl 4-(3-ethoxy-2,2-difluoro-3-oxo-propyl)piperidine-1-carboxylate (5.1 g, 15.87 mmol, 1 equiv.) in THF (60 mL) was added LAH (602.27 mg, 15.87 mmol, 1 equiv.), and then the mixture was stirred at 0° C. for 1 hour under N₂ atmosphere. The reaction mixture was quenched by addition H₂O (0.7 mL) at 0° C. The aqueous phase was adjusted to pH=4-5 with 1M HCl. The reaction mixture was filtered. After filtration, the filter cake was diluted in ethyl acetate. The combined organic layers were dried over Na₂SO₄, filtered, and concentrated under reduced pressure to give a residue. The residue was purified by flash silica gel chromatography (eluent of 0-18% ethyl acetate/petroleum ether) to give tert-butyl 4-(2,2-difluoro-3-hydroxy-propyl)piperidine-1-carboxylate (3.5 g, 11.3 mmol, 71.1% yield, 90% purity) as a colorless oil.

¹H NMR (400 MHz, DMSO-d₆) δ=5.47 (t, J=6.0 Hz, 1H), 3.89 (d, J=12.4 Hz, 2H), 3.61-3.49 (m, 2H), 2.71 (s, 2H), 1.90-1.73 (m, 3H), 1.68 (d, J=13.6 Hz, 2H), 1.38 (s, 9H), 1.14-1.00 (m, 2H).

### Step E. Procedure for Preparation of tert-butyl 4-[3-(3-bromo-2-methyl-phenoxy)-2,2-difluoro-propyl]piperidine-1-carboxylate

A mixture of tert-butyl 4-(2,2-difluoro-3-hydroxy-propyl)piperidine-1-carboxylate (500 mg, 1.79 mmol, 1 equiv.), 1,3-dibromo-2-methyl-benzene (894.77 mg, 3.58 mmol, 2 equiv.), 2,2,6,6-tetramethylheptane-3,5-dione (82.47 mg, 447.51 μmol, 92.14 μL, 0.25 equiv.), CuI (170.46 mg, 895.02 μmol, 0.5 equiv.), and Cs₂CO₃ (699.88 mg, 2.15 mmol, 1.2 equiv.) in NMP (5 mL) was degassed and purged with N₂ 3 times, and then the mixture was stirred at 120° C. for 12 hours under N₂ atmosphere. The reaction mixture was quenched by addition water (50 mL), and then extracted with EtOAc (50 mL×3). The combined organic layers were washed with brine (50 mL×3), dried over anhydrous sulfate sodium, filtered, and concentrated under reduced pressure to give a residue. The residue was purified by column chromatography (SiO₂, petroleum ether/ethyl acetate 1/0 to 4/1) to give tert-butyl 4-[3-(3-bromo-2-methyl-phenoxy)-2,2-difluoro-propyl]piperidine-1-carboxylate (1.78 g, crude) as a yellow oil.

MS (ESI) m/z: 394.2 [M−56+H]⁺

¹H NMR (400 MHz, DMSO-d₆) δ=7.24 (d, J=8.0 Hz, 1H), 7.13 (t, J=8.4 Hz, 1H), 7.05 (d, J=8.0 Hz, 1H), 4.40-4.25 (m, 2H), 3.89 (d, J=12.0 Hz, 2H), 2.83-2.61 (m, 2H), 1.99 (s, 3H), 1.90-1.78 (m, 1H), 1.76-1.64 (m, 2H), 1.38 (s, 9H), 1.16-1.01 (m, 4H).

### Step F. Procedure for Preparation of 4-[3-(3-bromo-2-methyl-phenoxy)-2,2-difluoro-propyl]piperidine

A solution of tert-butyl 4-[3-(3-bromo-2-methyl-phenoxy)-2,2-difluoro-propyl]piperidine-1-carboxylate (1.78 g, 3.97 mmol, 1 equiv.) in HCl/EtOAc (4 M, 15 mL, 15.11 equiv.) was stirred at 25° C. for 2 hours. The reaction mixture was concentrated under reduced pressure to give 4-[3-(3-bromo-2-methyl-phenoxy)-2,2-difluoro-propyl]piperidine (2 g, crude) as a yellow solid.

MS (ESI) m/z: 348.2 [M+H]⁺

### Step G. Procedure for Preparation of ethyl 2-[4-[3-(3-bromo-2-methyl-phenoxy)-2,2-difluoro-propyl]-1-piperidyl]acetate

To a solution of 4-[3-(3-bromo-2-methyl-phenoxy)-2,2-difluoro-propyl]piperidine (500 mg, 1.44 mmol, 1 equiv.) and ethyl 2-bromoacetate (239.79 mg, 1.44 mmol, 158.80 μL, 1 equiv.) in MeCN (5 mL) was added K₂CO₃ (595.33 mg, 4.31 mmol, 3 equiv.). The mixture was stirred at 60° C. for 1 hour. The reaction mixture was filtered and then concentrated under reduced pressure to give a residue. The residue was purified by column chromatography (SiO₂, petroleum ether/ethyl acetate 1/0 to 1/1) to give ethyl 2-[4-[3-(3-bromo-2-methyl-phenoxy)-2,2-difluoro-propyl]-1-piperidyl]acetate (380 mg, 874.9 μmol, 60.9% yield) as a yellow oil.

MS (ESI) m/z: 436.0 [M+H]⁺

¹H NMR (400 MHz, DMSO-d₆) δ=7.23 (d, J=8.0 Hz, 1H), 7.12 (t, J=8.0 Hz, 1H), 7.05 (d, J=7.6 Hz, 1H), 4.38-4.25 (m, 2H), 4.06 (q, J=7.2 Hz, 2H), 3.15 (s, 2H), 2.78 (d, J=10.8 Hz, 2H), 2.25 (s, 2H), 2.14 (t, J=11.2 Hz, 2H), 2.06-1.91 (m, 2H), 1.72-1.63 (m, 2H), 1.34-1.21 (m, 2H), 1.21-1.14 (m, 5H).

### Step H. Procedure for Preparation of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]-2,2-difluoro-propoxy]-2-methyl-phenyl]pyridine-2-carboxylate

Ethyl 2-[4-[3-(3-bromo-2-methyl-phenoxy)-2,2-difluoro-propyl]-1-piperidyl]acetate (380 mg, 874.94 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (535.94 mg, 874.94 μmol, 1 equiv.), [2-(2-aminophenyl)phenyl]palladium(1+); bis(1-adamantyl)-butyl-phosphane; methanesulfonate (63.71 mg, 874.94 μmol, 0.1 equiv.), and KF (1.5 M, 1.75 mL, 3 equiv.) were taken up into a microwave tube in dioxane (2 mL). The sealed tube was heated at 100° C. for 60 minutes under microwave. The reaction mixture was concentrated under reduced pressure to give a residue. The residue was purified by column chromatography (SiO₂, petroleum ether/ethyl acetate 10/1 to 1/1) to give tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]-2,2-difluoro-propoxy]-2-methyl-phenyl]pyridine-2-carboxylate (512 mg, 609.5 μmol, 69.6% yield) as a yellow solid.

MS (ESI) m/z: 840.5 [M+H]⁺

¹H NMR (400 MHz, DMSO-d₆,) 6=12.92-12.81 (m, 1H), 8.02 (d, J=8.4 Hz, 1H), 7.77 (d, J=8.4 Hz, 1H), 7.60 (d, J=7.6 Hz, 1H), 7.45 (d, J=8.8 Hz, 3H), 7.40-7.31 (m, 3H), 7.14 (t, J=8.0 Hz, 1H), 7.00 (d, J=8.4 Hz, 1H), 6.95 (d, J=8.4 Hz, 1H), 6.65 (d, J=7.6 Hz, 1H), 4.98 (s, 2H), 4.35-4.21 (m, 2H), 4.06-4.03 (m, 2H), 3.87 (t, J=5.6 Hz, 2H), 3.14 (s, 2H), 3.03 (t, J=5.6 Hz, 2H), 2.76 (d, J=11.2 Hz, 2H), 2.12 (t, J=10.8 Hz, 2H), 1.99 (s, 3H), 1.89 (s, 3H), 1.71-1.58 (m, 3H), 1.17 (d, J=2.0 Hz, 3H), 1.00 (s, 9H).

### Step I. Procedure for preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]-2,2-difluoro-propoxy]-2-methyl-phenyl]pyridine-2-carboxylate (512 mg, 609.53 μmol, 1 equiv.) in H₂O (2.5 mL) and THF (2.5 mL) was added LiOH (72.99 mg, 3.05 mmol, 5 equiv.). The mixture was stirred at 25° C. for 2 hours. The reaction mixture was quenched by addition HCl (1M) (10 mL), and then extracted with CH₂Cl₂ (20 mL×3). The combined organic layers were dried over anhydrous sodium sulfate, filtered, and concentrated under reduced pressure to give 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-1-piperidyl]acetic acid (600 mg, crude) as a yellow solid.

### Step J. Procedure for Preparation of tert-butyl 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-2,2-difluoropropoxy)-2-methylphenyl)picolinate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-1-piperidyl]acetic acid (100 mg, 123.16 μmol, 1 equiv.) in DMF (2 mL) was added HATU (56.20 mg, 147.80 μmol, 1.2 equiv.), DIPEA (47.75 mg, 369.49 μmol, 64.36 μL, 3 equiv.), and 2-(2,6-dioxopiperidin-3-yl)-5-fluoro-6-(piperazin-1-yl)isoindoline-1,3-dione (44.38 mg, 123.16 μmol, 1 equiv.). The mixture was stirred at 25° C. for 1.5 hours. The reaction mixture was quenched by addition H₂O (2 mL). The reaction mixture was filtered and the filter cake was washed with 5 mL of water. After filtration, the filter cake was diluted in DCM (10 mL).

The combined organic layers were dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-2,2-difluoropropoxy)-2-methylphenyl)picolinate (100 mg, crude) as a yellow solid.

MS (ESI) m/z: 1154.9 [M+H]⁺

### Step K. Procedure for Preparation of 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-2,2-difluoropropoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-2,2-difluoropropoxy)-2-methylphenyl)picolinate (100 mg, 86.64 μmol, 1 equiv.) in DCM (1 mL) was added TFA (9.88 mg, 86.64 μmol, 6.41 μL, 1 equiv.). The mixture was stirred at 25° C. for 12 hours. The reaction mixture was filtered and then concentrated under reduced pressure to give a residue. The crude product was purified by prep-HPLC to give 6-(8-(benzo[d]thiazol-2-ylcarbamoyl)-3,4-dihydroisoquinolin-2(1H)-yl)-3-(3-(3-(1-(2-(4-(2-(2,6-dioxopiperidin-3-yl)-6-fluoro-1,3-dioxoisoindolin-5-yl)piperazin-1-yl)-2-oxoethyl)piperidin-4-yl)-2,2-difluoropropoxy)-2-methylphenyl)picolinic acid (28.7 mg, 25.5 μmol, 29.4% yield, 96.7% purity) as a yellow solid.

MS (ESI) m/z: 1098.8 [M+H]⁺.

¹H NMR (400 MHz, DMSO-d₆) δ=11.10 (s, 1H), 8.15 (s, 1H), 8.03 (d, J=7.6 Hz, 1H), 7.83-7.70 (m, 2H), 7.62 (d, J=7.2 Hz, 1H), 7.53-7.41 (m, 4H), 7.40-7.29 (m, 2H), 7.16-7.07 (m, 1H), 6.96 (d, J=8.8 Hz, 2H), 6.70 (d, J=7.6 Hz, 1H), 5.11 (dd, J=5.6, 7.2 Hz, 1H), 4.98 (s, 2H), 4.27 (t, J=12.4 Hz, 2H), 3.92 (t, J=5.6 Hz, 2H), 3.73 (s, 2H), 3.61 (s, 2H), 3.26 (s, 2H), 3.21 (s, 2H), 3.15 (s, 2H), 3.02 (t, J=5.6 Hz, 2H), 2.95-2.86 (m, 1H), 2.85-2.76 (m, 2H), 2.65-2.54 (m, 2H), 2.11-1.95 (m, 5H), 1.91 (s, 3H), 1.72 (d, J=13.6 Hz, 2H), 1.66 (d, J=3.6 Hz, 1H), 1.35-1.22 (m, 2H).

### Example 21. Preparation of Compound 128a

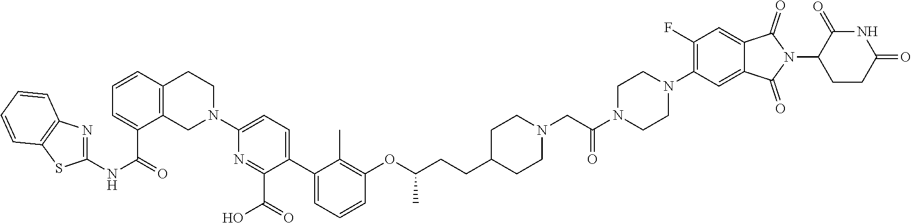 [SMILES: Cc1c(O[C@@H](C)CCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((2S)-4-(1-(2-(4-(2-(2,6-dioxopiperidin-3-yl)-6-fluoro-1,3-dioxoisoindolin-5-yl)piperazin-1-yl)-2-oxoethyl)piperidin-4-yl)butan-2-yl)oxy)-2-methylphenyl)picolinic acid

### Step A. Procedure for Preparation of tert-butyl 4-[3-[methoxy(methyl)amino]-3-oxo-propyl]piperidine-1-carboxylate

To a solution of 3-(1-tert-butoxycarbonyl-4-piperidyl)propanoic acid (4 g, 15.54 mmol, 1 equiv.) and N-methoxymethanamine (2.27 g, 23.32 mmol, 1.5 equiv., HCl) in DMF (10 mL) was added DIEA (6.03 g, 46.63 mmol, 8.12 mL, 3 equiv.) and HATU (8.87 g, 23.32 mmol, 1.5 equiv.). The mixture was stirred at 25° C. for 12 hours. The reaction mixture was quenched by addition H₂O (30 mL), and then extracted with ethyl acetate (30 mL×3). The combined organic layers were washed with brine (30 mL×3), dried over anhydrous sodium sulfate, filtered, and concentrated under reduced pressure to give a residue. The residue was purified by column chromatography (SiO₂, petroleum ether/ethyl acetate 10/1 to 1/1) to give tert-butyl 4-[3-[methoxy(methyl)amino]-3-oxo-propyl]piperidine-1-carboxylate (4.5 g, 14.9 mmol, 96.3% yield) as a yellow oil.

¹H NMR (400 MHz, CDCl₃,) 6=3.69 (s, 3H), 3.18 (s, 3H), 2.68 (t, J=12.0 Hz, 2H), 2.45 (t, J=8.0 Hz, 2H), 1.69 (s, 1H), 1.66 (s, 2H), 1.63-1.56 (m, 2H), 1.45 (s, 11H), 1.18-1.05 (m, 2H).

### Step B. Procedure for Preparation of tert-butyl 4-(3-oxobutyl)piperidine-1-carboxylate

A mixture of tert-butyl 4-[3-[methoxy(methyl)amino]-3-oxo-propyl]piperidine-1-carboxylate (4.5 g, 14.98 mmol, 1 equiv.) in THF (50 mL) was degassed and purged with N₂ 3 times, and then MeMgBr (3 M, 29.96 mL, 6 equiv.) was added dropwise to the mixture and stirred at 0° C. for 4 hours under N₂ atmosphere. The reaction mixture was quenched by addition saturated ammonium chloride (50 mL) at 0° C., and then diluted with water (50 mL) and extracted with CH₂Cl₂ (50 mL×3). The combined organic layers were washed with brine (50 mL×3), dried over anhydrous sodium sulfate, filtered, and concentrated under reduced pressure to give tert-butyl 4-(3-oxobutyl)piperidine-1-carboxylate (3.23 g, 12.65 mmol, 84.44% yield) as a white solid.

¹H NMR (400 MHz, DMSO-d₆,) 6=3.90 (d, J=11.2 Hz, 2H), 2.64-2.63 (m, 2H), 2.44 (t, J=7.2 Hz, 2H), 2.07 (s, 3H), 1.59 (d, J=12.4 Hz, 2H), 1.41-1.35 (m, 12H), 0.98-0.86 (m, 2H).

### Step C. Procedure for Preparation of tert-butyl 4-(3-hydroxybutyl)piperidine-1-carboxylate

To a solution of tert-butyl 4-(3-oxobutyl)piperidine-1-carboxylate (2 g, 7.83 mmol, 1 equiv.) in EtOH (20 mL) was added NaBH₄ (296.32 mg, 7.83 mmol, 1 equiv.). The mixture was stirred at 0° C. for 1 hour. The reaction mixture was concentrated under reduced pressure to remove EtOH, and then diluted with EtOAc (10 mL) and saturated ammonium chloride solution (10 mL) and extracted with EtOAc (10 mL×3). The combined organic layers were washed with brine (10 mL×3), dried over anhydrous sodium sulfate, filtered, and concentrated under reduced pressure to give tert-butyl 4-(3-hydroxybutyl)piperidine-1-carboxylate (1.8 g, 6.99 mmol, 89.30% yield) as a white solid. 1H NMR (400 MHz, DMSO-d₆,) 6=4.31 (d, J=4.4 Hz, 1H), 3.90 (d, J=12.4 Hz, 2H), 3.58-3.48 (m, 1H), 2.73-2.61 (m, 2H), 1.61 (d, J=12.4 Hz, 2H), 1.38 (s, 12H), 1.33-1.22 (m, 4H), 1.02 (d, J=6.0 Hz, 3H).

### Step D. Procedure for Preparation of tert-butyl (S)-4-(3-hydroxybutyl)piperidine-1-carboxylate

Tert-butyl 4-(3-hydroxybutyl)piperidine-1-carboxylate (3 g, 11.66 mmol, 1 equiv.) was separated by SFC to give tert-butyl (S)-4-(3-hydroxybutyl)piperidine-1-carboxylate (1.2 g, 4.66 mmol, 40.00% yield) was obtained as a colorless oil, which was confirmed by HNMR and tert-butyl 4-[(3R)-3-hydroxybutyl]piperidine-1-carboxylate (1.08 g, 4.20 mmol, 36.00% yield) as a pink oil.

¹H NMR (400 MHz, DMSO-d₆,) 6=4.29 (d, J=4.4 Hz, 1H), 3.90 (d, J=12.4 Hz, 2H), 3.61-3.46 (m, 1H), 2.66-2.65 (m, 2H), 1.61 (d, J=12.8 Hz, 2H), 1.38 (s, 9H), 1.35-1.23 (m, 4H), 1.21-1.11 (m, 1H), 1.02 (d, J=6.0 Hz, 3H), 0.99-0.85 (m, 2H).

### Step E. Procedure for Preparation of tert-butyl 4-[(3S)-3-(3-bromo-2-methyl-phenoxy)butyl]piperidine-1-carboxylate

To a solution of tert-butyl (S)-4-(3-hydroxybutyl)piperidine-1-carboxylate (1.1 g, 4.27 mmol, 1 equiv.) and 3-bromo-2-methyl-phenol (959.27 mg, 5.13 mmol, 1.2 equiv.) in toluene (10 mL) was added 2-(tributyl-λ5-phosphanylidene)acetonitrile (1.55 g, 6.41 mmol, 1.5 equiv.). The mixture was stirred at 120° C. for 12 hours. The reaction mixture was concentrated under reduced pressure to give a residue. The residue was purified by column chromatography (SiO₂, petroleum ether/ethyl acetate 0/1 to 10/1) to give tert-butyl 4-[(3S)-3-(3-bromo-2-methyl-phenoxy)butyl]piperidine-1-carboxylate (1.86 g, crude) as a colorless oil.

¹H NMR (400 MHz, CDCl₃,) 6=7.13 (d, J=8.0 Hz, 1H), 6.98 (t, J=8.0 Hz, 1H), 6.76 (d, J=8.4 Hz, 1H), 4.08 (d, J=12.4 Hz, 2H), 2.68 (t, J=11.2 Hz, 2H), 2.30 (s, 3H), 1.70-1.58 (m, 4H), 1.46 (s, 9H), 1.29 (d, J=6.4 Hz, 5H), 1.15-1.07 (m, 2H), 0.94-0.81 (m, 2H).

### Step F. Procedure for Preparation of 4-[(3S)-3-(3-bromo-2-methyl-phenoxy)butyl]piperidine

A solution of tert-butyl 4-[(3S)-3-(3-bromo-2-methyl-phenoxy)butyl]piperidine-1-carboxylate (1.86 g, 4.36 mmol, 1 equiv.) in HCl/EtOAc (4 M, 10 mL, 9.17 equiv.) was stirred at 25° C. for 1 hour. The reaction mixture was concentrated under reduced pressure to give a residue. The residue was purified by column chromatography (SiO₂, petroleum ether/ethyl acetate=1/1 to DCM:MeOH=10:1) to give 4-[(3S)-3-(3-bromo-2-methyl-phenoxy)butyl]piperidine (1.55 g, crude) as a light yellow oil.

### Step G. Procedure for Preparation of ethyl 2-[4-[(3S)-3-(3-bromo-2-methyl-phenoxy)butyl]-1-piperidyl]acetate

To a solution of 4-[(3S)-3-(3-bromo-2-methyl-phenoxy)butyl]piperidine (0.8 g, 2.45 mmol, 1 equiv.) and ethyl 2-bromoacetate (409.48 mg, 2.45 mmol, 271.18 μL, 1 equiv.) in ACN (10 mL) was added K₂CO₃ (1.02 g, 7.36 mmol, 3 equiv.). The mixture was stirred at 60° C. for 12 hours. The reaction mixture was filtered and then concentrated under reduced pressure to give a residue. The residue was purified by column chromatography (SiO₂, petroleum ether/ethyl acetate=0/1 to DCM:MeOH=10/1) to give ethyl 2-[4-[(3S)-3-(3-bromo-2-methyl-phenoxy)butyl]-1-piperidyl]acetate (575 mg, 1.3 mmol, 56.8% yield) as a yellow oil.

MS (ESI) m/z: 414.1 [M+H]⁺

¹H NMR (400 MHz, CDCl₃,) 6=7.13 (d, J=8.0 Hz, 1H), 6.98 (t, J=8.0 Hz, 1H), 6.76 (d, J=8.0 Hz, 1H), 4.35-4.27 (m, 1H), 4.24-4.15 (m, 2H), 3.21 (s, 2H), 2.94 (d, J=10.8 Hz, 2H), 2.30 (s, 3H), 2.18-2.15 (m, 2H), 1.75-1.62 (m, 4H), 1.40-1.25 (m, 11H).

### Step H. Procedure for Preparation of tert-butyl 6-[8-(1,3-benzothiazol-2-ylcarbamoyl)-3,4-dihydro-1H-isoquinolin-2-yl]-3-[3-[(1S)-3-[1-(2-ethoxy-2-oxo-ethyl)-4-piperidyl]-1-methyl-propoxy]-2-methyl-phenyl]pyridine-2-carboxylate

Ethyl 2-[4-[(3S)-3-(3-bromo-2-methyl-phenoxy)butyl]-1-piperidyl]acetate (575 mg, 1.39 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (854.14 mg, 1.39 mmol, 1 equiv.), [2-(2-aminophenyl)phenyl]palladium(1+); bis(1-adamantyl)-butyl-phosphane; methanesulfonate (203.10 mg, 278.88 μmol, 0.2 equiv.), and KF (1.5 M, 2.79 mL, 3 equiv.) were taken up into a microwave tube in dioxane (5 mL). The sealed tube was heated at 100° C. for 60 minutes under microwave. The reaction mixture was concentrated under reduced pressure to give a residue. The residue was purified by column chromatography (SiO₂, petroleum ether/ethyl acetate 0/1 to 1/1) to give tert-butyl 6-[8-(1,3-benzothiazol-2-ylcarbamoyl)-3,4-dihydro-1H-isoquinolin-2-yl]-3-[3-[(1S)-3-[1-(2-ethoxy-2-oxo-ethyl)-4-piperidyl]-1-methyl-propoxy]-2-methyl-phenyl]pyridine-2-carboxylate (0.616 g, 753.03 μmol, 54.00% yield) as a yellow oil.

MS (ESI) m/z: 818.6 [M+H]⁺

¹H NMR (400 MHz, DMSO-d₆,) δ=12.86 (s, 1H), 8.02 (d, J=7.6 Hz, 1H), 7.78 (d, J=8.0 Hz, 1H), 7.60 (d, J=7.6 Hz, 1H), 7.49-7.42 (m, 3H), 7.41-7.31 (m, 2H), 7.13-7.04 (m, 1H), 6.97-6.88 (m, 2H), 6.54 (d, J=7.2 Hz, 1H), 4.97 (s, 2H), 4.42-4.41 (m, 1H), 4.07-4.01 (m, 4H), 3.87 (t, J=5.2 Hz, 2H), 3.30 (s, 4H), 3.13 (s, 2H), 3.03 (t, J=6.0 Hz, 3H), 2.77 (d, J=9.2 Hz, 2H), 2.12-2.04 (m, 2H), 1.99 (s, 4H), 1.85 (s, 3H), 1.65-1.53 (m, 4H), 1.00 (s, 9H).

### Step I. Procedure for preparation of 2-[4-[(3S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(1S)-3-[1-(2-ethoxy-2-oxo-ethyl)-4-piperidyl]-1-methyl-propoxy]-2-methyl-phenyl]pyridine-2-carboxylate (0.616 g, 753.03 μmol, 1 equiv.) in THF (3 mL) and H₂O (3 mL) was added LiOH·H₂O (158.00 mg, 3.77 mmol, 5 equiv.). The mixture was stirred at 25° C. for 2 hours. The reaction mixture was quenched by addition water (10 mL), and then filtered and concentrated under reduced pressure to give 2-[4-[(3S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (640 mg, crude) as a yellow solid.

### Step J. Procedure for Preparation of tert-butyl 6-(8-(benzo[d]thiazol-2-ylcarbamoyl)-3,4-dihydroisoquinolin-2(1H)-yl)-3-(3-(((2S)-4-(1-(2-(4-(2-(2,6-dioxopiperidin-3-yl)-6-fluoro-1,3-dioxoisoindolin-5-yl)piperazin-1-yl)-2-oxoethyl)piperidin-4-yl)butan-2-yl)oxy)-2-methylphenyl)picolinate

To a solution of 2-[4-[(3S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (100 mg, 126.59 μmol, 1 equiv.) and 2-(2,6-dioxopiperidin-3-yl)-5-fluoro-6-(piperazin-1-yl)isoindoline-1,3-dione (45.61 mg, 126.59 μmol, 1 equiv.) in pyridine (2 mL) was added EDCI (36.40 mg, 189.88 μmol, 1.5 equiv.). The mixture was stirred at 25° C. for 12 hours. The reaction mixture was quenched by addition of H₂O (2 mL). The reaction mixture was filtered, and the filter cake was washed with 5 mL of water. After filtration, the filter cake was diluted in DCM (10 mL). The combined organic layers were dried over Na₂SO₄, filtered, and concentrated under reduced pressure to give a residue. The residue was purified by prep-TLC (SiO2, DCM:MeOH=10:1) to give tert-butyl 6-(8-(benzo[d]thiazol-2-ylcarbamoyl)-3,4-dihydroisoquinolin-2(1H)-yl)-3-(3-(((2S)-4-(1-(2-(4-(2-(2,6-dioxopiperidin-3-yl)-6-fluoro-1,3-dioxoisoindolin-5-yl)piperazin-1-yl)-2-oxoethyl)piperidin-4-yl)butan-2-yl)oxy)-2-methylphenyl)picolinate (50 mg, 41.9 μmol, 33.1% yield, 95.0% purity) as a yellow solid.

MS (ESI) m/z: 1132.3 [M+H]⁺

### Step K. Procedure for Preparation of 6-(8-(benzo[d]thiazol-2-ylcarbamoyl)-3,4-dihydroisoquinolin-2(1H)-yl)-3-(3-(((2S)-4-(1-(2-(4-(2-(2,6-dioxopiperidin-3-yl)-6-fluoro-1,3-dioxoisoindolin-5-yl)piperazin-1-yl)-2-oxoethyl)piperidin-4-yl)butan-2-yl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((2S)-4-(1-(2-(4-(2-(2,6-dioxopiperidin-3-yl)-6-fluoro-1,3-dioxoisoindolin-5-yl)piperazin-1-yl)-2-oxoethyl)piperidin-4-yl)butan-2-yl)oxy)-2-methylphenyl)picolinate (50 mg, 44.16 μmol, 1 equiv.) in DCM (0.5 mL) was added TFA (5.04 mg, 44.16 μmol, 3.27 μL, 1 equiv.). The mixture was stirred at 25° C. for 12 hours. The reaction mixture was filtered and concentrated under reduced pressure to give a residue. The crude product was purified by prep-HPLC to give 6-(8-(benzo[d]thiazol-2-ylcarbamoyl)-3,4-dihydroisoquinolin-2(1H)-yl)-3-(3-(((2S)-4-(1-(2-(4-(2-(2,6-dioxopiperidin-3-yl)-6-fluoro-1,3-dioxoisoindolin-5-yl)piperazin-1-yl)-2-oxoethyl)piperidin-4-yl)butan-2-yl)oxy)-2-methylphenyl)picolinic acid (21.4 mg, 19.8 μmol, 44.8% yield, 99.3% purity) as a yellow solid.

MS (ESI) m/z: 1076.8 [M+H]⁺.

¹H NMR (400 MHz, DMSO-d₆) δ=13.01-12.61 (m, 1H), 11.11 (s, 1H), 8.14 (s, 1H), 8.03 (d, J=7.6 Hz, 1H), 7.83-7.71 (m, 2H), 7.62 (d, J=7.2 Hz, 1H), 7.51-7.41 (m, 4H), 7.40-7.31 (m, 2H), 7.11-7.04 (m, 1H), 6.96 (d, J=8.8 Hz, 1H), 6.88 (d, J=8.4 Hz, 1H), 6.61 (d, J=7.6 Hz, 1H), 5.11 (dd, J=5.6, 7.6 Hz, 1H), 4.97 (s, 2H), 4.40-4.39 (m, 1H), 3.91 (t, J=5.6 Hz, 2H), 3.65 (d, J=16.0 Hz, 4H), 3.29 (s, 4H), 3.23 (s, 4H), 3.02 (t, J=6.0 Hz, 2H), 2.95-2.81 (m, 2H), 2.64-2.53 (m, 2H), 2.08-2.00 (m, 1H), 1.87 (s, 3H), 1.73-1.55 (m, 4H), 1.44-1.13 (m, 9H).

### Example 22. Preparation of Compound 128b

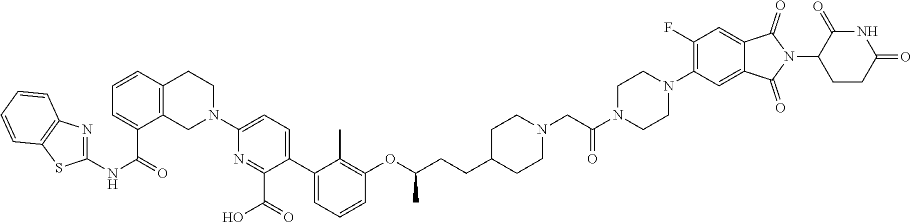 [SMILES: Cc1c(O[C@H](C)CCC2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[(1R)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]-1-methyl-propoxy]-2-methyl-phenyl]pyridine-2-carboxylic acid

### Step A. Procedure for Preparation of tert-butyl 4-[(3R)-3-(3-bromo-2-methyl-phenoxy)butyl]piperidine-1-carboxylate

A mixture of tert-butyl 4-[(3R)-3-hydroxybutyl]piperidine-1-carboxylate (1.08 g, 4.20 mmol, 1 equiv.), 3-bromo-2-methyl-phenol (784.85 mg, 4.20 mmol, 1 equiv.), 2-(tributyl-X⁵-phosphanylidene)acetonitrile (1.22 g, 5.04 mmol, 1.2 equiv.) in toluene (2 mL) was degassed and purged with N₂ 3 times, and then the mixture was stirred at 120° C. for 1 hour under N₂ atmosphere. The reaction mixture was concentrated under reduced pressure to give a residue. The residue was purified by column chromatography (SiO₂, petroleum ether/ethyl acetate 10/1 to 3/1) to give tert-butyl 4-[(3R)-3-(3-bromo-2-methyl-phenoxy)butyl]piperidine-1-carboxylate (1.3 g, 3.0 mmol, 72.6% yield) as a colorless oil.

¹H NMR (400 MHz, CDCl₃,) 6=7.13 (t, J=8.4 Hz, 1H), 6.98 (t, J=8.0 Hz, 1H), 6.76 (d, J=8.4 Hz, 1H), 4.36-4.25 (m, 1H), 4.16-4.01 (m, 2H), 2.68 (t, J=12.4 Hz, 2H), 2.30 (s, 3H), 1.80-1.58 (m, 5H), 1.47 (s, 9H), 1.44-1.35 (m, 2H), 1.29 (d, J=6.0 Hz, 3H), 1.16-1.05 (m, 2H).

### Step B. Procedure for Preparation of 4-[(3R)-3-(3-bromo-2-methyl-phenoxy)butyl]piperidine

A solution of tert-butyl 4-[(3R)-3-(3-bromo-2-methyl-phenoxy)butyl]piperidine-1-carboxylate (1.3 g, 3.05 mmol, 1 equiv.) in HCl/EtOAc (1 mL) was stirred at 25° C. for 12 hours. The reaction mixture was concentrated under reduced pressure to give a residue. The residue was purified by column chromatography (SiO₂, petroleum ether/ethyl acetate 10/1 to 3/1) to give 4-[(3R)-3-(3-bromo-2-methyl-phenoxy)butyl]piperidine (650 mg, 1.9 mmol, 65.3% yield) as a colorless oil.

¹H NMR (400 MHz, CDCl₃,) 6=9.59 (s, 1H), 7.13 (d, J=8.0 Hz, 1H), 6.98 (t, J=8.0 Hz, 1H), 6.74 (d, J=8.4 Hz, 1H), 4.39-4.20 (m, 1H), 3.48 (d, J=12.4 Hz, 2H), 2.84 (m, J=12.0 Hz, 2H), 2.28 (s, 3H), 1.90 (d, J=13.6 Hz, 2H), 1.71-1.37 (m, 7H), 1.28 (d, J=6.0 Hz, 3H).

### Step C. Procedure for Preparation of ethyl 2-[4-[(3R)-3-(3-bromo-2-methyl-phenoxy) butyl]-1-piperidyl]acetate

To a solution of 4-[(3R)-3-(3-bromo-2-methyl-phenoxy)butyl]piperidine (650 mg, 1.99 mmol, 1 equiv.), ethyl 2-bromoacetate (332.70 mg, 1.99 mmol, 220.33 μL, 1 equiv.) in MeCN (3 mL) was added K₂CO₃ (275.34 mg, 1.99 mmol, 1 equiv.). The mixture was stirred at 60° C. for 2 hours. The reaction mixture was filtered and then concentrated under reduced pressure to give a residue. The residue was purified by column chromatography (SiO₂, petroleum ether/ethyl acetate 10/1 to 2/1) to give ethyl 2-[4-[(3R)-3-(3-bromo-2-methyl-phenoxy) butyl]-1-piperidyl]acetate (600 mg, 1.46 mmol, 73.04% yield) as a yellow oil.

MS (ESI) m/z: 414.2 [M+H]⁺

¹H NMR (400 MHz, CDCl₃,) 6=7.13 (d, J=8.0 Hz, 1H), 6.98 (t, J=8.0 Hz, 1H), 6.76 (d, J=8.4 Hz, 1H), 4.35-4.26 (m, 1H), 4.19 (q, J=7.2 Hz, 2H), 3.19 (s, 2H), 2.93 (d, J=11.2 Hz, 2H), 2.30 (s, 3H), 2.13 (t, J=11.2 Hz, 2H), 1.80-1.56 (m, 6H), 1.36-1.26 (m, 9H).

### Step D. Procedure for Preparation of tert-butyl 6-[8-(1,3-benzothiazol-2-ylcarbamoyl)-3,4-dihydro-1H-isoquinolin-2-yl]-3-[3-[(1R)-3-[1-(2-ethoxy-2-oxo-ethyl)-4-piperidyl]-1-methyl-propoxy]-2-methyl-phenyl]pyridine-2-carboxylate

A mixture of ethyl 2-[4-[(3R)-3-(3-bromo-2-methyl-phenoxy) butyl]-1-piperidyl]acetate (700 mg, 1.70 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.04 g, 1.70 mmol, 1 equiv.), [2-(2-aminophenyl)phenyl]palladium(1+); bis(1-adamantyl)-butyl-phosphane; methanesulfonate (123.63 mg, 169.75 μmol, 0.1 equiv.), and KF (1.5 M, 3.40 mL, 3 equiv.) in dioxane (5 mL) and water (1 mL) was degassed and purged with N₂ 3 times, and then the mixture was stirred at 100° C. for 1 hour under microwave. The reaction mixture was filtered and then concentrated under reduced pressure to give a residue. The residue was purified by column chromatography (SiO₂, petroleum ether/ethyl acetate 10/1 to 2/1) to give tert-butyl 6-[8-(1,3-benzothiazol-2-ylcarbamoyl)-3,4-dihydro-1H-isoquinolin-2-yl]-3-[3-[(1R)-3-[1-(2-ethoxy-2-oxo-ethyl)-4-piperidyl]-1-methyl-propoxy]-2-methyl-phenyl]pyridine-2-carboxylate (850 mg, 1.0 mmol, 61.21% yield) as a yellow oil.

MS (ESI) m/z: 818.7 [M+H]⁺

¹H NMR (400 MHz, CDCl₃,) 6=7.86 (d, J=8.0 Hz, 1H), 7.63 (d, J=8.0 Hz, 1H), 7.57 (d, J=7.2 Hz, 1H), 7.43-7.28 (m, 5H), 7.08 (t, J=8.0 Hz, 1H), 6.90 (d, J=8.8 Hz, 1H), 6.80 (d, J=8.0 Hz, 1H), 6.67 (d, J=7.6 Hz, 1H), 5.23-4.85 (m, 2H), 4.42-4.29 (m, 1H), 4.14-4.10 (m, 4H), 3.22 (s, 2H), 3.07 (t, J=6.4 Hz, 2H), 3.01-2.88 (m, 2H), 2.25-2.09 (m, 2H), 2.05 (s, 5H), 1.68 (s, 4H), 1.50-1.41 (m, 2H), 1.32 (d, J=5.6 Hz, 3H), 1.26 (d, J=3.2 Hz, 4H), 1.14 (s, 9H).

### Step E. Procedure for Preparation of 2-[4-[(3R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(1R)-3-[1-(2-ethoxy-2-oxo-ethyl)-4-piperidyl]-1-methyl-propoxy]-2-methyl-phenyl]pyridine-2-carboxylate (200 mg, 244.49 μmol, 1 equiv.) in THF (0.5 mL), H₂O (0.5 mL) was added LiOH·H₂O (51.30 mg, 1.22 mmol, 5 equiv.). The mixture was stirred at 25° C. for 2 hours. The pH was adjusted to 6 with 1M HCl. The residue was extracted with DCM 6 mL (2 mL×3). The combined organic layers were filtered and then concentrated under reduced pressure to give a residue. The residue was purified by column chromatography (SiO₂, petroleum ether/ethyl acetate 10/1 to 3/1) to give 2-[4-[(3R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (160 mg, 202.54 μmol, 82.84% yield) as a yellow solid.

MS (ESI) m/z: 790.7 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[(1R)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]-1-methyl-propoxy]-2-methyl-phenyl]pyridine-2-carboxylate

To a solution of 2-[4-[(3R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (190 mg, 240.51 μmol, 1 equiv.) and 2-(2,6-dioxo-3-piperidyl)-5-fluoro-6-piperazin-1-yl-isoindoline-1,3-dione (104.00 mg, 288.62 μmol, 1.2 equiv.) in pyridine (0.5 mL) was added EDCI (69.16 mg, 360.77 μmol, 1.5 equiv.). The mixture was stirred at 25° C. for 1 hour. The mixture was quenched by water (2 mL) at 25° C. and precipitated in water and then filtered and concentrated under reduced pressure to give a residue to give tert-butyl 6-[8-(1,3-benzothiazol-2-ylcarbamoyl)-3,4-dihydro-1H-isoquinolin-2-yl]-3-[3-[(1R)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]-1-methyl-propoxy]-2-methyl-phenyl]pyridine-2-carboxylate (170 mg, 150.1 μmol, 62.4% yield) as a yellow solid.

MS (ESI) m/z: 1132.8 [M+H]⁺

### Step G. Procedure for Preparation of 6-[8-(1,3-benzothiazol-2-ylcarbamoyl)-3,4-dihydro-1H-isoquinolin-2-yl]-3-[3-[(1R)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]-1-methyl-propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[(1R)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]-1-methyl-propoxy]-2-methyl-phenyl]pyridine-2-carboxylate (170 mg, 150.14 μmol, 1 equiv.) in DCM (1 mL) and TFA (1 mL) was stirred at 25° C. for 12 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[(1R)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]-1-methyl-propoxy]-2-methyl-phenyl]pyridine-2-carboxylic acid (36.9 mg, 33.5 μmol, 22.3% yield, 97.6% purity) as a yellow solid.

MS (ESI) m/z: 1076.7 [M+H]⁺

¹HNMR (400 MHz, DMSO-d₆) δ=13.08-12.63 (m, 1H), 11.11 (s, 1H), 8.13 (s, 1H), 8.03 (d, J=8.0 Hz, 1H), 7.81-7.75 (m, 2H), 7.62 (d, J=7.6 Hz, 1H), 7.51-7.42 (m, 4H), 7.40-7.32 (m, 2H), 7.12-7.05 (m, 1H), 6.96 (d, J=8.8 Hz, 1H), 6.89 (d, J=8.4 Hz, 1H), 6.61 (d, J=7.6 Hz, 1H), 5.11 (dd, J=5.6, 12.8 Hz, 1H), 4.98 (s, 2H), 4.42 (d, J=2.8 Hz, 1H), 3.91 (t, J=5.6 Hz, 2H), 3.91-3.80 (m, 1H), 3.67 (s, 2H), 3.59 (s, 2H), 3.27 (s, 6H), 3.02 (t, J=5.6 Hz, 2H), 2.94-2.84 (m, 2H), 2.70-2.53 (m, 4H), 2.08-2.01 (m, 1H), 1.88 (s, 3H), 1.77 (d, J=14.8 Hz, 2H), 1.68-1.57 (m, 2H), 1.41 (d, J=1.6 Hz, 5H), 1.24 (d, J=5.6 Hz, 4H).

### Example 23. Preparation of Compound 129a

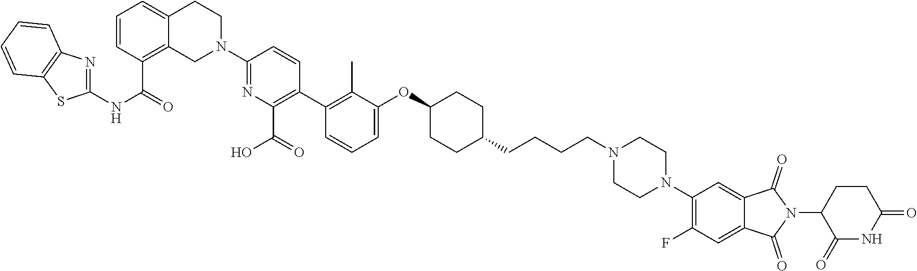 [SMILES: Cc1c(O[C@H]2CC[C@H](CCCCN3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)butyl)cyclohexyl)oxy)-2-methylphenyl)picolinic acid

### Step A. Procedure for Preparation of 1-(((1r,4r)-4-((E)-4-(benzyloxy)but-1-en-1-yl)cyclohexyl)oxy)-3-bromo-2-methylbenzene

To a mixture of 3-benzyloxypropyl(triphenyl)phosphonium; bromide (5.56 g, 11.31 mmol, 1.2 equiv.) in THF (30 mL) was added LiHMDS (1 M, 12.25 mL, 1.3 equiv.) at −70° C. under N₂ atmosphere for 1 hour. Then 4-(3-bromo-2-methyl-phenoxy)cyclohexanecarbaldehyde (2.8 g, 9.42 mmol, 1 equiv.) was added the mixture at −70° C. under N₂ atmosphere. The mixture was stirred at 25° C. for 11 hours under N₂ atmosphere. The reaction mixture was quenched by addition NH₄Cl (100 mL) at 0° C. and then diluted with H₂O (80 mL) and extracted with ethyl acetate. The combined organic layers were washed with, dried over Na₂SO₄, filtered, and concentrated under reduced pressure to give a residue. The residue was purified by column chromatography (SiO₂, petroleum ether/ethyl acetate 30/1 to 10/1) to give 1-(((1r,4r)-4-((E)-4-(benzyloxy)but-1-en-1-yl)cyclohexyl)oxy)-3-bromo-2-methylbenzene (3 g, 6.9 mmol, 74.1% yield) as a yellow oil.

¹H NMR (400 MHz, CDCl₃) δ=7.21-7.12 (m, 5H), 6.98 (d, J=8.0 Hz, 1H), 6.81 (t, J=8.0 Hz, 1H), 6.65 (d, J=8.0 Hz, 1H), 5.25-5.06 (m, 2H), 4.42-4.33 (m, 2H), 4.00-3.87 (m, 1H), 3.33 (t, J=6.8 Hz, 2H), 2.29-2.21 (m, 2H), 2.14 (s, 3H), 1.97 (d, J=10.4 Hz, 2H), 1.66-1.50 (m, 2H), 1.41-1.26 (m, 3H), 1.10-0.97 (m, 2H)

### Step B. Procedure for Preparation of 4-((1s,4r)-4-(3-bromo-2-methylphenoxy)cyclohexyl)butan-1-ol

To a solution of 1-(((1r,4r)-4-((E)-4-(benzyloxy)but-1-en-1-yl)cyclohexyl)oxy)-3-bromo-2-methylbenzene (2.6 g, 6.06 mmol, 1 equiv.) in EtOAc (20 mL) was added PtO₂ (1.37 g, 6.06 mmol, 1.0 equiv.) under N₂ atmosphere. The suspension was degassed and purged with H₂. The mixture was stirred under H₂ (15 psi) at 40° C. for 12 hours. The reaction mixture was filtered and then concentrated under reduced pressure to give a residue. The residue was purified by prep-HPLC to give 4-((1s,4r)-4-(3-bromo-2-methylphenoxy)cyclohexyl)butan-1-ol (1 g, 2.3 mmol, 48.3% yield) as a yellow oil.

¹H NMR (400 MHz, CDCl₃) δ=7.06 (d, J=7.6 Hz, 1H), 6.89 (t, J=8.0 Hz, 1H), 6.76-6.69 (m, 1H), 4.06-3.97 (m, 1H), 3.58 (t, J=6.8 Hz, 2H), 2.22 (s, 3H), 2.08-2.01 (m, 2H), 1.80-1.74 (m, 2H), 1.51-1.47 (m, 2H), 1.39-1.28 (m, 4H), 1.23-1.14 (m, 3H), 1.00-0.88 (m, 2H)

### Step C. Procedure for Preparation of 4-((1s,4r)-4-(3-bromo-2-methylphenoxy)cyclohexyl)butanal

To a solution of oxalyl dichloride (371.92 mg, 2.93 mmol, 256.50 μL, 2 equiv.) in DCM (5 mL) was added dropwise to a solution of DMSO (457.86 mg, 5.86 mmol, 457.86 μL, 4 equiv.) in DCM (5 mL) at −70° C. under N₂ atmosphere. The mixture was stirred at −70° C. for 30 minutes. After which time 4-((1s,4r)-4-(3-bromo-2-methylphenoxy)cyclohexyl)butan-1-ol (500 mg, 1.47 mmol, 1 equiv.) in DCM (5 mL) was added dropwise at −70° C. The solution was stirred for 30 minutes at −70° C. Then TEA (889.50 mg, 8.79 mmol, 1.22 mL, 6 equiv.) was added into the solution. The solution was stirred at −70° C. for 2 hours under N₂ atmosphere. The reaction was diluted with water (50 mL) and extracted with DCM 80 mL (80 mL×2). The combined organic layers were washed with brine 60 mL (60 mL×1), dried over Na₂SO₄, filtered, and concentrated under reduced pressure to give a residue. The residue was purified by column chromatography (SiO₂, petroleum ether/ethyl acetate 100/1 to 15/1) to give 4-((1s,4r)-4-(3-bromo-2-methylphenoxy)cyclohexyl)butanal (400 mg, 1.8 mmol, 80.4% yield) as a yellow oil.

### Step D. Procedure for Preparation of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (400 mg, 653.01 μmol, 1 equiv.), 4-((1s,4r)-4-(3-bromo-2-methylphenoxy)cyclohexyl)butanal (243.70 mg, 718.31 μmol, 1.1 equiv.), [2-(2-aminophenyl)phenyl]palladium (1+); bis(1-adamantyl)-butyl-phosphane; methanesulfonate (47.56 mg, 65.30 μmol, 0.1 equiv.), and K₂CO₃ (270.75 mg, 1.96 mmol, 3 equiv.) in dioxane (4 mL) and H₂O (1 mL) was degassed and purged with N₂ and then stirred at 100° C. for 1 hour under N₂ atmosphere. The reaction mixture was concentrated under reduced pressure to give a residue. The residue was purified by column chromatography (SiO₂, petroleum ether/ethyl acetate 20/1 to 2/1) to give tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (120 mg, 161.0 μmol, 24.7% yield) as a yellow solid.

MS (ESI) m/z: 745.5 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)butyl)cyclohexyl)oxy)-2-methylphenyl)picolinate

To a solution of 2-(2,6-dioxo-3-piperidyl)-5-fluoro-6-piperazin-1-yl-isoindoline-1,3-dione (29.02 mg, 80.54 μmol, 1.2 equiv.) in DCM (2 mL) was added NaBH(OAc)₃ (42.68 mg, 201.36 μmol, 3 equiv.), AcOH (8.06 mg, 134.24 μmol, 7.68 μL, 2 equiv.), and tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (50 mg, 67.12 μmol, 1 equiv.) at 0° C. The mixture was stirred at 25° C. for 1 hour. The reaction was diluted with water (20 mL) and extracted with DCM. The combined organic layers were washed with brine 30 mL (30 mL×1), dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)butyl)cyclohexyl)oxy)-2-methylphenyl)picolinate (65 mg, 59.7 μmol, 88.9% yield) as a yellow oil.

MS (ESI) m/z: 1098.7 [M+H]⁺.

### Step F. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)butyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)butyl)cyclohexyl)oxy)-2-methylphenyl)picolinate (65 mg, 59.67 μmol, 1 equiv.) in DCM (0.5 mL) was added TFA (770.00 mg, 6.75 mmol, 0.5 mL, 113.17 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)butyl)cyclohexyl)oxy)-2-methylphenyl)picolinic acid (28.1 mg, 26.6 μmol, 44.7% yield, 97.8% purity) as a pink solid.

MS (ESI) m/z: 1033.7 [M+H]⁺.

¹H NMR (400 MHz, DMSO-d₆) δ=12.96-12.76 (m, 1H), 11.11 (s, 1H), 8.03 (d, J=8.0 Hz, 1H), 7.82-7.71 (m, 2H), 7.62 (d, J=7.6 Hz, 1H), 7.50-7.42 (m, 4H), 7.40-7.32 (m, 2H), 7.10-7.04 (m, 1H), 6.99-6.88 (m, 2H), 6.61 (d, J=7.6 Hz, 1H), 5.14-5.07 (m, 1H), 4.98 (s, 2H), 4.26-4.12 (m, 1H), 3.96-3.88 (m, 2H), 3.28-3.22 (m, 4H), 3.02 (t, J=5.6 Hz, 2H), 2.93-2.84 (m, 1H), 2.63-2.52 (m, 6H), 2.47-2.35 (m, 2H), 2.09-2.02 (m, 2H), 1.87 (s, 3H), 1.78 (d, J=12.0 Hz, 2H), 1.51-1.42 (m, 2H), 1.39-1.18 (m, 8H), 1.11-0.99 (m, 2H)

### Example 24. Preparation of Compound 130a

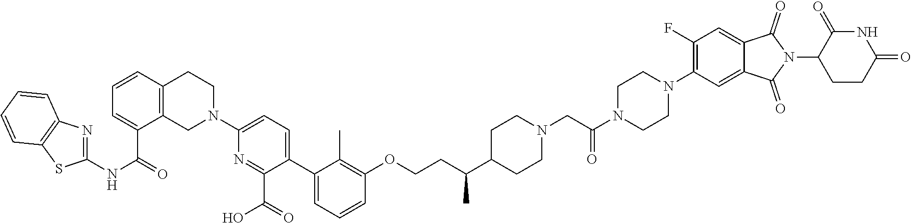 [SMILES: Cc1c(OCC[C@H](C)C2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl-3-[3-[(3S)-3-1-[2-[4-[2-(2,6-dioxo-3-piperidyl)-6-fluoro-1,3-dioxo-isoindolin-5-yl]piperazin-1-yl]-2-oxo-ethyl]-4-piperidyl]butoxy]-2-methyl-phenyl]pyridine-2-carboxylic acid

### Step A. Procedure for Preparation of 4-[(1S)-3-(3-bromo-2-methyl-phenoxy)-1-methyl-propyl]piperidine

A mixture of tert-butyl 4-[(1S)-3-(3-bromo-2-methyl-phenoxy)-1-methyl-propyl]piperidine-1-carboxylate (700 mg, 1.64 mmol, 1 equiv.) and HCl/EtOAc (4 M, 7.00 mL, 17.06 equiv.) was stirred at 25° C. for 2 hours. The mixture was concentrated under reduced pressure to give a residue. The residue was triturated with EtOAc (3 mL) at 25° C. for 10 min to give 4-[(1S)-3-(3-bromo-2-methyl-phenoxy)-1-methyl-propyl]piperidine (550 mg, 1.5 mmol, 91.4% yield, 98.9% purity, HCl) as a yellow solid.

MS (ESI) m/z: 327.8 [M+3]⁺ (⁸⁰Br).

### Step B. Procedure for Preparation of ethyl 2-[4-[(1S)-3-(3-bromo-2-methyl-phenoxy)-1-methyl-propyl]-1-piperidyl]acetate

To a solution of 4-[(1S)-3-(3-bromo-2-methyl-phenoxy)-1-methyl-propyl]piperidine (550 mg, 1.52 mmol, 1 equiv., HCl) and ethyl 2-bromoacetate (227.90 mg, 1.36 mmol, 150.93 μL, 0.9 equiv.) in MeCN (6 mL) was added K₂CO₃ (628.67 mg, 4.55 mmol, 3 equiv.) The mixture was stirred at 60° C. for 1 hour. The mixture was filtered to give a filtrate, which was concentrated under reduced pressure to give a residue. The residue was purified by flash silica gel chromatography (eluent of 0-10% methanol/dichloromethane) to give ethyl 2-[4-[(1S)-3-(3-bromo-2-methyl-phenoxy)-1-methyl-propyl]-1-piperidyl]acetate (480 mg, 1.2 mmol, 76.3% yield, 99.5% purity) as a yellow oil.

MS (ESI) m/z: 414.0 [M+3]⁺ (⁸⁰Br).

¹H NMR (400 MHz, DMSO-d₆) δ=7.17-7.12 (m, 1H), 7.12-7.06 (m, 1H), 6.98 (d, J=8.0 Hz, 1H), 4.10-4.04 (m, 2H), 4.04-3.92 (m, 2H), 3.14 (s, 2H), 2.84 (d, J=11.2 Hz, 2H), 2.22 (s, 3H), 2.14-2.03 (m, 2H), 1.90-1.80 (m, 1H), 1.60-1.46 (m, 4H), 1.34-1.20 (m, 1H), 1.18 (t, J=7.2 Hz, 4H), 0.88 (d, J=6.4 Hz, 3H)

### Step C. Procedure for Preparation of tert-butyl 6-[8-(1,3-benzothiazol-2-ylcarbamoyl)-3,4-dihydro-1H-isoquinolin-2-yl]-3-[3-[(3S)-3-[1-(2-ethoxy-2-oxo-ethyl)-4-piperidyl]butoxy]-2-methyl-phenyl]pyridine-2-carboxylate

Ethyl 2-[4-[(1S)-3-(3-bromo-2-methyl-phenoxy)-1-methyl-propyl]-1-piperidyl]acetate (400 mg, 970.03 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (594.19 mg, 970.03 μmol, 1 equiv.), Ad₂nBuP Pd G₃ (70.64 mg, 97.00 μmol, 0.1 equiv.), and KF (169.07 mg, 2.91 mmol, 68.17 μL, 3 equiv.) were taken up into a microwave tube in dioxane (4 mL) and H₂O (0.4 mL). The sealed tube was heated at 100° C. for 1 hour under microwave. The mixture was concentrated under reduced pressure to give a residue. The residue was purified by flash silica gel chromatography (eluent of 0˜50% ethyl acetate/petroleum ether) to give tert-butyl 6-[8-(1,3-benzothiazol-2-ylcarbamoyl)-3,4-dihydro-1H-isoquinolin-2-yl]-3-[3-[(3S)-3-[1-(2-ethoxy-2-oxo-ethyl)-4-piperidyl]butoxy]-2-methyl-phenyl]pyridine-2-carboxylate (530 mg, 595.7 μmol, 61.4% yield, 91.9% purity) as a yellow solid.

MS (ESI) m/z: 818.7 [M+H]⁺

¹H NMR (400 MHz, DMSO-d₆) δ=12.85 (s, 1H), 8.01 (d, J=8.0 Hz, 1H), 7.77 (d, J=8.0 Hz, 1H), 7.60 (d, J=7.6 Hz, 1H), 7.50-7.39 (m, 3H), 7.38-7.31 (m, 2H), 7.13-7.06 (m, 1H), 6.96-6.89 (m, 2H), 6.57 (d, J=7.6 Hz, 1H), 4.97 (s, 2H), 4.08-4.03 (m, 2H), 4.02-3.92 (m, 2H), 3.87 (t, J=5.6 Hz, 2H), 3.13 (s, 2H), 3.02 (t, J=5.6 Hz, 2H), 2.83 (d, J=10.0 Hz, 2H), 2.06 (t, J=10.4 Hz, 2H), 1.86 (s, 3H), 1.84-1.75 (m, 1H), 1.60-1.44 (m, 4H), 1.34-1.20 (m, 2H), 1.18-1.14 (m, 4H), 1.03-0.98 (m, 9H), 0.88-0.82 (m, 3H)

### Step D. Procedure for Preparation of 2-[4-[(1S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(3S)-3-[1-(2-ethoxy-2-oxo-ethyl)-4-piperidyl]butoxy]-2-methyl-phenyl]pyridine-2-carboxylate (530 mg, 647.90 μmol, 1 equiv.) in THF (4 mL) was added LiOH (1 M, 1.94 mL, 3 equiv.). The mixture was stirred at 25° C. for 12 hours. The mixture was concentrated under reduced pressure to remove THF, HCl (1 M) was added to pH=6, and the mixture was filtered to give 2-[4-[(1S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid (500 mg, crude) as a yellow solid.

MS (ESI) m/z: 790.3 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[3-[(3S)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]butoxy]-2-methyl-phenyl]pyridine-2-carboxylate

To a solution of 2-[4-[(1S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid (150 mg, 189.88 μmol, 1 equiv.) and HATU (86.64 mg, 227.85 μmol, 1.2 equiv.) in DMF (1.5 mL) was added DIEA (73.62 mg, 569.64 μmol, 99.22 μL, 3 equiv.) and 2-(2,6-dioxo-3-piperidyl)-5-fluoro-6-piperazin-1-yl-isoindoline-1,3-dione (82.10 mg, 227.85 μmol, 1.2 equiv.). The mixture was stirred at 25° C. for 1 hour. The mixture was added into H₂O (5 mL), filtered to give a filter cake, which was concentrated under reduced pressure to give a residue. The residue was purified by prep-TLC (SiO₂, DCM:MeOH=10:1) to give tert-butyl 6-[8-(1,3-benzothiazol-2-ylcarbamoyl)-3,4-dihydro-1H-isoquinolin-2-yl]-3-[3-[(3S)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]butoxy]-2-methyl-phenyl]pyridine-2-carboxylate (110 mg, 95.1 μmol, 50.0% yield, 97.9% purity) as a yellow solid.

MS (ESI) m/z: 1132.3 [M+H]⁺

### Step F. Procedure for Preparation of 6-[8-(1,3-benzothiazol-2-ylcarbamoyl)-3,4-dihydro-1H-isoquinolin-2-yl]-3-[3-[(3S)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]butoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[(3S)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]butoxy]-2-methyl-phenyl]pyridine-2-carboxylate (100 mg, 88.32 μmol, 1 equiv.) in DCM (0.5 mL) was added TFA (770.00 mg, 6.75 mmol, 500.00 μL, 76.46 equiv.). The mixture was stirred at 40° C. for 12 hours. The mixture was concentrated under reduced pressure to remove DCM and diluted with DMF (2 mL). The mixture was purified by prep-HPLC to give 6-[8-(1,3-benzothiazol-2-ylcarbamoyl)-3,4-dihydro-1H-isoquinolin-2-yl]-3-[3-[(3S)-3-[1-[2-[4-[2-(2,6-dioxo-3-piperidyl)-6-fluoro-1,3-dioxo-isoindolin-5-yl]piperazin-1-yl]-2-oxo-ethyl]-4-piperidyl]butoxy]-2-methyl-phenyl]pyridine-2-carboxylic acid (70.1 mg, 60.7 μmol, 68.8% yield, 93.3% purity) as a yellow solid.

MS (ESI) m/z: 1076.5 [M+H]⁺

¹H NMR (400 MHz, DMSO-d₆) δ=11.17-11.03 (m, 1H), 8.23 (s, 1H), 8.01 (d, J=8.0 Hz, 1H), 7.82-7.71 (m, 2H), 7.62 (d, J=7.2 Hz, 1H), 7.49-7.40 (m, 3H), 7.38-7.31 (m, 2H), 7.12-7.03 (m, 1H), 6.89 (t, J=8.8 Hz, 2H), 6.64 (d, J=7.6 Hz, 1H), 5.16-5.06 (m, 1H), 4.97 (s, 2H), 4.03-3.95 (m, 2H), 3.90 (t, J=5.6 Hz, 2H), 3.75-3.72 (m, 2H), 3.61 (s, 2H), 3.28 (s, 2H), 3.22-3.20 (m, 2H), 3.14 (s, 2H), 3.02 (t, J=5.6 Hz, 2H), 2.92-2.82 (m, 4H), 2.61-2.56 (m, 2H), 2.09-1.91 (m, 4H), 1.88 (s, 3H), 1.60-1.53 (m, 3H), 1.30-1.16 (m, 3H), 0.89 (d, J=6.0 Hz, 3H)

### Example 25. Preparation of Compound 132

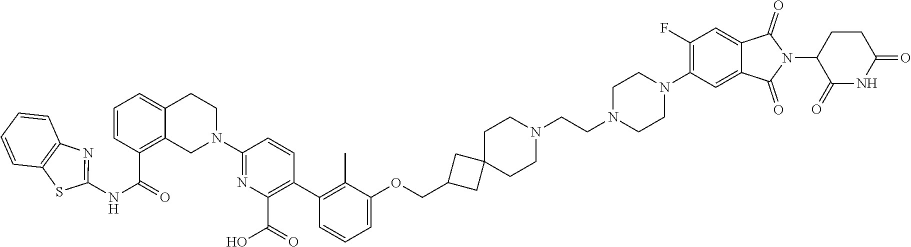 [SMILES: Cc1c(OCC2CC3(CCN(CCN4CCN(c5cc6c(cc5F)C(=O)N(C5CCC(=O)NC5=O)C6=O)CC4)CC3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-((7-(2-(4-(2-(2,6-dioxopiperidin-3-yl)-6-fluoro-1,3-dioxoisoindolin-5-yl)piperazin-1-yl)ethyl)-7-azaspiro[3.5]nonan-2-yl)methoxy)-2-methylphenyl)picolinic acid

### Step A. Procedure for Preparation of tert-butyl 2-((3-bromo-2-methylphenoxy)methyl)-7-azaspiro[3.5]nonane-7-carboxylate

A mixture of tert-butyl 2-(hydroxymethyl)-7-azaspiro[3.5]nonane-7-carboxylate (1 g, 3.92 mmol, 1.2 equiv.), 3-bromo-2-methylphenol (610.38 mg, 3.26 mmol, 1 equiv.), and 2-(tributyl-λ⁵-phosphanylidene)acetonitrile (1.18 g, 4.90 mmol, 1.5 equiv.), in toluene (10 mL) was stirred at 120° C. for 2 hours under N₂ atmosphere. The reaction mixture was filtered and then concentrated under reduced pressure to give a residue. The residue was purified by flash silica gel chromatography (eluent of 0˜8% ethyl acetate/petroleum ether) to give tert-butyl 2-((3-bromo-2-methylphenoxy)methyl)-7-azaspiro[3.5]nonane-7-carboxylate (1.3 g, 3.1 mmol, 96.7% yield) as a white solid.

¹H NMR (400 MHz, DMSO-d₆) δ=7.16 (d, J=8.0 Hz, 1H), 7.09 (t, J=8.0 Hz, 1H), 6.96 (d, J=8.0 Hz, 1H), 3.95 (d, J=6.2 Hz, 2H), 3.29-3.25 (m, 2H), 3.22-3.15 (m, 2H), 2.77-2.64 (m, 1H), 2.24 (s, 3H), 1.94-1.86 (m, 2H), 1.69-1.62 (m, 2H), 1.56-1.50 (m, 2H), 1.44-1.40 (m, 2H), 1.38 (s, 9H)

### Step B. Procedure for Preparation of 2-((3-bromo-2-methylphenoxy)methyl)-7-azaspiro[3.5]nonane

A mixture of tert-butyl 2-((3-bromo-2-methylphenoxy)methyl)-7-azaspiro[3.5]nonane-7-carboxylate (1.34 g, 3.16 mmol, 1 equiv.) in HCl/dioxane (15 mL) was stirred at 25° C. for 1 hour. The reaction solution was concentrated under reduced pressure to give a residue. The residue was used for the next step without other purification. 2-((3-bromo-2-methylphenoxy)methyl)-7-azaspiro[3.5]nonane (1.32 g, crude, HCl) was obtained as a white solid.

¹H NMR (400 MHz, DMSO-d₆) δ=9.0-8.75 (m, 2H), 7.15 (d, J=7.2 Hz, 1H), 7.09 (t, J=8.0 Hz, 1H), 6.96 (d, J=8.0 Hz, 1H), 3.95 (d, J=6.2 Hz, 2H), 3.02-2.93 (m, 2H), 2.93-2.84 (m, 2H), 2.74-2.63 (m, 1H), 2.23 (s, 3H), 2.00-1.92 (m, 2H), 1.82-1.77 (m, 2H), 1.74-1.66 (m, 4H)

### Step C. Procedure for Preparation of 2-((3-bromo-2-methylphenoxy)methyl)-7-(2,2-diethoxyethyl)-7-azaspiro[3.5]nonane

A mixture of 2-((3-bromo-2-methylphenoxy)methyl)-7-azaspiro[3.5]nonane (1.22 g, 3.38 mmol, 1 equiv., HCl), 2-bromo-1,1-diethoxy-ethane (666.52 mg, 3.38 mmol, 508.80 μL, 1 equiv.), K₂CO₃ (2.34 g, 16.91 mmol, 5 equiv.), and KI (280.72 mg, 1.69 mmol, 0.5 equiv.) in MeCN (10 mL) was stirred at 80° C. for 16 hours. The reaction solution was concentrated under reduced pressure to give a residue. The mixture was purified by flash silica gel chromatography (eluent of 0˜100% ethyl acetate/petroleum ether) to give 2-((3-bromo-2-methylphenoxy)methyl)-7-(2,2-diethoxyethyl)-7-azaspiro[3.5]nonane (1.3 g, 2.9 mmol, 87.2% yield) as a yellow oil.

MS (ESI) m/z: 442.4[M+H]⁺.

### Step D. Procedure for Preparation of tert-butyl 6-(8-(benzo[d]thiazol-2-ylcarbamoyl)-3,4-dihydroisoquinolin-2(1H)-yl)-3-(3-((7-(2-ethoxy-2-methoxyethyl)-7-azaspiro[3.5]nonan-2-yl)methoxy)-2-methylphenyl)picolinate

A mixture of 2-((3-bromo-2-methylphenoxy)methyl)-7-(2,2-diethoxyethyl)-7-azaspiro[3.5]nonane (400 mg, 908.24 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (723.24 mg, 1.18 mmol, 1.3 equiv.), [2-(2-aminophenyl)phenyl]palladium(1+); bis(1-adamantyl)-butyl-phosphane; methanesulfonate (132.29 mg, 181.65 μmol, 0.2 equiv.), and K₂CO₃ (1.5 M, 1.98 mL, 3 equiv.) in dioxane (4 mL) was degassed and purged with N₂ and stirred at 100° C. for 1 hour under N₂ atmosphere. The reaction mixture was filtered and then concentrated under reduced pressure to give a residue. The mixture was purified by flash silica gel chromatography (eluent of 50˜60% ethyl acetate/petroleum ether) to give tert-butyl 6-(8-(benzo[d]thiazol-2-ylcarbamoyl)-3,4-dihydroisoquinolin-2(1H)-yl)-3-(3-((7-(2-ethoxy-2-methoxyethyl)-7-azaspiro[3.5]nonan-2-yl)methoxy)-2-methylphenyl)picolinate (595 mg, 703.2 μmol, 77.4% yield) as a yellow oil.

MS (ESI) m/z: 846.7[M+H]⁺.

### Step E. Procedure for Preparation of 6-(8-(benzo[d]thiazol-2-ylcarbamoyl)-3,4-dihydroisoquinolin-2(1H)-yl)-3-(2-methyl-3-((7-(2-oxoethyl)-7-azaspiro[3.5]nonan-2-yl)methoxy)phenyl)picolinic acid

A mixture of tert-butyl 6-(8-(benzo[d]thiazol-2-ylcarbamoyl)-3,4-dihydroisoquinolin-2(1H)-yl)-3-(3-((7-(2-ethoxy-2-methoxyethyl)-7-azaspiro[3.5]nonan-2-yl)methoxy)-2-methylphenyl)picolinate (245 mg, 289.57 μmol, 1 equiv.) in HCOOH (3 mL) was stirred at 90° C. for 1.5 hours. The reaction solution was concentrated under reduced pressure to give a residue. The residue was used for the next step without other purification. 6-(8-(benzo[d]thiazol-2-ylcarbamoyl)-3,4-dihydroisoquinolin-2(1H)-yl)-3-(2-methyl-3-((7-(2-oxoethyl)-7-azaspiro[3.5]nonan-2-yl)methoxy)phenyl)picolinic acid (200 mg, crude) was obtained as a yellow oil.

MS (ESI) m/z: 716.6[M+H]⁺.

### Step F. Procedure for Preparation of 6-(8-(benzo[d]thiazol-2-ylcarbamoyl)-3,4-dihydroisoquinolin-2(1H)-yl)-3-(3-((7-(2-(4-(2-(2,6-dioxopiperidin-3-yl)-6-fluoro-1,3-dioxoisoindolin-5-yl)piperazin-1-yl)ethyl)-7-azaspiro[3.5]nonan-2-yl)methoxy)-2-methylphenyl)picolinic acid

A mixture of 2-(2,6-dioxo-3-piperidyl)-5-fluoro-6-piperazin-1-yl-isoindoline-1,3-dione (30.20 mg, 83.82 μmol, 1.2 equiv.), NaBH(OAc)₃ (44.41 mg, 209.54 μmol, 3.0 equiv.), and NMM (14.13 mg, 139.69 μmol, 15.36 μL, 2.0 equiv.) in DCM (0.5 mL) and isopropanol (0.5 mL) was stirred at 0° C. for 5 min, then 6-(8-(benzo[d]thiazol-2-ylcarbamoyl)-3,4-dihydroisoquinolin-2(1H)-yl)-3-(2-methyl-3-((7-(2-oxoethyl)-7-azaspiro[3.5]nonan-2-yl)methoxy)phenyl)picolinic acid (50 mg, 69.85 μmol, 1.0 equiv.) was added to the mixture, and the mixture was stirred at 0° C. for 30 min. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-((7-(2-(4-(2-(2,6-dioxopiperidin-3-yl)-6-fluoro-1,3-dioxoisoindolin-5-yl)piperazin-1-yl)ethyl)-7-azaspiro[3.5]nonan-2-yl)methoxy)-2-methylphenyl)picolinic acid (28.8 mg, 26.8 μmol, 19.2% yield, 98.6% purity) as a yellow solid.

MS (ESI) m/z: 1060.4 [M+H]⁺

¹H NMR (400 MHz, DMSO-d₆) δ=11.13-11.06 (m, 1H), 8.01 (d, J=7.6 Hz, 1H), 7.77 (d, J=7.6 Hz, 1H), 7.71 (d, J=11.2 Hz, 1H), 7.63 (d, J=8.0 Hz, 1H), 7.46-7.32 (m, 6H), 7.07 (t, J=7.2 Hz, 1H), 6.91-6.83 (m, 2H), 6.66 (d, J=8.0 Hz, 1H), 5.10 (dd, J=5.2, 12.6 Hz, 1H), 4.98 (s, 2H), 3.94-3.89 (m, 4H), 3.24-3.20 (m, 6H), 3.04-3.00 (m, 2H), 2.94-2.79 (m, 2H), 2.69-2.65 (m, 2H), 2.57-2.55 (m, 4H), 2.44-2.41 (m, 2H), 2.34-2.31 (m, 2H), 2.10-1.97 (m, 2H), 1.94-1.81 (m, 6H), 1.67-1.58 (m, 4H), 1.50-1.45 (m, 2H)

¹⁹F NMR (400 MHz, DMSO-d₆) δ=−111.952 ppm

### Example 26. Preparation of Compound 133

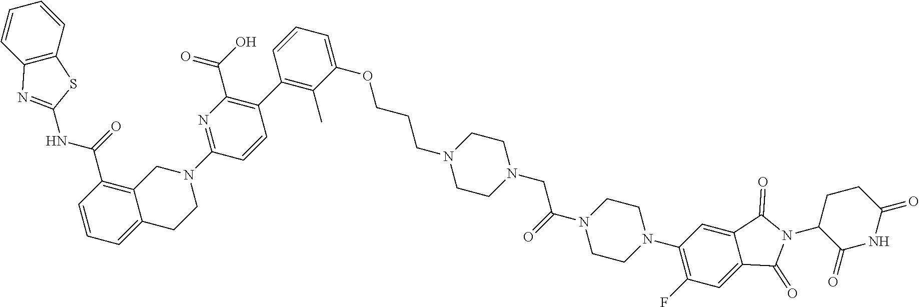 [SMILES: Cc1c(OCCCN2CCN(CC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[4-[2-[4-[2-(2,6-dioxo-3-piperidyl)-6-fluoro-1,3-dioxo-isoindolin-5-yl]piperazin-1-yl]-2-oxo-ethyl]piperazin-1-yl]propoxy]-2-methyl-phenyl]pyridine-2-carboxylic acid

### Step A. Procedure for Preparation of 2-(tert-butyl) 8-methyl 3,4-dihydroisoquinoline-2,8(1H)-dicarboxylate

To a solution of 2-tert-butoxycarbonyl-3,4-dihydro-1H-isoquinoline-8-carboxylic acid (62 g, 223.57 mmol, 1 equiv.) in DMF (600 mL) was added Mel (38.08 g, 268.29 mmol, 16.70 mL, 1.2 equiv.) and K₂CO₃ (61.80 g, 447.15 mmol, 2 equiv.). The mixture was stirred at 50° C. for 2 hours. The mixture was poured into ice water (800 mL) (pH>8) and extracted with EtOAc (300 mL×3). The organic layers were combined and washed with brine (300 mL×3), dried over Na₂SO₄, and concentrated in vacuo to give product. The crude product 2-(tert-butyl) 8-methyl 3,4-dihydroisoquinoline-2,8(1H)-dicarboxylate (66 g, crude) as a brown oil was used into the next step without further purification.

### Step B. Procedure for Preparation of tert-butyl 8-(1,3-benzothiazol-2-ylcarbamoyl)-3,4-dihydro-1H-isoquinoline-2-carboxylate

To a solution of 1,3-benzothiazol-2-amine (35.73 g, 237.87 mmol, 1.05 equiv.) in 2-MeTHF (350 mL) was added LiHMDS (1 M, 453.08 mL, 2 equiv.) at 0° C., and the solution was stirred at 0° C. for 30 min. Then a solution of 2-(tert-butyl) 8-methyl 3,4-dihydroisoquinoline-2,8(1H)-dicarboxylate (66.87 g, 226.54 mmol, 98.7% purity, 1 equiv.) in 2-MeTHF (350 mL) was added to the above solution at 0° C. and stirred for 30 min again. The resulting mixture was warmed to 60° C. and stirred for 17 hours. The reaction mixture was poured into sat. NH₄Cl (1 L), extracted with EtOAc (500 mL×3). The combined organic layer was washed with brine, dried over Na₂SO₄, and concentrated in vacuo to give a crude product. The crude product was purified by re-crystallization from EtOAc (500 mL) at 25° C. to give 36 g product. The filtrate was concentrated in vacuo to give a residue. The residue was dissolved EtOAc (300 mL), then the solution was washed with 10% citric acid (100 mL×3) and brine (150 mL), dried over Na₂SO₄, and concentrated under reduced pressure to give a residue. The residue was purified by re-crystallization from ethyl acetate/petroleum ether=1/1 (600 ml) at 25° C. to afford 25.17 g product. The second recrystallized mother liquor was concentrated in vacuo to give residue (about 40 g). The residue was purified by flash silica gel chromatography (eluent of 0˜40% DCM/petroleum ether) to give the title product (5 g). Combined different batches of products to afford tert-butyl 8-(1,3-benzothiazol-2-ylcarbamoyl)-3,4-dihydro-1H-isoquinoline-2-carboxylate (66.7 g, 162.88 mmol, 72.85% yield of two steps) as a light yellow solid.

MS (ESI) m/z: 410.0 [M+H]⁺.

¹H NMR (400 MHz, CDCl₃) δ=7.87-7.79 (m, 1H), 7.51 (d, J=7.4 Hz, 1H), 7.33-7.26 (m, 3H), 7.22 (d, J=7.4 Hz, 1H), 7.17-7.10 (m, 1H), 4.91 (s, 2H), 3.64 (s, 2H), 2.88 (s, 2H), 1.57-1.34 (m, 9H)

### Step C. Procedure for Preparation of N-(1,3-benzothiazol-2-yl)-1,2,3,4-tetrahydroisoquinoline-8-carboxamide

To a mixture of tert-butyl 8-(1,3-benzothiazol-2-ylcarbamoyl)-3,4-dihydro-1H-isoquinoline-2-carboxylate (61.7 g, 150.67 mmol, 1 equiv.) in DCM (1000 mL) was added HCl/dioxane (600 mL). The mixture was stirred at 25° C. for 6 hours. The reaction mixture was concentrated under reduced pressure to give N-(1,3-benzothiazol-2-yl)-1,2,3,4-tetrahydroisoquinoline-8-carboxamide (59 g, crude,) as a white solid. The solid was used into the next step without further purification.

¹H NMR (400 MHz, DMSO-d₆) δ=9.63 (br s, 2H), 8.02 (d, J=7.8 Hz, 1H), 7.87-7.61 (m, 2H), 7.55-7.22 (m, 4H), 4.47-4.36 (m, 2H), 3.56 (s, 3H), 3.43-3.28 (m, 2H), 3.11 (t, J=5.8 Hz, 2H)

### Step D. Procedure for Preparation of tert-butyl 6-[8-(1,3-benzothiazol-2-ylcarbamoyl)-3,4-dihydro-1H-isoquinolin-2-yl]-3-bromo-pyridine-2-carboxylate

To a mixture of N-(1,3-benzothiazol-2-yl)-1,2,3,4-tetrahydroisoquinoline-8-carboxamide (30 g, 73.76 mmol, 94% purity, 2HCl) in DMA (300 mL) was added Cs₂CO₃ (120.17 g, 368.81 mmol) and tert-butyl 3-bromo-6-chloro-pyridine-2-carboxylate (21.80 g, 74.50 mmol, 1.01 equiv.). The mixture was stirred at 120° C. for 12 hours. Then tert-butyl 3-bromo-6-chloro-pyridine-2-carboxylate (5.8 g,) was added to the reaction mixture and the resulting mixture was stirred at 120° C. for 7 hours. The reaction mixture was cooled to room temperature, and poured into ice water (1 L), then the mixture was stirred for 10 min and filtered off. The cake was washed with EtOH, and the cake was collected. The cake was triturated with EtOH (200 mL) at 25° C. for 30 min to give tert-butyl 6-[8-(1,3-benzothiazol-2-ylcarbamoyl)-3,4-dihydro-1H-isoquinolin-2-yl]-3-bromo-pyridine-2-carboxylate (24.6 g, 43.50 mmol, 58.98% yield) as a white solid.

MS (ESI) m/z: 567.1 [M+H]⁺ (⁸¹Br).

MS (ESI) m/z: 565.0 [M+H]⁺

¹H NMR (400 MHz, DMSO-d₆) δ=12.70 (brs, 1H), 8.01 (d, J=7.8 Hz, 1H), 7.77 (dd, J=2.8, 8.4 Hz, 2H), 7.58 (d, J=7.4 Hz, 1H), 7.50-7.39 (m, 2H), 7.39-7.30 (m, 2H), 6.86 (d, J=9.2 Hz, 1H), 4.93 (s, 2H), 3.77 (t, J=6.0 Hz, 2H), 3.00 (t, J=5.8 Hz, 2H), 1.34 (s, 9H)

### Step E. Procedure for Preparation of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate

Tert-butyl 6-[8-(1,3-benzothiazol-2-ylcarbamoyl)-3,4-dihydro-1H-isoquinolin-2-yl]-3-bromo-pyridine-2-carboxylate (1.5 g, 2.65 mmol, 1 equiv.), 4,4,5,5-tetramethyl-1,3,2-dioxaborolane (746.84 mg, 5.84 mmol, 846.76 μL, 2.2 equiv.), TEA (836.05 mg, 8.26 mmol, 1.15 mL, 3.11 equiv.), and Pd(dppf)Cl₂·CH₂Cl₂ (108.31 mg, 132.63 μmol, 0.05 equiv.) were taken up into a microwave tube in CH₃CN (15 mL) and THE (5 mL). The sealed tube was heated at 100° C. for 30 min under microwave. The reaction mixture was poured into water (200 mL) and extracted with ethyl acetate (150 mL×2). The combined organic phase was concentrated in vacuo to give a residue. The residue was purified by column chromatography (SiO₂, petroleum ether/ethyl acetate 6/1 to 1/2) to afford tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate (9.2 g, 14.27 mmol, 55.0% yield, 95% purity) as a white solid.

MS (ESI) m/z: 613.7 [M+H]⁺.

¹H NMR (400 MHz, DMSO-d₆) δ=12.84 (s, 1H), 8.03 (d, J=7.6 Hz, 1H), 7.78 (d, J=8.2 Hz, 1H), 7.68 (d, J=8.6 Hz, 1H), 7.56 (d, J=7.2 Hz, 1H), 7.50-7.39 (m, 2H), 7.38-7.30 (m, 2H), 6.86 (d, J=8.8 Hz, 1H), 5.01 (s, 2H), 3.82 (t, J=5.8 Hz, 2H), 3.00 (t, J=5.8 Hz, 2H), 1.28 (s, 9H), 1.22 (s, 12H)

### Step F. Procedure for Preparation of 1-bromo-3-(3-bromopropoxy)-2-methyl-benzene

A mixture of 3-bromo-2-methyl-phenol (2 g, 10.69 mmol, 1 equiv.), 1,3-dibromopropane (10.79 g, 53.47 mmol, 5.45 mL, 5 equiv.), and Cs₂CO₃ (10.45 g, 32.08 mmol, 3 equiv.) in MeCN (25 mL) was stirred at 25° C. for 5 hours. The reaction mixture was filtered and then concentrated under reduced pressure to give a residue. The residue was purified by flash silica gel chromatography (eluent of 0˜1% ethyl acetate/petroleum ether). 1-Bromo-3-(3-bromopropoxy)-2-methyl-benzene (2.4 g, 7.0 mmol, 65.8% yield, 90% purity) was obtained as a white solid.

### Step G. Procedure for Preparation of ethyl 2-[4-[3-(3-bromo-2-methyl-phenoxy)propyl]piperazin-1-yl]acetate

A mixture of 1-bromo-3-(3-bromopropoxy)-2-methyl-benzene (2.15 g, 6.97 mmol, 1 equiv.), ethyl 2-piperazin-1-ylacetate (1.2 g, 6.97 mmol, 1 equiv.), and DIPEA (3.60 g, 27.87 mmol, 4.85 mL, 4 equiv.) in DMF (25 mL) was stirred at 60° C. for 3 hours. The reaction mixture was filtered and then concentrated under reduced pressure to give a residue. The residue was purified by flash silica gel chromatography (eluent of 0˜10% ethyl acetate/petroleum ether) to give ethyl 2-[4-[3-(3-bromo-2-methyl-phenoxy)propyl]piperazin-1-yl]acetate (1.3 g, 3.2 mmol, 46.0% yield, 95% purity) as a white solid.

MS (ESI) m/z: 401.1 [M+H]⁺ (⁸⁰Br).

### Step H. Procedure for Preparation of tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)piperazin-1-yl]propoxy]-2-methyl-phenyl]pyridine-2-carboxylate

A mixture of ethyl 2-[4-[3-(3-bromo-2-methyl-phenoxy)propyl]piperazin-1-yl]acetate (380 mg, 951.61 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate (816.07 mg, 1.33 mmol, 1.4 equiv.), [2-(2-aminophenyl)phenyl]palladium(1+); bis(1-adamantyl)-butyl-phosphane; methanesulfonate (138.61 mg, 190.32 μmol, 0.2 equiv.), and K₂CO₃ (1.5 M, 951.61 μL, 1.5 equiv.) in dioxane (3 mL) was degassed and purged with N₂ 3 times, and then the mixture was stirred at 100° C. for 1 hour under microwave. The reaction mixture was filtered and then concentrated under reduced pressure to give a residue. The residue was purified by flash silica gel chromatography (eluent of 0-10% DCM/MeOH) to give tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)piperazin-1-yl]propoxy]-2-methyl-phenyl]pyridine-2-carboxylate (546 mg, 610.4 μmol, 64.1% yield, 90% purity) as a white solid.

MS (ESI) m/z: 805.5 [M+H]⁺.

¹H NMR (400 MHz, CD₃OD) δ=7.90 (d, J=8.0 Hz, 1H), 7.75 (d, J=8.0 Hz, 1H), 7.58 (d, J=7.6 Hz, 1H), 7.42 (d, J=8.4 Hz, 1H), 7.37-7.34 (m, 1H), 7.14-7.06 (m, 2H), 7.02 (t, J=8.0 Hz, 1H), 6.94 (d, J=8.8 Hz, 1H), 6.89 (dd, J=4.4, 8.0 Hz, 2H), 6.63 (d, J=7.6 Hz, 1H), 5.02 (s, 2H), 4.17 (dq, J=2.8, 7.2 Hz, 4H), 3.98 (t, J=6.0 Hz, 2H), 3.25 (d, J=3.6 Hz, 3H), 3.08 (t, J=6.0 Hz, 2H), 2.67-2.63 (m, 10H), 2.28 (s, 2H), 1.93 (s, 3H), 1.80 (s, 2H), 1.08 (s, 9H)

### Step I. Procedure for preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]piperazin-1-yl]acetic acid

A mixture of tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)piperazin-1-yl]propoxy]-2-methyl-phenyl]pyridine-2-carboxylate (230 mg, 285.72 μmol, 1 equiv.) and LiOH·H₂O (35.97 mg, 857.15 μmol, 3 equiv.) in THF (1.2 mL) and H₂O (0.4 mL) was stirred at 25° C. for 1.5 hours. The mixture was concentrated and redissolved by 2 mL H₂O, then 1M HCl was added to adjust the pH to 2, and the residue was filtered and dried. The residue was used for next step without further purification. 2-[4-[3-[3-[6-[8-(1,3-Benzothiazol-2-ylcarbamoyl)-3,4-dihydro-1H-isoquinolin-2-yl]-2-tert-butoxycarbonyl-3-pyridyl]-2-methyl-phenoxy]propyl]piperazin-1-yl]acetic acid (206 mg, 225.3 μmol, 78.8% yield) was obtained as a white solid.

### Step J. Procedure for Preparation of tert-butyl 6-[8-(1,3-benzothiazol-2-ylcarbamoyl)-3,4-dihydro-1H-isoquinolin-2-yl]-3-[3-[3-[4-[2-[4-[2-(2,6-dioxo-3-piperidyl)-6-fluoro-1,3-dioxo-isoindolin-5-yl]piperazin-1-yl]-2-oxo-ethyl]piperazin-1-yl]propoxy]-2-methyl-phenyl]pyridine-2-carboxylate

A mixture of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]piperazin-1-yl]acetic acid (90 mg, 115.84 μmol, 1 equiv.), 2-(2,6-dioxo-3-piperidyl)-5-fluoro-6-piperazin-1-yl-isoindoline-1,3-dione (45.92 mg, 127.42 μmol, 1.1 equiv.), HATU (44.05 mg, 115.84 μmol, 1 equiv.), and DIPEA (44.91 mg, 347.52 μmol, 60.53 μL, 3 equiv.) in DMF (1 mL) was stirred at 25° C. for 12 hours. The reaction mixture was quenched by addition H₂O (2 mL), and then extracted with ethyl acetate 6 mL (2 mL×3). The combined organic layers were washed with saturated sodium chloride (3 mL), dried over anhydrous sodium sulfate, filtered, and concentrated under reduced pressure to give a residue. The residue was used for next step without other purification. Tert-butyl 6-[8-(1,3-benzothiazol-2-ylcarbamoyl)-3,4-dihydro-1H-isoquinolin-2-yl]-3-[3-[3-[4-[2-[4-[2-(2,6-dioxo-3-piperidyl)-6-fluoro-1,3-dioxo-isoindolin-5-yl]piperazin-1-yl]-2-oxo-ethyl]piperazin-1-yl]propoxy]-2-methyl-phenyl]pyridine-2-carboxylate (120 mg, 91.1 μmol, 78.6% yield) was obtained as a white solid.

### Step K. Procedure for Preparation of 6-[8-(1,3-benzothiazol-2-ylcarbamoyl)-3,4-dihydro-1H-isoquinolin-2-yl]-3-[3-[3-[4-[2-[4-[2-(2,6-dioxo-3-piperidyl)-6-fluoro-1,3-dioxo-isoindolin-5-yl]piperazin-1-yl]-2-oxo-ethyl]piperazin-1-yl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[4-[2-[4-[2-(2,6-dioxo-3-piperidyl)-6-fluoro-1,3-dioxo-isoindolin-5-yl]piperazin-1-yl]-2-oxo-ethyl]piperazin-1-yl]propoxy]-2-methyl-phenyl]pyridine-2-carboxylate (120 mg, 107.21 μmol, 1 equiv.) and TFA (308.00 mg, 2.70 mmol, 0.2 mL, 25.19 equiv.) in DCM (0.6 mL) was stirred at 25° C. for 10 hours. The reaction mixture was filtered and concentrated under reduced pressure to give a residue. The residue was purified by prep-HPLC. 6-[8-(1,3-Benzothiazol-2-ylcarbamoyl)-3,4-dihydro-1H-isoquinolin-2-yl]-3-[3-[3-[4-[2-[4-[2-(2,6-dioxo-3-piperidyl)-6-fluoro-1,3-dioxo-isoindolin-5-yl]piperazin-1-yl]-2-oxo-ethyl]piperazin-1-yl]propoxy]-2-methyl-phenyl]pyridine-2-carboxylic acid (24.5 mg, 21.9 μmol, 20.4% yield, 95% purity) was obtained as a white solid.

MS (ESI) m/z: 1063.6 [M+H]⁺.

¹H NMR (400 MHz, CD₃OD) δ=11.11 (s, 1H), 8.15 (s, 1H), 8.03 (d, J=8.4 Hz, 1H), 7.82-7.73 (m, 2H), 7.62 (d, J=7.2 Hz, 1H), 7.56-7.29 (m, 7H), 7.09 (t, J=7.6 Hz, 1H), 6.96 (d, J=9.2 Hz, 1H), 6.87 (d, J=8.4 Hz, 1H), 6.67-6.61 (m, 1H), 5.11 (dd, J=5.2, 12.8 Hz, 1H), 4.98 (s, 2H), 4.03-3.95 (m, 2H), 3.91 (s, 2H), 3.81-3.56 (m, 6H), 3.24-3.16 (m, 6H), 3.02 (s, 3H), 2.96-2.76 (m, 2H), 2.74-2.59 (m, 4H), 2.39-2.22 (m, 4H), 2.06-1.99 (m, 1H), 1.89 (s, 5H).

FNMR (400 MHz, CD₃OD) δ=−112.06

### Example 27 Preparation of Compound 135a

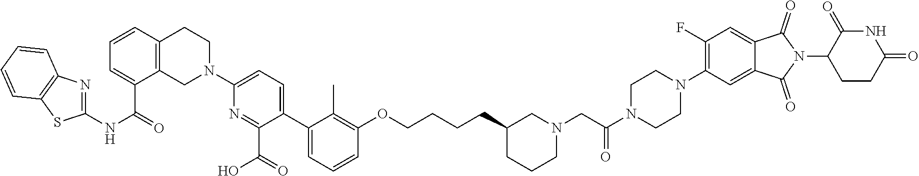 [SMILES: Cc1c(OCCCC[C@@H]2CCCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[(3R)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylic acid

### Step A. Procedure for Preparation of tert-butyl (3R)-3-[4-(3-bromo-2-methyl-phenoxy)butyl]piperidine-1-carboxylate

A mixture of tert-butyl (3R)-3-(4-hydroxybutyl)piperidine-1-carboxylate (1.3 g, 5.05 mmol, 1 equiv.), 3-bromo-2-methyl-phenol (755.79 mg, 4.04 mmol, 0.8 equiv.), and 2-(tributyl-λ5-phosphanylidene)acetonitrile (1.46 g, 6.06 mmol, 1.2 equiv.) in toluene (30 mL) was stirred at 120° C. for 2 hours under N₂ atmosphere. The reaction mixture was filtered and then concentrated under reduced pressure to give a residue. The residue was purified by flash silica gel chromatography (eluent of 10% ethyl acetate/petroleum ether). Then the residue was further separated by SFC to give tert-butyl (3R)-3-[4-(3-bromo-2-methyl-phenoxy)butyl]piperidine-1-carboxylate (1.1 g, 2.5 mmol, 50.7% yield, 99.3% purity) as a yellow oil.

MS (ESI) m/z: 328.0 [M−100+H]⁺.

¹H NMR (400 MHz, DMSO-d₆) δ=7.17-7.13 (m, 1H), 7.12-7.06 (m, 1H), 6.97 (d, J=8.0 Hz, 1H), 3.98 (t, J=6.4 Hz, 2H), 3.85-3.68 (m, 2H), 2.80-2.73 (m, 1H), 2.49-2.26 (m, 1H), 2.23 (s, 3H), 1.78-1.69 (m, 3H), 1.60-1.53 (m, 1H), 1.49-1.43 (m, 2H), 1.38 (s, 9H), 1.36-1.27 (m, 2H), 1.27-1.20 (m, 2H), 1.14-1.01 (m, 1H).

### Step B. Procedure for Preparation of (3R)-3-[4-(3-bromo-2-methyl-phenoxy)butyl]piperidine

A mixture of tert-butyl (3R)-3-[4-(3-bromo-2-methyl-phenoxy)butyl]piperidine-1-carboxylate (400 mg, 938.12 μmol, 1 equiv.) in DCM (2.5 mL) and HCl/dioxane (12 mL) was stirred at 25° C. for 16 hours. The reaction mixture was concentrated under reduced pressure to give (3R)-3-[4-(3-bromo-2-methyl-phenoxy)butyl]piperidine (349 mg, crude, HCl) as a white solid.

MS (ESI) m/z: 328.1 [M+H]⁺.

¹H NMR (400 MHz, DMSO-d₆) δ=7.17-7.13 (m, 1H), 7.12-7.06 (m, 1H), 6.96 (d, J=8.0 Hz, 1H), 3.98 (t, J=6.4 Hz, 2H), 3.18 (d, J=12.0 Hz, 2H), 2.78-2.67 (m, 1H), 2.48-2.45 (m, 1H), 2.23 (s, 3H), 1.83-1.52 (m, 7H), 1.48-1.39 (m, 2H), 1.32-1.22 (m, 2H), 1.16-1.04 (m, 1H).

### Step C. Procedure for Preparation of ethyl 2-[(3R)-3-[4-(3-bromo-2-methyl-phenoxy)butyl]-1-piperidyl]acetate

A mixture of (3R)-3-[4-(3-bromo-2-methyl-phenoxy)butyl]piperidine (349 mg, 1.07 mmol, 1 equiv.), ethyl 2-bromoacetate (142.91 mg, 855.73 μmol, 94.64 μL, 0.8 equiv.), and K₂CO₃ (443.50 mg, 3.21 mmol, 3 equiv.) in DMF (10 mL) was stirred at 60° C. for 2 hours under N₂ atmosphere. The reaction mixture was concentrated under reduced pressure to give a residue. The crude product was purified by reverse-phase HPLC (with 0.1% HCOOH) to give ethyl 2-[(3R)-3-[4-(3-bromo-2-methyl-phenoxy)butyl]-1-piperidyl]acetate (374 mg, 896.9 μmol, 83.8% yield, 98.9% purity) as a yellow oil.

MS (ESI) m/z: 414.0 [M+H]⁺.

¹H NMR (400 MHz, DMSO-d₆) δ=8.14 (s, 1H), 7.16-7.12 (m, 1H), 7.11-7.05 (m, 1H), 6.95 (d, J=7.6 Hz, 1H), 4.12 (q, J=7.2 Hz, 2H), 3.97 (t, J=6.4 Hz, 2H), 3.51 (s, 2H), 3.05-2.92 (m, 2H), 2.44-2.33 (m, 1H), 2.22 (s, 3H), 2.13 (t, J=10.8 Hz, 1H), 1.73-1.69 (m, 2H), 1.66-1.58 (m, 2H), 1.43 (td, J=7.6, 14.8 Hz, 2H), 1.33-1.09 (m, 6H), 0.84-0.95 (m, 1H).

### Step D. Procedure for Preparation of tert-butyl 6-[8-(1,3-benzothiazol-2-ylcarbamoyl)-3,4-dihydro-1H-isoquinolin-2-yl]-3-[3-[4-[(3R)-1-(2-ethoxy-2-oxo-ethyl)-3-piperidyl]butoxy]-2-methyl-phenyl]pyridine-2-carboxylate

Ethyl 2-[(3R)-3-[4-(3-bromo-2-methyl-phenoxy)butyl]-1-piperidyl]acetate (421 mg, 1.02 mmol, 1.05 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (596.69 mg, 974.12 μmol, 1 equiv.), Ad₂nBuP Pd G₃(cataCXium® A Pd G₃) (70.94 mg, 97.41 μmol, 0.1 equiv.), and KF (1.5 M, 1.95 mL, 3 equiv.) were taken up into a microwave tube in 1,4-dioxane (10 mL). The sealed tube was heated at 100° C. for 60 minutes under microwave. The reaction mixture was filtered and concentrated under reduced pressure to give a residue. The residue was purified by prep-HPLC to give tert-butyl 6-[8-(1,3-benzothiazol-2-ylcarbamoyl)-3,4-dihydro-1H-isoquinolin-2-yl]-3-[3-[4-[(3R)-1-(2-ethoxy-2-oxo-ethyl)-3-piperidyl]butoxy]-2-methyl-phenyl]pyridine-2-carboxylate (511 mg, 593.5 μmol, 60.9% yield, 95.0% purity) as a yellow oil.

MS (ESI) m/z: 818.4 [M+H]⁺.

¹H NMR (400 MHz, CDCl₃) δ=7.80 (d, J=7.6 Hz, 1H), 7.59 (dd, J=7.6, 15.2 Hz, 2H), 7.41-7.35 (m, 1H), 7.34-7.29 (m, 3H), 7.27 (d, J=7.6 Hz, 1H), 7.03 (t, J=8.0 Hz, 1H), 6.82 (d, J=8.8 Hz, 1H), 6.73 (d, J=8.0 Hz, 1H), 6.64 (d, J=7.6 Hz, 1H), 5.24 (s, 1H), 5.05-4.91 (m, 2H), 4.18 (q, J=7.2 Hz, 2H), 4.03 (t, J=5.6 Hz, 2H), 3.90 (t, J=6.0 Hz, 3H), 3.81 (s, 2H), 3.01 (t, J=5.6 Hz, 3H), 2.04-1.95 (m, 2H), 1.91 (s, 5H), 1.76-1.71 (m, 2H), 1.54-1.38 (m, 3H), 1.27-1.22 (m, 4H), 1.20 (d, J=6.0 Hz, 3H), 1.08 (s, 9H).

### Step E. Procedure for Preparation of 2-[(3R)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid

A mixture of tert-butyl 6-[8-(1,3-benzothiazol-2-ylcarbamoyl)-3,4-dihydro-1H-isoquinolin-2-yl]-3-[3-[4-[(3R)-1-(2-ethoxy-2-oxo-ethyl)-3-piperidyl]butoxy]-2-methyl-phenyl]pyridine-2-carboxylate (112 mg, 136.91 μmol, 1 equiv.) and LiOH·H₂O (17.24 mg, 410.74 μmol, 3 equiv.) in THF (2 mL) and H₂O (1 mL) was stirred at 25° C. for 16 hours. The reaction mixture was quenched by water (5 mL), and then extracted with ethyl acetate (10 mL×3), and the organic layers were combined and concentrated under reduced pressure to give 2-[(3R)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (105 mg, crude) as a yellow oil.

MS (ESI) m/z: 790.4 [M+H]⁺.

### Step F. Procedure for Preparation of tert-butyl 6-[8-(1,3-benzothiazol-2-ylcarbamoyl)-3,4-dihydro-1H-isoquinolin-2-yl]-3-[3-[4-[(3R)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylate

A mixture of 2-[(3R)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (100 mg, 126.59 μmol, 1 equiv.), 2-(2,6-dioxo-3-piperidyl)-5-fluoro-6-piperazin-1-yl-isoindoline-1,3-dione (54.74 mg, 151.90 μmol, 1.2 equiv.), HATU (57.76 mg, 151.90 μmol, 1.2 equiv.), and DIEA (49.08 mg, 379.76 μmol, 66.15 μL, 3 equiv.) in DMF (3 mL) was stirred at 25° C. for 12 hours. The reaction mixture was quenched by addition water (3 mL), and then extracted with ethyl acetate (3 mL×3), and the organic layers were combined and concentrated under reduced pressure to give tert-butyl 6-[8-(1,3-benzothiazol-2-ylcarbamoyl)-3,4-dihydro-1H-isoquinolin-2-yl]-3-[3-[4-[(3R)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylate (100 mg, crude) as a yellow oil.

MS (ESI) m/z: 1133.7 [M+H]⁺.

### Step G. Procedure for Preparation of 6-[8-(1,3-benzothiazol-2-ylcarbamoyl)-3,4-dihydro-1H-isoquinolin-2-yl]-3-[3-[4-[(3R)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[(3R)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylate (100 mg, 88.32 μmol, 1 equiv.) in TFA (1 mL) and DCM (1 mL) was stirred at 25° C. for 16 hours. The reaction mixture was concentrated under reduced pressure to remove solvent to give a residue. The residue was purified by prep-HPLC to give 6-[8-(1,3-benzothiazol-2-ylcarbamoyl)-3,4-dihydro-1H-isoquinolin-2-yl]-3-[3-[4-[(3R)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylic acid (37.4 mg, 34.7 μmol, 39.3% yield, 99.8% purity) as a yellow solid.

MS (ESI) m/z: 1077.8 [M+H]⁺.

¹H NMR (400 MHz, DMSO-d₆) δ=13.27-12.23 (m, 1H), 11.11 (s, 1H), 8.14 (s, 1H), 8.03 (d, J=8.0 Hz, 1H), 7.78 (d, J=8.0 Hz, 1H), 7.73 (d, J=11.2 Hz, 1H), 7.62 (d, J=7.6 Hz, 1H), 7.49-7.41 (m, 4H), 7.39-7.32 (m, 2H), 7.07 (t, J=8.0 Hz, 1H), 6.96 (d, J=8.8 Hz, 1H), 6.85 (d, J=8.4 Hz, 1H), 6.61 (d, J=7.6 Hz, 1H), 5.10 (dd, J=5.2, 12.8 Hz, 1H), 4.98 (s, 2H), 3.80-4.00 (m, 4H), 3.70 (s, 2H), 3.61 (d, J=2.8 Hz, 2H), 3.26 (d, J=3.2 Hz, 4H), 3.19 (s, 4H), 3.02 (t, J=5.6 Hz, 2H), 2.93-2.75 (m, 4H), 2.17-1.95 (m, 3H), 1.87 (s, 4H), 1.70 (d, J=6.4 Hz, 4H), 1.53-1.42 (m, 3H), 1.29-1.21 (m, 2H).

### Example 28. Preparation of Compound 135b

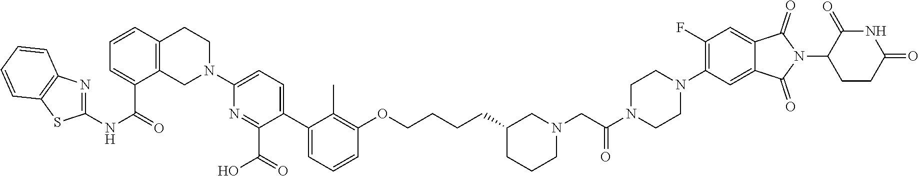 [SMILES: Cc1c(OCCCC[C@H]2CCCN(CC(=O)N3CCN(c4cc5c(cc4F)C(=O)N(C4CCC(=O)NC4=O)C5=O)CC3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[(3S)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylic acid

### Step A. Procedure for Preparation of tert-butyl (3S)-3-formylpiperidine-1-carboxylate

A mixture of tert-butyl (3S)-3-(hydroxymethyl)piperidine-1-carboxylate (4 g, 18.58 mmol, 1 equiv.) and DMP (8.67 g, 20.44 mmol, 6.33 mL, 1.1 equiv.) in DCM (50 mL) was stirred at 20° C. for 1 hour under N₂ atmosphere. The mixture was filtered and then concentrated under reduced pressure to give a residue. The residue was purified by column chromatography (SiO₂, petroleum ether/ethyl acetate 50/1 to 3/1) to give tert-butyl (3S)-3-formylpiperidine-1-carboxylate (3 g, 14.0 mmol, 75.7% yield) as a colorless oil.

¹H NMR ¹H NMR (400 MHz, DMSO-d₆) δ=9.59 (s, 1H), 3.72 (d, J=8.0 Hz, 1H), 3.43 (s, 1H), 3.14-3.01 (m, 1H), 2.49-2.41 (m, 1H), 1.89-1.80 (m, 1H), 1.65-1.48 (m, 2H), 1.39 (s, 11H)

### Step B. Procedure for Preparation of tert-butyl (3R)-3-[(E)-4-benzyloxybut-1-enyl]piperidine-1-carboxylate

A mixture of 3-benzyloxypropyl(triphenyl)phosphonium; bromide (3.46 g, 7.03 mmol, 1 equiv.) in THF (5 mL) was degassed and purged with N₂ 3 times and cooled to −70° C. LiHMDS (1 M, 10.55 mL, 1.5 equiv.) was added dropwise at −70° C. After stirring at −70° C. for 1 hour, tert-butyl (3S)-3-formylpiperidine-1-carboxylate (1.5 g, 7.03 mmol, 1 equiv.) in THF (3 mL) was added dropwise at −70° C., then the mixture was stirred at −70° C. for 1 hour. The reaction mixture was quenched by addition of saturated NH₄Cl solution (20 mL) dropwise at 0° C., and then extracted with EtOAc (20 mL×3). The combined organic layers were washed with brine (10 mL×3), dried over anhydrous Na₂SO₄, filtered, and concentrated under reduced pressure to give a residue. The residue was purified by column chromatography (SiO2, petroleum ether/ethyl acetate 200/1 to 10/1) to give tert-butyl (3R)-3-[(E)-4-benzyloxybut-1-enyl]piperidine-1-carboxylate (1.8 g, 4.6 mmol, 65.9% yield, 89% purity) as a colorless oil.

¹H NMR (400 MHz, DMSO-d₆) δ=7.39-7.18 (m, 5H), 5.48-5.36 (m, 1H), 5.28-5.18 (m, 1H), 4.46 (s, 2H), 3.84-3.64 (m, 2H), 3.44 (t, J=6.8 Hz, 2H), 2.72 (s, 1H), 2.63-2.51 (m, 1H), 2.41-2.25 (m, 3H), 1.66 (d, J=12.4 Hz, 1H), 1.60-1.51 (m, 1H), 1.38 (s, 10H), 1.28-1.17 (m, 1H)

### Step C. Procedure for Preparation of tert-butyl (3S)-3-(4-hydroxybutyl)piperidine-1-carboxylate

A mixture of tert-butyl (3R)-3-[(E)-4-benzyloxybut-1-enyl]piperidine-1-carboxylate (1.8 g, 5.21 mmol, 1 equiv.), Pd/C (0.1 g, 521.02 μmol, 10% purity, 0.1 equiv.), and Pd(OH)₂ (0.1 g, 71.21 μmol, 10% purity, 0.0137 equiv.) in MeOH (200 mL) was degassed and purged with H₂ (15 psi) 3 times, and then the mixture was stirred at 20° C. for 16 hours under H₂ (15 psi) atmosphere. The mixture was filtered and then concentrated under reduced pressure to give crude tert-butyl (3S)-3-(4-hydroxybutyl)piperidine-1-carboxylate (1.3 g, 4.0 mmol, 77.5% yield, 80% purity) as a gray oil.

¹H NMR (400 MHz, DMSO-d₆) δ=3.72 (s, 2H), 3.38 (q, J=6.0 Hz, 1H), 2.84-2.70 (m, 1H), 2.50 (s, 1H), 1.74 (d, J=10.4 Hz, 1H), 1.62-1.50 (m, 1H), 1.38 (s, 11H), 1.34-1.11 (m, 7H), 1.08 (d, J=7.6 Hz, 1H), 0.87 (t, J=6.4 Hz, 1H)

### Step D. Procedure for Preparation of tert-butyl (3S)-3-[4-(3-bromo-2-methyl-phenoxy)butyl]piperidine-1-carboxylate

A mixture of 3-bromo-2-methyl-phenol (399.69 mg, 2.14 mmol, 1.1 equiv.) and tert-butyl (3S)-3-(4-hydroxybutyl)piperidine-1-carboxylate (0.5 g, 1.94 mmol, 1 equiv.) in toluene (5 mL) was degassed and purged with N₂ 3 times, and then 2-(tributyl-λ⁵-phosphanylidene)acetonitrile (515.78 mg, 2.14 mmol, 2.14 mL, 1.1 equiv.) was added dropwise. The mixture was stirred at 110° C. for 16 hours under N₂ atmosphere. The reaction mixture was concentrated under reduced pressure to give a residue. The residue was purified by flash silica gel chromatography (eluent of 0˜25% ethyl acetate/petroleum ether) to give tert-butyl (3S)-3-[4-(3-bromo-2-methyl-phenoxy)butyl]piperidine-1-carboxylate (0.5 g, 1.1 mmol, 55.5% yield, 92% purity) as a colorless oil.

MS (ESI) m/z: 326.2 [M−100+H]+

### Step E. Procedure for Preparation of (3S)-3-[4-(3-bromo-2-methyl-phenoxy)butyl]piperidine

To a solution of tert-butyl (3S)-3-[4-(3-bromo-2-methyl-phenoxy)butyl]piperidine-1-carboxylate (0.5 g, 1.17 mmol, 1 equiv.) in HCl/dioxane (5 mL) was stirred at 20° C. for 1 hour. The reaction mixture was concentrated under reduced pressure to give a residue. The crude product was triturated with petroleum ether/ethyl acetate=5:1 (2 mL) at 20° C. for 30 min to give (3S)-3-[4-(3-bromo-2-methyl-phenoxy)butyl]piperidine (0.3 g, 919.4 μmol, 78.4% yield) as a white solid.

¹H NMR (400 MHz, DMSO-d₆) δ=8.81-8.58 (m, 1H), 8.52-8.30 (m, 1H), 7.19-7.14 (m, 1H), 7.13-7.07 (m, 1H), 6.97 (d, J=7.6 Hz, 1H), 3.99 (br t, J=6.4 Hz, 2H), 2.83-2.68 (m, 2H), 2.24 (s, 3H), 1.82-1.71 (m, 4H), 1.63-1.55 (m, 1H), 1.50-1.41 (m, 2H), 1.35-1.22 (m, 5H), 1.18-1.05 (m, 1H)

### Step F. Procedure for Preparation of ethyl 2-[(3S)-3-[4-(3-bromo-2-methyl-phenoxy)butyl]-1-piperidyl]acetate

To a solution of ethyl 2-bromoacetate (91.16 mg, 545.86 μmol, 60.37 μL, 0.9 equiv.) and (3S)-3-[4-(3-bromo-2-methyl-phenoxy)butyl]piperidine (220 mg, 606.51 μmol, 1 equiv., HCl) in MeCN (2 mL) was added K₂CO₃ (251.47 mg, 1.82 mmol, 3 equiv.). The mixture was stirred at 60° C. for 1 hour. The reaction mixture was filtered and then concentrated under reduced pressure to give a residue. The residue was purified by flash silica gel chromatography (eluent of 0˜25% ethyl acetate/petroleum ether) to give ethyl 2-[(3S)-3-[4-(3-bromo-2-methyl-phenoxy)butyl]-1-piperidyl]acetate (0.18 g, 436.5 μmol, 71.9% yield) as a light yellow oil.

¹H NMR (400 MHz, CDCl₃) δ=7.06 (d, J=8.0 Hz, 1H), 6.91 (t, J=8.0 Hz, 1H), 6.68 (d, J=8.0 Hz, 1H), 4.11 (q, J=7.2 Hz, 2H), 3.86 (t, J=6.4 Hz, 2H), 3.12 (s, 2H), 2.85-2.77 (m, 2H), 2.23 (s, 3H), 2.08-1.99 (m, 1H), 1.73-1.67 (m, 3H), 1.63-1.55 (m, 3H), 1.48-1.36 (m, 2H), 1.23-1.18 (m, 5H), 0.84-0.75 (m, 2H)

### Step G. Procedure for Preparation of tert-butyl 6-[8-(1,3-benzothiazol-2-ylcarbamoyl)-3,4-dihydro-1H-isoquinolin-2-yl]-3-[3-[4-[(3S)-1-(2-ethoxy-2-oxo-ethyl)-3-piperidyl]butoxy]-2-methyl-phenyl]pyridine-2-carboxylate

A mixture of ethyl 2-[(3S)-3-[4-(3-bromo-2-methyl-phenoxy)butyl]-1-piperidyl]acetate (0.18 g, 436.51 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (267.38 mg, 436.51 μmol, 1 equiv.), K₃PO₄ (1.5 M, 873.02 μL, 3 equiv.), and [2-(2-aminophenyl)phenyl]palladium(1+); bis(1-adamantyl)-butyl-phosphane; methanesulfonate (31.79 mg, 43.65 μmol, 0.1 equiv.) in dioxane (3 mL) was degassed and purged with N₂ 3 times, and then the mixture was stirred at 80° C. for 1 hour under N₂ atmosphere. The reaction mixture was concentrated under reduced pressure to give a residue. The residue was purified by flash silica gel chromatography (eluent of 0˜30% ethyl acetate/petroleum ether) to give tert-butyl 6-[8-(1,3-benzothiazol-2-ylcarbamoyl)-3,4-dihydro-1H-isoquinolin-2-yl]-3-[3-[4-[(3S)-1-(2-ethoxy-2-oxo-ethyl)-3-piperidyl]butoxy]-2-methyl-phenyl]pyridine-2-carboxylate (0.24 g, 228.8 μmol, 52.4% yield, 78% purity) as a yellow oil.

MS (ESI) m/z: 381.7 [(M−56)/2+H]⁺

### Step H. Procedure for Preparation of 2-[(3S)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[4-[(3S)-1-(2-ethoxy-2-oxo-ethyl)-3-piperidyl]butoxy]-2-methyl-phenyl]pyridine-2-carboxylate (0.24 g, 228.84 μmol, 78% purity, 1 equiv.) in THF (1 mL) was added LiOH·H₂O (1.5 M, 762.81 μL, 5 equiv.). The mixture was stirred at 20° C. for 1 hour. The mixture was concentrated under reduced pressure and then adjusted to pH<7 and filtered to give crude 2-[(3S)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (0.16 g, 202.5 μmol, 88.5% yield) as a brown solid

¹H NMR (400 MHz, DMSO-d₆) δ=13.32-12.25 (m, 1H), 8.01 (d, J=7.6 Hz, 1H), 7.77 (d, J=8.0 Hz, 1H), 7.60 (d, J=7.6 Hz, 1H), 7.49-7.40 (m, 3H), 7.40-7.31 (m, 2H), 7.14-7.06 (m, 1H), 6.93 (dd, J=8.4, 12.4 Hz, 2H), 6.57 (d, J=7.6 Hz, 1H), 4.98 (s, 2H), 3.97 (t, J=6.0 Hz, 2H), 3.87 (t, J=6.0 Hz, 2H), 3.18 (s, 2H), 3.15 (d, J=2.0 Hz, 2H), 3.03 (t, J=6.0 Hz, 2H), 2.24 (t, J=11.2 Hz, 1H), 1.87 (s, 3H), 1.76-1.56 (m, 7H), 1.50-1.37 (m, 2H), 1.29-1.18 (m, 3H), 1.00 (s, 9H), 0.95-0.88 (m, 1H)

### Step I. Procedure for preparation of tert-butyl 6-[8-(1,3-benzothiazol-2-ylcarbamoyl)-3,4-dihydro-1H-isoquinolin-2-yl]-3-[3-[4-[(3S)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylate

A mixture of 2-[(3S)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (50 mg, 63.29 μmol, 1 equiv.), 2-(2,6-dioxo-3-piperidyl)-5-fluoro-6-piperazin-1-yl-isoindoline-1,3-dione (22.81 mg, 63.29 μmol, 1 equiv.), and HATU (28.88 mg, 75.95 μmol, 1.2 equiv.), in DMF (0.5 mL) and DIEA (0.05 mL) was degassed and purged with N₂ and stirred at 40° C. for 12 hours under N₂ atmosphere. After completion, H₂O was added into the reaction mixture, and the mixture was filtered and then concentrated under reduced pressure to give crude tert-butyl 6-[8-(1,3-benzothiazol-2-ylcarbamoyl)-3,4-dihydro-1H-isoquinolin-2-yl]-3-[3-[4-[(3S)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylate (60 mg, crude) as a yellow solid.

### Step J. Procedure for Preparation of 6-[8-(1,3-benzothiazol-2-ylcarbamoyl)-3,4-dihydro-1H-isoquinolin-2-yl]-3-[3-[4-[(3S)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[(3S)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylate (60 mg, 52.99 μmol, 1 equiv.) in TFA (0.5 mL) and DCM (0.5 mL) was degassed and purged with N₂ and stirred at 25° C. for 12 hours under N₂ atmosphere. The reaction mixture was concentrated under reduced pressure to remove DCM and TFA to give a residue. The residue was purified by prep-HPLC to give 6-[8-(1,3-benzothiazol-2-ylcarbamoyl)-3,4-dihydro-1H-isoquinolin-2-yl]-3-[3-[4-[(3S)-1-[2-[4-[2-(2,6-dioxo-3-piperidyl)-6-fluoro-1,3-dioxo-isoindolin-5-yl]piperazin-1-yl]-2-oxo-ethyl]-3-piperidyl]butoxy]-2-methyl-phenyl]pyridine-2-carboxylic acid (19.4 mg, 16.8 μmol, 31.8% yield, 93.2% purity) as a yellow solid.

MS (ESI) m/z: 1076.7 [M+H]⁺.

¹H NMR (400 MHz, DMSO-d₆) δ=12.92-12.71 (m, 1H), 11.10 (s, 1H), 8.14 (s, 1H), 8.02 (d, J=8.0 Hz, 1H), 7.78 (d, J=8.4 Hz, 1H), 7.72 (d, J=11.6 Hz, 1H), 7.64-7.60 (m, 1H), 7.49-7.41 (m, 4H), 7.39-7.32 (m, 2H), 7.09-7.05 (m, 1H), 6.96 (d, J=8.8 Hz, 1H), 6.85 (d, J=7.6 Hz, 1H), 6.61 (d, J=7.6 Hz, 1H), 5.13-5.07 (m, 1H), 4.98 (s, 2H), 3.96-3.88 (m, 5H), 3.80-3.67 (m, 3H), 3.66-3.54 (m, 3H), 3.19-3.13 (m, 3H), 3.07-2.97 (m, 3H), 2.69-2.65 (m, 3H), 2.34-2.31 (m, 2H), 2.06-1.95 (m, 2H), 1.86 (s, 3H), 1.73-1.63 (m, 4H), 1.50-1.39 (m, 4H), 1.23 (s, 2H), 0.96-0.77 (m, 1H)

¹⁹F NMR (400 MHz, DMSO-d₆) δ=−112.03

### Example 29. Preparation of Compound 137

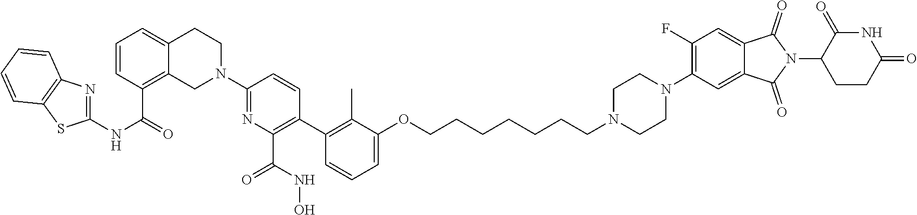 [SMILES: Cc1c(OCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1C(=O)NO]

### N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)-6-(hydroxycarbamoyl)pyridin-2-yl)-1,2,3,4-tetrahydroisoquinoline-8-carboxamide

### Step A. Procedure for Preparation of N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)-6-(((tetrahydro-2H-pyran-2-yl)oxy)carbamoyl)pyridin-2-yl)-1,2,3,4-tetrahydroisoquinoline-8-carboxamide

To a solution of 6-(8-(benzo[d]thiazol-2-ylcarbamoyl)-3,4-dihydroisoquinolin-2(1H)-yl)-3-(3-((7-(4-(2-(2,6-dioxopiperidin-3-yl)-6-fluoro-1,3-dioxoisoindolin-5-yl)piperazin-1-yl)heptyl)oxy)-2-methylphenyl) picolinic acid (20 mg, 20.14 μmol, 1 equiv.) in DCM (1 mL) was added HATU (15.31 mg, 40.28 μmol, 2 equiv.), DIEA (7.81 mg, 60.42 μmol, 10.52 μL, 3 equiv.), and O-tetrahydropyran-2-ylhydroxylamine (7.08 mg, 60.42 μmol, 2 equiv.). The mixture was stirred at 25° C. for 2 hours. The reaction mixture was concentrated under reduced pressure to remove solvent and to give N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)-6-(((tetrahydro-2H-pyran-2-yl)oxy)carbamoyl)pyridin-2-yl)-1,2,3,4-tetrahydroisoquinoline-8-carboxamide (30 mg, crude) as yellow oil.

MS (ESI) m/z: 1092.5 [M+H]⁺

### Step B. Procedure for Preparation of N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)-6-(hydroxycarbamoyl)pyridin-2-yl)-1,2,3,4-tetrahydroisoquinoline-8-carboxamide

To a solution of N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)-6-(((tetrahydro-2H-pyran-2-yl)oxy)carbamoyl)pyridin-2-yl)-1,2,3,4-tetrahydroisoquinoline-8-carboxamide (20 mg, 18.31 μmol, 1 equiv.) in DCM (1 mL) was added TFA (2.09 mg, 18.31 μmol, 1.36 μL, 1 equiv.). The mixture was stirred at 25° C. for 1 hour. The reaction mixture was concentrated under reduced pressure to remove solvent. The residue was diluted with DMF (1 mL). The residue was purified by prep-HPLC to give N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)-6-(hydroxycarbamoyl)pyridin-2-yl)-1,2,3,4-tetrahydroisoquinoline-8-carboxamide (11.1 mg, 4.9 μmol, 26.8% yield, 97.5% purity) as a yellow solid.

MS (ESI) m/z: 1008.7 [M+H]⁺

¹H NMR (400 MHz, DMSO-d₆) δ=11.10 (s, 1H), 10.72 (s, 1H), 8.96-8.78 (m, 1H), 8.15 (s, 1H), 8.02 (d, J=7.6 Hz, 1H), 7.78 (d, J=8.4 Hz, 1H), 7.71 (d, J=10.8 Hz, 1H), 7.63 (d, J=7.6 Hz, 1H), 7.52-7.28 (m, 7H), 7.09-7.02 (m, 1H), 6.92 (d, J=8.4 Hz, 1H), 6.84 (d, J=8.8 Hz, 1H), 6.64 (d, J=7.6 Hz, 1H), 5.10 (m, 1H), 4.95 (m, 2H), 3.98-3.91 (m, 4H), 3.42-3.34 (m, 4H), 3.23 (m, 4H), 3.01 (t, J=5.6 Hz, 2H), 2.60 (m, 2H), 2.32 (m, 2H), 2.10-1.95 (m, 2H), 1.88 (s, 3H), 1.77-1.71 (m, 2H), 1.46 (d, J=5.2 Hz, 4H), 1.40-1.31 (m, 4H)

### Example 30. Preparation of Compound 139a

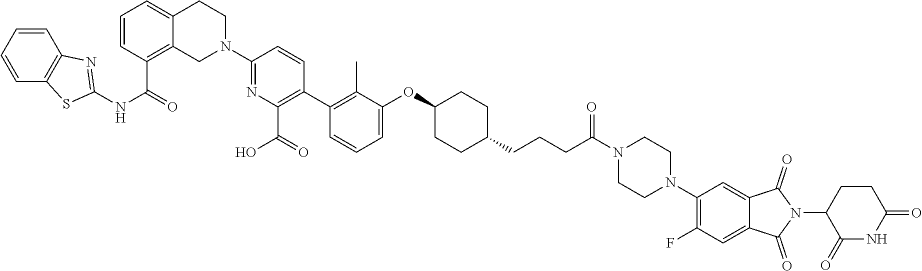 [SMILES: Cc1c(O[C@H]2CC[C@H](CCCC(=O)N3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)-4-oxobutyl)cyclohexyl)oxy)-2-methylphenyl)picolinic acid

### Step A. Procedure for Preparation of 1-(((1r,4r)-4-((E)-4-(benzyloxy)but-1-en-1-yl)cyclohexyl)oxy)-3-bromo-2-methylbenzene

To a mixture of 3-benzyloxypropyl(triphenyl)phosphonium; bromide (5.56 g, 11.31 mmol, 1.2 equiv.) in THF (30 mL) was added LiHMDS (1 M, 12.25 mL, 1.3 equiv.) at −70° C. under N₂ atmosphere for 1 hour. Then 4-(3-bromo-2-methyl-phenoxy)cyclohexanecarbaldehyde (2.8 g, 9.42 mmol, 1 equiv.) was added to the mixture at −70° C. under N₂ atmosphere. The mixture was stirred at 25° C. for 11 hours under N₂ atmosphere. The reaction mixture was quenched by addition of NH₄Cl (100 mL) at 0° C. and then diluted with H₂O (80 mL) and extracted with ethyl acetate (80 mL×3). The combined organic layers were washed with aqueous NaCl (100 mL×3), dried over Na₂SO₄, filtered, and concentrated under reduced pressure to give a residue. The residue was purified by column chromatography (SiO₂, petroleum ether/ethyl acetate 30/1 to 10/1) to give 1-(((1r,4r)-4-((E)-4-(benzyloxy)but-1-en-1-yl)cyclohexyl)oxy)-3-bromo-2-methylbenzene (3 g, 6.9 mmol, 74.1% yield) as a yellow oil.

¹H NMR (400 MHz, CDCl₃) δ=7.21-7.12 (m, 5H), 6.98 (d, J=8.0 Hz, 1H), 6.81 (t, J=8.0 Hz, 1H), 6.65 (d, J=8.0 Hz, 1H), 5.25-5.06 (m, 2H), 4.42-4.33 (m, 2H), 4.00-3.87 (m, 1H), 3.33 (t, J=6.8 Hz, 2H), 2.29-2.21 (m, 2H), 2.14 (s, 3H), 1.97 (d, J=10.4 Hz, 2H), 1.66-1.50 (m, 2H), 1.41-1.26 (m, 3H), 1.10-0.97 (m, 2H)

### Step B. Procedure for Preparation of 4-((1s,4r)-4-(3-bromo-2-methylphenoxy)cyclohexyl)butan-1-ol

To a solution of 1-(((1r,4r)-4-((E)-4-(benzyloxy)but-1-en-1-yl)cyclohexyl)oxy)-3-bromo-2-methylbenzene (2.6 g, 6.06 mmol, 1 equiv.) in EtOAc (20 mL) was added PtO₂ (1.37 g, 6.06 mmol, 1.0 equiv.) under N₂ atmosphere. The suspension was degassed and purged with H₂. The mixture was stirred under H₂ (15 psi) at 40° C. for 12 hours. The reaction mixture was filtered and then concentrated under reduced pressure to give a residue. The residue was purified by prep-HPLC to give 4-((1s,4r)-4-(3-bromo-2-methylphenoxy)cyclohexyl)butan-1-ol (1 g, 2.9 mmol, 48.3% yield) as a yellow oil.

¹H NMR (400 MHz, CDCl₃) δ=7.06 (d, J=7.6 Hz, 1H), 6.89 (t, J=8.0 Hz, 1H), 6.76-6.69 (m, 1H), 4.06-3.97 (m, 1H), 3.58 (t, J=6.8 Hz, 2H), 2.22 (s, 3H), 2.08-2.01 (m, 2H), 1.80-1.74 (m, 2H), 1.51-1.47 (m, 2H), 1.39-1.28 (m, 4H), 1.23-1.14 (m, 3H), 1.00-0.88 (m, 2H)

### Step C. Procedure for Preparation of 4-((1s,4r)-4-(3-bromo-2-methylphenoxy)cyclohexyl)butanal

To a solution of oxalyl dichloride (371.92 mg, 2.93 mmol, 256.50 μL, 2 equiv.) in DCM (5 mL) was added dropwise to a solution of DMSO (457.86 mg, 5.86 mmol, 457.86 μL, 4 equiv.) in DCM (5 mL) at −70° C. under N₂ atmosphere. The mixture was stirred at −70° C. for 30 minutes, then 4-((1s,4r)-4-(3-bromo-2-methylphenoxy)cyclohexyl)butan-1-ol (500 mg, 1.47 mmol, 1 equiv.) in DCM (5 mL) was added dropwise at −70° C. The solution was stirred for 30 minutes at −70° C. Then TEA (889.50 mg, 8.79 mmol, 1.22 mL, 6 equiv.) was added into the solution. The solution was stirred at −70° C. for 2 hours under N₂ atmosphere. The reaction was diluted with water (50 mL) and extracted with DCM (40 mL×2). The combined organic layers were washed with brine 60 mL (60 mL×1), dried over Na₂SO₄, filtered, and concentrated under reduced pressure to give a residue. The residue was purified by column chromatography (SiO₂, petroleum ether/ethyl acetate 100/1 to 15/1) to give 4-((1s,4r)-4-(3-bromo-2-methylphenoxy)cyclohexyl)butanal (400 mg, 1.1 mmol, 80.4% yield) as a yellow oil.

### Step D. Procedure for Preparation of 4-((1s,4r)-4-(3-bromo-2-methylphenoxy)cyclohexyl)butanoic acid

A mixture of 4-((1s,4r)-4-(3-bromo-2-methylphenoxy)cyclohexyl)butanal (500 mg, 1.47 mmol, 1 equiv.), NaH₂PO4 (529.11 mg, 4.41 mmol, 3 equiv.), sodium chlorite (398.85 mg, 4.41 mmol, 3 equiv.), and 2-methylbut-2-ene (618.55 mg, 8.82 mmol, 934.36 μL, 6 equiv.) in t-BuOH (6 mL) and THF (2 mL), H₂O (1 mL). The mixture was stirred at 25° C. for 12 hours under N₂ atmosphere. The reaction was diluted with water (50 mL) and extracted with DCM (40 mL×2). The combined organic layers were washed with brine 60 mL (60 mL×1), dried over Na₂SO₄, filtered, and concentrated under reduced pressure to give a residue. The residue was purified by column chromatography (SiO₂, petroleum ether/ethyl acetate 20/1 to 8/1) to give 4-((1s,4r)-4-(3-bromo-2-methylphenoxy)cyclohexyl)butanoic acid (400 mg, 1.1 mmol, 76.5% yield) as a white solid.

¹H NMR (400 MHz, DMSO-d₆) δ=11.98 (s, 1H), 7.17-7.11 (m, 1H), 7.10-6.99 (m, 2H), 4.30-4.16 (m, 1H), 2.21 (s, 3H), 2.20-2.17 (m, 1H), 2.04 (d, J=11.6 Hz, 2H), 1.76 (d, J=11.6 Hz, 2H), 1.56-1.47 (m, 2H), 1.42-1.33 (m, 2H), 1.31-1.15 (m, 4H), 1.09-0.98 (m, 2H)

### Step E. Procedure for Preparation of 4-((1s,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)butanoic acid

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (600 mg, 979.52 μmol, 1 equiv.), 4-((1s,4r)-4-(3-bromo-2-methylphenoxy)cyclohexyl)butanoic acid (382.79 mg, 1.08 mmol, 1.1 equiv.), [2-(2-aminophenyl)phenyl]palladium(1+); bis(1-adamantyl)-butyl-phosphane; methanesulfonate (71.34 mg, 97.95 μmol, 0.1 equiv.), and KF (170.73 mg, 2.94 mmol, 68.84 μL, 3 equiv.) in dioxane (6 mL) and H₂O (2 mL) was degassed and purged with N₂ and then stirred at 100° C. for 12 hours under N₂ atmosphere. The reaction mixture was concentrated under reduced pressure to give a residue. The residue was purified by column chromatography (SiO₂, dichloromethane:methanol 20/1 to 10/1) to give 4-((1s,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)butanoic acid (400 mg, 525.7 μmol, 53.7% yield) as a yellow solid MS (ESI) m/z: 761.8 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)-4-oxobutyl)cyclohexyl)oxy)-2-methylphenyl)picolinate

To a solution of 4-((1s,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)butanoic acid (80 mg, 105.13 μmol, 1 equiv.) in DMF (1 mL) was added DIEA (40.76 mg, 315.40 μmol, 54.94 μL, 3 equiv.), HATU (47.97 mg, 126.16 μmol, 1.2 equiv.), and 2-(2,6-dioxo-3-piperidyl)-5-fluoro-6-piperazin-1-yl-isoindoline-1,3-dione (45.46 mg, 126.16 μmol, 1.2 equiv.). The mixture was stirred at 25° C. for 2 hours. The reaction was diluted with water (20 mL) and extracted with DCM (20 mL×2). The combined organic layers were washed with brine 30 mL (30 mL×1), dried over Na₂SO₄, filtered, and concentrated under reduced pressure to give a residue. The residue was purified by prep-TLC (SiO₂, petroleum ether:ethyl acetate 0:1) to give tert-butyl 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)-4-oxobutyl)cyclohexyl)oxy)-2-methylphenyl)picolinate (30 mg, 27.1 μmol, 25.8% yield) as a yellow oil.

MS (ESI) m/z: 1103.7 [M+H]⁺.

### Step G. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)-4-oxobutyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)-4-oxobutyl)cyclohexyl)oxy)-2-methylphenyl)picolinate (30 mg, 27.19 μmol, 1 equiv.) in DCM (0.3 mL) was added TFA (462.00 mg, 4.05 mmol, 0.3 mL, 149.01 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(2-(2,6-dioxopiperidin-3-yl)-6-fluoro-1,3-dioxoisoindolin-5-yl)piperazin-1-yl)-4-oxobutyl)cyclohexyl)oxy)-2-methylphenyl)picolinic acid (11.1 mg, 10.5 μmol, 38.7% yield, 99.2% purity) as a pink solid.

MS (ESI) m/z: 1047.7 [M+H]⁺.

¹H NMR (400 MHz, DMSO-d₆) δ=12.86-12.69 (m, 1H), 11.05 (s, 1H), 7.97 (d, J=8.0 Hz, 1H), 7.75-7.67 (m, 2H), 7.56 (d, J=7.6 Hz, 1H), 7.44-7.37 (m, 4H), 7.33-7.26 (m, 2H), 7.04-6.98 (m, 1H), 6.93-6.84 (m, 2H), 6.55 (d, J=7.2 Hz, 1H), 5.07-5.02 (m, 1H), 4.91 (s, 2H), 4.16-4.10 (m, 1H), 3.85 (t, J=5.6 Hz, 2H), 3.56 (s, 4H), 3.20 (s, 2H), 3.14 (s, 2H), 2.96 (t, J=5.6 Hz, 2H), 2.85-2.80 (m, 1H), 2.55 (d, J=1.2 Hz, 2H), 2.30-2.25 (m, 2H), 2.01-1.95 (m, 2H), 1.80 (s, 3H), 1.74-1.68 (m, 2H), 1.50-1.44 (m, 2H), 1.34-1.26 (m, 2H), 1.23-1.14 (m, 4H), 1.04-0.94 (m, 2H)

### Example 31. Preparation of Compound 140a

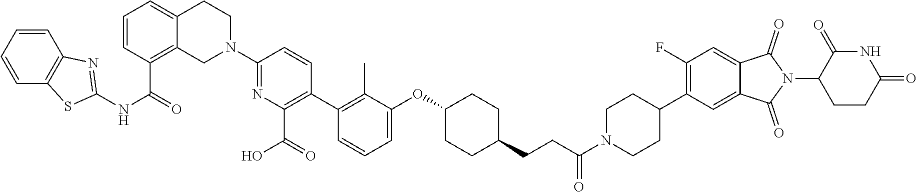 [SMILES: Cc1c(O[C@H]2CC[C@H](CCC(=O)N3CCC(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperidin-1-yl)-3-oxopropyl)cyclohexyl)oxy)-2-methylphenyl)picolinic acid

### Step A. Procedure for Preparation of ethyl 4-(3-bromo-2-methyl-phenoxy)cyclohexanecarboxylate

To a solution of ethyl 4-hydroxycyclohexanecarboxylate (10 g, 58.06 mmol, 1 equiv.), 3-bromo-2-methyl-phenol (11.95 g, 63.87 mmol, 1.1 equiv.), and PPh₃ (24.37 g, 92.90 mmol, 1.6 equiv.) in THF (100 mL) was added DIAD (18.79 g, 92.90 mmol, 18.06 mL, 1.6 equiv.) under N₂ at 0° C. Then the mixture was stirred at 25° C. for 16 hours. The reaction mixture was concentrated under reduced pressure to give a residue. The residue was purified by flash silica gel chromatography (eluent of 0˜10% ethyl acetate/petroleum ether) to give ethyl 4-(3-bromo-2-methyl-phenoxy)cyclohexanecarboxylate (3.6 g, 10.55 mmol, 18.17% yield) as a white solid.

¹H NMR (400 MHz, CDCl₃) δ=7.14 (d, J=8.0 Hz, 1H), 6.97 (t, J=8.0 Hz, 1H), 6.80 (d, J=8.0 Hz, 1H), 4.20-4.11 (m, 3H), 2.39-2.35 (m, 2H), 2.30 (s, 3H), 2.19-2.14 (m, 2H), 2.09-2.04 (m, 2H), 1.62-1.52 (m, 5H), 1.26 (t, J=6.8 Hz, 3H).

### Step B. Procedure for Preparation of [4-(3-bromo-2-methyl-phenoxy)cyclohexyl]methanol

To a solution of ethyl 4-(3-bromo-2-methyl-phenoxy)cyclohexanecarboxylate (3.6 g, 10.55 mmol, 1 equiv.) in THF (15 mL) was added LAH (480.49 mg, 12.66 mmol, 1.2 equiv.) under N₂ at 0° C. The mixture was stirred at 25° C. for 1.5 hours. The reaction mixture was quenched by addition Na₂SO₄·10H₂O (1 g) and then extracted with ethyl acetate 30 mL (10 mL×3). The combined organic layers were dried over Na₂SO₄, filtered, and concentrated under reduced pressure to give [4-(3-bromo-2-methyl-phenoxy)cyclohexyl]methanol (2.96 g, crude) as a white solid.

¹H NMR (400 MHz, CDCl₃) δ=7.13 (d, J=8.0 Hz, 1H), 6.97 (t, J=8.0 Hz, 1H), 6.81 (d, J=8.4 Hz, 1H), 4.13-4.08 (m, 1H), 3.50 (d, J=6.4 Hz, 2H), 2.30 (s, 3H), 2.20-2.14 (m, 2H), 1.95-1.86 (m, 2H), 1.53-1.45 (m, 3H), 1.14-1.04 (m, 2H).

### Step C. Procedure for Preparation of 4-(3-bromo-2-methyl-phenoxy)cyclohexanecarbaldehyde

To a solution of DMSO (3.09 g, 39.57 mmol, 3.09 mL, 4 equiv.) in DCM (10 mL) was added dropwise a solution of (COCl)₂ (2.51 g, 19.79 mmol, 1.73 mL, 2 equiv.) in DCM (2 mL) at −70° C. under N₂ atmosphere. The mixture was stirred at −70° C. for 1 hour. After that, [4-(3-bromo-2-methyl-phenoxy)cyclohexyl]methanol (2.96 g, 9.89 mmol, 1 equiv.) in DCM (10 mL) was added dropwise at −70° C. The solution was stirred for 1 hour at −70° C. Then TEA (6.01 g, 59.36 mmol, 8.26 mL, 6 equiv.) was added into the solution. The solution was stirred at −70° C. for 0.5 hour under N₂ atmosphere. The reaction mixture was quenched by addition H₂O (10 mL), and then diluted with DCM (10 mL) and extracted with DCM (10 mL×3). The combined organic layers were washed with H₂O (5 mL×3), dried over anhydrous sodium sulfate, filtered, and concentrated under reduced pressure to give 4-(3-bromo-2-methyl-phenoxy)cyclohexanecarbaldehyde (3 g, crude) as a white solid.

¹H NMR (400 MHz, DMSO-d₆) δ=9.62-9.59 (m, 1H), 7.16-7.01 (m, 3H), 4.36-4.23 (m, 1H), 2.39-2.31 (m, 1H), 2.25-2.18 (m, 3H), 2.03-1.92 (m, 4H), 1.50-1.38 (m, 4H).

### Step D. Procedure for Preparation of ethyl (E)-3-[4-(3-bromo-2-methyl-phenoxy)cyclohexyl]prop-2-enoate

To a solution of NaH (56.52 mg, 1.41 mmol, 60% purity, 2.1 equiv.) in THF (3 mL) was added ethyl 2-diethoxyphosphorylacetate (301.75 mg, 1.35 mmol, 267.04 μL, 2 equiv.) under 0° C. Then 4-(3-bromo-2-methyl-phenoxy)cyclohexanecarbaldehyde (200 mg, 672.98 μmol, 1 equiv.) was added, and the mixture was stirred at 25° C. for 6 hours. The reaction mixture was quenched by addition of sat. NH₄Cl (5 mL) at 0° C., and then diluted with ethyl acetate (5 mL) and extracted with DCM (10 mL×3). The combined organic layers were washed with H₂O (10 mL), dried over Na₂SO₄, filtered, and concentrated under reduced pressure to give a residue. The residue was purified by flash silica gel chromatography (eluent of 0˜10% ethyl acetate/petroleum ether) to give ethyl (E)-3-[4-(3-bromo-2-methyl-phenoxy)cyclohexyl]prop-2-enoate (140 mg, 362.1 μmol, 53.8% yield, 95% purity) as a colorless oil.

¹H NMR (400 MHz, CDCl₃) δ=7.14 (d, J=8.0 Hz, 1H), 7.01-6.87 (m, 2H), 6.80 (d, J=8.4 Hz, 1H), 5.82 (dd, J=1.2, 15.6 Hz, 1H), 4.21-4.15 (m, 2H), 4.16-4.07 (m, 1H), 2.30 (s, 3H), 2.26-2.16 (m, 3H), 1.97-1.89 (m, 2H), 1.58-1.48 (m, 2H), 1.33-1.26 (m, 5H).

### Step E. Procedure for Preparation of ethyl 3-[4-(3-bromo-2-methyl-phenoxy)cyclohexyl]propanoate

A mixture of ethyl (E)-3-[4-(3-bromo-2-methyl-phenoxy)cyclohexyl]prop-2-enoate (1.6 g, 4.36 mmol, 1 equiv.) and PtO₂ (98.92 mg, 435.64 μmol, 0.1 equiv.) in EtOH (15 mL) was degassed and purged with H₂ 3 times, and then the mixture was stirred at 25° C. for 3 hours under H₂ (15 psi) atmosphere. The reaction mixture was filtered and then concentrated under reduced pressure to give a residue. The residue was purified by prep-HPLC to give ethyl 3-[4-(3-bromo-2-methyl-phenoxy)cyclohexyl]propanoate (1.4 g, 3.7 mmol, 87.0% yield) as a white solid.

¹H NMR (400 MHz, DMSO-d₆) δ=7.17-7.10 (m, 1H), 7.06 (t, J=8.0 Hz, 1H), 7.03-6.99 (m, 1H), 4.27-4.17 (m, 1H), 4.11-4.04 (m, 2H), 2.33-2.26 (m, 2H), 2.20 (s, 3H), 2.03 (d, J=10.0 Hz, 2H), 1.75 (d, J=12.0 Hz, 2H), 1.55-1.42 (m, 2H), 1.39-1.21 (m, 3H), 1.17 (t, J=7.2 Hz, 3H), 1.11-0.97 (m, 2H).

### Step F. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate

A mixture of ethyl 3-[4-(3-bromo-2-methyl-phenoxy)cyclohexyl]propanoate (200 mg, 541.58 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (546.40 mg, 758.21 μmol, 85% purity, 1.4 equiv.), [2-(2-aminophenyl)phenyl]palladium(1+); bis(1-adamantyl)-butyl-phosphane; methanesulfonate (78.88 mg, 108.32 μmol, 0.2 equiv.), and K₂CO₃ (1.5 M, 541.58 μL, 1.5 equiv.) in dioxane (2.5 mL) was degassed and purged with N₂ and then stirred at 100° C. for 1 hour under microwave. The reaction mixture was filtered and then concentrated under reduced pressure to give a residue. The residue was purified by flash silica gel chromatography (eluent of 0˜50% ethyl acetate/petroleum ether) to give tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (440 mg, crude) as a white solid.

¹H NMR (400 MHz, DMSO-d₆) δ=8.02 (d, J=7.6 Hz, 1H), 7.78 (d, J=8.0 Hz, 1H), 7.60 (d, J=7.5 Hz, 1H), 7.50-7.40 (m, 3H), 7.40-7.30 (m, 2H), 7.12-7.03 (m, 1H), 6.99-6.89 (m, 2H), 6.59-6.49 (m, 1H), 5.03-4.90 (m, 2H), 4.27-4.14 (m, 1H), 4.03 (d, J=6.8 Hz, 2H), 3.87 (t, J=5.6 Hz, 2H), 3.29 (s, 2H), 3.03 (t, J=5.6 Hz, 2H), 2.29 (t, J=7.6 Hz, 2H), 2.05 (d, J=11.2 Hz, 1H), 1.84 (s, 3H), 1.79-1.70 (m, 2H), 1.50-1.42 (m, 2H), 1.23 (d, J=8.4 Hz, 2H), 1.19-1.17 (m, 3H), 1.07 (s, 2H), 1.00 (s, 9H).

### Step G. Procedure for Preparation of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid

A mixture of tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (440 mg, 567.77 μmol, 1 equiv.), and LiOH·H₂O (71.47 mg, 1.70 mmol, 3 equiv.) in THF (1.2 mL) and H₂O (0.4 mL) was stirred at 25° C. for 1.5 hours. The mixture was concentrated and redissolved by 2 mL H₂O, then 1M HCl was added to adjust the pH to 2, and the residue was filtered and then dried to give 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (380 mg, 508.7 μmol, 89.6% yield) was obtained as a gray solid.

### Step H. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperidin-1-yl)-3-oxopropyl)cyclohexyl)oxy)-2-methylphenyl)picolinate

A mixture of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (100 mg, 133.88 μmol, 1 equiv.), 2-(2,6-dioxo-3-piperidyl)-5-fluoro-6-(4-piperidyl)isoindoline-1,3-dione (57.73 mg, 160.66 μmol, 1.2 equiv.), and EDCI (38.50 mg, 200.83 μmol, 1.5 equiv.) in pyridine (1 mL) was stirred at 25° C. for 3 hours. The mixture was diluted with water (2 mL) and extracted with ethyl acetate 3 mL (1 mL×3). The combined organic layers were filtered and then concentrated under reduced pressure to give a residue. The residue was used for the next step without other purification. Tert-butyl 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperidin-1-yl)-3-oxopropyl)cyclohexyl)oxy)-2-methylphenyl)picolinate (100 mg, crude) was obtained as a yellow oil.

MS (ESI) m/z: 1088.7 [M+H]⁺.

### Step I. Procedure for preparation of 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperidin-1-yl)-3-oxopropyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperidin-1-yl)-3-oxopropyl)cyclohexyl)oxy)-2-methylphenyl)picolinate (90 mg, 82.70 μmol, 1 equiv.) in TFA (2.5 mL) and DCM (0.5 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-(4-(2-(2,6-dioxopiperidin-3-yl)-6-fluoro-1,3-dioxoisoindolin-5-yl)piperidin-1-yl)-3-oxopropyl)cyclohexyl)oxy)-2-methylphenyl)picolinic acid (18.3 mg, 18.1 μmol, 22.0% yield, 99.2% purity) as a white solid.

MS (ESI) m/z: 1032.5 [M+H]⁺.

¹H NMR (400 MHz, DMSO-d₆) δ=12.89-12.54 (m, 2H), 11.12 (s, 1H), 8.03 (d, J=8.0 Hz, 1H), 7.95 (d, 1H), 7.82-7.80 (m, 2H), 7.62 (d, J=7.6 Hz, 1H), 7.46-7.43 (m, 3H), 7.37-7.34 (m, 2H), 7.07 (t, J=8.0 Hz, 1H), 6.94 (dd, J=8.4, 15.6 Hz, 2H), 6.61 (d, J=7.6 Hz, 1H), 5.14 (dd, J=5.2, 12.8 Hz, 1H), 4.98 (s, 2H), 4.63-4.55 (m, 1H), 4.25-4.15 (m, 1H), 4.04-3.98 (m, 1H), 3.92-3.90 (m, 2H), 3.23-3.14 (m, 2H), 3.04-3.02 (m, 2H), 2.93-2.84 (m, 1H), 2.67-2.63 (m, 2H), 2.39-2.35 (m, 2H), 2.1-2.03 (m, 3H), 1.86 (s, 3H), 1.83-1.79 (m, 5H), 1.64-1.44 (m, 4H), 1.38-1.28 (m, 3H), 1.2-1.03 (m, 2H)

¹⁹F NMR (400 MHz, DMSO-d₆) δ=−107.869.

### Example 32. Preparation of Compound 142

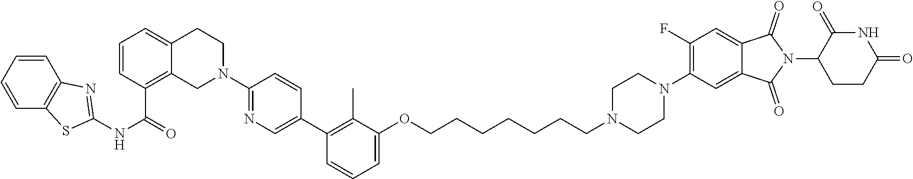 [SMILES: Cc1c(OCCCCCCCN2CCN(c3cc4c(cc3F)C(=O)N(C3CCC(=O)NC3=O)C4=O)CC2)cccc1-c1ccc(N2CCc3cccc(C(=O)Nc4nc5ccccc5s4)c3C2)nc1]

### N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)pyridin-2-yl)-1,2,3,4-tetrahydroisoquinoline-8-carboxamide

### Step A. Procedure for Preparation of N-(1,3-benzothiazol-2-yl)-2-(5-bromo-2-pyridyl)-3,4-dihydro-1H-isoquinoline-8-carboxamide

To a solution of N-(1,3-benzothiazol-2-yl)-1,2,3,4-tetrahydroisoquinoline-8-carboxamide (1 g, 2.89 mmol, 1 equiv., HCl) in DMSO (10 mL) was added Cs₂CO₃ (2.83 g, 8.67 mmol, 3 equiv.) and 5-bromo-2-chloro-pyridine (612.08 mg, 3.18 mmol, 1.1 equiv.). The mixture was stirred at 120° C. for 12 hours. The reaction was diluted with water (100 mL) and extracted with ethyl acetate (100 mL×3). The combined organic layers were washed with brine 100 mL (100 mL×1), dried over Na₂SO₄, filtered, and concentrated under reduced pressure to give a residue. The residue was purified by column chromatography (SiO₂, petroleum ether/ethyl acetate 100/1 to 3/1) to give N-(1,3-benzothiazol-2-yl)-2-(5-bromo-2-pyridyl)-3,4-dihydro-1H-isoquinoline-8-carboxamide (580 mg, 1.2 mmol, 43.1% yield) as a white solid.

MS (ESI) m/z: 467.1 [M+H]⁺.

¹H NMR (400 MHz, DMSO-d₆) δ=12.94 (s, 1H), 8.23 (d, J=2.4 Hz, 1H), 8.11 (d, J=7.6 Hz, 1H), 7.86 (d, J=8.0 Hz, 1H), 7.79-7.74 (m, 1H), 7.66 (d, J=7.6 Hz, 1H), 7.54 (t, J=7.6 Hz, 1H), 7.50-7.46 (m, 1H), 7.45-7.39 (m, 2H), 6.89 (d, J=9.2 Hz, 1H), 4.98 (s, 2H), 3.85 (t, J=5.6 Hz, 2H), 3.05 (t, J=5.6 Hz, 2H)

### Step B. Procedure for Preparation of 2-(5-(3-((6-(1,3-dioxolan-2-yl)hexyl)oxy)-2-methylphenyl)pyridin-2-yl)-N-(benzo[d]thiazol-2-yl)-1,2,3,4-tetrahydroisoquinoline-8-carboxamide

A mixture of N-(1,3-benzothiazol-2-yl)-2-(5-bromo-2-pyridyl)-3,4-dihydro-1H-isoquinoline-8-carboxamide (300 mg, 644.66 μmol, 1 equiv.), N-(benzo[d]thiazol-2-yl)-2-(5-bromopyridin-2-yl)-1,2,3,4-tetrahydroisoquinoline-8-carboxamide (251.62 mg, 644.66 μmol, 1 equiv.), [2-(2-aminophenyl)phenyl]palladium(1+); bis(1-adamantyl)-butyl-phosphane; methanesulfonate (46.95 mg, 64.47 μmol, 0.1 equiv.), and KF (112.36 mg, 1.93 mmol, 45.31 μL, 3 equiv.) in dioxane (3 mL) and H₂O (1 mL) was degassed and purged with N₂ and then stirred at 100° C. for 1 hour under N₂ atmosphere. The reaction mixture was concentrated under reduced pressure to give a residue. The residue was purified by column chromatography (SiO₂, petroleum ether/ethyl acetate 20/1 to 2/1) to give 2-(5-(3-((6-(1,3-dioxolan-2-yl)hexyl)oxy)-2-methylphenyl)pyridin-2-yl)-N-(benzo[d]thiazol-2-yl)-1,2,3,4-tetrahydroisoquinoline-8-carboxamide (120 mg, 184.9 μmol, 28.6% yield) as a yellow solid.

MS (ESI) m/z: 649.4 [M+H]⁺.

¹H NMR (400 MHz, DMSO-d₆) δ=12.95 (s, 1H), 8.15-8.01 (m, 2H), 7.80 (d, J=8.0 Hz, 1H), 7.61 (d, J=7.6 Hz, 1H), 7.57-7.52 (m, 1H), 7.51-7.42 (m, 2H), 7.40-7.32 (m, 2H), 7.19-7.13 (m, 1H), 6.90 (d, J=8.8 Hz, 2H), 6.76 (d, J=7.6 Hz, 1H), 4.99 (s, 2H), 4.75 (t, J=4.8 Hz, 1H), 3.99-3.93 (m, 2H), 3.90-3.82 (m, 4H), 3.78-3.71 (m, 2H), 3.03 (t, J=5.6 Hz, 2H), 2.06 (s, 3H), 1.77-1.66 (m, 2H), 1.59-1.51 (m, 2H), 1.49-1.40 (m, 2H), 1.40-1.33 (m, 4H)

### Step C. Procedure for Preparation of N-(benzo[d]thiazol-2-yl)-2-(5-(2-methyl-3-((7-oxoheptyl)oxy)phenyl)pyridin-2-yl)-1,2,3,4-tetrahydroisoquinoline-8-carboxamide

To 2-(5-(3-((6-(1,3-dioxolan-2-yl)hexyl)oxy)-2-methylphenyl)pyridin-2-yl)-N-(benzo[d]thiazol-2-yl)-1,2,3,4-tetrahydroisoquinoline-8-carboxamide (100 mg, 154.13 μmol, 1 equiv.) was added HCOOH (14.81 mg, 308.26 μmol, 0.5 mL, 2 equiv.). The mixture was stirred at 60° C. for 20 minutes. The reaction mixture was concentrated under reduced pressure to remove solvent to give N-(benzo[d]thiazol-2-yl)-2-(5-(2-methyl-3-((7-oxoheptyl)oxy)phenyl)pyridin-2-yl)-1,2,3,4-tetrahydroisoquinoline-8-carboxamide (70 mg, 115.7 μmol, 75.1% yield) as a yellow oil.

MS (ESI) m/z: 605.4 [M+H]⁺.

### Step D. Procedure for Preparation of N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)pyridin-2-yl)-1,2,3,4-tetrahydroisoquinoline-8-carboxamide

To a solution of N-(benzo[d]thiazol-2-yl)-2-(5-(2-methyl-3-((7-oxoheptyl)oxy)phenyl)pyridin-2-yl)-1,2,3,4-tetrahydroisoquinoline-8-carboxamide (70 mg, 115.75 μmol, 1 equiv.) in DCM (1 mL) was added HCOOH (5.56 mg, 115.75 μmol, 1.3 equiv.) and 2-(2,6-dioxo-3-piperidyl)-5-fluoro-6-piperazin-1-yl-isoindoline-1,3-dione (50.52 mg, 127.32 μmol, 1.1 equiv., HCl). The mixture was stirred at 25° C. for 11 hours. NaBH(OAc)₃ (73.60 mg, 347.25 μmol, 3 equiv.) was added to the mixture and stirred at 25° C. for 1 hour. The reaction was diluted with water (10 mL) and extracted with DCM (10 mL×3). The combined organic layers were washed with brine (10 mL), dried over Na₂SO₄, filtered, and concentrated under reduced pressure to give a residue. The residue was purified by prep-HPLC to give N-(benzo[d]thiazol-2-yl)-2-(5-(3-((7-(4-(2-(2,6-dioxopiperidin-3-yl)-6-fluoro-1,3-dioxoisoindolin-5-yl)piperazin-1-yl)heptyl)oxy)-2-methylphenyl)pyridin-2-yl)-1,2,3,4-tetrahydroisoquinoline-8-carboxamide (46.3 mg, 45.9 μmol, 39.6% yield, 98.5% purity) as a yellow solid).

MS (ESI) m/z: 949.6 [M+H]⁺.

¹H NMR (400 MHz, DMSO-d₆) δ=13.32-12.54 (m, 1H), 11.11 (s, 1H), 8.20 (s, 1H), 8.08 (d, J=2.4 Hz, 1H), 8.03 (d, J=7.6 Hz, 1H), 7.78 (d, J=8.0 Hz, 1H), 7.71 (d, J=11.6 Hz, 1H), 7.61 (d, J=6.8 Hz, 1H), 7.56-7.51 (m, 1H), 7.50-7.41 (m, 3H), 7.39-7.31 (m, 2H), 7.19-7.12 (m, 1H), 6.90 (t, J=7.6 Hz, 2H), 6.76 (d, J=7.6 Hz, 1H), 5.14-5.07 (m, 1H), 4.99 (s, 2H), 3.98 (t, J=6.4 Hz, 2H), 3.86 (t, J=6.0 Hz, 2H), 3.22 (s, 6H), 3.02 (t, J=5.6 Hz, 2H), 2.94-2.82 (m, 1H), 2.63-2.52 (m, 4H), 2.32 (t, J=7.2 Hz, 2H), 2.06 (s, 3H), 2.05-1.99 (m, 1H), 1.80-1.71 (m, 2H), 1.51-1.41 (m, 4H), 1.40-1.27 (m, 4H)

### Example 33. Preparation of Compound 143

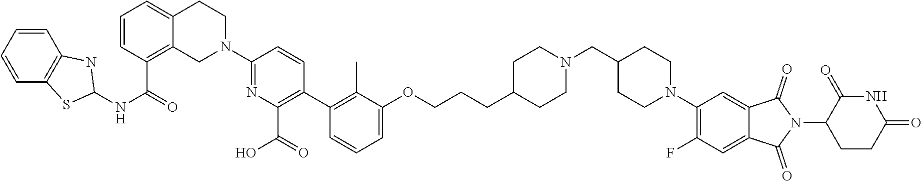 [SMILES: Cc1c(OCCCC2CCN(CC3CCN(c4cc5c(cc4F)C(=O)N(C4CCC(=O)NC4=O)C5=O)CC3)CC2)cccc1-c1ccc(N2CCc3cccc(C(=O)NC4Nc5ccccc5S4)c3C2)nc1C(=O)O]

### 6-[8-(1,3-benzothiazol-2-ylcarbamoyl)-3,4-dihydro-1H-isoquinolin-2-yl]-3-[3-[3-[1-[[1-[2-(2,6-dioxo-3-piperidyl)-6-fluoro-1,3-dioxo-isoindolin-5-yl]-4-piperidyl]methyl]-4-piperidyl]propoxy]-2-methyl-phenyl]pyridine-2-carboxylic acid

### Step A. Procedure for Preparation of 2-(2,6-dioxo-3-piperidyl)-5,6-difluoro-isoindoline-1,3-dione

To a solution of 5,6-difluoroisobenzofuran-1,3-dione (2.5 g, 13.58 mmol, 1 equiv.) and 3-aminopiperidine-2,6-dione (1.83 g, 14.26 mmol, 1.05 equiv.) in HOAc (50 mL) was added KOAc (1.47 g, 14.94 mmol, 1.1 equiv.). The mixture was stirred at 90° C. for 16 hours. The reaction mixture was concentrated under reduced pressure to give a residue. The crude product was triturated with water at 25° C. for 30 minutes to give 2-(2,6-dioxo-3-piperidyl)-5,6-difluoro-isoindoline-1,3-dione (6.2 g, 20.8 mmol, 76.7% yield, 98.9% purity) as a gray solid.

MS (ESI) m/z: 294.9 [M+H]⁺.

### Step B. Procedure for Preparation of 2-(2,6-dioxo-3-piperidyl)-5-fluoro-6-[4-(hydroxymethyl)-1-piperidyl]isoindoline-1,3-dione

To a solution of 2-(2,6-dioxo-3-piperidyl)-5,6-difluoro-isoindoline-1,3-dione (2 g, 6.80 mmol, 1 equiv.) and 4-piperidylmethanol (782.93 mg, 6.80 mmol, 1 equiv.) in NMP (20 mL) was added DIEA (878.58 mg, 6.80 mmol, 1.18 mL, 1 equiv.). The mixture was stirred at 110° C. for 16 hours. The reaction mixture was diluted with water (30 mL) and filtered to give a residue. The crude product was triturated with water at 25° C. for 30 minutes to give 2-(2,6-dioxo-3-piperidyl)-5-fluoro-6-[4-(hydroxymethyl)-1-piperidyl]isoindoline-1,3-dione (2 g, 4.3 mmol, 63.6% yield, 84.2% purity) as a white solid.

MS (ESI) m/z: 390.1[M+H]⁺.

### Step C. Procedure for Preparation of 1-[2-(2,6-dioxo-3-piperidyl)-6-fluoro-1,3-dioxo-isoindolin-5-yl]piperidine-4-carbaldehyde

A solution of DMSO (802.65 mg, 10.27 mmol, 802.65 μL, 4 equiv.) in DCM (20 mL) was added dropwise to a solution of oxalyl dichloride (651.94 mg, 5.14 mmol, 449.62 μL, 2 equiv.) in DCM (2 mL) at −70° C. under N₂ atmosphere. The mixture was stirred at −70° C. for 30 minutes, then 2-(2,6-dioxo-3-piperidyl)-5-fluoro-6-[4-(hydroxymethyl)-1-piperidyl]isoindoline-1,3-dione (1 g, 2.57 mmol, 1 equiv.) in DCM (2 mL) was added dropwise at −70° C. The solution was stirred for 30 minutes at −70° C. TEA (1.56 g, 15.41 mmol, 2.14 mL, 6 equiv.) was added into the solution. The solution was stirred at 25° C. for 2 hours under N₂ atmosphere. The reaction mixture was quenched by addition H₂O (15 mL) at 25° C., and then diluted with extracted with DCM 60 mL (20 mL×3). The combined organic layers were dried over Na₂SO₄, filtered, and concentrated under reduced pressure to give a residue. The residue was purified by flash silica gel chromatography (eluent of 0-10% ethyl acetate/petroleum ether) to give 1-[2-(2,6-dioxo-3-piperidyl)-6-fluoro-1,3-dioxo-isoindolin-5-yl]piperidine-4-carbaldehyde (1.4 g, 2.5 mmol, 97.8% yield, 69.5% purity) as a yellow oil.

MS (ESI) m/z: 387.9[M+H]⁺.

¹H NMR (400 MHz, DMSO-d₆) δ=11.10 (s, 1H), 7.68 (d, J=11.6 Hz, 1H), 7.43 (d, J=7.6 Hz, 1H), 5.09 (dd, J=5.2, 12.8 Hz, 1H), 4.52 (t, J=5.2 Hz, 1H), 3.61 (d, J=12.0 Hz, 2H), 3.30 (t, J=5.6 Hz, 2H), 2.89-2.84 (m, 2H), 2.59 (d, J=18.8 Hz, 1H), 2.08-1.99 (m, 1H), 1.77 (d, J=12.0 Hz, 2H), 1.37-1.23 (m, 2H)

### Step D. Procedure for Preparation of 6-[8-(1,3-benzothiazol-2-ylcarbamoyl)-3,4-dihydro-1H-isoquinolin-2-yl]-3-[3-[3-[1-[[1-[2-(2,6-dioxo-3-piperidyl)-6-fluoro-1,3-dioxo-isoindolin-5-yl]-4-piperidyl]methyl]-4-piperidyl]propoxy]-2-methyl-phenyl]pyridine-2-carboxylic acid

A solution of 6-[8-(1,3-benzothiazol-2-ylcarbamoyl)-3,4-dihydro-1H-isoquinolin-2-yl]-3-[2-methyl-3-[3-(4-piperidyl)propoxy]phenyl]pyridine-2-carboxylic acid (110 mg, 166.21 μmol, 1 equiv.), 1-[2-(2,6-dioxo-3-piperidyl)-6-fluoro-1,3-dioxo-isoindolin-5-yl]piperidine-4-carbaldehyde (64.38 mg, 166.21 μmol, 1 equiv.), and HOAc (998.13 μg, 16.62 μmol, 9.51e-1 μL, 0.1 equiv.) in DCM (1.5 mL) at was stirred at 25° C. for 15.5 hours. NaBH(OAc)₃ (105.68 mg, 498.63 μmol, 3 equiv.) was added. The reaction mixture was stirred at 25° C. for 0.5 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[1-[[1-[2-(2,6-dioxo-3-piperidyl)-6-fluoro-1,3-dioxo-isoindolin-5-yl]-4-piperidyl]methyl]-4-piperidyl]propoxy]-2-methyl-phenyl]pyridine-2-carboxylic acid (28.9 mg, 27.1 μmol, 16.3% yield, 96.9% purity) as a yellow solid.

MS (ESI) m/z: 517.5 [1/2M+H]⁺.

¹H NMR (400 MHz, DMSO-d₆) δ=11.10 (s, 1H), 8.17 (s, 1H), 8.02 (d, J=8.0 Hz, 1H), 7.78 (d, J=8.0 Hz, 1H), 7.69 (d, J=11.6 Hz, 1H), 7.62 (d, J=8.0 Hz, 1H), 7.49-7.45 (m, 1H), 7.44-7.39 (m, 3H), 7.39-7.33 (m, 2H), 7.11-7.05 (m, 1H), 6.93 (d, J=8.8 Hz, 1H), 6.87 (d, J=8.4 Hz, 1H), 6.63 (d, J=7.6 Hz, 1H), 5.09 (dd, J=5.6, 12.8 Hz, 1H), 5.00-4.95 (m, 2H), 3.98-3.87 (m, 4H), 3.63-3.54 (m, 4H), 3.02 (t, J=5.6 Hz, 3H), 2.90 (s, 1H), 2.85 (d, J=12.4 Hz, 4H), 2.61 (d, J=3.2 Hz, 1H), 2.56 (s, 1H), 2.52 (s, 2H), 2.19 (d, J=7.2 Hz, 2H), 2.06-1.99 (m, 1H), 1.89 (s, 3H), 1.83-1.77 (m, 2H), 1.74 (s, 2H), 1.64 (d, J=12.0 Hz, 2H), 1.42-1.32 (m, 2H), 1.28-1.22 (m, 2H), 1.16-1.12 (m, 1H)

### Example 34. Preparation of Compound 144

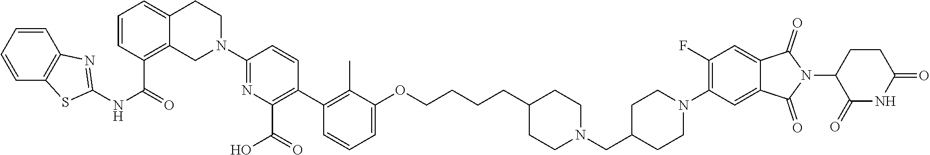 [SMILES: Cc1c(OCCCCC2CCN(CC3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[1-[[1-[2-(2,6-dioxo-3-piperidyl)-6-fluoro-1,3-dioxo-isoindolin-5-yl]-4-piperidyl]methyl]-4-piperidyl]butoxy]-2-methyl-phenyl]pyridine-2-carboxylic acid

### Step A. Procedure for Preparation of tert-butyl 4-[4-(3-bromo-2-methyl-phenoxy)butyl]piperidine-1-carboxylate

A mixture of 3-bromo-2-methyl-phenol (650 mg, 3.48 mmol, 1.0 equiv.), tert-butyl 4-(4-hydroxybutyl)piperidine-1-carboxylate (1.07 g, 4.17 mmol, 1.2 equiv.), and 2-(tributyl-λ5-phosphanylidene)acetonitrile (1.26 g, 5.21 mmol, 1.5 equiv.) in toluene (8 mL) was degassed and purged with N₂ and then stirred at 120° C. for 2 hours under N₂ atmosphere. The residue was spin-dried and the crude product was purified by reverse-phase HPLC (0.1% FA condition) to give tert-butyl 4-[4-(3-bromo-2-methyl-phenoxy)butyl]piperidine-1-carboxylate (1.1 g, 2.5 mmol, 74.2% yield) as a brown oil.

MS (ESI) m/z: 335.1 [M+H]⁺.

1H NMR (400 MHz, DMSO-d6) δ=7.17-7.12 (m, 1H), 7.11-7.05 (m, 1H), 6.95 (d, J=8.0 Hz, 1H), 3.97 (t, J=6.4 Hz, 2H), 3.91 (d, J=12.0 Hz, 2H), 2.66 (d, J=2.0 Hz, 2H), 2.22 (s, 3H), 1.75-1.67 (m, 2H), 1.62 (d, J=12.0 Hz, 2H), 1.48-1.40 (m, 3H), 1.38 (s, 9H), 1.28-1.22 (m, 2H), 1.02-0.88 (m, 2H).

### Step B. Procedure for Preparation of tert-butyl 6-[8-(1,3-benzothiazol-2-ylcarbamoyl)-3,4-dihydro-1H-isoquinolin-2-yl]-3-[3-[4-(1-tert-butoxycarbonyl-4-piperidyl)butoxy]-2-methyl-phenyl]pyridine-2-carboxylate

Tert-butyl 4-[4-(3-bromo-2-methyl-phenoxy)butyl]piperidine-1-carboxylate (500 mg, 1.17 mmol, 1.0 equiv.), tert-butyl.6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (718.30 mg, 1.17 mmol, 1.0 equiv.), Ad₂nBuP Pd G₃ (85.40 mg, 117.26 μmol, 0.1 equiv.), and K₂CO₃ (486.20 mg, 3.52 mmol, 3.0 equiv.) were taken up into a microwave tube in dioxane (6 mL) and H₂O (2 mL). The sealed tube was heated at 100° C. for 1 hour under microwave. The residue was concentrated. The residue was purified by flash silica gel chromatography (eluent of 33% ethyl acetate/petroleum ether) to give tert-butyl 6-[8-(1,3-benzothiazol-2-ylcarbamoyl)-3,4-dihydro-1H-isoquinolin-2-yl]-3-[3-[4-(1-tert-butoxycarbonyl-4-piperidyl)butoxy]-2-methyl-phenyl]pyridine-2-carboxylate (700 mg, 841.2 μmol, 71.7% yield) as a brown oil.

MS (ESI) m/z: 832.1 [M+H]⁺.

1H NMR (400 MHz, DMSO-d₆) δ=12.86 (s, 1H), 8.02 (d, J=7.6 Hz, 1H), 7.80-7.75 (m, 1H), 7.60 (d, J=7.6 Hz, 1H), 7.48-7.42 (m, 3H), 7.39-7.34 (m, 2H), 7.12-7.07 (m, 1H), 6.93 (d, J=8.4, 14.1 Hz, 2H), 6.57 (d, J=7.6 Hz, 1H), 4.98 (s, 2H), 4.03 (d, J=7.2 Hz, 3H), 3.03 (t, J=5.6 Hz, 2H), 1.99 (s, 4H), 1.87 (s, 2H), 1.37-1.36 (m, 9H), 1.18 (t, J=7.2 Hz, 5H), 1.07 (s, 8H), 1.00 (s, 9H).

### Step C. Procedure for Preparation of 6-[8-(1,3-benzothiazol-2-ylcarbamoyl)-3,4-dihydro-1H-isoquinolin-2-yl]-3-[2-methyl-3-[4-(4-piperidyl)butoxy]phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[4-(1-tert-butoxycarbonyl-4-piperidyl)butoxy]-2-methyl-phenyl]pyridine-2-carboxylate (130 mg, 156.24 μmol, 1.0 equiv.) in HCOOH (2 mL) was stirred at 90° C. for 2 hours. The residue was spin-dried and used in the next step without further purification. 6-[8-(1,3-Benzothiazol-2-ylcarbamoyl)-3,4-dihydro-1H-isoquinolin-2-yl]-3-[2-methyl-3-[4-(4-piperidyl)butoxy]phenyl]pyridine-2-carboxylic acid (81 mg, 119.8 μmol, 76.7% yield) was obtained as a brown oil.

MS (ESI) m/z: 676.2 [M+H]⁺.

### Step D. Procedure for Preparation of 6-[8-(1,3-benzothiazol-2-ylcarbamoyl)-3,4-dihydro-1H-isoquinolin-2-yl]-3-[3-[4-[1-[[1-[2-(2,6-dioxo-3-piperidyl)-6-fluoro-1,3-dioxo-isoindolin-5-yl]-4-piperidyl]methyl]-4-piperidyl]butoxy]-2-methyl-phenyl]pyridine-2-carboxylic acid

To a solution of 6-[8-(1,3-benzothiazol-2-ylcarbamoyl)-3,4-dihydro-1H-isoquinolin-2-yl]-3-[2-methyl-3-[4-(4-piperidyl)butoxy]phenyl]pyridine-2-carboxylic acid (67 mg, 99.14 μmol, 1.0 equiv.) in DCM (0.3 mL) and IPA (0.3 mL) was added 1-[2-(2,6-dioxo-3-piperidyl)-6-fluoro-1,3-dioxo-isoindolin-5-yl]piperidine-4-carbaldehyde (46.08 mg, 118.96 μmol, 1.2 equiv.) at 25° C. for 16 hours. After addition, the mixture was stirred at 25° C., and then NaBH(OAc)₃ (63.03 mg, 297.41 μmol, 3.0 equiv.) was added at 0° C. The resulting mixture was stirred at 25° C. for 1 hour. The residue was spin-dried and purified by prep-HPLC to give 6-[8-(1,3-benzothiazol-2-ylcarbamoyl)-3,4-dihydro-1H-isoquinolin-2-yl]-3-[3-[4-[1-[[1-[2-(2,6-dioxo-3-piperidyl)-6-fluoro-1,3-dioxo-isoindolin-5-yl]-4-piperidyl]methyl]-4-piperidyl]butoxy]-2-methyl-phenyl]pyridine-2-carboxylic acid (13.60 mg, 12.5 μmol, 12.6% yield, 96.8% purity) as a yellow solid.

MS (ESI) m/z: 524.4 [M/2+H]⁺.

1H NMR (400 MHz, DMSO-d₆) δ=11.09 (s, 1H), 8.19 (s, 1H), 8.01 (d, J=8.0 Hz, 1H), 7.77 (d, J=8.0 Hz, 1H), 7.68 (d, J=11.2 Hz, 1H), 7.62 (d, J=8.4 Hz, 1H), 7.48-7.30 (m, 6H), 7.07 (t, J=8.0 Hz, 1H), 6.88 (dd, J=8.4, 16.0 Hz, 2H), 6.64 (d, J=7.6 Hz, 1H), 5.09 (dd, J=5.6, 12.8 Hz, 1H), 4.97 (s, 2H), 4.00-3.88 (m, 4H), 3.63-3.53 (m, 4H), 3.01 (t, J=5.6 Hz, 2H), 2.88-2.82 (m, 4H), 2.63-2.60 (m, 1H), 2.56 (s, 1H), 2.19 (d, J=6.4 Hz, 2H), 2.06-1.99 (m, 1H), 1.94-1.84 (m, 5H), 1.84-1.76 (m, 2H), 1.76-1.68 (m, 3H), 1.65-1.57 (m, 2H), 1.48-1.41 (m, 2H), 1.28-1.21 (m, 4H), 1.17-1.06 (m, 2H).

### Example 35. Preparation of Compound 145

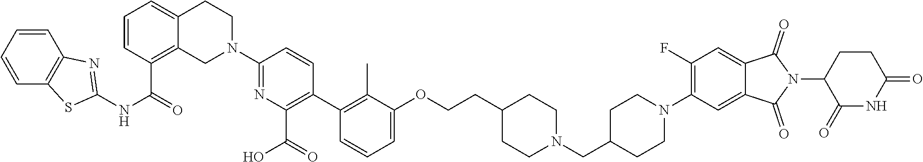 [SMILES: Cc1c(OCCC2CCN(CC3CCN(c4cc5c(cc4F)C(=O)N(C4CCC(=O)NC4=O)C5=O)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[[1-[2-(2,6-dioxo-3-piperidyl)-6-fluoro-1,3-dioxo-isoindolin-5-yl]-4-piperidyl]methyl]-4-piperidyl]ethoxy]-2-methyl-phenyl]pyridine-2-carboxylic acid

### Step A. Procedure for Preparation of tert-butyl 4-[2-(3-bromo-2-methyl-phenoxy)ethyl]piperidine-1-carboxylate

To a solution of tert-butyl 4-(2-bromoethyl)piperidine-1-carboxylate (1 g, 3.42 mmol, 1.0 equiv.) and 3-bromo-2-methyl-phenol (704.07 mg, 3.76 mmol, 1.1 equiv.) in MeCN (10 mL) was added K₂CO₃ (1.42 g, 10.27 mmol, 3.0 equiv.). The mixture was stirred at 60° C. for 16 hours. The mixture was filtered to give a filtrate, which was concentrated under reduced pressure to give a residue. The residue was purified by flash silica gel chromatography (eluent of 0-4% ethyl acetate/petroleum ether) to give tert-butyl 4-[2-(3-bromo-2-methyl-phenoxy)ethyl]piperidine-1-carboxylate (1.2 g, 2.9 mmol, 86.9% yield, 98.8% purity) as a yellow oil.

MS (ESI) m/z: 298.2 [M−100+H]⁺

¹H NMR (400 MHz, CD₃OD) δ=7.13-7.09 (m, 1H), 7.02 (t, J=8.0 Hz, 1H), 6.89 (d, J=8.0 Hz, 1H), 4.10-4.07 (m, 1H), 4.07-4.03 (m, 3H), 2.84-2.69 (m, 2H), 2.28 (s, 3H), 1.79-1.73 (m, 5H), 1.45 (s, 9H), 1.22-1.11 (m, 2H)

### Step B. Procedure for Preparation of tert-butyl 6-[8-(1,3-benzothiazol-2-ylcarbamoyl)-3,4-dihydro-1H-isoquinolin-2-yl]-3-[3-[2-(1-tert-butoxycarbonyl-4-piperidyl)ethoxy]-2-methyl-phenyl]pyridine-2-carboxylate

Tert-butyl 4-[2-(3-bromo-2-methyl-phenoxy)ethyl]piperidine-1-carboxylate (1.18 g, 2.96 mmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.81 g, 2.96 mmol, 1.0 equiv.), KF (516.31 mg, 8.89 mmol, 208.19 μL, 3.0 equiv.), and Ad₂nBuP Pd G₃ (215.74 mg, 296.23 μmol, 0.1 equiv.) were taken up into a microwave tube in dioxane (10 mL) and H₂O (1 mL). The sealed tube was heated at 100° C. for 2 hours under microwave. The mixture was concentrated under reduced pressure to give a residue. The residue was purified by flash silica gel chromatography (eluent of 0˜30% ethyl acetate/petroleum ether) to give tert-butyl 6-[8-(1,3-benzothiazol-2-ylcarbamoyl)-3,4-dihydro-1H-isoquinolin-2-yl]-3-[3-[2-(1-tert-butoxycarbonyl-4-piperidyl)ethoxy]-2-methyl-phenyl]pyridine-2-carboxylate (1.7 g, 2.0 mmol, 69.2% yield, 96.9% purity) as a yellow solid.

MS (ESI) m/z: 804.5 [M+H]⁺

¹H NMR (400 MHz, DMSO-d₆) δ=12.86 (s, 1H), 8.01 (d, J=7.6 Hz, 1H), 7.78 (d, J=8.0 Hz, 1H), 7.60 (d, J=7.6 Hz, 1H), 7.49-7.41 (m, 3H), 7.39-7.31 (m, 2H), 7.09 (t, J=8.0 Hz, 1H), 6.97-6.89 (m, 2H), 6.57 (d, J=7.6 Hz, 1H), 4.98 (s, 2H), 4.04-3.97 (m, 2H), 3.96-3.83 (m, 4H), 3.07-2.99 (m, 2H), 2.76-2.57 (m, 2H), 1.87 (s, 3H), 1.66 (s, 5H), 1.37 (s, 9H), 1.11-1.03 (m, 2H), 1.00 (s, 9H)

### Step C. Procedure for Preparation of 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[2-(1-tert-butoxycarbonyl-4-piperidyl)ethoxy]-2-methyl-phenyl]pyridine-2-carboxylate (1.7 g, 2.11 mmol, 1.0 equiv.) and HCl/dioxane (4 M, 17 mL, 32.2 equiv.) was stirred at 25° C. for 2 hours. The mixture was concentrated under reduced pressure to give 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid (1.5 g, 1.8 mmol, 88.5% yield, 85.4% purity, HCl) as a yellow solid.

### Step D. Procedure for Preparation of 6-[8-(1,3-benzothiazol-2-ylcarbamoyl)-3,4-dihydro-1H-isoquinolin-2-yl]-3-[3-[2-[1-[[1-[2-(2,6-dioxo-3-piperidyl)-6-fluoro-1,3-dioxo-isoindolin-5-yl]-4-piperidyl]methyl]-4-piperidyl]ethoxy]-2-methyl-phenyl]pyridine-2-carboxylic acid

A solution of 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid (150 mg, 231.56 μmol, 1.0 equiv.) and 1-[2-(2,6-dioxo-3-piperidyl)-6-fluoro-1,3-dioxo-isoindolin-5-yl]piperidine-4-carbaldehyde (107.64 mg, 277.87 μmol, 1.2 equiv.) in DCM (1 mL) and IPA (1 mL) was stirred at 25° C. for 12 hours. NaBH(OAc)₃ (147.23 mg, 694.68 μmol, 3.0 equiv.) was added at 0° C. After addition, the mixture was warmed to 25° C. and stirred at 25° C. for 0.5 hours. The mixture was concentrated under reduced pressure to give a residue, which was diluted with DMF (2 mL). The mixture was purified by prep-HPLC to give 6-[8-(1,3-benzothiazol-2-ylcarbamoyl)-3,4-dihydro-1H-isoquinolin-2-yl]-3-[3-[2-[1-[[1-[2-(2,6-dioxo-3-piperidyl)-6-fluoro-1,3-dioxo-isoindolin-5-yl]-4-piperidyl]methyl]-4-piperidyl]ethoxy]-2-methyl-phenyl]pyridine-2-carboxylic acid (32.7 mg, 28.3 μmol, 12.2% yield, 92.1% purity) as a yellow solid.

MS (ESI) m/z: 1019.5 [M+H]⁺

¹H NMR (400 MHz, DMSO-d₆) δ=11.10 (s, 1H), 8.02 (d, J=8.0 Hz, 1H), 7.78 (d, J=8.0 Hz, 1H), 7.69 (d, J=11.6 Hz, 1H), 7.62 (d, J=7.2 Hz, 1H), 7.53-7.25 (m, 6H), 7.08 (t, J=8.0 Hz, 1H), 7.00-6.81 (m, 2H), 6.64 (d, J=7.6 Hz, 1H), 5.13-5.06 (m, 1H), 4.98 (s, 2H), 3.99 (t, J=6.0 Hz, 2H), 3.91 (t, J=6.0 Hz, 2H), 3.61-3.57 (m, 2H), 3.02 (t, J=5.6 Hz, 2H), 2.91-2.80 (m, 5H), 2.63-2.55 (m, 2H), 2.17 (d, J=6.8 Hz, 2H), 2.06-1.99 (m, 1H), 1.95-1.83 (m, 5H), 1.83-1.77 (m, 2H), 1.76-1.61 (m, 5H), 1.55-1.43 (m, 1H), 1.29-1.17 (m, 4H)

¹⁹F NMR (377 MHz, DMSO-d₆) δ=−112.14 (s, 1F)

### Example B1

### HiBiT Based Degradation Assay

Human BCL-X_{L} protein coding open reading frame fused with N-terminal HiBiT coding sequence was synthesized from Integrated DNA Technologies (IDT). Next, N-HiBiT-BCL-X_{L} sequence was cloned into pLV-UBC-PGK-Puro, a lentivirus plasmid purchased from Vectorbuilder, to generate pLV-UBC-N-HiBiT-BCL-X_{L}-PGK-puro. Lentiviral particles were generated from Lenti-X™ 293T cells (Clontech) by co-transfection of pLV-UBC-N-HiBiT-BCL-X_{L}-PGK-puro plasmids and lentiviral packaging plasmid mix (Cellecta). HT-1080 [HT1080](ATCC CCL-121) cells were infected with the lentivirus, and HT1080 cells stably integrated with the lentiviral vectors were established by incubation with 1 g/mL puromycin (Thermofisher).

HT1080 cells expressing N-terminal HiBiT tagged BCL-X_{L} were dispensed into a 384-well plate pre-spotted with compounds at varying concentrations. Three thousand cells were seeded into each well in 40 μL of RPMI 1640 media. After 6 hours of incubation at 37° C. with 5% CO₂, 30 μL of the NANO-GLO® HiBiT Lytic Detection System working solution (Promega) was added to each well and incubated at room temperature for 30 min. After incubation, luminescence was read on a PHERAstar FSX Plate Reader (BMG). BCL-X_{L} degradation at each indicated concentration was normalized with the DMSO control. The BCL-X_{L} degradation curves were plotted using a four-parameter logistic model. For compounds displaying hook effects, the curve fitting was performed using the bottom point of the hook curves (i.e., the point of Yₘᵢₙ) as the bottom plateau to calculate EC₅₀. Yₘᵢₙ and EC₅₀ values for certain compounds are shown in Table B1.

TABLE B1

| Compound | Yₘᵢₙ | EC₅₀ | Yₘᵢₙ | EC₅₀ |
|---|---|---|---|---|
| No. | (24 h) | (24 h) | (6 h) | (6 h) |
| 101 | | | B | + |
| 102 | | | A | ++ |
| 103 | | | B | ++ |
| 104 | B | ++ | B | + |
| 105 | | | A | ++ |
| 106 | | | B | +++ |
| 107 | | | B | + |
| 108 | | | B | + |
| 109 | | | B | +++ |
| 110 | B | ++ | B | +++ |
| 111a | B | + | B | +++ |
| 112a | C | ++ | B | +++ |
| 113 | B | ++ | B | +++ |
| 114 | | | B | ++ |
| 115 | B | +++ | C | + |
| 116 | A | +++ | | |
| 117 | B | +++ | B | ++ |
| 118 | A | +++ | | |
| 119 | B | ++ | C | + |
| 120a | B | + | | |
| 120b | C | + | | |
| 121 | A | +++ | | |
| 122a | B | ++ | | |
| 122b | B | ++ | | |
| 123 | B | +++ | | |
| 124a | B | +++ | | |
| 124b | B | + | | |
| 125 | | | | |
| 126 | B | + | | |
| 127 | B | + | | |
| 128a | B | ++ | C | +++ |
| 128b | B | +++ | | |
| 129a | B | +++ | | |
| 130a | B | +++ | | |
| 130b | C | | | |
| 132 | B | + | | |
| 133 | | | | |
| 135a | B | +++ | | |
| 135b | B | ++ | | |
| 137 | B | +++ | C | + |
| 138 | B | +++ | C | ++ |
| 139a | B | ++ | | |
| 140a | A | + | C | + |
| 141 | B | ++ | B | ++ |
| 142 | | | | |
| 143 | B | +++ | | |
| 144 | A | +++ | | |
| 145 | A | +++ | | |

Notes:
(1) “A”: Yₘᵢₙ < 50%; “B”: 50% ≤ Yₘᵢₙ < 70%; “C”: Yₘᵢₙ ≥ 70%
(2) “+++”: EC₅₀ < 100 nM; “++”: 100 nM ≤ EC₅₀ < 1000 nM; “+”: 1000 nM ≤ EC₅₀ < 10,000 nM

### Example B2

### Western Blotting Based Degradation Assay

BCL-X_{L} degradation in human cancer cell lines is measured using traditional Western blotting methods as well as with the automated “Jess” instrument from ProteinSimple. Cells are seeded at 0.5-1 million cells per well in up to 2 mL of growth media (RPMI+10% FBS) in 6-well plates. The plates are treated with compounds at varying concentrations for up to 5 days. Cells are harvested and lysed using RIPA Buffer (#9806S, Cell Signaling Technology) with Halt Protease & Phosphatase Inhibitor Cocktail (#78441, Thermo Fisher). Protein concentrations of each sample are quantified using Pierce™ BCA Protein Assay Kit (#23225, Thermo Fisher). For the Jess instrument the standard protocol provided by the manufacturer using the Jess Separation Module SM-W004 is followed. For traditional Western blotting, samples resolved on a 4-20% Criterion TGX gel (#5671094, BioRad) and transferred to a PVDF membrane using the Trans-Blot Turbo Transfer system (#1704157, BioRad). Chemiluminescent detection of BCL-X_{L} is performed using a BCL-X_{L} antibody (#2764, Cell Signaling Technology) according to the manufacturer's instructions.

### Example B3

### MOLT-4 Proliferation Assay

MOLT-4 (ATCC CRL-1582) cells are seeded at 4,000 cells per well in 50 μL RPMI 1640 media in black 384-well plates. The plates are pre-spotted with compounds at varying concentrations. After 3 days of incubation at 37° C. with 5% CO₂, cells are assessed using CELLTITERGLO® Cell Viability Assay kit according to manufacturer's instructions (Promega). Relative cell proliferation at each concentration is normalized against the DMSO control.

### Example B4

### Cell Line Proliferation Assay

Human cancer cell lines are seeded at 1,000-4,000 cells per well in 50 μL growth media (following culture conditions recommended by cell line sources) in 384-well plates. The plates are treated with compounds at varying concentrations using a D300e Digital Dispenser (Tecan) and then incubated at 37° C. with 5% CO₂ for 3-7 days. Relative cell viability is assessed using CELLTITERGLO® Cell Viability Assay (#G9243, Promega) according to the manufacturer's instructions or Incucyte SX5 Live-Cell Analysis Instrument (Sartorius) with images recorded at 4-12 hour intervals.

### Example B5

### Apoptosis Assay

Human cancer cell lines are seeded at 1,000-4,000 cells per well in 50 μL growth media (following culture conditions recommended by cell line sources) in 384-well plates. The plates are treated with compounds at varying concentrations using a D300e Digital Dispenser (Tecan) and then incubated at 37° C. with 5% CO₂ for 3-7 days. Relative caspase-3/7 activity is assessed using Caspase-Glo 3/7 Assay System (#G8093, Promega) according to the manufacturer's instructions or Incucyte SX5 Live-Cell Analysis Instrument (Sartorius) with Green Incucyte Caspase-3/7 Dye (#4440, Sartorius) added to the media at between 1:500 and 1:1000 dilution at the time of seeding and images recorded at 4-12 hour intervals. Relative annexin V positivity is assessed using Incucyte SX5 Live-Cell Analysis Instrument (Sartorius) with Orange Incucyte Annexin V Dye (#4759, Sartorius) added to the media at a 1:200 dilution at the time of seeding and images recorded at 4-12 hour intervals.

### Example B6

### Platelet Viability Assay

Human platelet-rich plasma (PRP, SER-PRP-SDS) is obtained from Zenbio. Sodium citrate is used as anticoagulant. To remove any contaminating blood cells, the samples are spun at 200×g at room temperature for 20 min in 50 m1 falcon tubes, and the supernatant is transferred to a new tube. One hundred microliter PRP are added to each well of a 96-well plate spotted with compounds at varying concentrations. Plates are incubated at 37° C. with 5% CO₂ for 24 hours, and platelet viability are read with the CELLTITERGLO® Cell Viability Assay kit according to manufacturer's instructions (Promega). Relative viability at each concentration is normalized against the DMSO control.

### EXEMPLARY EMBODIMENTS

### P01 Embodiments

Embodiment 1. A compound of Formula (I) or (II):

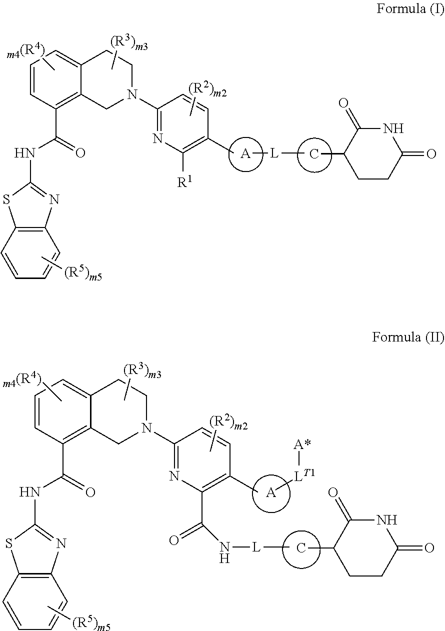

or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b},
L^{T1} is a bond or C₁₋₃ alkylene optionally substituted with 1-3 substituents independently selected from the group consisting of: oxo and R^{c}, wherein one CH₂ unit of the C₁₋₃ alkylene is optionally replaced with —O— or —N(R^{d})—;
A* is selected from the group consisting of:
(a) C₃₋₁₅ cycloalkyl or 3-15 membered heterocyclyl, each of which is optionally substituted with 1-6 substituents independently selected from the group consisting of: R^{a} and R^{b};
(b) C₆₋₁₅ aryl or 5-15 membered heteroaryl, each of which is optionally substituted with 1-6 substituents independently selected from the group consisting of: R^{a} and R^{b}; and
(c) H;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):

### AA

n1 is an integer from 3 to 15; and
each L^{A} is independently selected from the group consisting of: L^{A1}, L^{A3}, and L^{A4} provided that 1-3 occurrences of L^{A} is L^{A4};

### BB

n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R′)₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{e};
Ring C is selected from the group consisting of:

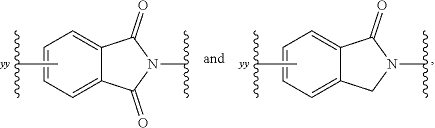

each of which is optionally substituted with 1-3 substituents independently selected from the group consisting of: R^{a} and R^{b}, wherein yy is the point of attachment to L;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e}
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{h} is independently selected from the group consisting of: —O—; —N(H)—; —N(C₁₋₃ alkyl)-; —S(O)₀₋₂—; C(═O); and C₁₋₃ alkylene, and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂;
provided that one or both of (i) and (ii) applies:
(i) Ring C is substituted with 1-3 substituents independently selected from the group consisting of: R^{a} and R^{b}; and
(ii) in Formula (I), L does not contain an adamantylene group, and in Formula (II), A* is other than adamantyl.

Embodiment 2. The compound of Embodiment 1, wherein the compound is a compound of Formula (I), or a pharmaceutically acceptable salt thereof.

Embodiment 3. The compound of Embodiment 1, wherein the compound is a compound of Formula (II), or a pharmaceutically acceptable salt thereof.

Embodiment 4. The compound of any one of Embodiments 1-3, wherein Ring A is phenylene optionally substituted with 1-3 R^{a}.

Embodiment 5. The compound of any one of Embodiments 1-4, wherein Ring A is

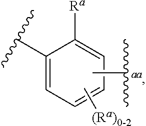

wherein aa represents the point of attachment to L or L^{T1}.

Embodiment 6. The compound of any one of Embodiments 1-5, wherein Ring A is

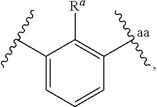

wherein aa represents the point of attachment to L or L^{T1}.

Embodiment 7. The compound of any one of Embodiments 1-5, wherein Ring A is

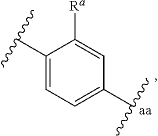

wherein aa represents the point of attachment to L or L^{T1}.

Embodiment 8. The compound of any one of Embodiments 1-3, wherein Ring A is 5-6 membered heteroarylene optionally substituted with 1-3 R^{a}.

Embodiment 9. The compound of any one of Embodiments 1-3 or 8, wherein Ring A is 5-6 membered heteroarylene optionally substituted with 1-2 R^{a}.

Embodiment 10. The compound of any one of Embodiments 1-3 or 8-9, wherein Ring A is 5-membered heteroarylene optionally substituted with 1-2 R^{a}.

Embodiment 11. The compound of any one of Embodiments 1-3 or 8-10, wherein Ring A is pyrazolylene optionally substituted with 1-2 R^{a}.

Embodiment 12. The compound of any one of Embodiments 1-3 or 8-11, wherein Ring A is selected from the group consisting of:

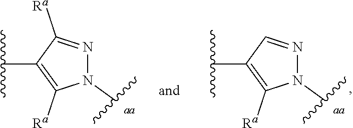

wherein aa represents the point of attachment to L or L^{T1}.

Embodiment 13. The compound of any one of Embodiments 1-3, wherein Ring A is C₃₋₁₀ cycloalkylene optionally substituted with 1-6 R^{a}.

Embodiment 14. The compound of any one of Embodiments 1-13, wherein one R^{a} present on Ring A is C₁₋₃ alkyl optionally substituted with 1-3 F.

Embodiment 15. The compound of any one of Embodiments 1-14, wherein one R^{a} present on Ring A is methyl or CF₃.

Embodiment 16. The compound of any one of Embodiments 1-2 or 4-15, wherein R¹ is C(O)OH.

Embodiment 17. The compound of any one of Embodiments 1-2 or 4-15, wherein R¹ is C(O)NHR^{e}.

Embodiment 18. The compound of any one of Embodiments 1 or 3-15, wherein L^{T1} is C₁₋₃ alkylene.

Embodiment 19. The compound of any one of Embodiments 1, 3-15, or 18, wherein L^{T1} is —CH₂—.

Embodiment 20. The compound of any one of Embodiments 1, 3-15, or 18-19, wherein A* is H.

Embodiment 21. The compound of any one of Embodiments 1, 3-15, or 18-19, wherein A* is C₃₋₁₅ cycloalkyl or 3-15 membered heterocyclyl, each of which is optionally substituted with 1-6 substituents independently selected from the group consisting of: R^{a} and R^{b}.

Embodiment 22. The compound of any one of Embodiments 1, 3-15, 18-19, or 21, wherein A* is C₃₋₁₅ cycloalkyl optionally substituted with 1-3 R^{a}.

Embodiment 23. The compound of any one of Embodiments 1, 3-15, 18-19, or 21-22, wherein A* is adamantyl optionally substituted with 1-3 R^{a}.

Embodiment 24. The compound of any one of Embodiments 1-23, wherein m2 is 0.

Embodiment 25. The compound of any one of Embodiments 1-24, wherein m3 is 0.

Embodiment 26. The compound of any one of Embodiments 1-25, wherein m4 is 0.

Embodiment 27. The compound of any one of Embodiments 1-26, wherein m5 is 0.

Embodiment 28. The compound of any one of Embodiments 1-23, wherein m2 is 0; m3 is 0; m4 is 0; and m5 is 0.

Embodiment 29. The compound of any one of Embodiments 1-28, wherein Ring C is selected from the group consisting of:

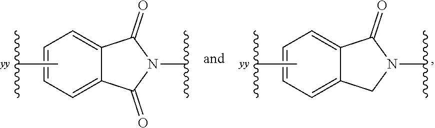

each of which is optionally substituted with 1-3 R^{a}, wherein yy is the point of attachment to L.

Embodiment 30. The compound of any one of Embodiments 1-29, wherein Ring C is selected from the group consisting of:

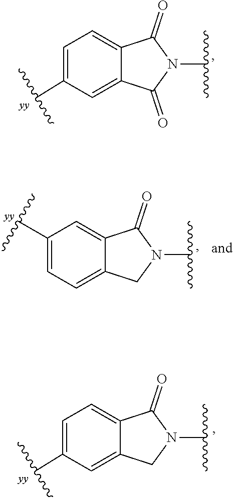 [SMILES: *c1ccc2c(c1)C(=O)N(*)C2=O ; *c1ccc2c(c1)C(=O)N(*)C2 ; *c1ccc2c(c1)CN(*)C2=O]

each of which is substituted with 1-3 R^{a}, wherein yy is the point of attachment to L.

Embodiment 31. The compound of any one of Embodiments 1-30, wherein Ring C is

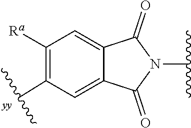

further optionally substituted with 1-2 R^{a} at one or more ring carbon atoms, wherein yy is the point of attachment to L.

Embodiment 32. The compound of any one of Embodiments 1-31, wherein Ring C is

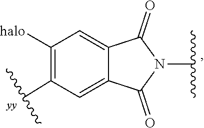

wherein yy is the point of attachment to L.

Embodiment 33. The compound of any one of Embodiments 1-32, wherein Ring C is

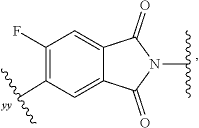

wherein yy is the point of attachment to L.

Embodiment 34. The compound of any one of Embodiments 1-33, wherein L is -(L^{A})ₙ₁-; and L^{A} and n1 are defined according to (AA).

Embodiment 35. The compound of any one of Embodiments 1-34, wherein n1 is an integer from 3 to 5.

Embodiment 36. The compound of any one of Embodiments 1-34, wherein n1 is an integer from 5 to 9.

Embodiment 37. The compound of any one of Embodiments 1-34 or 36, wherein n1 is 6, 7, or 8.

Embodiment 38. The compound of any one of Embodiments 1-34, wherein n1 is an integer from 9 to 12.

Embodiment 39. The compound of any one of Embodiments 1-38, wherein 1-2 occurrences of L^{A} is L^{A4}.

Embodiment 40. The compound of any one of Embodiments 1-39, wherein one occurrence of L^{A} is L^{A4}.

Embodiment 41. The compound of any one of Embodiments 1-39, wherein two occurrences of L^{A} are L^{A4}.

Embodiment 42. The compound of any one of Embodiments 1-41, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 43. The compound of any one of Embodiments 1-42, wherein 1-4 occurrences of L^{A} is L^{A3}.

Embodiment 44. The compound of any one of Embodiments 1-43, wherein 1-3 occurrences of L^{A} is L^{A3}.

Embodiment 45. The compound of any one of Embodiments 1-44, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 46. The compound of any one of Embodiments 1-45, wherein 2-7 occurrences of L^{A} are L^{A1}.

Embodiment 47. The compound of any one of Embodiments 1-46, wherein 2-5 occurrences of L^{A} are L^{A1}.

Embodiment 48. The compound of any one of Embodiments 1-45, wherein 8-13 occurrences of L^{A} are L^{A1}.

Embodiment 49. The compound of any one of Embodiments 1-48, wherein 0-2 occurrences of L^{A1} is —CHR^{L}— or —C(R^{L})₂—; and each remaining occurrence of L^{A1} is —CH₂—.

Embodiment 50. The compound of any one of Embodiments 1-49, wherein each occurrence of L^{A1} is —CH₂—.

Embodiment 51. The compound of any one of Embodiments 1-49, wherein one occurrence of L^{A1} is —CHR^{L}— or —C(R^{L})₂—; and each remaining occurrence of L^{A1} is —CH₂—.

Embodiment 52. The compound of any one of Embodiments 1-51, wherein each R^{L} is independently selected from the group consisting of: —F and —C₁₋₃ alkyl optionally substituted with 1-3 F.

Embodiment 53. The compound of any one of Embodiments 1-34, wherein L is -(L^{A})ₙ₁- and L^{A} and n1 are defined according to (AA), wherein:
n1 is an integer from 5 to 9;
2 occurrences of L^{A} are L^{A4};
2-7 occurrences of L^{A} are L^{A1}; and
1-3 occurrences of L^{A} is L^{A3}.

Embodiment 54. The compound of any one of Embodiments 1-34, wherein L is -(L^{A})ₙ₁- and L^{A} and n1 are defined according to (AA), wherein;
n1 is an integer from 5 to 13;
1 occurrence of L^{A} is L^{A4};
2-11 occurrences of L^{A} are L^{A1}; and
1-3 occurrences of L^{A} is L^{A3}.

Embodiment 55. The compound of any one of Embodiments 1-34, wherein L is selected from the group consisting of:
(i) -(L^{A3})₀₋₂-(L^{A1})₀₋₅-L^{A4}-(L^{A1})₀₋₅-L^{A3}-L^{A4}-_{bb}; and
(ii) -(L^{A3})₀₋₂-(L^{A1})₀₋₅-L^{A4}-(L^{A1})₀₋₅-L^{A4}-_{bb};
provided that L contains 2-7 L^{A1}; and wherein bb represents the point of attachment to Ring C.

Embodiment 56. The compound of any one of Embodiments 1-34, wherein L is:
(L^{A3})₀₋₂-(L^{A1})₁₋₁₁-(L^{A3})₀₋₁-L^{A4}-_{bb},
wherein bb represents the point of attachment to Ring C.

Embodiment 57. The compound of any one of Embodiments 53-56, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 58. The compound of any one of Embodiments 53-57, wherein 0-1 occurrence of L^{A1} is —CHR^{L}— or —C(R^{L})₂—; and each remaining occurrence of L^{A1} is —CH₂—.

Embodiment 59. The compound of any one of Embodiments 53-58, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 60. The compound of any one of Embodiments 1-34 or 55, wherein L is:
-(L^{A3})₀₋₁-(L^{A1})₀₋₅-L^{A4}-(L^{A1})₀₋₅-L^{A4}-bb; or
-(L^{A3})₀₋₁-(L^{A1})₀₋₅-L^{A4}-(L^{A1})₀₋₅-L^{A3}-L^{A4}-_{bb};
provided that L contains 2-7 L^{A1}; and wherein bb represents the point of attachment to Ring C.

Embodiment 61. The compound of Embodiment 60, wherein L is:
-(L^{A3})₀₋₁-(L^{A1})₀₋₅-L^{A4}-(L^{A1})₀₋₅-(4-10 membered heterocyclylene)-_{bb}; or
-(L^{A3})₀₋₁-(L^{A1})₀₋₅-L^{A4}-(L^{A1})₀₋₅-C(═O)-(4-10 membered heterocyclylene)-_{bb};
provided that L contains 2-7 L^{A1}; and wherein bb represents the point of attachment to Ring C.

Embodiment 62. The compound of Embodiments 60 or 61, wherein L is:
-(L^{A3})₀₋₁-(L^{A1})₀₋₅-L^{A4}-(L^{A1})₀₋₅-(6-membered heterocyclylene)-_{bb}; or
-(L^{A3})₀₋₁-(L^{A1})₀₋₅-L^{A4}-(L^{A1})₀₋₅-C(═O)-(6-membered heterocyclylene)-_{bb};
provided that L contains 2-5 L^{A1}; and wherein bb represents the point of attachment to Ring C.

Embodiment 63. The compound of any one of Embodiments 60-62, wherein L is a divalent group of Formula (L-1) or (L-2):

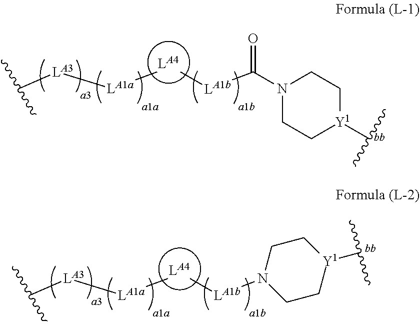

wherein:
Y¹ is N or CH;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

Embodiment 64. The compound of Embodiment 63, wherein a3 is 1.

Embodiment 65. The compound of Embodiments 63 or 64, wherein L^{A3} is —O—.

Embodiment 66. The compound of any one of Embodiments 63-65, wherein a1a+a1b is 3 or 4.

Embodiment 67. The compound of any one of Embodiments 63-65, wherein a1a+a1b is 2.

Embodiment 68. The compound of any one of Embodiments 63-67, wherein each occurrence of L^{A1a} and L^{A1b} is —CH₂—.

Embodiment 69. The compound of any one of Embodiments 63-67, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 70. The compound of any one of Embodiments 63-67, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 71. The compound of any one of Embodiments 63-70, L^{A4} is 4-10 membered heterocyclylene optionally substituted with 1-3 R^{a}.

Embodiment 72. The compound of any one of Embodiments 63-71, wherein L^{A4} is selected from the group consisting of:

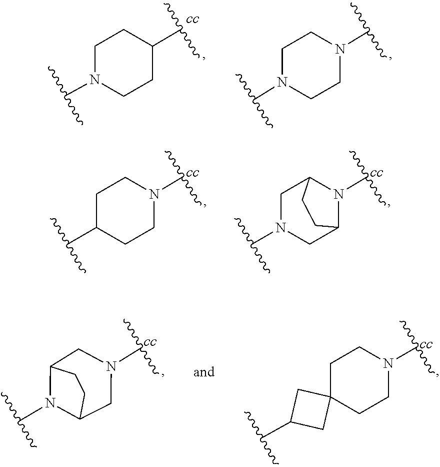 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}.

Embodiment 73. The compound of Embodiments 71 or 72, wherein each R^{a} present on L^{A4} is C₁₋₃ alkyl optionally substituted with 1-3 F.

Embodiment 74. The compound of any one of Embodiments 63-70, wherein L^{A4} is C₃₋₁₀ cycloalkylene optionally substituted with 1-3 R^{a}.

Embodiment 75. The compound of any one of Embodiments 63-70 or 74, wherein L^{A4} is 1,4-cyclohexylene optionally substituted with 1-3 R^{a}.

Embodiment 76. The compound of any one of Embodiments 63-70, wherein L^{A4} is phenylene or 5-6 membered heteroarylene, each of which is optionally substituted with 1-3 R^{a}.

Embodiment 77. The compound of any one of Embodiments 63-70 or 76, wherein L^{A4} is 1,4-phenylene optionally substituted with 1-3 R^{a}.

Embodiment 78. The compound of any one of Embodiments 63-70 or 76, wherein L^{A4} is 1,2-phenylene or 1,3-phenylene, each of which is optionally substituted with 1-3 R^{a}.

Embodiment 79. The compound of any one of Embodiments 1-34 or 56, wherein L is:
-(L^{A3})₀₋₁-(L^{A1})₁₋₁₁-(L^{A3})₀₋₁-L^{A4}-_{bb},
wherein bb represents the point of attachment to Ring C.

Embodiment 80. The compound of Embodiment 79, wherein L is:
-(L^{A3})₀₋₁-(L^{A1})₁₋₁₁-(C(═O))₀₋₁-(4-10 membered heterocyclylene)-_{bb},
wherein bb represents the point of attachment to Ring C.

Embodiment 81. The compound of Embodiments 79 or 80, wherein L is:
-(L^{A3})₀₋₁-(L^{A1})₁₋₁₁-(C(═O))₀₋₁-(6 membered heterocyclylene)-_{bb},
wherein bb represents the point of attachment to Ring C.

Embodiment 82. The compound of any one of Embodiments 79-81, wherein L is a divalent group of Formula (L-3):

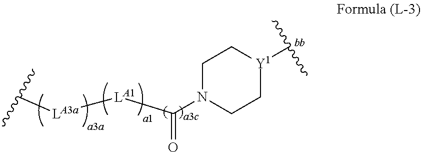

wherein:
Y¹ is N or CH;
a3a is 0 or 1;
L^{A3a} is selected from the group consisting of: —O—, —N(H)—, and —N(C₁₋₃ alkyl)-;
a1 is an integer from 1 to 11;
a3c is 0 or 1; and
bb represents the point of attachment to Ring C.

Embodiment 83. The compound of Embodiment 82, wherein a3a is 1.

Embodiment 84. The compound of Embodiments 82 or 83, wherein L^{A3a} is —O—.

Embodiment 85. The compound of Embodiment 82, wherein a3a is 0.

Embodiment 86. The compound of any one of Embodiments 82-85, wherein a1 is 1 or 2.

Embodiment 87. The compound of any one of Embodiments 82-85, wherein a1 is an integer from 3 to 5.

Embodiment 88. The compound of any one of Embodiments 82-85, wherein a1 is an integer from 5 to 9.

Embodiment 89. The compound of any one of Embodiments 82-85, wherein a1 is an integer from 9 to 11.

Embodiment 90. The compound of any one of Embodiments 82-89, wherein 0-1 occurrence of L^{A1} is —CHR^{L}— or —C(R^{L})₂—; and each remaining occurrence of L^{A1} is —CH₂—.

Embodiment 91. The compound of any one of Embodiments 82-90, wherein a3c is 0.

Embodiment 92. The compound of any one of Embodiments 82-90, wherein a3c is 1.

Embodiment 93. The compound of any one of Embodiments 1-34 or 60-63, wherein L is selected from the group consisting of:

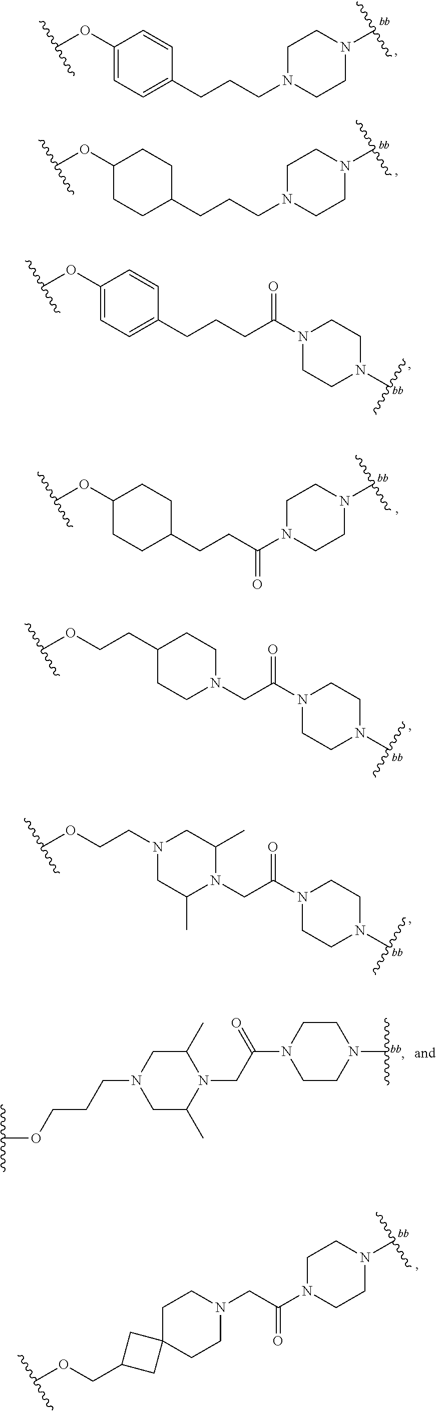

wherein bb represents the point of attachment to Ring C.

Embodiment 94. The compound of any one of Embodiments 1-34 or 79-82, wherein L is selected from the group consisting of:

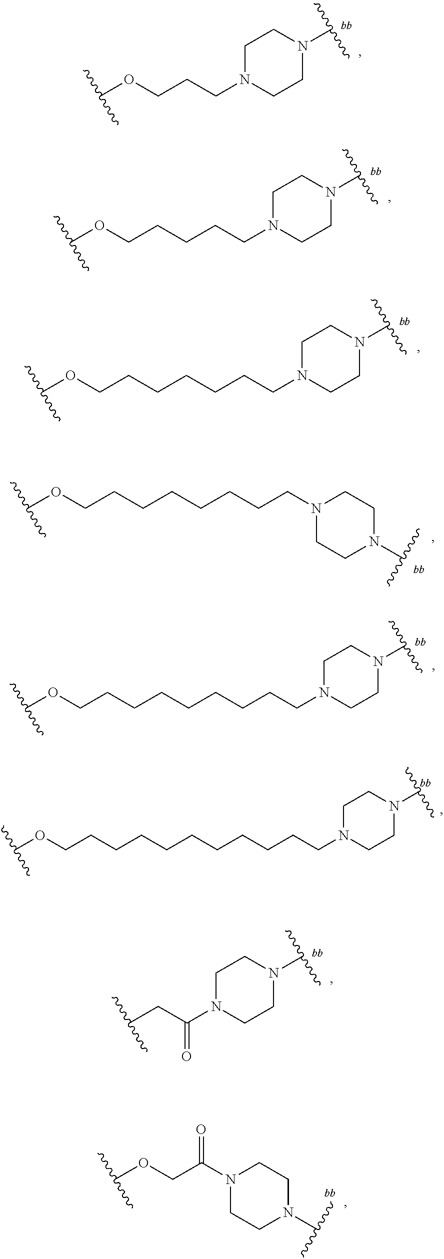

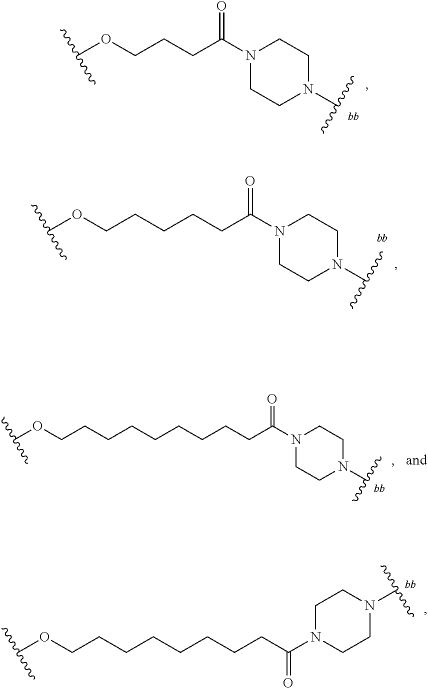

wherein bb represents the point of attachment to Ring C.

Embodiment 95. The compound of Embodiment 63, wherein the compound is a compound of Formula (I-A):

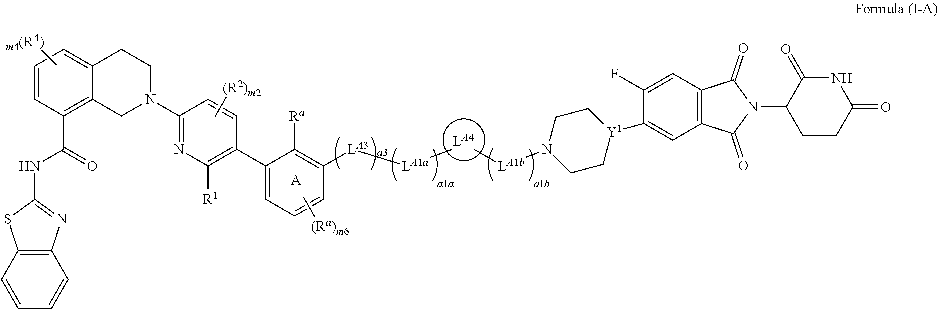

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Y¹ is N or CH.

Embodiment 96. The compound of Embodiment 63, wherein the compound is a compound of Formula (I-B):

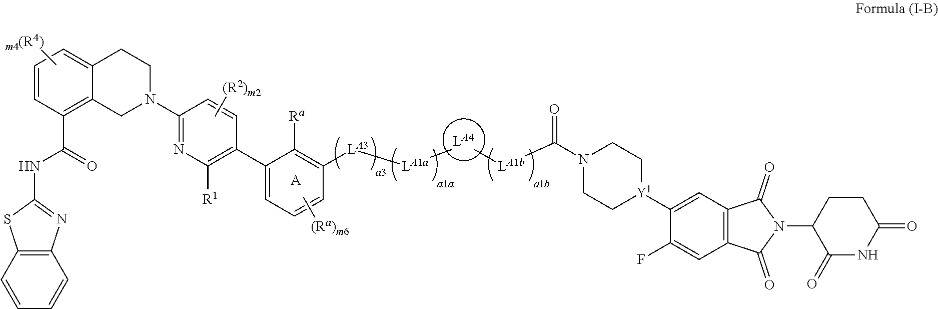

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Y¹ is N or CH.

Embodiment 97. The compound of Embodiments 95 or 96, wherein a3 is 1.

Embodiment 98. The compound of any one of Embodiments 95-97, wherein L^{A3} is —O—.

Embodiment 99. The compound of Embodiments 95 or 96, wherein a3 is 0.

Embodiment 100. The compound of any one of Embodiments 95-99, wherein a1a+a1b is 3.

Embodiment 101. The compound of any one of Embodiments 95-99, wherein a1a+a1b is 2.

Embodiment 102. The compound of any one of Embodiments 95-101, wherein each occurrence of L^{A1a} and L^{A1b} is —CH₂—.

Embodiment 103. The compound of any one of Embodiments 95-101, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 104. The compound of any one of Embodiments 95-101, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 105. The compound of any one of Embodiments 95-104, wherein L^{A4} is 4-10 membered heterocyclylene optionally substituted with 1-3 R^{a}.

Embodiment 106. The compound of any one of Embodiments 95-105, wherein L^{A4} is selected from the group consisting of:

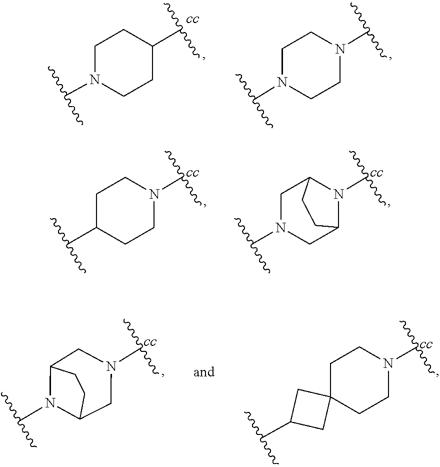 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}.

Embodiment 107. The compound of Embodiments 105 or 106, wherein each R^{a} present on L^{A4} is C₁₋₃ alkyl optionally substituted with 1-3 F.

Embodiment 108. The compound of any one of Embodiments 95-104, wherein L^{A4} is C₃₋₁₀ cycloalkylene optionally substituted with 1-3 R^{a}.

Embodiment 109. The compound of any one of Embodiments 95-104 or 108, wherein L^{A4} is 1,4-cyclohexylene optionally substituted with 1-3 R^{a}.

Embodiment 110. The compound of any one of Embodiments 95-104, wherein L^{A4} is phenylene or 5-6 membered heteroarylene, each of which is optionally substituted with 1-3 R^{a}.

Embodiment 111. The compound of any one of Embodiments 95-104 or 110, wherein L^{A4} is 1,4-phenylene optionally substituted with 1-3 R^{a}.

Embodiment 112. The compound of any one of Embodiments 95-104 or 110, wherein L^{A4} is 1,2-phenylene or 1,3-phenylene, each of which is optionally substituted with 1-3 R^{a}.

Embodiment 113. The compound of Embodiment 82, wherein the compound is a compound of Formula (I-C):

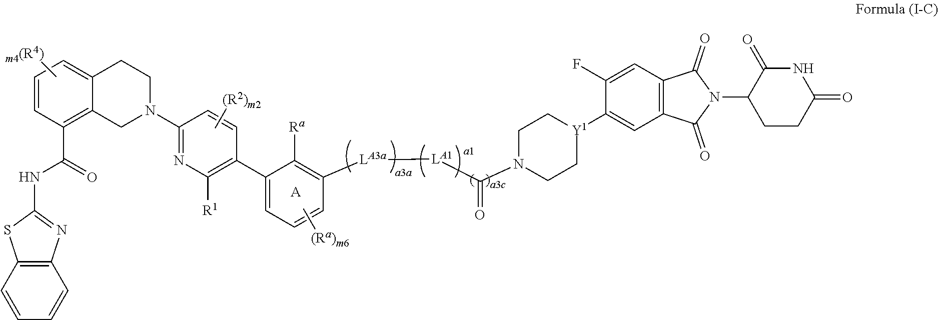

or a pharmaceutically acceptable salt thereof, wherein:
m6 is 0 or 1;
a3a is 0 or 1;
L^{A3a} is selected from the group consisting of: —O—, —N(H)—, and —N(C₁₋₃ alkyl)-;
a1 is an integer from 1 to 11;
a3c is 0 or 1; and
Y¹ is N or CH.

Embodiment 114. The compound of Embodiment 113, wherein a3a is 1.

Embodiment 115. The compound of Embodiments 113 or 114, wherein L^{A3a} is —O—.

Embodiment 116. The compound of any one of Embodiments 113-115, wherein a1 is 1 or 2.

Embodiment 117. The compound of any one of Embodiments 113-115, wherein a1 is an integer from 3 to 5.

Embodiment 118. The compound of any one of Embodiments 113-115, wherein a1 is an integer from 6 to 8.

Embodiment 119. The compound of any one of Embodiments 113-115, wherein a1 is an integer from 9 to 11.

Embodiment 120. The compound of any one of Embodiments 113-119, wherein 0-1 occurrence of L^{A1} is —CHR^{L}— or —C(R^{L})₂—; and each remaining occurrence of L^{A1} is —CH₂—.

Embodiment 121. The compound of any one of Embodiments 113-120, wherein a3c is 0.

Embodiment 122. The compound of any one of Embodiments 113-120, wherein a3c is 1.

Embodiment 123. The compound of any one of Embodiments 95-122, wherein m6 is 0.

Embodiment 124. The compound of any one of Embodiments 95-122, wherein the R^{a} present on Ring A is C₁₋₃ alkyl optionally substituted with 1-3 —F.

Embodiment 125. The compound of any one of Embodiments 95-124, wherein R¹ is C(O)OH.

Embodiment 126. The compound of any one of Embodiments 95-125, wherein m2 is 0.

Embodiment 127. The compound of any one of Embodiments 95-126, wherein m4 is 0.

Embodiment 128. The compound of any one of Embodiments 95-127, wherein Y is N.

Embodiment 129. The compound of any one of Embodiments 95-127, wherein Y is CH.

Embodiment 130. The compound of any one of Embodiments 1-129, wherein the compound is selected from the group consisting of 101, 102, 103, 104, 105, 106, 107, 108, 109, 110, 111, 111a, 112, 112a, and 113 as depicted in Table C1, or a pharmaceutically acceptable salt thereof.

Embodiment 131. A pharmaceutical composition comprising a compound of any one of Embodiments 1-130, or a pharmaceutically acceptable salt thereof, and a pharmaceutically acceptable excipient.

Embodiment 132. A Bcl-xL protein non-covalently bound with a compound of any one of Embodiments 1-130, or a pharmaceutically acceptable salt thereof.

Embodiment 133. A ternary complex comprising a Bcl-xL protein, a compound of any one of Embodiments 1-130, or a pharmaceutically acceptable salt thereof, and a CRBN protein.

Embodiment 134. A method for treating a cancer in a subject in need thereof, the method comprising administering to the subject a therapeutically effective amount of a compound of any one of Embodiments 1-130, or a pharmaceutically acceptable salt thereof, or a pharmaceutical composition according to Embodiment 131.

Embodiment 135. A method for inducing degradation of a Bcl-xL protein in a mammalian cell, the method comprising contacting the mammalian cell with an effective amount of a compound of any one of Embodiments 1-130, or a pharmaceutically acceptable salt thereof.

Embodiment 136. The method of Embodiment 135, wherein the contacting occurs in vivo.

Embodiment 137. The method of Embodiment 135, wherein the contacting occurs in vitro.

Embodiment 138. The method of any one of Embodiments 135-137, wherein the mammalian cell is a mammalian cancer cell.

### P04 Embodiments

Embodiment 1. A compound of Formula (I):

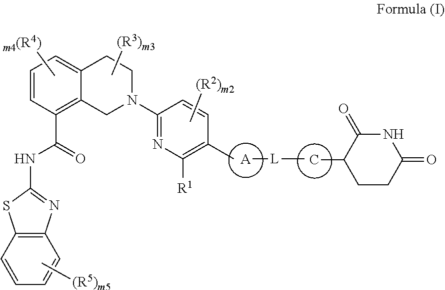

or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b};
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):

### AA

n1 is an integer from 3 to 15; and
each L^{A} is independently selected from the group consisting of: L^{A1}, L^{A}M, and L^{A4} provided that 1-3 occurrences of L^{A} is L^{A4};

### BB

n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any O—O, N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{e};
Ring C is selected from the group consisting of

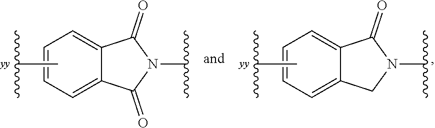

each of which is optionally substituted with 1-3 substituents independently selected from the group consisting of: R^{a} and R^{b}, wherein yy is the point of attachment to L;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene, and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂;
provided that one or both of (i) and (ii) applies:
(i) Ring C is substituted with 1-3 substituents independently selected from the group consisting of: R^{a} and R^{h}; and
(ii) L does not contain an adamantylene group.

Embodiment 2. The compound of claim 1, wherein Ring A is phenylene optionally substituted with 1-3 R^{a}.

Embodiment 3. The compound of claim 1 or 2, wherein Ring A is

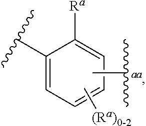

wherein aa represents the point of attachment to L or L^{T1}.

Embodiment 4. The compound of any one of claims 1-3, wherein Ring A is

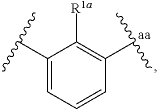

wherein aa represents the point of attachment to L or L^{T1}.

Embodiment 5. The compound of any one of claims 1-3, wherein Ring A is

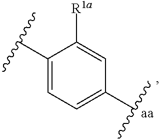

wherein aa represents the point of attachment to L or L^{T1}.

Embodiment 6. The compound of any one of claims 1-5, wherein one R^{a} present on Ring A is C₁₋₃ alkyl optionally substituted with 1-3 F.

Embodiment 7. The compound of any one of claims 1-6, wherein one R^{a} present on Ring A is methyl or CF₃.

Embodiment 8. The compound of any one of claims 1-7, wherein R¹ is C(O)OH.

Embodiment 9. The compound of any one of claims 1-8, wherein m2 is 0.

Embodiment 10. The compound of any one of claims 1-9, wherein m3 is 0.

Embodiment 11. The compound of any one of claims 1-10, wherein m4 is 0.

Embodiment 12. The compound of any one of claims 1-11, wherein m5 is 0.

Embodiment 13. The compound of any one of claims 1-8, wherein m2 is 0; m3 is 0; m4 is 0; and m5 is 0.

Embodiment 14. The compound of any one of claims 1-13, wherein Ring C is selected from the group consisting of:

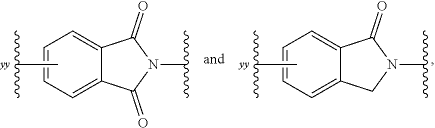

each of which is optionally substituted with 1-3 R^{a}, wherein yy is the point of attachment to L.

Embodiment 15. The compound of any one of claims 1-14, wherein Ring C is selected from the group consisting of:

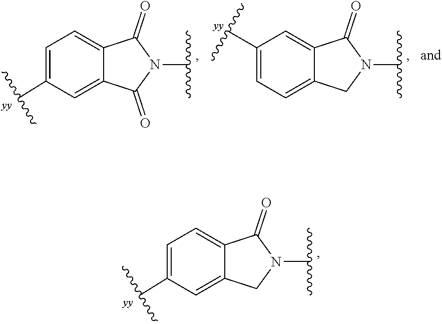 [SMILES: *c1ccc2c(c1)C(=O)N(*)C2=O ; *c1ccc2c(c1)C(=O)N(*)C2 ; *c1ccc2c(c1)CN(*)C2=O]

each of which is substituted with 1-3 R^{a}, wherein yy is the point of attachment to L.

Embodiment 16. The compound of any one of claims 1-15, wherein Ring C is

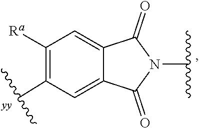

further optionally substituted with 1-2 R^{a} at one or more ring carbon atoms, wherein yy is the point of attachment to L.

Embodiment 17. The compound of any one of claims 1-16, wherein Ring C is

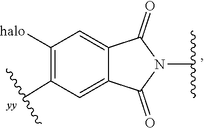

wherein yy is the point of attachment to L.

Embodiment 18. The compound of any one of claims 1-17, wherein Ring C is

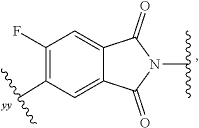

wherein yy is the point of attachment to L.

Embodiment 19. The compound of any one of claims 1-18, wherein L is -(L^{A})ₙ₁-; and L^{A} and n1 are defined according to (AA).

Embodiment 20. The compound of any one of claims 1-19, wherein n1 is an integer from 3 to 5.

Embodiment 21. The compound of any one of claims 1-19, wherein n1 is an integer from 5 to 9.

Embodiment 22. The compound of any one of claims 1-19 or 21, wherein n1 is 6, 7, or 8.

Embodiment 23. The compound of any one of claims 1-19, wherein n1 is an integer from 9 to 12.

Embodiment 24. The compound of any one of claims 1-23, wherein 1-2 occurrences of L^{A} is L^{A4}.

Embodiment 25. The compound of any one of claims 1-24, wherein one occurrence of L^{A} is L^{A4}.

Embodiment 26. The compound of any one of claims 1-24, wherein two occurrences of L^{A} are L^{A4}.

Embodiment 27. The compound of any one of claims 1-26, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 28. The compound of any one of claims 1-27, wherein 1-4 occurrences of L^{A} is L^{A3}.

Embodiment 29. The compound of any one of claims 1-28, wherein 1-3 occurrences of L^{A} is L^{A3}.

Embodiment 30. The compound of any one of claims 1-29, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 31. The compound of any one of claims 1-30, wherein 2-7 occurrences of L^{A} are L^{A1}.

Embodiment 32. The compound of any one of claims 1-31, wherein 2-5 occurrences of L^{A} are L^{A1}.

Embodiment 33. The compound of any one of claims 1-30, wherein 8-13 occurrences of L^{A} are L^{A1}.

Embodiment 34. The compound of any one of claims 1-33, wherein 0-2 occurrences of L^{A1} is —CHR^{L}— or —C(R^{L})₂—; and each remaining occurrence of L^{A1} is —CH₂—.

Embodiment 35. The compound of any one of claims 1-34, wherein each occurrence of L^{A1} is —CH₂—.

Embodiment 36. The compound of any one of claims 1-34, wherein one occurrence of L^{A1} is —CHR^{L}— or —C(R^{L})₂—; and each remaining occurrence of L^{A1} is —CH₂—.

Embodiment 37. The compound of any one of claims 1-36, wherein each R^{L} is independently selected from the group consisting of: —F and —C₁₋₃ alkyl optionally substituted with 1-3 F.

Embodiment 38. The compound of any one of claims 1-19, wherein L is -(L^{A})ₙ₁-, and L^{A} and n1 are defined according to (AA), wherein:
n1 is an integer from 5 to 9;
2 occurrences of L^{A} are L^{A4};
2-7 occurrences of L^{A} are L^{A1}; and
1-3 occurrences of L^{A} are L^{A3}.

Embodiment 39. The compound of any one of claims 1-19, wherein L is -(L^{A})ₙ₁-, and L^{A} and n1 are defined according to (AA), wherein;
n1 is an integer from 5 to 13;
1 occurrence of L^{A} is L^{A4};
2-11 occurrences of L^{A} are L^{A1}; and
1-3 occurrences of L^{A} are L^{A3}.

Embodiment 40. The compound of any one of claims 1-19, wherein L is selected from the group consisting of:
(i) -(L^{A3})₀₋₂-(L^{A1})₀₋₅-L^{A4}-(L^{A1})₀₋₅-L^{A3}-L^{A4}-_{bb}; and
(ii) -(L^{A3})₀₋₂-(L^{A1})₀₋₅-L^{A4}-(L^{A1})₀₋₅-L^{A4}-_{bb};
provided that L contains 2-7 L^{A1}; and wherein bb represents the point of attachment to Ring C.

Embodiment 41. The compound of any one of claims 1-19, wherein L is:
(L^{A3})₀₋₂-(L^{A1})₁₋₁₁-(L^{A3})₀₋₁-L^{A4}-_{bb},
wherein bb represents the point of attachment to Ring C.

Embodiment 42. The compound of any one of claims 38-41, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 43. The compound of any one of claims 38-42, wherein 0-1 occurrence of L^{A}1 is —CHR^{L}— or —C(R^{L})₂—; and each remaining occurrence of L^{A}1 is —CH₂—.

Embodiment 44. The compound of any one of claims 38-43, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 45. The compound of any one of claims 1-19 or 40, wherein L is:
-(L^{A3})₀₋₁-(L^{A1})₀₋₅-L^{A4}-(L^{A1})₀₋₅-L^{A4}-_{bb}; or
-(L^{A3})₀₋₁-(L^{A1})₀₋₅-L^{A4}-(L^{A1})₀₋₅-L^{A3}-L^{A4}-_{bb};
provided that L contains 2-7 L^{A1}; and wherein bb represents the point of attachment to Ring C.

Embodiment 46. The compound of claim 45, wherein L is:
-(L^{A3})₀₋₁-(L^{A1})₀₋₅-L^{A4}-(L^{A1})₀₋₅-(4-10 membered heterocyclylene)-_{bb}; or
-(L^{A3})₀₋₁-(L^{A1})₀₋₅-L^{A4}-(L^{A1})₀₋₅-C(═O)-(4-10 membered heterocyclylene)-_{bb};
provided that L contains 2-7 L^{A1}; and wherein bb represents the point of attachment to Ring C.

Embodiment 47. The compound of claim 45 or 46, wherein L is:
-(L^{A3})₀₋₁-(L^{A1})₀₋₅-L^{A4}-(L^{A1})₀₋₅-(6-membered heterocyclylene)-_{bb}; or
-(L^{A3})₀₋₁-(L^{A1})₀₋₅-L^{A4}-(L^{A1})₀₋₅-C(═O)-(6-membered heterocyclylene)-_{bb};
provided that L contains 2-5 L^{A1}; and wherein bb represents the point of attachment to Ring C.

Embodiment 48. The compound of any one of claims 45-47, wherein L is a divalent group of Formula (L-1) or (L-2):

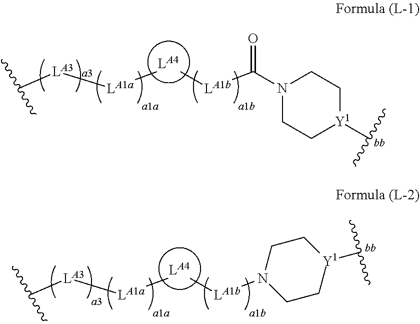

wherein:
Y¹ is N or CH;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

Embodiment 49. The compound of claim 48, wherein a3 is 1.

Embodiment 50. The compound of claim 48 or 49, wherein L^{A3} is —O—.

Embodiment 51. The compound of any one of claims 48-50, wherein a1a+a1b is 3 or 4.

Embodiment 52. The compound of any one of claims 48-50, wherein a1a+a1b is 2.

Embodiment 53. The compound of any one of claims 48-52, wherein each occurrence of L^{A1a} and L^{A1b} is —CH₂—.

Embodiment 54. The compound of any one of claims 48-52, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 55. The compound of any one of claims 48-52, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 56. The compound of any one of claims 48-55, L^{A4} is 4-10 membered heterocyclylene optionally substituted with 1-3 R^{a}.

Embodiment 57. The compound of any one of claims 48-56, wherein L^{A4} is selected from the group consisting of

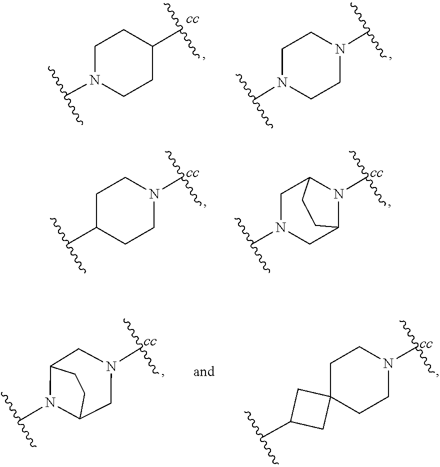 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}.

Embodiment 58. The compound of claim 56 or 57, wherein each R^{a} present on L^{A4} is C₁₋₃ alkyl optionally substituted with 1-3 F.

Embodiment 59. The compound of any one of claims 48-55, wherein L^{A4} is C₃₋₁₀ cycloalkylene optionally substituted with 1-3 R^{a}.

Embodiment 60. The compound of any one of claims 48-55 or 59, wherein L^{A4} is 1,4-cyclohexylene optionally substituted with 1-3 R^{a}.

Embodiment 61. The compound of any one of claims 48-55, wherein L^{A4} is phenylene or 5-6 membered heteroarylene, each of which is optionally substituted with 1-3 R^{a}.

Embodiment 62. The compound of any one of claims 48-55 or 61, wherein L^{A4} is 1,4-phenylene optionally substituted with 1-3 R^{a}.

Embodiment 63. The compound of any one of claims 48-55 or 61, wherein L^{A4} is 1,2-phenylene or 1,3-phenylene, each of which is optionally substituted with 1-3 R^{a}.

Embodiment 64. The compound of any one of claims 1-19 or 41, wherein L is:
-(L^{A3})₀₋₁-(L^{A1})₁₋₁₁-(L^{A3})₀₋₁-L^{A4}-_{bb},
wherein bb represents the point of attachment to Ring C.

Embodiment 65. The compound of claim 64, wherein L is:
-(L^{A3})₀₋₁-(L^{A1})₁₋₁₁-(C(═O))₀₋₁-(4-10 membered heterocyclylene)-_{bb},
wherein bb represents the point of attachment to Ring C.

Embodiment 66. The compound of claim 64 or 65, wherein L is:
-(L^{A3})₀₋₁-(L^{A1})₁₋₁₁-(C(═O))₀₋₁-(6 membered heterocyclylene)-_{bb},
wherein bb represents the point of attachment to Ring C.

Embodiment 67. The compound of any one of claims 64-66, wherein L is a divalent group of Formula (L-3):

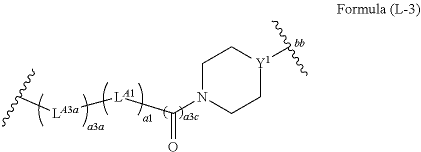

wherein:
Y is N or CH;
a3a is 0 or 1;
L^{A3a} is selected from the group consisting of: —O—, —N(H)—, and —N(C₁₋₃ alkyl)-;
a1 is an integer from 1 to 11;
a3c is 0 or 1; and
bb represents the point of attachment to Ring C.

Embodiment 68. The compound of claim 67, wherein a3a is 1.

Embodiment 69. The compound of claim 67 or 68, wherein L^{A3a} is —O—.

Embodiment 70. The compound of claim 67, wherein a3a is 0.

Embodiment 71. The compound of any one of claims 67-70, wherein a1 is 1 or 2.

Embodiment 72. The compound of any one of claims 67-70, wherein a1 is an integer from 3 to 5.

Embodiment 73. The compound of any one of claims 67-70, wherein a1 is an integer from 5 to 9.

Embodiment 74. The compound of any one of claims 67-70, wherein a1 is an integer from 9 to 11.

Embodiment 75. The compound of any one of claims 67-74, wherein 0-1 occurrence of L^{A1} is —CHR^{L}— or —C(R^{L})₂—; and each remaining occurrence of L^{A} is —CH₂—.

Embodiment 76. The compound of any one of claims 67-75, wherein a3c is 0.

Embodiment 77. The compound of any one of claims 67-75, wherein a3c is 1.

Embodiment 78. The compound of any one of claims 1-19 or 45-48, wherein L is selected from the group consisting of:

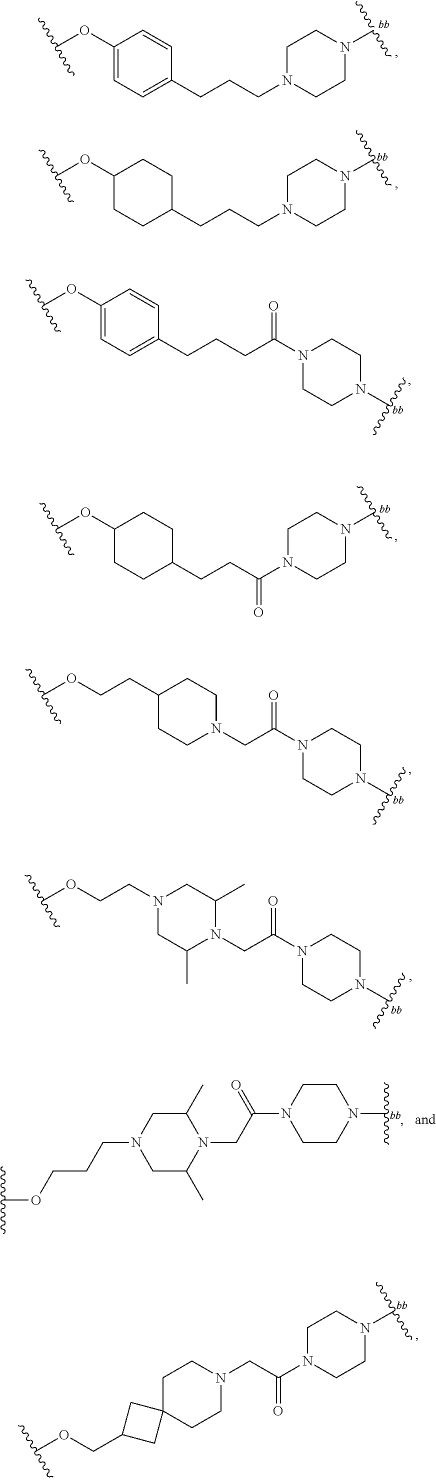

wherein bb represents the point of attachment to Ring C.

Embodiment 79. The compound of any one of claims 1-19 or 45-48, wherein L is selected from the group consisting of:

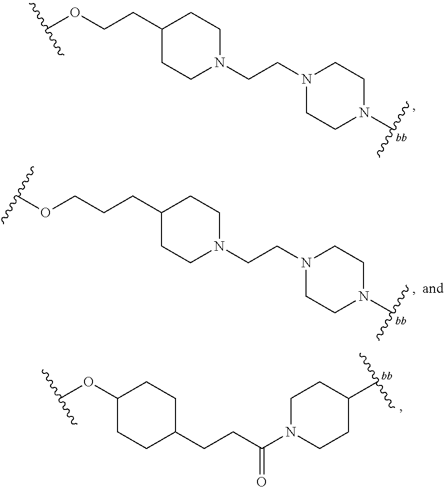

wherein bb represents the point of attachment to Ring C.

Embodiment 80. The compound of any one of claims 1-19 or 64-67, wherein L is selected from the group consisting of:

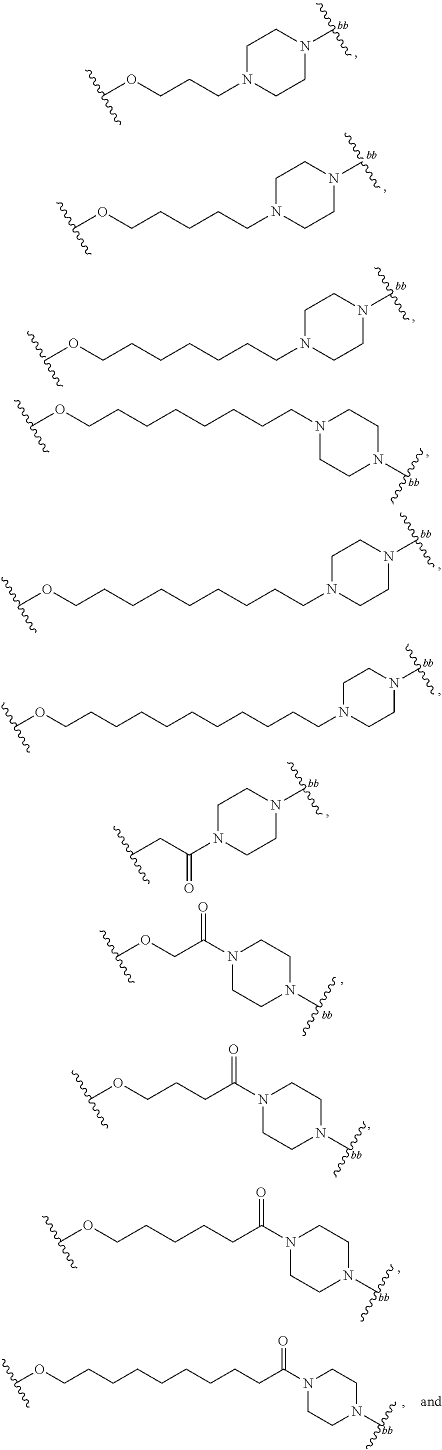

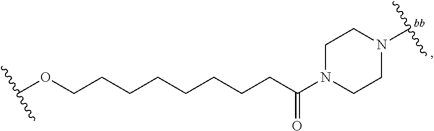

wherein bb represents the point of attachment to Ring C.

Embodiment 81. The compound of claim 48, wherein the compound is a compound of Formula (I-A):

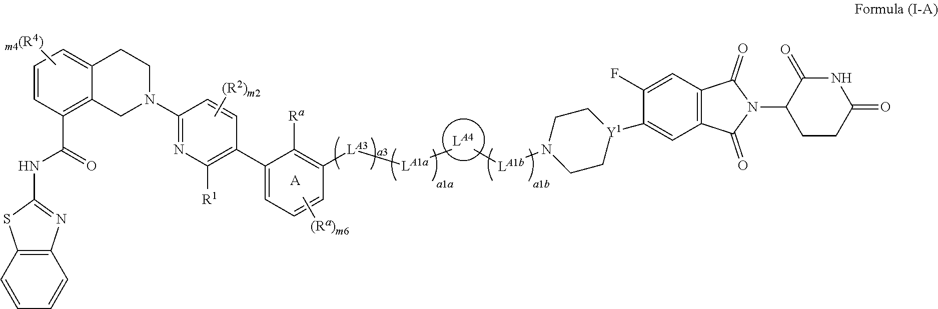

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}- and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Y¹ is N or CH.

Embodiment 82. The compound of claim 48, wherein the compound is a compound of Formula (I-B):

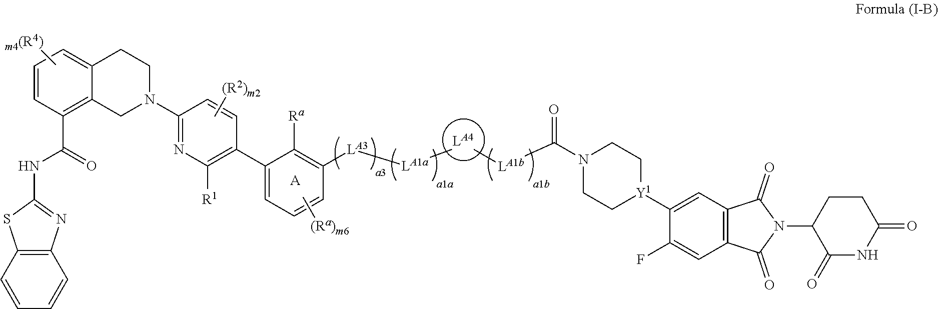

or a pharmaceutically acceptable salt thereof, wherein:
m6 is 0 or 1;
a3 is 0 or 1;
L^{A}3 is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Y¹ is N or CH.

Embodiment 83. The compound of claim 81 or 82, wherein a3 is 1.

Embodiment 84. The compound of any one of claims 81-83, wherein L^{A3} is —O—.

Embodiment 85. The compound of claim 81 or 82, wherein a3 is 0.

Embodiment 86. The compound of any one of claims 81-84, wherein a1a+a1b is 3.

Embodiment 87. The compound of any one of claims 81-84, wherein a1a+a1b is 2.

Embodiment 88. The compound of any one of claims 81-87, wherein each occurrence of L^{A1a} and L^{A1b} is —CH₂—.

Embodiment 89. The compound of any one of claims 81-87, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 90. The compound of any one of claims 81-87, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 91. The compound of any one of claims 81-90, wherein L^{A4} is 4-10 membered heterocyclylene optionally substituted with 1-3 R^{a}.

Embodiment 92. The compound of any one of claims 81-91, wherein L^{A4} is selected from the group consisting of:

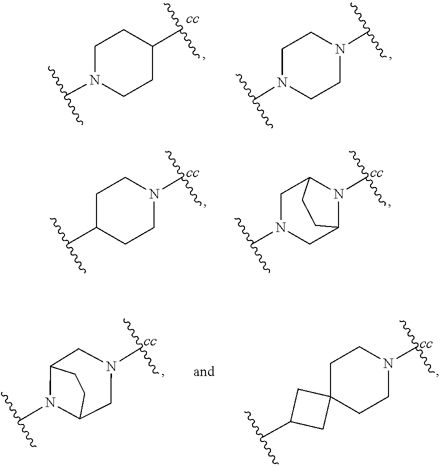 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each of which is optionally substituted with 1-3 R^{a} at one or more ring carbon atoms, wherein cc represents the point of attachment to L^{A1b}.

Embodiment 93. The compound of claim 91 or 92, wherein each R^{a} present on L^{A4} is C₁₋₃ alkyl optionally substituted with 1-3 F.

Embodiment 94. The compound of any one of claims 81-90, wherein L^{A4} is C₃₋₁₀ cycloalkylene optionally substituted with 1-3 R^{a}.

Embodiment 95. The compound of any one of claims 81-90 or 94, wherein L^{A4} is 1,4-cyclohexylene optionally substituted with 1-3 R^{a}.

Embodiment 96. The compound of any one of claims 81-90, wherein L^{A4} is phenylene or 5-6 membered heteroarylene, each of which is optionally substituted with 1-3 R^{a}.

Embodiment 97. The compound of any one of claims 81-90 or 96, wherein L^{A4} is 1,4-phenylene optionally substituted with 1-3 R^{a}.

Embodiment 98. The compound of any one of claims 81-90 or 96, wherein L^{A4} is 1,2-phenylene or 1,3-phenylene, each of which is optionally substituted with 1-3 R^{a}.

Embodiment 99. The compound of claim 67, wherein the compound is a compound of Formula (I-C):

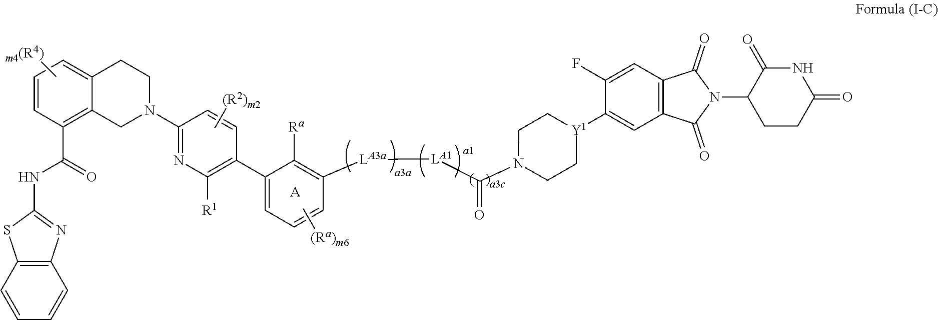

or a pharmaceutically acceptable salt thereof, wherein:
m6 is 0 or 1;
a3a is 0 or 1;
L^{A3a} is selected from the group consisting of: —O—, —N(H)—, and —N(C₁₋₃ alkyl)-;
a1 is an integer from 1 to 11;
a3c is 0 or 1; and
Y¹ is N or CH.

Embodiment 100. The compound of claim 99, wherein a3a is 1.

Embodiment 101. The compound of claim 99 or 100, wherein L^{A3a} is —O—.

Embodiment 102. The compound of any one of claims 99-101, wherein a1 is 1 or 2.

Embodiment 103. The compound of any one of claims 99-101, wherein a1 is an integer from 3 to 5.

Embodiment 104. The compound of any one of claims 99-101, wherein a1 is an integer from 6 to 8.

Embodiment 105. The compound of any one of claims 99-101, wherein a1 is an integer from 9 to 11.

Embodiment 106. The compound of any one of claims 99-105, wherein 0-1 occurrence of L^{A1} is —CHR^{L}— or —C(R^{L})₂—; and each remaining occurrence of L^{A} is —CH₂—.

Embodiment 107. The compound of any one of claims 99-106, wherein a3c is 0.

Embodiment 108. The compound of any one of claims 99-106, wherein a3c is 1.

Embodiment 109. The compound of any one of claims 81-108, wherein m6 is 0.

Embodiment 110. The compound of any one of claims 81-109, wherein the R^{a} present on Ring A is C₁₋₃ alkyl optionally substituted with 1-3 —F.

Embodiment 111. The compound of any one of claims 81-110, wherein R¹ is C(O)OH.

Embodiment 112. The compound of any one of claims 81-111, wherein m2 is 0.

Embodiment 113. The compound of any one of claims 81-112, wherein m4 is 0.

Embodiment 114. The compound of any one of claims 81-113, wherein Y is N.

Embodiment 115. The compound of any one of claims 81-113, wherein Y is CH.

Embodiment 116. The compound of any one of claims 1-115, wherein the compound is selected from the group consisting of 101, 102, 103, 104, 105, 106, 107, 108, 109, 110, 111, 111a, 112, 112a, 113, 114, 115, 116, 117, 118, 119, 120, 120a, 121, 122, 122a, 122b, 123, 124, 124a, 124b, 125, 126, 127, 128, 128a, 128b, 129, 129a, 130, 130a, 132, 133, 135, 135a, 135b, 137, 138, 139, 139a, 140, 140a, 141, 142, 143, 144, and 145 as depicted in Table C1, or a pharmaceutically acceptable salt thereof, provided that the compound does not exhibit Yₘᵢₙ (6 h)≥70% and/or Yₘᵢₙ (24 h)≥70% under conditions described in Example B1.

Embodiment 117. A pharmaceutical composition comprising a compound as claimed in any one of claims 1-116, or a pharmaceutically acceptable salt thereof, and a pharmaceutically acceptable excipient.

Embodiment 118. A BCL-X_{L} protein non-covalently bound with a compound as claimed in any one of claims 1-116, or a pharmaceutically acceptable salt thereof.

Embodiment 119. A ternary complex comprising a BCL-X_{L} protein, a compound as claimed in any one of claims 1-116, or a pharmaceutically acceptable salt thereof, and a CRBN protein.

Embodiment 120. A method for treating a cancer in a subject in need thereof, the method comprising administering to the subject a therapeutically effective amount of a compound of any one of claims 1-116, or a pharmaceutically acceptable salt thereof, or a pharmaceutical composition according to claim 117.

Embodiment 121. The method of claim 120, further comprising administering an additional therapy or therapeutic agent.

Embodiment 122. The method of claim 121, wherein the additional therapy or therapeutic agent is an ALK inhibitor, a BCL-2 inhibitor, a BCR-Abl inhibitor, a BRaf inhibitor, a CDK2 inhibitor, CDK 4/6 inhibitor, a CDK7 inhibitor, a CDK9 inhibitor, an EGFR inhibitor, an anti-EGFR antibody, an ERK inhibitor, a FGFR1 inhibitor, a FGFR2 inhibitor, a FGFR3 inhibitor, a FGFR4 inhibitor, a HER2 inhibitor, a JAK2 inhibitor, a KRas inhibitor, a MEK inhibitor, a MET inhibitor, a PARP inhibitor, an LSD1 inhibitor, a BET inhibitor, a telomerase inhibitor, a TORC1/2 inhibitor, chemotherapy, radiotherapy, or a combination thereof.

Embodiment 123. The method of any one of claims 120-122, wherein the cancer is breast cancer, colorectal cancer, bile duct cancer, colorectal cancer, gastrointestinal stromal tumor, pancreatic cancer, bladder cancer, kidney cancer, cervical cancer, ovarian cancer, uterine cancer, head and neck cancer, hematological cancer, lung cancer, skin cancer, or a combination thereof.

Embodiment 124. The method of claim 123, wherein the hematological cancer is acute lymphocytic leukemia (ALL), acute myeloid leukemia (AML), chronic lymphocytic leukemia (CLL), follicular lymphoma (FL), small lymphocytic lymphoma (SLL), essential thrombocythemia, polycythemia vera, myelofibrosis, or a combination thereof.

Embodiment 125. A method for inducing degradation of a BCL-X_{L} protein in a mammalian cell, the method comprising contacting the mammalian cell with an effective amount of a compound of any one of claims 1-116, or a pharmaceutically acceptable salt thereof.

Embodiment 126. The method of claim 125, wherein the contacting occurs in vivo.

Embodiment 127. The method of claim 125, wherein the contacting occurs in vitro.

Embodiment 128. The method of any one of claims 125-127, wherein the mammalian cell is a mammalian cancer cell.

What is claimed is:

1. A compound of Formula (I): 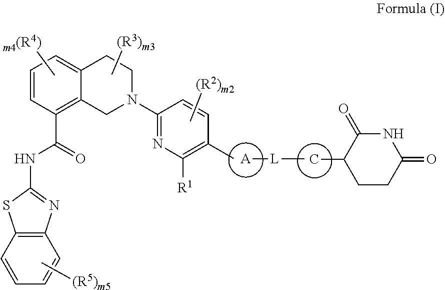
or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b},
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{e};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
(AA)
n1 is an integer from 3 to 15; and
each L^{A} is independently selected from the group consisting of: L^{A1}, L^{A3}, and L^{A4} provided that 1-3 occurrences of L^{A} is L^{A4};
(BB)
n1 is an integer from 0 to 20; and
each L^{A} is independently selected from the group consisting of: L^{A1} and L^{A3};
each L^{A}1 is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any O—O, N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
Ring C is selected from the group consisting of: 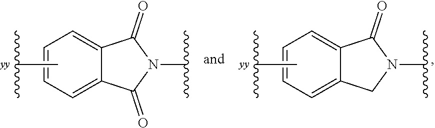
each of which is optionally substituted with 1-3 substituents independently selected from the group consisting of: R^{a} and R^{b}, wherein yy is the point of attachment to L;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene, and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂;
provided that one or both of (i) and (ii) applies:
(i) Ring C is substituted with 1-3 substituents independently selected from the group consisting of: R^{a} and R^{b}; and
(ii) L does not contain an adamantylene group.

2. The compound of claim 1, wherein Ring A is phenylene optionally substituted with 1-3 R^{a}.

3. The compound of claim 1 or 2, wherein Ring A is 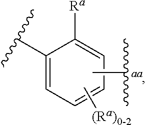
wherein aa represents the point of attachment to L or L^{T1}; or
Ring A is 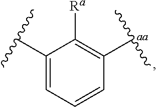
wherein aa represents the point of attachment to L or L^{T1}; or
Ring A is 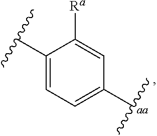
wherein aa represents the point of attachment to L or L^{T1}.

4. The compound of any one of claims 1-3, wherein one R^{a} present on Ring A is C₁₋₃ alkyl optionally substituted with 1-3 F; or
one R^{a} present on Ring A is methyl or CF₃.

5. The compound of any one of claims 1-4, wherein R¹ is C(O)OH.

6. The compound of any one of claims 1-5, wherein m2 is 0.

7. The compound of any one of claims 1-6, wherein m3 is 0.

8. The compound of any one of claims 1-7, wherein m4 is 0.

9. The compound of any one of claims 1-8, wherein m5 is 0.

10. The compound of any one of claims 1-5, wherein m2 is 0; m3 is 0; m4 is 0; and m5 is 0.

11. The compound of any one of claims 1-10, wherein Ring C is selected from the group consisting of: 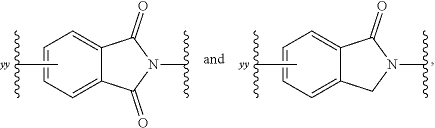
each of which is optionally substituted with 1-3 R^{a}, wherein yy is the point of attachment to L.

12. The compound of any one of claims 1-11, wherein Ring C is selected from the group consisting of 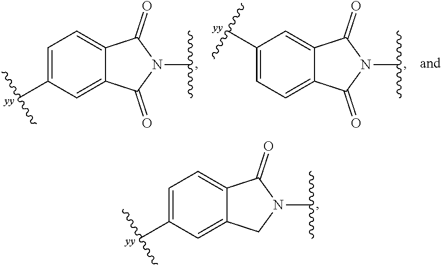 [SMILES: *c1ccc2c(c1)C(=O)N(*)C2=O ; *c1ccc2c(c1)C(=O)N(*)C2=O ; *c1ccc2c(c1)CN(*)C2=O]
each of which is substituted with 1-3 R^{a}, wherein yy is the point of attachment to L; or
Ring C is 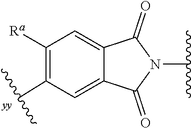
further optionally substituted with 1-2 R^{a} at one or more ring carbon atoms, wherein yy is the point of attachment to L; or
Ring C is 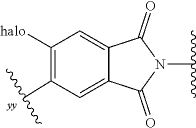
wherein yy is the point of attachment to L; or
Ring C is 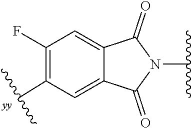
wherein yy is the point of attachment to L.

13. The compound of any one of claims 1-12, wherein L is -(L^{A})ₙ₁-; and L^{A} and n1 are defined according to (AA).

14. The compound of any one of claims 1-13, wherein n1 is an integer from 3 to 5; or
n1 is an integer from 5 to 9; or
n1 is 6, 7, or 8; or
n1 is an integer from 9 to 12.

15. The compound of any one of claims 1-14, wherein 1-2 occurrences of L^{A} is L^{A4}; or
one occurrence of L^{A} is L^{A4}; or
two occurrences of L^{A} are L^{A4}.

16. The compound of any one of claims 1-15, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

17. The compound of any one of claims 1-16, wherein 1-4 occurrences of L^{A} is L^{A3}; or
1-3 occurrences of L^{A} is L^{A3}.

18. The compound of any one of claims 1-17, wherein 0-1 occurrence of L^{A3} is C(═O); and each remaining occurrence of L^{A3} is independently selected from the group consisting of: —O—; —N(H)—; and —N(C₁₋₃ alkyl)-.

19. The compound of any one of claims 1-30, wherein 2-7 occurrences of L^{A} are L^{A1}; or
2-5 occurrences of L^{A} are L^{A1}; or
8-13 occurrences of L^{A} are L^{A1}.

20. The compound of any one of claims 1-19, wherein 0-2 occurrences of L^{A1} is —CHR^{L}— or —C(R^{L})₂—; and each remaining occurrence of L^{A1} is —CH₂—; or
each occurrence of L^{A1} is —CH₂—; or
one occurrence of L^{A1} is —CHR^{L}— or —C(R^{L})₂—; and each remaining occurrence of L^{A1} is —CH₂—.

21. The compound of any one of claims 1-20, wherein each R^{L} is independently selected from the group consisting of: —F and —C₁₋₃ alkyl optionally substituted with 1-3 F.

22. The compound of any one of claims 1-13, wherein L is -(L^{A})ₙ₁-, and L^{A} and n1 are defined according to (AA), wherein:
n1 is an integer from 5 to 9;
2 occurrences of L^{A} are L^{A4};
2-7 occurrences of L^{A} are L^{A1}; and
1-3 occurrences of L^{A} are L^{A3}.

23. The compound of any one of claims 1-13, wherein L is -(L^{A})ₙ₁-, and L^{A} and n1 are defined according to (AA), wherein;
n1 is an integer from 5 to 13;
1 occurrence of L^{A} is L^{A4};
2-11 occurrences of L^{A} are L^{A1}; and
1-3 occurrences of L^{A} are L^{A3}.

24. The compound of any one of claims 1-13, wherein L is selected from the group consisting of:
(i) -(L^{A3})₀₋₂-(L^{A1})₀₋₅-L^{A4}-(L^{A1})₀₋₅-L^{A3}-L^{A4}-_{bb}; and
(ii) -(L^{A3})₀₋₂-(L^{A1})₀₋₅-L^{A4}-(L^{A1})₀₋₅-L^{A4}-_{bb};
provided that L contains 2-7 L^{A}1; and wherein bb represents the point of attachment to Ring C.

25. The compound of any one of claims 1-13, wherein L is:
-(L^{A3})₀₋₂-(L^{A1})₁₋₁₁-(L^{A3})₀₋₁-L^{A4}-_{bb},
wherein bb represents the point of attachment to Ring C.

26. The compound of any one of claims 22-25, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

27. The compound of any one of claims 22-26, wherein 0-1 occurrence of L^{A}1 is —CHR^{L}— or —C(R^{L})₂—; and each remaining occurrence of L^{A}1 is —CH₂—.

28. The compound of any one of claims 22-27, wherein 0-1 occurrence of L^{A3} is C(═O); and each remaining occurrence of L^{A3} is independently selected from the group consisting of: —O—; —N(H)—; and —N(C₁₋₃ alkyl)-.

29. The compound of any one of claims 1-13 or 28, wherein L is:
-(L^{A3})₀₋₁-(L^{A1})₀₋₅-L^{A4}-(L^{A1})₀₋₅-L^{A4}-_{bb}; or
-(L^{A3})₀₋₁-(L^{A1})₀₋₅-L^{A4}-(L^{A1})₀₋₅-L^{A3}-L^{A4}-_{bb};
provided that L contains 2-7 L^{A1}; and wherein bb represents the point of attachment to Ring C.

30. The compound of claim 29, wherein L is:
-(L^{A3})₀₋₁-(L^{A1})₀₋₅-L^{A4}-(L^{A1})₀₋₅-(4-10 membered heterocyclylene)-_{bb}; or
-(L^{A3})₀₋₁-(L^{A1})₀₋₅-L^{A4}-(L^{A1})₀₋₅-C(═O)-(4-10 membered heterocyclylene)-_{bb};
provided that L contains 2-7 L^{A1}; and wherein bb represents the point of attachment to Ring C.

31. The compound of claim 29 or 30, wherein L is:
-(L^{A3})₀₋₁-(L^{A1})₀₋₅-L^{A4}-(L^{A1})₀₋₅-(6-membered heterocyclylene)-_{bb}; or
-(L^{A3})₀₋₁-(L^{A1})₀₋₅-L^{A4}-(L^{A1})₀₋₅-C(═O)-(6-membered heterocyclylene)-_{bb};
provided that L contains 2-5 L^{A1}; and wherein bb represents the point of attachment to Ring C.

32. The compound of any one of claims 29-31, wherein L is a divalent group of Formula (L-1) or (L-2): 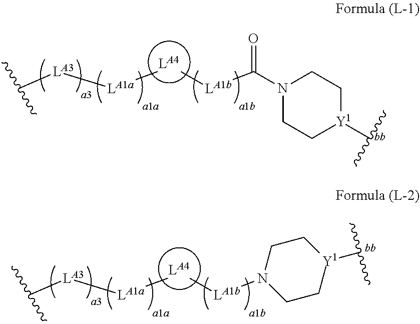
wherein:
Y is N or CH;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

33. The compound of claim 32, wherein a3 is 1.

34. The compound of claim 32 or 33, wherein L^{A3} is —O—.

35. The compound of any one of claims 32-34, wherein a1a+a1b is 3 or 4; or
a1a+a1b is 2.

36. The compound of any one of claims 32-35, wherein each occurrence of L^{A1a} and L^{A1b} is —CH₂—.

37. The compound of any one of claims 32-35, wherein one occurrence of L^{A1a} is —CHR^{L}— or —C(R^{L})₂—; each remaining occurrence of L^{A1a} is —CH₂—; and each occurrence of L^{A1b} is —CH₂—; or
one occurrence of L^{A1b} is —CHR^{L}— or —C(R^{L})₂—; each remaining occurrence of L^{A1b} is —CH₂—; and each occurrence of L^{A1a} is —CH₂—.

38. The compound of any one of claims 32-37, L^{A4} is 4-10 membered heterocyclylene optionally substituted with 1-3 R^{a}.

39. The compound of any one of claims 32-38, wherein L^{A4} is selected from the group consisting of: 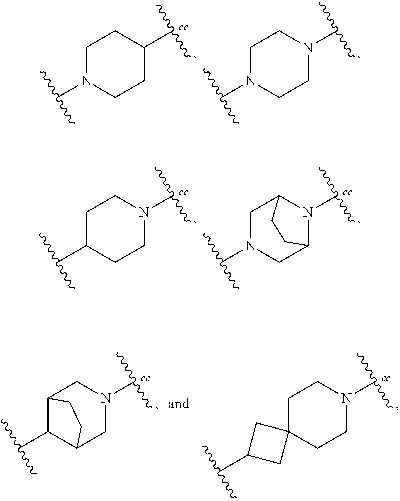 [SMILES: *C1CCN(*)CC1 ; *N1CCN(*)CC1 ; *C1CCN(*)CC1 ; *N1CC2CCC(C1)N2* ; *C1C2CCC1CN(*)C2 ; *C1CC2(CCN(*)CC2)C1]
each optionally substituted with 1-3 R^{a} at one or more ring carbon atoms, wherein cc represents the point of attachment to L^{A1b}.

40. The compound of claim 38 or 39, wherein each R^{a} present on L^{A4} is C₁₋₃ alkyl optionally substituted with 1-3 F.

41. The compound of any one of claims 32-37, wherein L^{A4} is C₃₋₁₀ cycloalkylene optionally substituted with 1-3 R^{a}.

42. The compound of any one of claims 32-37 or 41, wherein L^{A4} is 1,4-cyclohexylene optionally substituted with 1-3 R^{a}.

43. The compound of any one of claims 32-37, wherein L^{A4} is phenylene or 5-6 membered heteroarylene, each of which is optionally substituted with 1-3 R^{a}.

44. The compound of any one of claims 32-37 or 43, wherein L^{A4} is 1,4-phenylene optionally substituted with 1-3 R^{a}.

45. The compound of any one of claims 32-37 or 43, wherein L^{A4} is 1,2-phenylene or 1,3-phenylene, each of which is optionally substituted with 1-3 R^{a}.

46. The compound of any one of claims 1-13 or 25, wherein L is:
-(L^{A3})₀₋₁-(L^{A1})₁₋₁₁-(L^{A3})₀₋₁-L^{A4}-_{bb},
wherein bb represents the point of attachment to Ring C.

47. The compound of claim 46, wherein L is:
-(L^{A3})₀₋₁-(L^{A1})₁₋₁₁-(C(═O))₀₋₁-(4-10 membered heterocyclylene)-_{bb},
wherein bb represents the point of attachment to Ring C.

48. The compound of claim 46 or 47, wherein L is:
-(L^{A3})₀₋₁-(L^{A1})₁₋₁₁-(C(═O))₀₋₁-(6 membered heterocyclylene)-_{bb},
wherein bb represents the point of attachment to Ring C.

49. The compound of any one of claims 46-48, wherein L is a divalent group of 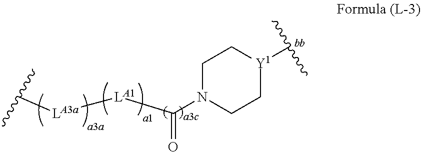
wherein:
Y¹ is N or CH;
a3a is 0 or 1;
L^{A3a} is selected from the group consisting of: —O—, —N(H)—, and —N(C₁₋₃ alkyl)-;
a1 is an integer from 1 to 11;
a3c is 0 or 1; and
bb represents the point of attachment to Ring C.

50. The compound of claim 49, wherein a3a is 1.

51. The compound of claim 49 or 50, wherein L^{A3a} is —O—.

52. The compound of claim 49, wherein a3a is 0.

53. The compound of any one of claims 49-52, wherein a1 is 1 or 2; or
a1 is an integer from 3 to 5; or
a1 is an integer from 5 to 9; or
a1 is an integer from 9 to 11.

54. The compound of any one of claims 49-53, wherein 0-1 occurrence of L^{A1} is —CHR^{L}— or —C(R^{L})₂—; and each remaining occurrence of L^{A1} is —CH₂—.

55. The compound of any one of claims 49-54, wherein a3c is 0.

56. The compound of any one of claims 49-54, wherein a3c is 1.

57. The compound of any one of claims 1-13 or 29-32, wherein L is selected from the group consisting of: 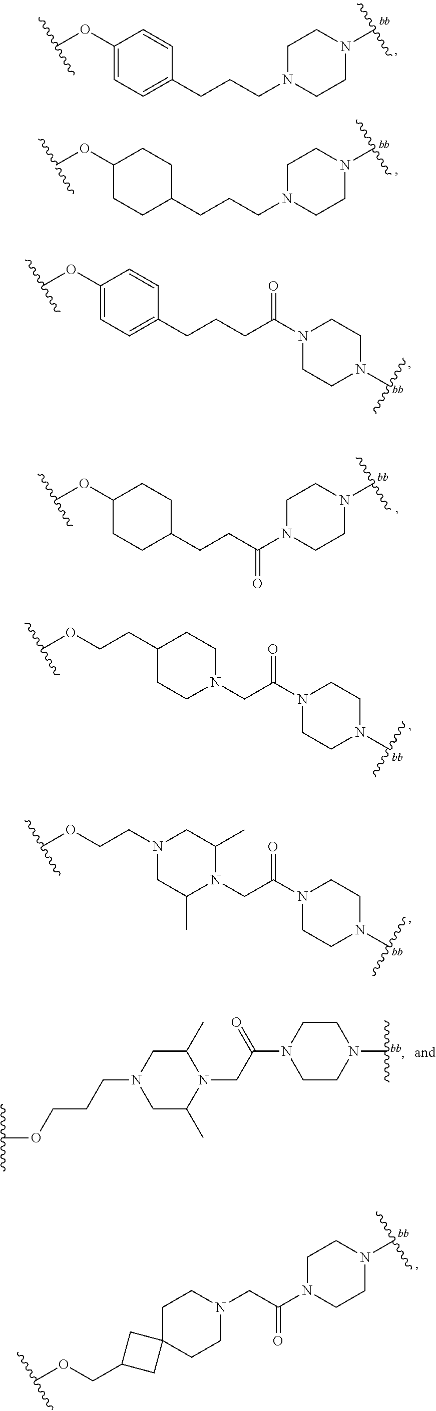
wherein bb represents the point of attachment to Ring C.

58. The compound of any one of claims 1-13 or 29-32, wherein L is selected from the group consisting of: 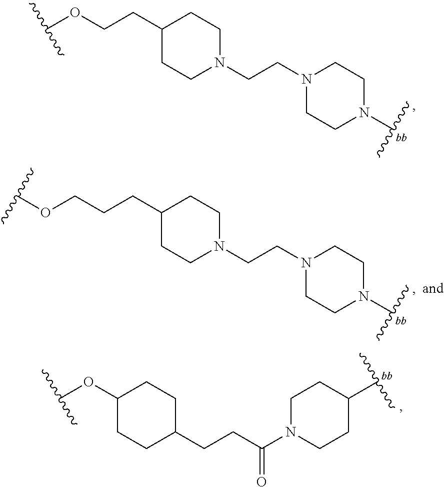
wherein bb represents the point of attachment to Ring C.

59. The compound of any one of claims 1-13 or 46-49, wherein L is selected from the group consisting of: 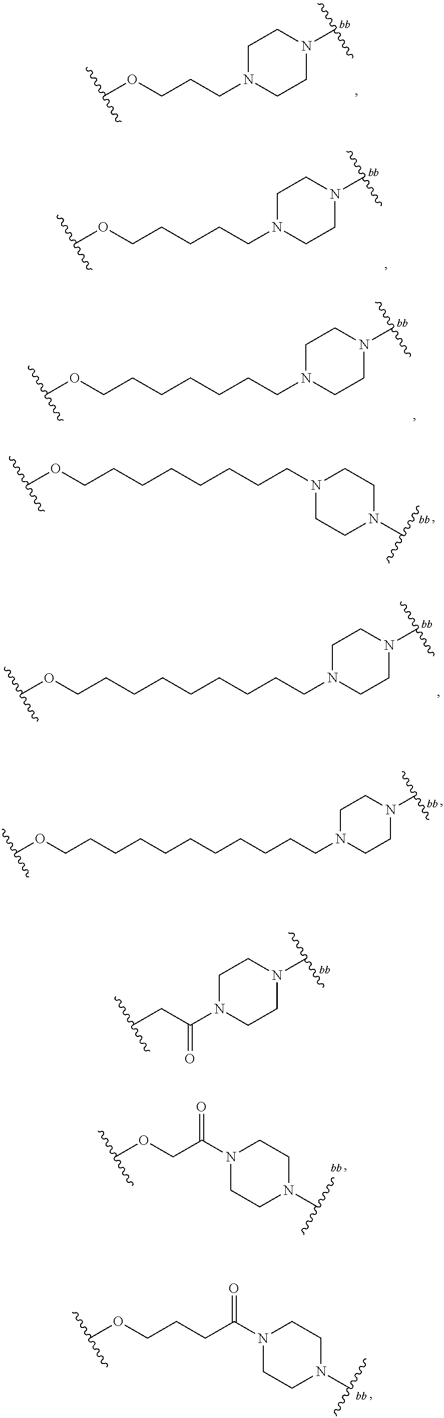 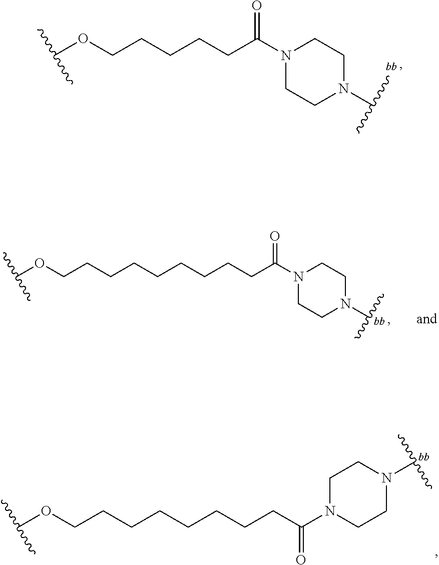
wherein bb represents the point of attachment to Ring C.

60. The compound of claim 1, wherein the compound is a compound of Formula (I-A): 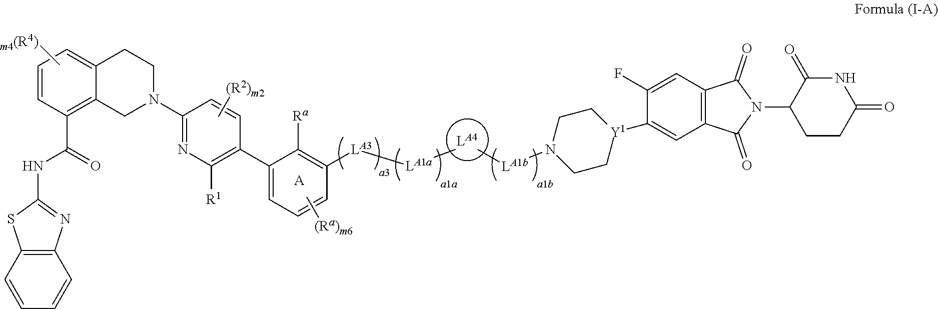
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Y¹ is N or CH; or
the compound is a compound of Formula (I-B): 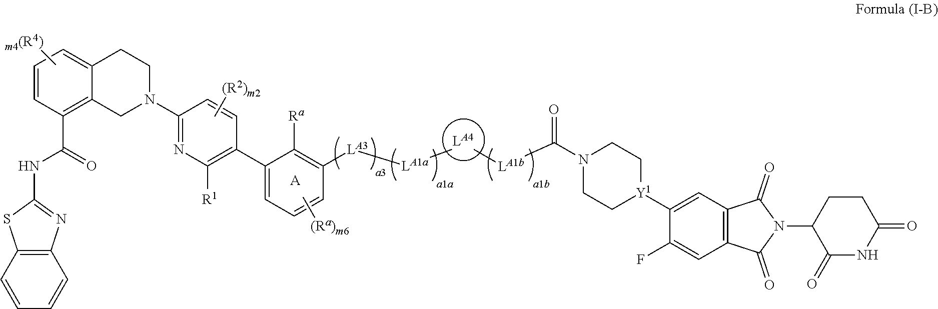
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—; and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Y¹ is N or CH; or
the compound is a compound of Formula (I-C): 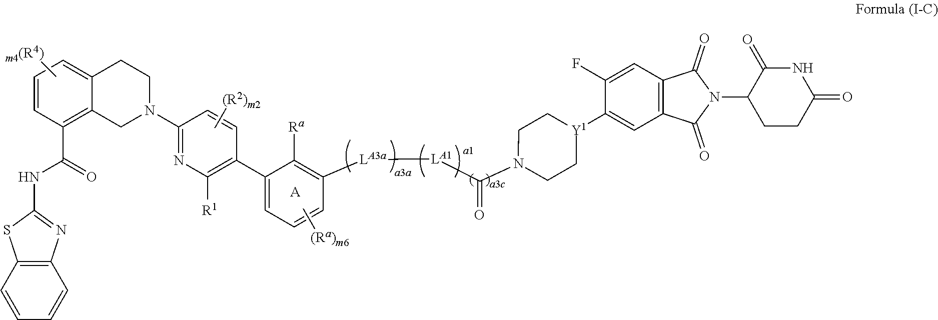
or a pharmaceutically acceptable salt thereof, wherein:
m6 is 0 or 1;
a3a is 0 or 1;
L^{A3a} is selected from the group consisting of: —O—, —N(H)—, and —N(C₁₋₃ alkyl)-;
a1 is an integer from 1 to 11;
a3c is 0 or 1; and
Y¹ is N or CH.

61. The compound of any one of claims 1-60, wherein the compound is selected from the group consisting of the compounds in Table C1, or a pharmaceutically acceptable salt thereof.

62. The compound of claim 61, wherein the compound does not exhibit Yₘᵢₙ (6 h)≥70% and/or Yₘᵢₙ (24 h)≥70% under conditions described in Example B1.

63. A pharmaceutical composition comprising a compound as claimed in any one of claims 1-62, or a pharmaceutically acceptable salt thereof, and a pharmaceutically acceptable excipient.

64. A method for treating a cancer in a subject in need thereof, the method comprising administering to the subject a therapeutically effective amount of a compound of any one of claims 1-62, or a pharmaceutically acceptable salt thereof, or a pharmaceutical composition according to claim 63.

65. The method of claim 64, wherein the method comprises administering an additional therapy or therapeutic agent.

66. The method of claim 65, wherein the additional therapy or therapeutic agent is an ALK inhibitor, a BCL-2 inhibitor, a BCR-Abl inhibitor, a BRaf inhibitor, a CDK2 inhibitor, CDK 4/6 inhibitor, a CDK7 inhibitor, a CDK9 inhibitor, an EGFR inhibitor, an anti-EGFR antibody or an anti-EGFR antibody-drug conjugate, an ERK inhibitor, a FGFR1 inhibitor, a FGFR2 inhibitor, a FGFR3 inhibitor, a FGFR4 inhibitor, a HER2 inhibitor, an anti-HER2 antibody or an anti-HER2 antibody-drug conjugate, a JAK inhibitor, a KRas inhibitor, a MEK inhibitor, a MET inhibitor, a PARP inhibitor, an LSD1 inhibitor, a BET inhibitor, a telomerase inhibitor, a TORC1/2 inhibitor, chemotherapy, radiotherapy, or a combination thereof.

67. The method of claim 66, wherein the additional therapy or therapeutic agent is a JAK inhibitor.

68. The method of claim 67, wherein the JAK inhibitor is fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), or a combination thereof.

69. The method of any one of claims 64-68, wherein the cancer is breast cancer, colorectal cancer, bile duct cancer, colorectal cancer, gastrointestinal stromal tumor, pancreatic cancer, bladder cancer, kidney cancer, cervical cancer, ovarian cancer, uterine cancer, head and neck cancer, hematological cancer, lung cancer, skin cancer, or a combination thereof.

70. The method of claim 69, wherein the hematological cancer is acute lymphocytic leukemia (ALL), acute myeloid leukemia (AML), chronic lymphocytic leukemia (CLL), follicular lymphoma (FL), small lymphocytic lymphoma (SLL), essential thrombocythemia, polycythemia vera, myelofibrosis, or a combination thereof.

71. The method of claim 70, wherein the hematological cancer is essential thrombocythemia, polycythemia vera, myelofibrosis, or a combination thereof.

72. The method of any one of claims 69-71, wherein the hematological cancer has a JAK2 mutation (e.g., JAK2 V617F).

73. Any of the compounds, methods, or uses as provided herein.
